(19) United States
(12) Patent Application Publication
(10) Pub. No.: US 2026/0042769 A1
(43) Pub. Date: Feb. 12, 2026

(54) TRIARYL AGONISTS OF ALPHA-2 ADRENERGIC RECEPTORS

(71) Applicant: ALCEPTOR THERAPEUTICS, INC., Newport Beach, CA (US)

(72) Inventors: Jotham W. COE, Niantic, CT (US); Robert L. DOW, Groton, CT (US); John E. DONELLO, San Clemente, CA (US); Bruce D. ROTH, Newport Beach, CA (US); Donald E. FRAIL, Natick, MA (US); Daniel W. GIL, Corona Del Mar, CA (US); Robert A. VOLKMANN, Mystic, CT (US)

(21) Appl. No.: 19/365,059

(22) Filed: Oct. 21, 2025

Related U.S. Application Data
(63) Continuation of application No. PCT/US2024/025588, filed on Apr. 19, 2024.
(60) Provisional application No. 63/497,518, filed on Apr. 21, 2023.

(51) Int. Cl. C07D 487/04; C07D 401/06; C07D 401/14; C07D 403/06; C07D 403/14; C07D 405/14; C07D 413/06; C07D 417/06; C07D 471/04

(52) U.S. Cl. CPC C07D 487/04; C07D 401/06; C07D 401/14; C07D 403/06; C07D 403/14; C07D 405/14; C07D 413/06; C07D 417/06; C07D 471/04

(57) ABSTRACT

The present disclosure relates to compounds of the formula:

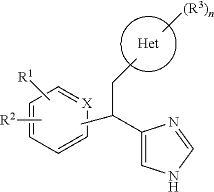

and pharmaceutically acceptable salts thereof, to processes for the preparation of, intermediates used in the preparation of, and compositions containing such compounds and the uses of such compounds for the treatment of pain or hypertension.

### CROSS-REFERENCE TO RELATED APPLICATIONS

This application is a continuation application of International Patent Application No. PCT/US2024/025588, filed Apr. 19, 2024, which claims priority to U.S. Provisional Application No. 63/497,518, filed Apr. 21, 2023, all of which are incorporated herein by reference.

### BACKGROUND

There is an ongoing need for new compounds that are effective in the treatment of pain. There is also an ongoing need for new compounds that are effective in the treatment of hypertension.

### SUMMARY

This disclosure relates to compounds of Formula I:

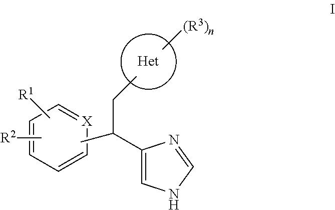

or a pharmaceutically acceptable salt thereof, wherein: X is N or CR¹; R¹ is H, F, Cl, Br, I, OH, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom; R² is H, F, Cl, Br, I, OH, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom; when R¹ and R² are on adjacent carbon atoms they, together with the phenyl ring to which they are attached, may form a bicyclic heteroaryl ring system that is optionally substituted with 1 or 2 substituents, wherein the substituents of the dihydrobenzofuranyl or the benzodioxolyl are independently F, Cl, Br, I, OH, NH₂, —OCH₃, —OCH₂CH₃, —CH₃, —C≡CH, —OCF₃, —OCHF₂, or —CF₃; Each R³ is independently H, F, Cl, Br, I, OH, ═O, or a moiety of a formula C₁₋₁₅H₀₋₃₅N₀₋₅O₀₋₆S₀₋₂F₀₋₁₆; which attaches to the phenyl ring with a carbon atom, a nitrogen atom, an oxygen atom, or a sulfur atom; n is 0, 1, 2, or 3; and Het is heteroaryl with one or more ring heteroatoms independently selected from 0, S and N.

Some embodiments include a compound of formula II:

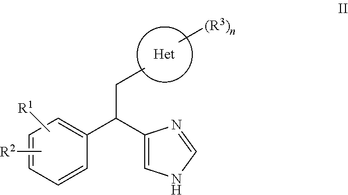

or a pharmaceutically acceptable salt thereof, wherein: R¹ is H, F, Cl, Br, I, OH, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom; R² is H, F, Cl, Br, I, OH, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom; when R¹ and R² are on adjacent carbon atoms they, together with the phenyl ring to which they are attached, may form a dihydrobenzofuranyl or a benzodioxolyl that is optionally substituted with 1 or 2 substituents, wherein the substituents of the dihydrobenzofuranyl or the benzodioxolyl are independently F, Cl, Br, I, OH, NH₂, —OCH₃, —OCH₂CH₃, —CH₃, —C≡CH, —OCF₃, —OCHF₂, or —CF₃; Each R³ is independently H, F, Cl, Br, I, OH, ═O, or a moiety of a formula C₁₋₁₅H₀₋₃₅N_{0.5}O_{0.6}S₀₋₂F₀₋₁₆ which attaches to the phenyl ring with a carbon atom, a nitrogen atom, an oxygen atom, or a sulfur atom; n is 0, 1, 2, or 3; and Het is heteroaryl with one or more ring heteroatoms independently selected from 0, S and N.

Some embodiments include a pharmaceutical composition comprising a compound described herein and a pharmaceutically acceptable carrier.

Some embodiments include the use of a compound described herein in the manufacture of a medicament to treat pain or hypertension.

Some embodiments include a method of treating pain or hypertension comprising administering, to a mammal in need thereof, a therapeutically effective amount of a compound described herein.

Some embodiments include a kit comprising 1) a compound described herein, and 2) instructions to use the compound to treat pain or hypertension.

### DETAILED DESCRIPTION

This present disclosure relates to novel compounds of Formula I, Formula Ia, Formula Ib, Formula Ic, Formula Id, Formula Ie, Formula II, Formula IIa, Formula IIb, Formula IIc, Formula IId, or Formula IIe, or pharmaceutically acceptable salts thereof.

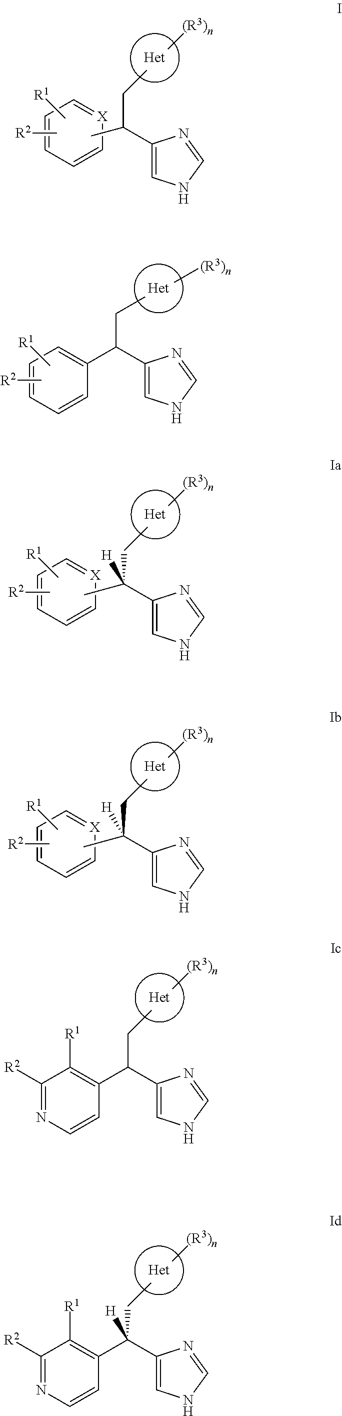

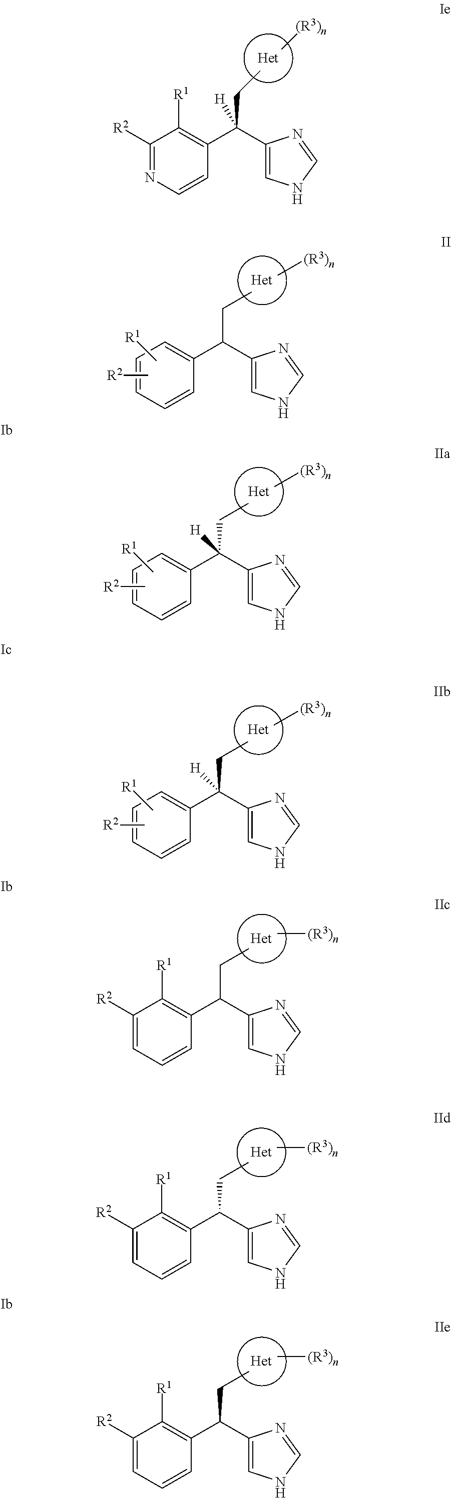

With respect to any relevant structural representation, such as Formula I, Formula Ia, Formula Ib, Formula Ic, Formula Id, Formula Ie, X is N or CR¹. In some embodiments, X is N. In some embodiments, X is CR¹.

With respect to any relevant structural representation, such as Formula I, Formula Ia, Formula Ib, Formula Ic, Formula Id, Formula Ie, Formula II, Formula IIa, Formula IIb, Formula IIc, Formula IId, or Formula IIe, R¹ is H, F, Cl, Br, I, OH, or a moiety of a formula C₁₋₁₅H₀₋₃₅N₀₋₅O₀₋₆S₀₋₂F₀₋₁₆, C₁₋₆H₀₋₁₅N₀₋₂O₀₋₃F₀₋₇, or C₁₋₃H₀₋₇O₀₋₃F₀₋₄, which attaches to the phenyl ring with a carbon atom, such as C₁₋₆alkyl (e.g., —CH₃), C₁₋₆alkynyl (e.g., —C≡CH), —CN, or C₁₋₆ fluoroalkyl (e.g., —CF₃), attaches to the phenyl ring with a nitrogen atom (e.g., —NHCH₃), attaches to the phenyl ring with an oxygen atom, such as C₁₋₆—O-alkyl (e.g., —OCH₃, —CH₂CH₃, etc.) or C₁₋₆—O-fluoroalkyl (e.g., —OCF₃, —OCHF₂, etc.), or attaches to the phenyl ring with a sulfur atom (e.g., —SCH₃, —SCH₂CH₃, etc.).

With respect to any relevant structural representation, such as Formula I, Formula Ia, Formula Ib, Formula Ic, Formula Id, Formula Ie, Formula II, Formula IIa, Formula IIb, Formula IIc, Formula IId, or Formula IIe, R² is H, F, Cl, Br, I, OH, or a moiety of a formula C₁₋₁₅H₀₋₃₅N₀₋₅O₀₋₆S₀₋₂F₀₋₁₆, C₁₋₆H₀₋₁₅N₀₋₂O₀₋₃F₀₋₇, or C₁₋₃H₀₋₇O₀₋₃F₀₋₄, which attaches to the phenyl ring with a carbon atom, such as C₁₋₆ alkyl (e.g., —CH₃), C₁0.6 alkynyl (e.g., —C≡CH), —CN, or C₁₋₆ fluoroalkyl (e.g., —CF₃), attaches to the phenyl ring with a nitrogen atom (e.g., —NHCH₃), attaches to the phenyl ring with an oxygen atom, such as C₁₋₆—O-alkyl (e.g., —OCH₃, —OCH₂CH₃, etc.) or C₁₋₆—O-fluoroalkyl (e.g., —OCF₃, —OCHF₂, etc.), or attaches to the phenyl ring with a sulfur atom (e.g., —SCH₃, —SCH₂CH₃, etc.).

With respect to any relevant structural representation, such as Formula I, Formula Ia, Formula Ib, Formula Ic, Formula Id, Formula Ie, Formula II, Formula IIa, Formula IIb, Formula IIc, Formula IId, or Formula IIe, in some embodiments, when R¹ and R² are on adjacent carbon atoms they, together with the phenyl ring to which they are attached, may form a bicyclic heteroaryl ring system, such as a dihydrobenzofuranyl or a benzodioxolyl, or an indolinyl, a quinolonyl, a dihydrobenzodioxinyl, an indazolyl, an indolyl, a benzoxazolyl, a dihydrobenzodioxepinyl, a quinolinyl, or a dihydroisobenzofuranyl that is optionally substituted with 1 or 2 substituents, wherein the substituents of the bicyclic heteroaryl ring system are independently F, Cl, Br, I, OH, NH₂, —OCH₃, —OCH₂CH₃, —CH₃, —C≡CH, —OCF₃, —OCHF₂, or —CF₃.

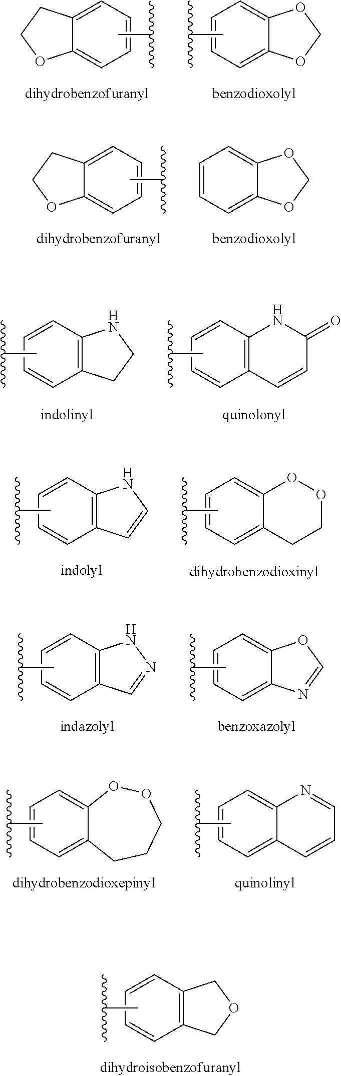

In some embodiments, R¹ and R², together with the phenyl ring to which they are attached, form a dihydrobenzofuranyl that is optionally substituted with 1 or 2 substituents listed above.

In some embodiments, R¹ and R², together with the phenyl ring to which they are attached, form a benzodioxolyl that is optionally substituted with 1 or 2 substituents listed above.

In some embodiments, R¹ and R², together with the phenyl ring to which they are attached, form an indoline that is optionally substituted with 1 or 2 substituents listed above.

In some embodiments, R¹ and R², together with the phenyl ring to which they are attached, form a quinolone that is optionally substituted with 1 or 2 substituents listed above.

In some embodiments, R¹ and R², together with the phenyl ring to which they are attached, form a dihydrobenzodioxinyl.

In some embodiments, R¹ and R², together with the phenyl ring to which they are attached, form an indazolyl.

In some embodiments, R¹ and R², together with the phenyl ring to which they are attached, form an indazolyl.

In some embodiments, R¹ and R², together with the phenyl ring to which they are attached, form an indolyl.

In some embodiments, R¹ and R², together with the phenyl ring to which they are attached, form a benzoxazolyl.

In some embodiments, R¹ and R², together with the phenyl ring to which they are attached, form a dihydrobenzodioxepinyl.

In some embodiments, R¹ and R², together with the phenyl ring to which they are attached, form a quinolinyl.

In some embodiments, R¹ and R², together with the phenyl ring to which they are attached, form a dihydroisobenzofuranyl

With respect to any relevant structural representation, such as Formula I, Formula Ia, Formula Ib, Formula Ic, Formula Id, Formula Ie, Formula II, Formula IIa, Formula IIb, Formula IIc, Formula IId, or Formula IIe, each R³ is independently H, F, Cl, Br, I, OH, ═O, or a moiety of a formula C₁₋₁₅H₀₋₃₅N₀₋₅O₀₋₆S₀₋₂F₀₋₁₆; C₁₋₆H₀₋₁₅N₀₋₂O₀₋₃F₀₋₇, or C₁₋₃H₀₋₇O₀₋₃F₀₋₄, which attaches to the phenyl ring with a carbon atom, such as C₁₋₆ alkyl (e.g., —CH₃), C₃₋₆ cycloalkyl (e.g., cyclopropyl), —C(═O)NH₂, or C₁₋₆ fluoroalkyl (e.g., —CF₃), attaches to the phenyl ring with an oxygen atom, such as C₁₋₆—O-alkyl (e.g., —OCH₃, —OCH₂CH₃, etc.), attaches to the phenyl ring with a nitrogen atom —NH(CH₂)₁₋₅OCH₃ (e.g., —NHCH₂CH₂OCH₃), or attaches to the phenyl ring with a sulfur atom (e.g., —SCH₃, —SCH₂CH₃, etc.).

With respect to any relevant structural representation, such as Formula I, Formula 1b, Formula Ic, Formula Id, or Formula Ie, n is 0, 1, 2, or 3. In some embodiments, n is 0. In some embodiments, n is 1. In some embodiments, n is 2. In some embodiments, n is 3.

With respect to any relevant structural representation, such as Formula I, Formula Ia, Formula Ib, Formula Ic, Formula Id, Formula Ie, Formula II, Formula IIa, Formula IIb, Formula IIc, Formula IId, or Formula IIe, Het is a monocyclic or fused-ring polycyclic heteroaryl group with one or more ring heteroatoms selected from O, S and N. In some embodiments, Het has 5 to 12 ring atoms including one to five heteroatoms independently selected from O, S, or N. In some embodiments, Het is pyridyl, pyrazinyl, pyrimidinyl, pyridazinyl, thienyl, furyl, imidazolyl, pyrrolyl, oxazolyl, thiazolyl, pyrazolyl, tetrazolyl, triazolyl, oxadiazolyl, thiadiazolyl, quinolyl, isoquinolyl, benzothienyl, benzofuryl, or indolyl. In some embodiments, Het is pyridyl, pyrimidinyl, pyridazinyl, pyrazolyl, pyrazinyl, thiazolyl, oxazolyl, or isoxazolyl. In some embodiments, Het is pyridazinonyl, azaindolyl, azaindazolyl, or imidazopyridinyl.

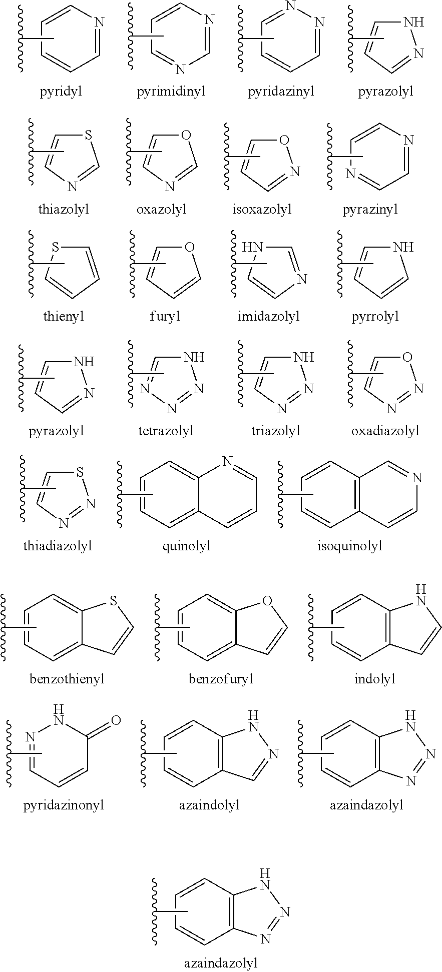

In some embodiments, Het is pyridyl.

In some embodiments, Het is pyrazinyl.

In some embodiments, Het is pyrimidinyl.

In some embodiments, Het is pyridazinyl.

In some embodiments, Het is thienyl.

In some embodiments, Het is furyl.

In some embodiments, Het is imidazolyl.

In some embodiments, Het is pyrrolyl.

In some embodiments, Het is oxazolyl.

In some embodiments, Het is thiazolyl.

In some embodiments, Het is pyrazolyl.

In some embodiments, Het is tetrazolyl.

In some embodiments, Het is triazolyl.

In some embodiments, Het is oxadiazolyl.

In some embodiments, Het is thiadiazolyl.

In some embodiments, Het is quinolyl.

In some embodiments, Het is isoquinolyl.

In some embodiments, Het is benzothienyl.

In some embodiments, Het is benzofuryl.

In some embodiments, Het is indolyl.

In some embodiments, Het is pyridyl.

In some embodiments, Het is pyrimidinyl.

In some embodiments, Het is pyridazinyl.

In some embodiments, Het is pyrazolyl.

In some embodiments, Het is pyrazinyl.

In some embodiments, Het is thiazolyl.

In some embodiments, Het is oxazolyl.

In some embodiments, Het is isoxazolyl.

In some embodiments, Het is pyridazinonyl.

In some embodiments, Het is azaindolyl.

In some embodiments, Het is azaindazolyl.

In some embodiments, Het is imidazopyridinyl.

As used herein, the term “alkyl” includes saturated aliphatic hydrocarbons including straight chains and branched chains and 1, 2, 3, 4, 5, or 6 carbon atoms. For example, as used herein, the term “(C₁-C₆)alkyl,” as well as the alkyl moieties of other groups referred to herein (e.g., (C₁-C₆)alkoxy), refers to linear or branched moieties of 1 to 6, carbon atoms, e.g., 1, 2, 3, 4, 5, or 6 carbon atoms (e.g., methyl, ethyl, n-propyl, isopropyl, n-butyl, iso-butyl, secondary-butyl, tertiary-butyl), optionally substituted by 1, 2, 3, 4, or 5 suitable substituents.

Whenever a numerical range is used in this application, for example when 1 to 6 is used in the definition of “alkyl” means that the alkyl group may contain 1, 2, 3, 4, 5, or 6 carbon atoms.

As used herein, the term “alkynyl” includes aliphatic hydrocarbons having at least one carbon-carbon triple bond, including straight chains and branched chains having at least one carbon-carbon triple bond and 2, 3, 4, 5 or 6 carbon atoms. For example, as used herein, the term “(C₂-C₆)alkynyl” is used herein to mean straight or branched hydrocarbon chain alkynyl moiety as defined above having 2, 3, 4, 5 or 6 carbon atoms and one triple bond; optionally substituted by 1, 2, 3, 4, or 5 suitable substituents.

As used herein, the term “cycloalkyl” includes saturated or unsaturated (non-aromatic) monocyclic or bicyclic hydrocarbon rings (e.g., cyclopropyl, cyclobutyl, cyclopentyl, cyclohexyl, cycloheptyl, cyclooctyl, cyclononyl); optionally substituted by 1, 2, 3, 4, or 5 suitable substituents. The cycloalkyl group has 3, 4, 5, 6, 7, 8, 9, 10, 11 or 12 carbon atoms. One group of monocyclic cycloalkyl rings have 3, 4, 5, or 6 carbon atoms. In another embodiment the cycloalkyl may optionally contain one, two or more non-cumulative nonaromatic double or triple bonds.

As used herein, the term “heteroaryl” includes monocyclic or fused-ring polycyclic aromatic heterocyclic groups with one or more heteroatoms selected from O, S and N in the ring. The heteroaryl group has 5 to 12 ring atoms, or 5, 6, 7, 8, 9, 10, 11, or 12 ring atoms, including one to five heteroatoms, (or 1, 2, 3, 4, or 5 heteroatoms) selected from O, S, and N. For example, as used herein, the term “5 to 12 membered heteroaryl” means aromatic moieties containing at least one ring heteroatom selected from O, S and N and from 1 to 11 carbon atoms, or 1, 2, 3, 4, 5, 6, 7, 8, 9, 10, or 11 carbon atoms, such as pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like. The heteroaryl group is optionally substituted by 1, 2, 3, 4, or 5 suitable substituents.

The compounds of Formula I, Formula Ia, Formula Ib, Formula Ic, Formula Id, or Formula Ie may exist in the form of pharmaceutically acceptable salts such as, e.g., acid addition salts and base addition salts of the compounds of Formula I, Formula Ia, Formula Ib, Formula Ic, Formula Id, or Formula Ie. The phrase “pharmaceutically acceptable salt(s)”, as used herein, unless otherwise indicated, includes salts of acidic or basic groups that are acceptable for pharmaceutical use. Reference to any compound herein, by name, structure, or any other means, includes pharmaceutically acceptable salts of the compound.

As used herein, any reference to a compound by structure, name, or any other indicator, is intended to include all forms of the compound, including hydrates, solvates, isomers (including optical, geometric and tautomeric isomers), crystalline and non-crystalline forms, isomorphs, polymorphs, metabolites, prodrugs, and isotopically enriched compounds thereof.

Compounds 1-409, depicted in Table A, Table B, Table C, Table D, Table E, Table F, Table G, Table H, Table I, and pharmaceutically acceptable salts thereof, are specifically contemplated herein.

Some embodiments include Compound 1 or a pharmaceutical acceptable salt thereof.

Some embodiments include Compound 2 or a pharmaceutical acceptable salt thereof.

Some embodiments include Compound 3 or a pharmaceutical acceptable salt thereof.

Some embodiments include Compound 4 or a pharmaceutical acceptable salt thereof.

Some embodiments include Compound 5 or a pharmaceutical acceptable salt thereof.

Some embodiments include Compound 6 or a pharmaceutical acceptable salt thereof.

Some embodiments include Compound 7 or a pharmaceutical acceptable salt thereof.

Some embodiments include Compound 8 or a pharmaceutical acceptable salt thereof.

Some embodiments include Compound 9 or a pharmaceutical acceptable salt thereof.

Some embodiments include Compound 10 or a pharmaceutical acceptable salt thereof.

Some embodiments include Compound 11 or a pharmaceutical acceptable salt thereof.

Some embodiments include Compound 12 or a pharmaceutical acceptable salt thereof.

Some embodiments include Compound 13 or a pharmaceutical acceptable salt thereof.

Some embodiments include Compound 14 or a pharmaceutical acceptable salt thereof.

Some embodiments include Compound 15 or a pharmaceutical acceptable salt thereof.

Some embodiments include Compound 16 or a pharmaceutical acceptable salt thereof.

Some embodiments include Compound 17 or a pharmaceutical acceptable salt thereof.

Some embodiments include Compound 18 or a pharmaceutical acceptable salt thereof.

Some embodiments include Compound 19 or a pharmaceutical acceptable salt thereof.

Some embodiments include Compound 20 or a pharmaceutical acceptable salt thereof.

Some embodiments include Compound 21 or a pharmaceutical acceptable salt thereof.

Some embodiments include Compound 22 or a pharmaceutical acceptable salt thereof.

Some embodiments include Compound 23 or a pharmaceutical acceptable salt thereof.

Some embodiments include Compound 24 or a pharmaceutical acceptable salt thereof.

Some embodiments include Compound 25 or a pharmaceutical acceptable salt thereof.

Some embodiments include Compound 26 or a pharmaceutical acceptable salt thereof.

Some embodiments include Compound 27 or a pharmaceutical acceptable salt thereof.

Some embodiments include Compound 28 or a pharmaceutical acceptable salt thereof.

Some embodiments include Compound 29 or a pharmaceutical acceptable salt thereof.

Some embodiments include Compound 30 or a pharmaceutical acceptable salt thereof.

Some embodiments include Compound 31 or a pharmaceutical acceptable salt thereof.

Some embodiments include Compound 32 or a pharmaceutical acceptable salt thereof.

Some embodiments include Compound 33 or a pharmaceutical acceptable salt thereof.

Some embodiments include Compound 34 or a pharmaceutical acceptable salt thereof.

Some embodiments include Compound 35 or a pharmaceutical acceptable salt thereof.

Some embodiments include Compound 36 or a pharmaceutical acceptable salt thereof.

Some embodiments include Compound 37 or a pharmaceutical acceptable salt thereof.

Some embodiments include Compound 38 or a pharmaceutical acceptable salt thereof.

Some embodiments include Compound 39 or a pharmaceutical acceptable salt thereof.

Some embodiments include Compound 40 or a pharmaceutical acceptable salt thereof.

Some embodiments include Compound 41 or a pharmaceutical acceptable salt thereof.

Some embodiments include Compound 42 or a pharmaceutical acceptable salt thereof.

Some embodiments include Compound 43 or a pharmaceutical acceptable salt thereof.

Some embodiments include Compound 44 or a pharmaceutical acceptable salt thereof.

Some embodiments include Compound 45 or a pharmaceutical acceptable salt thereof.

Some embodiments include Compound 46 or a pharmaceutical acceptable salt thereof.

Some embodiments include Compound 47 or a pharmaceutical acceptable salt thereof.

Some embodiments include Compound 48 or a pharmaceutical acceptable salt thereof.

Some embodiments include Compound 49 or a pharmaceutical acceptable salt thereof.

Some embodiments include Compound 50 or a pharmaceutical acceptable salt thereof.

Some embodiments include Compound 51 or a pharmaceutical acceptable salt thereof.

Some embodiments include Compound 52 or a pharmaceutical acceptable salt thereof.

Some embodiments include Compound 53 or a pharmaceutical acceptable salt thereof.

Some embodiments include Compound 54 or a pharmaceutical acceptable salt thereof.

Some embodiments include Compound 55 or a pharmaceutical acceptable salt thereof.

Some embodiments include Compound 56 or a pharmaceutical acceptable salt thereof.

Some embodiments include Compound 57 or a pharmaceutical acceptable salt thereof.

Some embodiments include Compound 58 or a pharmaceutical acceptable salt thereof.

Some embodiments include Compound 59 or a pharmaceutical acceptable salt thereof.

Some embodiments include Compound 60 or a pharmaceutical acceptable salt thereof.

Some embodiments include Compound 61 or a pharmaceutical acceptable salt thereof.

Some embodiments include Compound 62 or a pharmaceutical acceptable salt thereof.

Some embodiments include Compound 63 or a pharmaceutical acceptable salt thereof.

Some embodiments include Compound 64 or a pharmaceutical acceptable salt thereof.

Some embodiments include Compound 65 or a pharmaceutical acceptable salt thereof.

Some embodiments include Compound 66 or a pharmaceutical acceptable salt thereof.

Some embodiments include Compound 67 or a pharmaceutical acceptable salt thereof.

Some embodiments include Compound 68 or a pharmaceutical acceptable salt thereof.

Some embodiments include Compound 69 or a pharmaceutical acceptable salt thereof.

Some embodiments include Compound 70 or a pharmaceutical acceptable salt thereof.

Some embodiments include Compound 71 or a pharmaceutical acceptable salt thereof.

Some embodiments include Compound 72 or a pharmaceutical acceptable salt thereof.

Some embodiments include Compound 73 or a pharmaceutical acceptable salt thereof.

Some embodiments include Compound 74 or a pharmaceutical acceptable salt thereof.

Some embodiments include Compound 75 or a pharmaceutical acceptable salt thereof.

Some embodiments include Compound 76 or a pharmaceutical acceptable salt thereof.

Some embodiments include Compound 77 or a pharmaceutical acceptable salt thereof.

Some embodiments include Compound 78 or a pharmaceutical acceptable salt thereof.

Some embodiments include Compound 79 or a pharmaceutical acceptable salt thereof.

Some embodiments include Compound 80 or a pharmaceutical acceptable salt thereof.

Some embodiments include Compound 81 or a pharmaceutical acceptable salt thereof.

Some embodiments include Compound 82 or a pharmaceutical acceptable salt thereof.

Some embodiments include Compound 83 or a pharmaceutical acceptable salt thereof.

Some embodiments include Compound 84 or a pharmaceutical acceptable salt thereof.

Some embodiments include Compound 85 or a pharmaceutical acceptable salt thereof.

Some embodiments include Compound 86 or a pharmaceutical acceptable salt thereof.

Some embodiments include Compound 87 or a pharmaceutical acceptable salt thereof.

Some embodiments include Compound 88 or a pharmaceutical acceptable salt thereof.

Some embodiments include Compound 89 or a pharmaceutical acceptable salt thereof.

Some embodiments include Compound 90 or a pharmaceutical acceptable salt thereof.

Some embodiments include Compound 91 or a pharmaceutical acceptable salt thereof.

Some embodiments include Compound 92 or a pharmaceutical acceptable salt thereof.

Some embodiments include Compound 93 or a pharmaceutical acceptable salt thereof.

Some embodiments include Compound 94 or a pharmaceutical acceptable salt thereof.

Some embodiments include Compound 95 or a pharmaceutical acceptable salt thereof.

Some embodiments include Compound 96 or a pharmaceutical acceptable salt thereof.

Some embodiments include Compound 97 or a pharmaceutical acceptable salt thereof.

Some embodiments include Compound 98 or a pharmaceutical acceptable salt thereof.

Some embodiments include Compound 99 or a pharmaceutical acceptable salt thereof.

Some embodiments include Compound 100 or a pharmaceutical acceptable salt thereof.

Some embodiments include Compound 101 or a pharmaceutical acceptable salt thereof.

Some embodiments include Compound 102 or a pharmaceutical acceptable salt thereof.

Some embodiments include Compound 103 or a pharmaceutical acceptable salt thereof.

Some embodiments include Compound 104 or a pharmaceutical acceptable salt thereof.

Some embodiments include Compound 105 or a pharmaceutical acceptable salt thereof.

Some embodiments include Compound 106 or a pharmaceutical acceptable salt thereof.

Some embodiments include Compound 107 or a pharmaceutical acceptable salt thereof.

Some embodiments include Compound 108 or a pharmaceutical acceptable salt thereof.

Some embodiments include Compound 109 or a pharmaceutical acceptable salt thereof.

Some embodiments include Compound 110 or a pharmaceutical acceptable salt thereof.

Some embodiments include Compound 111 or a pharmaceutical acceptable salt thereof.

Some embodiments include Compound 112 or a pharmaceutical acceptable salt thereof.

Some embodiments include Compound 113 or a pharmaceutical acceptable salt thereof.

Some embodiments include Compound 114 or a pharmaceutical acceptable salt thereof.

Some embodiments include Compound 115 or a pharmaceutical acceptable salt thereof.

Some embodiments include Compound 116 or a pharmaceutical acceptable salt thereof.

Some embodiments include Compound 117 or a pharmaceutical acceptable salt thereof.

Some embodiments include Compound 118 or a pharmaceutical acceptable salt thereof.

Some embodiments include Compound 119 or a pharmaceutical acceptable salt thereof.

Some embodiments include Compound 120 or a pharmaceutical acceptable salt thereof.

Some embodiments include Compound 121 or a pharmaceutical acceptable salt thereof.

Some embodiments include Compound 122 or a pharmaceutical acceptable salt thereof.

Some embodiments include Compound 123 or a pharmaceutical acceptable salt thereof.

Some embodiments include Compound 124 or a pharmaceutical acceptable salt thereof.

Some embodiments include Compound 125 or a pharmaceutical acceptable salt thereof.

Some embodiments include Compound 126 or a pharmaceutical acceptable salt thereof.

Some embodiments include Compound 127 or a pharmaceutical acceptable salt thereof.

Some embodiments include Compound 128 or a pharmaceutical acceptable salt thereof.

Some embodiments include Compound 129 or a pharmaceutical acceptable salt thereof.

Some embodiments include Compound 130 or a pharmaceutical acceptable salt thereof.

Some embodiments include Compound 131 or a pharmaceutical acceptable salt thereof.

Some embodiments include Compound 132 or a pharmaceutical acceptable salt thereof.

Some embodiments include Compound 133 or a pharmaceutical acceptable salt thereof.

Some embodiments include Compound 134 or a pharmaceutical acceptable salt thereof.

Some embodiments include Compound 135 or a pharmaceutical acceptable salt thereof.

Some embodiments include Compound 136 or a pharmaceutical acceptable salt thereof.

Some embodiments include Compound 137 or a pharmaceutical acceptable salt thereof.

Some embodiments include Compound 138 or a pharmaceutical acceptable salt thereof.

Some embodiments include Compound 139 or a pharmaceutical acceptable salt thereof.

Some embodiments include Compound 140 or a pharmaceutical acceptable salt thereof.

Some embodiments include Compound 141 or a pharmaceutical acceptable salt thereof.

Some embodiments include Compound 142 or a pharmaceutical acceptable salt thereof.

Some embodiments include Compound 143 or a pharmaceutical acceptable salt thereof.

Some embodiments include Compound 144 or a pharmaceutical acceptable salt thereof.

Some embodiments include Compound 145 or a pharmaceutical acceptable salt thereof.

Some embodiments include Compound 146 or a pharmaceutical acceptable salt thereof.

Some embodiments include Compound 147 or a pharmaceutical acceptable salt thereof.

Some embodiments include Compound 148 or a pharmaceutical acceptable salt thereof.

Some embodiments include Compound 149 or a pharmaceutical acceptable salt thereof.

Some embodiments include Compound 150 or a pharmaceutical acceptable salt thereof.

Some embodiments include Compound 151 or a pharmaceutical acceptable salt thereof.

Some embodiments include Compound 152 or a pharmaceutical acceptable salt thereof.

Some embodiments include Compound 153 or a pharmaceutical acceptable salt thereof.

Some embodiments include Compound 154 or a pharmaceutical acceptable salt thereof.

Some embodiments include Compound 155 or a pharmaceutical acceptable salt thereof.

Some embodiments include Compound 156 or a pharmaceutical acceptable salt thereof.

Some embodiments include Compound 157 or a pharmaceutical acceptable salt thereof.

Some embodiments include Compound 158 or a pharmaceutical acceptable salt thereof.

Some embodiments include Compound 159 or a pharmaceutical acceptable salt thereof.

Some embodiments include Compound 160 or a pharmaceutical acceptable salt thereof.

Some embodiments include Compound 161 or a pharmaceutical acceptable salt thereof.

Some embodiments include Compound 162 or a pharmaceutical acceptable salt thereof.

Some embodiments include Compound 163 or a pharmaceutical acceptable salt thereof.

Some embodiments include Compound 164 or a pharmaceutical acceptable salt thereof.

Some embodiments include Compound 165 or a pharmaceutical acceptable salt thereof.

Some embodiments include Compound 166 or a pharmaceutical acceptable salt thereof.

Some embodiments include Compound 167 or a pharmaceutical acceptable salt thereof.

Some embodiments include Compound 168 or a pharmaceutical acceptable salt thereof.

Some embodiments include Compound 169 or a pharmaceutical acceptable salt thereof.

Some embodiments include Compound 170 or a pharmaceutical acceptable salt thereof.

Some embodiments include Compound 171 or a pharmaceutical acceptable salt thereof.

Some embodiments include Compound 172 or a pharmaceutical acceptable salt thereof.

Some embodiments include Compound 173 or a pharmaceutical acceptable salt thereof.

Some embodiments include Compound 174 or a pharmaceutical acceptable salt thereof.

Some embodiments include Compound 175 or a pharmaceutical acceptable salt thereof.

Some embodiments include Compound 176 or a pharmaceutical acceptable salt thereof.

Some embodiments include Compound 177 or a pharmaceutical acceptable salt thereof.

Some embodiments include Compound 178 or a pharmaceutical acceptable salt thereof.

Some embodiments include Compound 179 or a pharmaceutical acceptable salt thereof.

Some embodiments include Compound 180 or a pharmaceutical acceptable salt thereof.

Some embodiments include Compound 181 or a pharmaceutical acceptable salt thereof.

Some embodiments include Compound 182 or a pharmaceutical acceptable salt thereof.

Some embodiments include Compound 183 or a pharmaceutical acceptable salt thereof.

Some embodiments include Compound 184 or a pharmaceutical acceptable salt thereof.

Some embodiments include Compound 185 or a pharmaceutical acceptable salt thereof.

Some embodiments include Compound 186 or a pharmaceutical acceptable salt thereof.

Some embodiments include Compound 187 or a pharmaceutical acceptable salt thereof.

Some embodiments include Compound 188 or a pharmaceutical acceptable salt thereof.

Some embodiments include Compound 189 or a pharmaceutical acceptable salt thereof.

Some embodiments include Compound 190 or a pharmaceutical acceptable salt thereof.

Some embodiments include Compound 191 or a pharmaceutical acceptable salt thereof.

Some embodiments include Compound 192 or a pharmaceutical acceptable salt thereof.

Some embodiments include Compound 193 or a pharmaceutical acceptable salt thereof.

Some embodiments include Compound 194 or a pharmaceutical acceptable salt thereof.

Some embodiments include Compound 195 or a pharmaceutical acceptable salt thereof.

Some embodiments include Compound 196 or a pharmaceutical acceptable salt thereof.

Some embodiments include Compound 197 or a pharmaceutical acceptable salt thereof.

Some embodiments include Compound 198 or a pharmaceutical acceptable salt thereof.

Some embodiments include Compound 199 or a pharmaceutical acceptable salt thereof.

Some embodiments include Compound 200 or a pharmaceutical acceptable salt thereof.

Some embodiments include Compound 201 or a pharmaceutical acceptable salt thereof.

Some embodiments include Compound 202 or a pharmaceutical acceptable salt thereof.

Some embodiments include Compound 203 or a pharmaceutical acceptable salt thereof.

Some embodiments include Compound 204 or a pharmaceutical acceptable salt thereof.

Some embodiments include Compound 205 or a pharmaceutical acceptable salt thereof.

Some embodiments include Compound 206 or a pharmaceutical acceptable salt thereof.

Some embodiments include Compound 207 or a pharmaceutical acceptable salt thereof.

Some embodiments include Compound 208 or a pharmaceutical acceptable salt thereof.

Some embodiments include Compound 209 or a pharmaceutical acceptable salt thereof.

Some embodiments include Compound 210 or a pharmaceutical acceptable salt thereof.

Some embodiments include Compound 211 or a pharmaceutical acceptable salt thereof.

Some embodiments include Compound 212 or a pharmaceutical acceptable salt thereof.

Some embodiments include Compound 213 or a pharmaceutical acceptable salt thereof.

Some embodiments include Compound 214 or a pharmaceutical acceptable salt thereof.

Some embodiments include Compound 215 or a pharmaceutical acceptable salt thereof.

Some embodiments include Compound 216 or a pharmaceutical acceptable salt thereof.

Some embodiments include Compound 217 or a pharmaceutical acceptable salt thereof.

Some embodiments include Compound 218 or a pharmaceutical acceptable salt thereof.

Some embodiments include Compound 219 or a pharmaceutical acceptable salt thereof.

Some embodiments include Compound 220 or a pharmaceutical acceptable salt thereof.

Some embodiments include Compound 221 or a pharmaceutical acceptable salt thereof.

Some embodiments include Compound 222 or a pharmaceutical acceptable salt thereof.

Some embodiments include Compound 223 or a pharmaceutical acceptable salt thereof.

Some embodiments include Compound 224 or a pharmaceutical acceptable salt thereof.

Some embodiments include Compound 225 or a pharmaceutical acceptable salt thereof.

Some embodiments include Compound 226 or a pharmaceutical acceptable salt thereof.

Some embodiments include Compound 227 or a pharmaceutical acceptable salt thereof.

Some embodiments include Compound 228 or a pharmaceutical acceptable salt thereof.

Some embodiments include Compound 229 or a pharmaceutical acceptable salt thereof.

Some embodiments include Compound 230 or a pharmaceutical acceptable salt thereof.

Some embodiments include Compound 231 or a pharmaceutical acceptable salt thereof.

Some embodiments include Compound 232 or a pharmaceutical acceptable salt thereof.

Some embodiments include Compound 233 or a pharmaceutical acceptable salt thereof.

Some embodiments include Compound 234 or a pharmaceutical acceptable salt thereof.

Some embodiments include Compound 235 or a pharmaceutical acceptable salt thereof.

Some embodiments include Compound 236 or a pharmaceutical acceptable salt thereof.

Some embodiments include Compound 237 or a pharmaceutical acceptable salt thereof.

Some embodiments include Compound 238 or a pharmaceutical acceptable salt thereof.

Some embodiments include Compound 239 or a pharmaceutical acceptable salt thereof.

Some embodiments include Compound 240 or a pharmaceutical acceptable salt thereof.

Some embodiments include Compound 241 or a pharmaceutical acceptable salt thereof.

Some embodiments include Compound 242 or a pharmaceutical acceptable salt thereof.

Some embodiments include Compound 243 or a pharmaceutical acceptable salt thereof.

Some embodiments include Compound 244 or a pharmaceutical acceptable salt thereof.

Some embodiments include Compound 245 or a pharmaceutical acceptable salt thereof.

Some embodiments include Compound 246 or a pharmaceutical acceptable salt thereof.

Some embodiments include Compound 247 or a pharmaceutical acceptable salt thereof.

Some embodiments include Compound 248 or a pharmaceutical acceptable salt thereof.

Some embodiments include Compound 249 or a pharmaceutical acceptable salt thereof.

Some embodiments include Compound 250 or a pharmaceutical acceptable salt thereof.

Some embodiments include Compound 251 or a pharmaceutical acceptable salt thereof.

Some embodiments include Compound 252 or a pharmaceutical acceptable salt thereof.

Some embodiments include Compound 253 or a pharmaceutical acceptable salt thereof.

Some embodiments include Compound 254 or a pharmaceutical acceptable salt thereof.

Some embodiments include Compound 255 or a pharmaceutical acceptable salt thereof.

Some embodiments include Compound 256 or a pharmaceutical acceptable salt thereof.

Some embodiments include Compound 257 or a pharmaceutical acceptable salt thereof.

Some embodiments include Compound 258 or a pharmaceutical acceptable salt thereof.

Some embodiments include Compound 259 or a pharmaceutical acceptable salt thereof.

Some embodiments include Compound 260 or a pharmaceutical acceptable salt thereof.

Some embodiments include Compound 261 or a pharmaceutical acceptable salt thereof.

Some embodiments include Compound 262 or a pharmaceutical acceptable salt thereof.

Some embodiments include Compound 263 or a pharmaceutical acceptable salt thereof.

Some embodiments include Compound 264 or a pharmaceutical acceptable salt thereof.

Some embodiments include Compound 265 or a pharmaceutical acceptable salt thereof.

Some embodiments include Compound 266 or a pharmaceutical acceptable salt thereof.

Some embodiments include Compound 267 or a pharmaceutical acceptable salt thereof.

Some embodiments include Compound 268 or a pharmaceutical acceptable salt thereof.

Some embodiments include Compound 269 or a pharmaceutical acceptable salt thereof.

Some embodiments include Compound 270 or a pharmaceutical acceptable salt thereof.

Some embodiments include Compound 271 or a pharmaceutical acceptable salt thereof.

Some embodiments include Compound 272 or a pharmaceutical acceptable salt thereof.

Some embodiments include Compound 273 or a pharmaceutical acceptable salt thereof.

Some embodiments include Compound 274 or a pharmaceutical acceptable salt thereof.

Some embodiments include Compound 275 or a pharmaceutical acceptable salt thereof.

Some embodiments include Compound 276 or a pharmaceutical acceptable salt thereof.

Some embodiments include Compound 277 or a pharmaceutical acceptable salt thereof.

Some embodiments include Compound 278 or a pharmaceutical acceptable salt thereof.

Some embodiments include Compound 279 or a pharmaceutical acceptable salt thereof.

Some embodiments include Compound 280 or a pharmaceutical acceptable salt thereof.

Some embodiments include Compound 281 or a pharmaceutical acceptable salt thereof.

Some embodiments include Compound 282 or a pharmaceutical acceptable salt thereof.

Some embodiments include Compound 283 or a pharmaceutical acceptable salt thereof.

Some embodiments include Compound 284 or a pharmaceutical acceptable salt thereof.

Some embodiments include Compound 285 or a pharmaceutical acceptable salt thereof.

Some embodiments include Compound 286 or a pharmaceutical acceptable salt thereof.

Some embodiments include Compound 287 or a pharmaceutical acceptable salt thereof.

Some embodiments include Compound 288 or a pharmaceutical acceptable salt thereof.

Some embodiments include Compound 289 or a pharmaceutical acceptable salt thereof.

Some embodiments include Compound 290 or a pharmaceutical acceptable salt thereof.

Some embodiments include Compound 291 or a pharmaceutical acceptable salt thereof.

Some embodiments include Compound 292 or a pharmaceutical acceptable salt thereof.

Some embodiments include Compound 293 or a pharmaceutical acceptable salt thereof.

Some embodiments include Compound 294 or a pharmaceutical acceptable salt thereof.

Some embodiments include Compound 295 or a pharmaceutical acceptable salt thereof.

Some embodiments include Compound 296 or a pharmaceutical acceptable salt thereof.

Some embodiments include Compound 297 or a pharmaceutical acceptable salt thereof.

Some embodiments include Compound 298 or a pharmaceutical acceptable salt thereof.

Some embodiments include Compound 299 or a pharmaceutical acceptable salt thereof.

Some embodiments include Compound 300 or a pharmaceutical acceptable salt thereof.

Some embodiments include Compound 301 or a pharmaceutical acceptable salt thereof.

Some embodiments include Compound 302 or a pharmaceutical acceptable salt thereof.

Some embodiments include Compound 303 or a pharmaceutical acceptable salt thereof.

Some embodiments include Compound 304 or a pharmaceutical acceptable salt thereof.

Some embodiments include Compound 305 or a pharmaceutical acceptable salt thereof.

Some embodiments include Compound 306 or a pharmaceutical acceptable salt thereof.

Some embodiments include Compound 307 or a pharmaceutical acceptable salt thereof.

Some embodiments include Compound 308 or a pharmaceutical acceptable salt thereof.

Some embodiments include Compound 309 or a pharmaceutical acceptable salt thereof.

Some embodiments include Compound 310 or a pharmaceutical acceptable salt thereof.

Some embodiments include Compound 311 or a pharmaceutical acceptable salt thereof.

Some embodiments include Compound 312 or a pharmaceutical acceptable salt thereof.

Some embodiments include Compound 313 or a pharmaceutical acceptable salt thereof.

Some embodiments include Compound 314 or a pharmaceutical acceptable salt thereof.

Some embodiments include Compound 315 or a pharmaceutical acceptable salt thereof.

Some embodiments include Compound 316 or a pharmaceutical acceptable salt thereof.

Some embodiments include Compound 317 or a pharmaceutical acceptable salt thereof.

Some embodiments include Compound 318 or a pharmaceutical acceptable salt thereof.

Some embodiments include Compound 319 or a pharmaceutical acceptable salt thereof.

Some embodiments include Compound 320 or a pharmaceutical acceptable salt thereof.

Some embodiments include Compound 321 or a pharmaceutical acceptable salt thereof.

Some embodiments include Compound 322 or a pharmaceutical acceptable salt thereof.

Some embodiments include Compound 323 or a pharmaceutical acceptable salt thereof.

Some embodiments include Compound 324 or a pharmaceutical acceptable salt thereof.

Some embodiments include Compound 325 or a pharmaceutical acceptable salt thereof.

Some embodiments include Compound 326 or a pharmaceutical acceptable salt thereof.

Some embodiments include Compound 327 or a pharmaceutical acceptable salt thereof.

Some embodiments include Compound 328 or a pharmaceutical acceptable salt thereof.

Some embodiments include Compound 329 or a pharmaceutical acceptable salt thereof.

Some embodiments include Compound 330 or a pharmaceutical acceptable salt thereof.

Some embodiments include Compound 331 or a pharmaceutical acceptable salt thereof.

Some embodiments include Compound 332 or a pharmaceutical acceptable salt thereof.

Some embodiments include Compound 333 or a pharmaceutical acceptable salt thereof.

Some embodiments include Compound 334 or a pharmaceutical acceptable salt thereof.

Some embodiments include Compound 335 or a pharmaceutical acceptable salt thereof.

Some embodiments include Compound 336 or a pharmaceutical acceptable salt thereof.

Some embodiments include Compound 337 or a pharmaceutical acceptable salt thereof.

Some embodiments include Compound 338 or a pharmaceutical acceptable salt thereof.

Some embodiments include Compound 339 or a pharmaceutical acceptable salt thereof.

Some embodiments include Compound 340 or a pharmaceutical acceptable salt thereof.

Some embodiments include Compound 341 or a pharmaceutical acceptable salt thereof.

Some embodiments include Compound 342 or a pharmaceutical acceptable salt thereof.

Some embodiments include Compound 343 or a pharmaceutical acceptable salt thereof.

Some embodiments include Compound 344 or a pharmaceutical acceptable salt thereof.

Some embodiments include Compound 345 or a pharmaceutical acceptable salt thereof.

Some embodiments include Compound 346 or a pharmaceutical acceptable salt thereof.

Some embodiments include Compound 347 or a pharmaceutical acceptable salt thereof.

Some embodiments include Compound 348 or a pharmaceutical acceptable salt thereof.

Some embodiments include Compound 349 or a pharmaceutical acceptable salt thereof.

Some embodiments include Compound 350 or a pharmaceutical acceptable salt thereof.

Some embodiments include Compound 351 or a pharmaceutical acceptable salt thereof.

Some embodiments include Compound 352 or a pharmaceutical acceptable salt thereof.

Some embodiments include Compound 353 or a pharmaceutical acceptable salt thereof.

Some embodiments include Compound 354 or a pharmaceutical acceptable salt thereof.

Some embodiments include Compound 355 or a pharmaceutical acceptable salt thereof.

Some embodiments include Compound 356 or a pharmaceutical acceptable salt thereof.

Some embodiments include Compound 357 or a pharmaceutical acceptable salt thereof.

Some embodiments include Compound 358 or a pharmaceutical acceptable salt thereof.

Some embodiments include Compound 359 or a pharmaceutical acceptable salt thereof.

Some embodiments include Compound 360 or a pharmaceutical acceptable salt thereof.

Some embodiments include Compound 361 or a pharmaceutical acceptable salt thereof.

Some embodiments include Compound 362 or a pharmaceutical acceptable salt thereof.

Some embodiments include Compound 363 or a pharmaceutical acceptable salt thereof.

Some embodiments include Compound 364 or a pharmaceutical acceptable salt thereof.

Some embodiments include Compound 365 or a pharmaceutical acceptable salt thereof.

Some embodiments include Compound 366 or a pharmaceutical acceptable salt thereof.

Some embodiments include Compound 367 or a pharmaceutical acceptable salt thereof.

Some embodiments include Compound 368 or a pharmaceutical acceptable salt thereof.

Some embodiments include Compound 369 or a pharmaceutical acceptable salt thereof.

Some embodiments include Compound 370 or a pharmaceutical acceptable salt thereof.

Some embodiments include Compound 371 or a pharmaceutical acceptable salt thereof.

Some embodiments include Compound 372 or a pharmaceutical acceptable salt thereof.

Some embodiments include Compound 373 or a pharmaceutical acceptable salt thereof.

Some embodiments include Compound 374 or a pharmaceutical acceptable salt thereof.

Some embodiments include Compound 375 or a pharmaceutical acceptable salt thereof.

Some embodiments include Compound 376 or a pharmaceutical acceptable salt thereof.

Some embodiments include Compound 377 or a pharmaceutical acceptable salt thereof.

Some embodiments include Compound 378 or a pharmaceutical acceptable salt thereof.

Some embodiments include Compound 379 or a pharmaceutical acceptable salt thereof.

Some embodiments include Compound 380 or a pharmaceutical acceptable salt thereof.

Some embodiments include Compound 381 or a pharmaceutical acceptable salt thereof.

Some embodiments include Compound 382 or a pharmaceutical acceptable salt thereof.

Some embodiments include Compound 383 or a pharmaceutical acceptable salt thereof.

Some embodiments include Compound 384 or a pharmaceutical acceptable salt thereof.

Some embodiments include Compound 385 or a pharmaceutical acceptable salt thereof.

Some embodiments include Compound 386 or a pharmaceutical acceptable salt thereof.

Some embodiments include Compound 387 or a pharmaceutical acceptable salt thereof.

Some embodiments include Compound 388 or a pharmaceutical acceptable salt thereof.

Some embodiments include Compound 389 or a pharmaceutical acceptable salt thereof.

Some embodiments include Compound 390 or a pharmaceutical acceptable salt thereof.

Some embodiments include Compound 391 or a pharmaceutical acceptable salt thereof.

Some embodiments include Compound 392 or a pharmaceutical acceptable salt thereof.

Some embodiments include Compound 393 or a pharmaceutical acceptable salt thereof.

Some embodiments include Compound 394 or a pharmaceutical acceptable salt thereof.

Some embodiments include Compound 395 or a pharmaceutical acceptable salt thereof.

Some embodiments include Compound 396 or a pharmaceutical acceptable salt thereof.

Some embodiments include Compound 397 or a pharmaceutical acceptable salt thereof.

Some embodiments include Compound 398 or a pharmaceutical acceptable salt thereof.

Some embodiments include Compound 399 or a pharmaceutical acceptable salt thereof.

Some embodiments include Compound 400 or a pharmaceutical acceptable salt thereof.

Some embodiments include Compound 401 or a pharmaceutical acceptable salt thereof.

Some embodiments include Compound 402 or a pharmaceutical acceptable salt thereof.

Some embodiments include Compound 403 or a pharmaceutical acceptable salt thereof.

Some embodiments include Compound 404 or a pharmaceutical acceptable salt thereof.

Some embodiments include Compound 405 or a pharmaceutical acceptable salt thereof.

Some embodiments include Compound 406 or a pharmaceutical acceptable salt thereof.

Some embodiments include Compound 407 or a pharmaceutical acceptable salt thereof.

Some embodiments include Compound 408 or a pharmaceutical acceptable salt thereof.

Some embodiments include Compound 409 or a pharmaceutical acceptable salt thereof.

The compounds of the disclosure may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of Formula I, Formula Ia, Formula Ib, Formula Ic, Formula Id, or Formula Ie include references to salts, solvates, mufti-component complexes and liquid crystals thereof and to solvates, mufti-component complexes and liquid crystals of salts thereof.

The compounds of Formula I, Formula Ia, Formula Ib, Formula Ic, Formula Id, or Formula Ie may have asymmetric carbon atoms and may exist as two or more stereoisomers. The carbon-carbon bonds of the compounds of Formula I, Formula Ia, Formula Ib, Formula Ic Formula Id, or Formula Ie may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that the single isomer depicted is contemplated, that an excess of a single isomer has been obtained, but with unknown stereochemistry, or that an excess of a single isomer has been obtained and identified as that isomer.

The compounds of Formula I, Formula Ia, Formula Ib, Formula Ic, Formula Id, Formula Ie, Formula II, Formula IIa, Formula IIb, Formula IIc, Formula IId, or Formula IIe, or an acceptable salt thereof may be included in a useful composition such as a pharmaceutical composition. Accordingly, in one embodiment, such a pharmaceutical composition comprises a compound described herein and a pharmaceutically acceptable carrier. In some embodiments, a pharmaceutical composition contains a single active pharmaceutical ingredient.

The pharmaceutical acceptable carrier may comprise any conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

In one preferred embodiment the composition comprises a therapeutically effective amount of a compound of Formula I, Formula Ia, Formula Ib, Formula Ic, Formula Id, or Formula Ie and a pharmaceutically acceptable carrier.

Another embodiment of the disclosure is directed to a method for treating pain or hypertension in a mammal, such as a human, comprising administering to said mammal a therapeutically effective amount of a compound described herein.

The term “therapeutically effective amount” as used herein refers to that amount of the compound being administered which will relieve to some extent one or more of the symptoms of the disorder being treated.

The terms “treating,” or “treatment” broadly includes any kind of treatment activity, including the diagnosis, cure, mitigation, or prevention of disease in man or other animals, or any activity that otherwise affects the structure or any function of the body of man or other animals.

Administration of the compounds of Formula I, Formula Ia, Formula Ib, Formula Ic, Formula Id, or Formula Ie may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the active agent are well-known in the relevant art and would be understood to be encompassed by the skilled artisan once provided the teachings disclosed herein.

The amount of the compound of Formula I, Formula Ia, Formula Ib, Formula Ic, Formula Id, or Formula Ie administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

Compounds of the Formula I, Formula Ia, Formula Ib, Formula Ic, Formula Id, or Formula Ie may be prepared according to the following reaction schemes and accompanying discussion. Unless otherwise indicated, R¹ through R³, Het, and n, and structural Formula I, Formula Ia, Formula Ib, Formula Ic, Formula Id, or Formula Ie are as defined above in the reaction schemes and discussion that follow. In general, the compounds of this disclosure may be made by processes which include processes analogous to those known in the chemical arts, particularly in light of the description contained herein. Certain processes for the manufacture of the compounds of this disclosure are provided as further features of the disclosure and are illustrated by the following reaction schemes. Other processes may be described in the experimental section.

As an initial note, in the preparation of the compounds of Formula I, Formula Ia, Formula Ib, Formula Ic, Formula Id, or Formula Ie it is noted that some of the preparation methods useful for the preparation of the compounds described herein may require protection of remote functionality (e.g., primary amine, secondary amine, carboxyl in Formula I, Formula Ia, Formula Ib, Formula Ic, Formula Id, or Formula Ie precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I, Formula Ia, Formula Ib, Formula Ic, Formula Id, or Formula Ie compound.

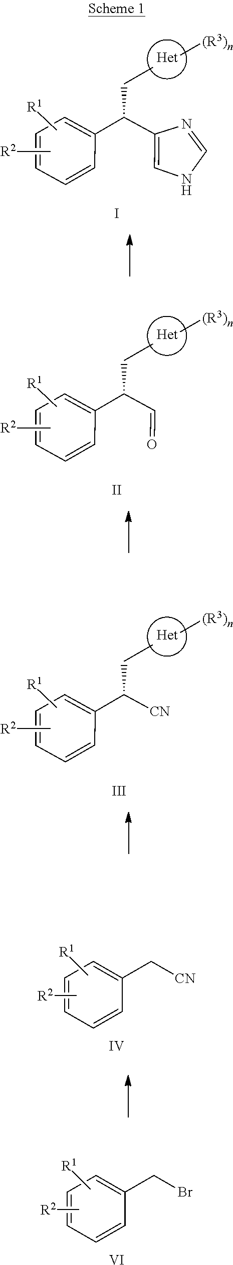

Scheme 1 refers to the synthesis of compounds of Formula I, wherein R¹, R², R³ and n are as defined hereinabove. Referring to Scheme 1, a compound of the Formula I may be prepared from a compound of Formula II (1.0 equiv.) in a polar solvent such as ethanol (10 vol.), by reaction with toluenesulfonylmethyl isocyanide (TosMIC) (1.3 equiv.) and stirred at room temperature for 4 hours. The ethanol was concentrated, and the obtained crude was dissolved in methanol (5 vol.) and the mixture was transferred into a high pressure steel vessel. A freshly prepared solution of ammonia in methanol (20 vol.) was added to the vessel and was then sealed and the mixture was stirred at 90° C. for 16 hours. The mixture was allowed to cool to room temperature and solvent was concentrated under vacuum. The obtained crude was dissolved with ethyl acetate, washed with brine solution, dried over anhydrous sodium sulphate, filtered and concentrated under reduced pressure to obtain the product of Formula I.

The compound of Formula II may be prepared by reaction of a compound of Formula III (1.0 equiv.) dissolved in an inert solvent such as toluene (10 vol.), to which was added diisobutylaluminium hydride (1M solution in cyclohexane) (3 equiv.) at −78° C., and the resulting mixture was stirred at same temperature for 2 minutes. The reaction mixture was immediately quenched with saturated ammonium chloride (10 vol.), and the mixture allowed to warm to room temperature and stirred for 15 minutes. Then the mixture was filtered through Celite® to remove the precipitate, and the filtrate was extracted with ethyl acetate. The organic layer was washed with brine solution, dried over anhydrous sodium sulphate, filtered and concentrated under reduced pressure to obtain crude, which was purified by flash column chromatography using ethyl acetate in hexane as an eluent to obtain compound of Formula II.

The compound of Formula III may be prepared from a compound of Formula IV (1.0 equiv.) dissolved in an inert solvent such as THF (10 vol) to which is added NaH (60% dispersion in mineral oil) (1 equiv.) at 0° C. and stirred for 30 minutes at same temperature. Then, a solution of a Heterocyclic halide ((Het-(CH₂)—X), 1 equiv) in DMF (3 vol.) was added slowly dropwise at 0° C. When the heterocyclic halide is a HCl salt, 1 equivalent of trimethylamine was used to neutralise. The resulting mixture was allowed to warm to room temperature and stirred for 16 hours. The progress of the reaction was monitored by TLC (20% ethyl acetate in hexane). After completion of reaction, the mixture was quenched with ice water and extracted with ethyl acetate. The organic layer was washed with brine solution, dried over anhydrous sodium sulphate, filtered and concentrated under reduced pressure to obtain crude, which was purified by flash column chromatography using ethyl acetate in hexane as an eluent to obtain the compound of Formula IIII.

Alternatively, the compound of Formula IIII may be prepared from a compound of Formula IV (1.0 equiv.) by a Knoevenagel reaction followed by reduction reaction. More specifically, a Heteroaryl-aldehyde (1 equiv.) of Formula

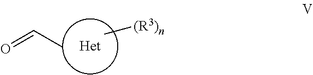

Is dissolved in ethanol (10 vol.) and stirred, to which is added sodium methoxide (0.2 equiv.) at room temperature and stirred for 2 hours. The progress of the reaction was monitored by LCMS. After completion of reaction, a reducing agent such as sodium borohydride (2 equiv.) was added, and the mixture stirred for 1 hour. The reaction mixture was quenched with ice water and the mixture was concentrated under reduced pressure to remove ethanol. The obtained crude was diluted with water and extracted with ethyl acetate. The organic layer was washed with brine solution, dried over anhydrous sodium sulphate, filtered and concentrated under reduced pressure to obtain crude, which was purified by flash column chromatography using ethyl acetate in hexane as an eluent to obtain compound of Formula III.

The compound of Formula IV may be prepared from a halo compound of Formula VI (1.0 equiv.) dissolved in a solvent such as acetonitrile to which is added potassium carbonate (2 equiv.) and trimethylsilyl cyanide (1.5 equiv.) slowly at room temperature. The mixture was heated to 80° C. and stirred for 16 hours. The progress of the reaction was monitored by TLC. After completion of reaction, the mixture was allowed to cool to room temperature, diluted with water and extracted with ethyl acetate. The organic layer was washed with brine solution, dried over anhydrous sodium sulphate, filtered and concentrated under reduced pressure to obtain crude, which was purified by flash column chromatography using ethyl acetate in hexane as an eluent to obtain compound of Formula IV.

Compounds of Formula I have chiral centers and thus exist as stereoisomers, such as racemates, enantiomers, or diastereomers. Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of Formula I (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g., “Stereochemistry of Organic Compounds” by E. L Eliel (Wiley, New York, 1994), the disclosure of which is incorporated herein by reference in its entirety.

In the present case, compounds of Formula I exist as a mixture of enantiomers of Formulae Ia and Ib.

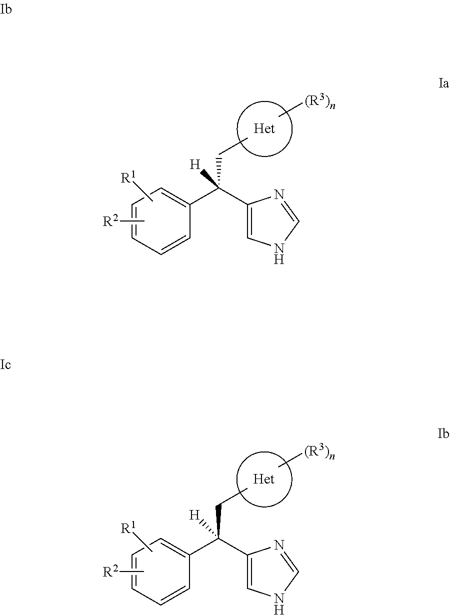

This mixture of isomers can be resolved through conventional methods described above or via chiral resolution by protection of the imidazole nitrogen followed by resolution through a chiral column followed by deprotection. Protection of the imidazole compounds of Formula Ia and Ib is performed according to methods well known to those skilled in the art. More specifically, to a stirred solution of compound of Formula I (i.e., a mixture of enantiomers of Formula Ia and Ib) (1.0 equiv.) in dichloromethane (10 vol.), was added triethylamine (3 equiv.) and BOC anhydride (1.5 equiv). The mixture was stirred at room temperature for 4 hours. The progress of the reaction was monitored by TLC, after completion of reaction, the mixture was diluted with water and extracted with dichloromethane. The organic layer was washed with brine solution, dried over anhydrous sodium sulphate, filtered & concentrated under reduced pressure to obtain crude, which was purified by flash column chromatography using ethyl acetate in hexane as an eluent to obtain compounds Via and VIIb.

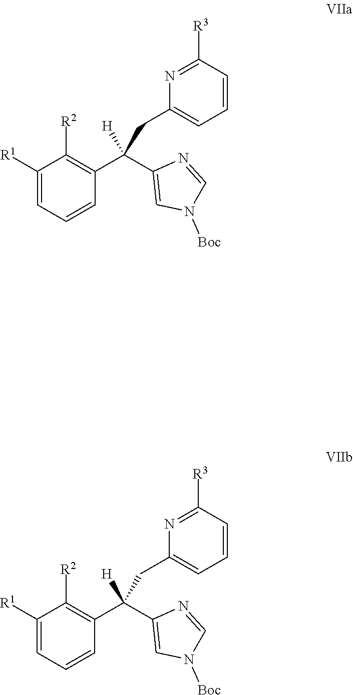

Most of the enantiomers (Via and VIIb) were separated as racemic compounds at this stage. A few enantiomers may be separated without BOC protection. Chiral methods used for separation are mentioned below.

### Method 1

Analytical conditions: Column: CHIRALPAK IG (100 mm×4.6 mm×3 μm).

Mobile phase: —n-Hexane:Ethanol with 0.1% DEA.

### Method 2

Analytical conditions: Column: CHIRALPAK IG (100 mm×4.6 mm×3 μm).

Mobile phase: —n-Hexane:IPA with 0.1% DEA.

### Method 3

Analytical conditions: Column: CHIRALPAK IC (100 mm×4.6 mm×3 μm)

Mobile phase: —n-Hexane:Ethanol with 0.1% DEA.

### Method 4

Analytical conditions: Column: CHIRALPAK IC (100 mm×4.6 mm×3 μm)

Mobile phase: —n-Hexane:IPA with 0.1% DEA.

### Method 5

Analytical conditions: Column: CHIRALPAK IC (100 mm×4.6 mm×3 μm)

Mobile phase: —MTBE:Ethanol with 0.1% DEA

### Method 6

Analytical conditions: Column: CHIRALCEL OJ-H (250 mm×4.6 mm×5 μm)

Mobile phase: —n-Hexane:Ethanol with 0.1% DEA

### Method 7

Analytical conditions: Column: CHIRALPAK IA (100 mm×4.6 mm×3 μm)

Mobile phase: —n-Hexane:IPA with 0.1% DEA.

The resolved compounds of Formula Via and VIIb may be deprotected according to methods well known to those skilled in the art. Specifically, to a stirred solution of compound Vila or VIIb (1.0 equiv.) in methanol (10 vol.), was added sodium carbonate (2 equiv.) at room temperature and the mixture was stirred for 2 hours. The progress of the reaction was monitored by TLC, after completion of reaction, the mixture was concentrated. The obtained crude was dissolved with water and extracted with ethyl acetate. The organic layer was washed with brine solution, dried over anhydrous sodium sulphate, filtered and concentrated under reduced pressure to obtain crude, which was purified by flash column chromatography using methanol in dichloromethane as an eluent to obtain single enantiomer compound of Formula Ib or Formula Ic.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the disclosure (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

All publications, including but not limited to, issued patents, patent applications, and journal articles, cited in this application are each herein incorporated by reference in their entirety.

Although the disclosure has been described above with reference to the disclosed embodiments, those skilled in the art will readily appreciate that the specific experiments detailed below are only illustrative of the disclosure. It should be understood that various modifications can be made without departing from the spirit of the disclosure. Accordingly, the disclosure is limited only by the claims.

In the following Examples and Preparations, “BOC”, “Boc” or “boc” means N-tert-butoxycarbonyl, “DCM” (CH₂Cl₂) means methylene chloride, “DIPEA” or “DIEA” means diisopropyl ethyl amine, “DMA” means N,N-dimethylacetamide, “DMF” means N—N-dimethyl formamide, “DMSO” means dimethylsulfoxide, “DPPP” means 1,3-bis(diphenylphosphino)propane, “HOAc” means acetic acid, “IPA” means isopropyl alcohol. “MTBE” means methyl t-butyl ether, “NMP” means 1-methyl 2-pyrrolidinone, “TEA” means triethyl amine, “TFA” means trifluoroacetic acid, “DCM” means dichloromethane, “EtOAc” means ethyl acetate, “MgSO₄” means magnesium sulphate, “NaSO₄” means sodium sulphate, “MeOH” means methanol, “EtOH” means ethanol, “H₂O” means water, “HCl” means hydrochloric acid, “POCl₃” means phosphorus oxychloride, “DMSO” means dimethyl sulfoxide, “K₂CO₃” means potassium carbonate, “N” means Normal, “M” means molar, “mL” means millilitre, “mmol” means millimoles, “μmol” means micromoles, “eq.” means equivalent, “° C.” means degrees Celsius, “Pa” means pascals.

### Methods and Procedures

### Example of Procedure A

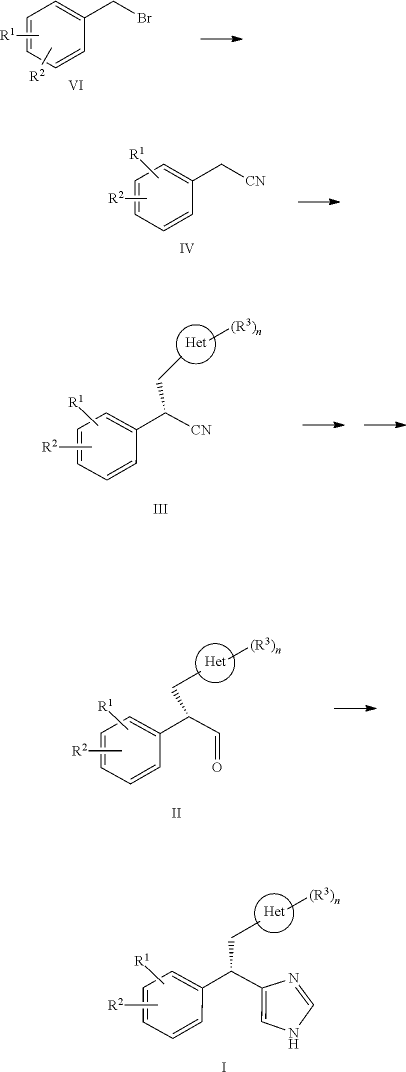

### Synthesis of Compounds 5 and 6

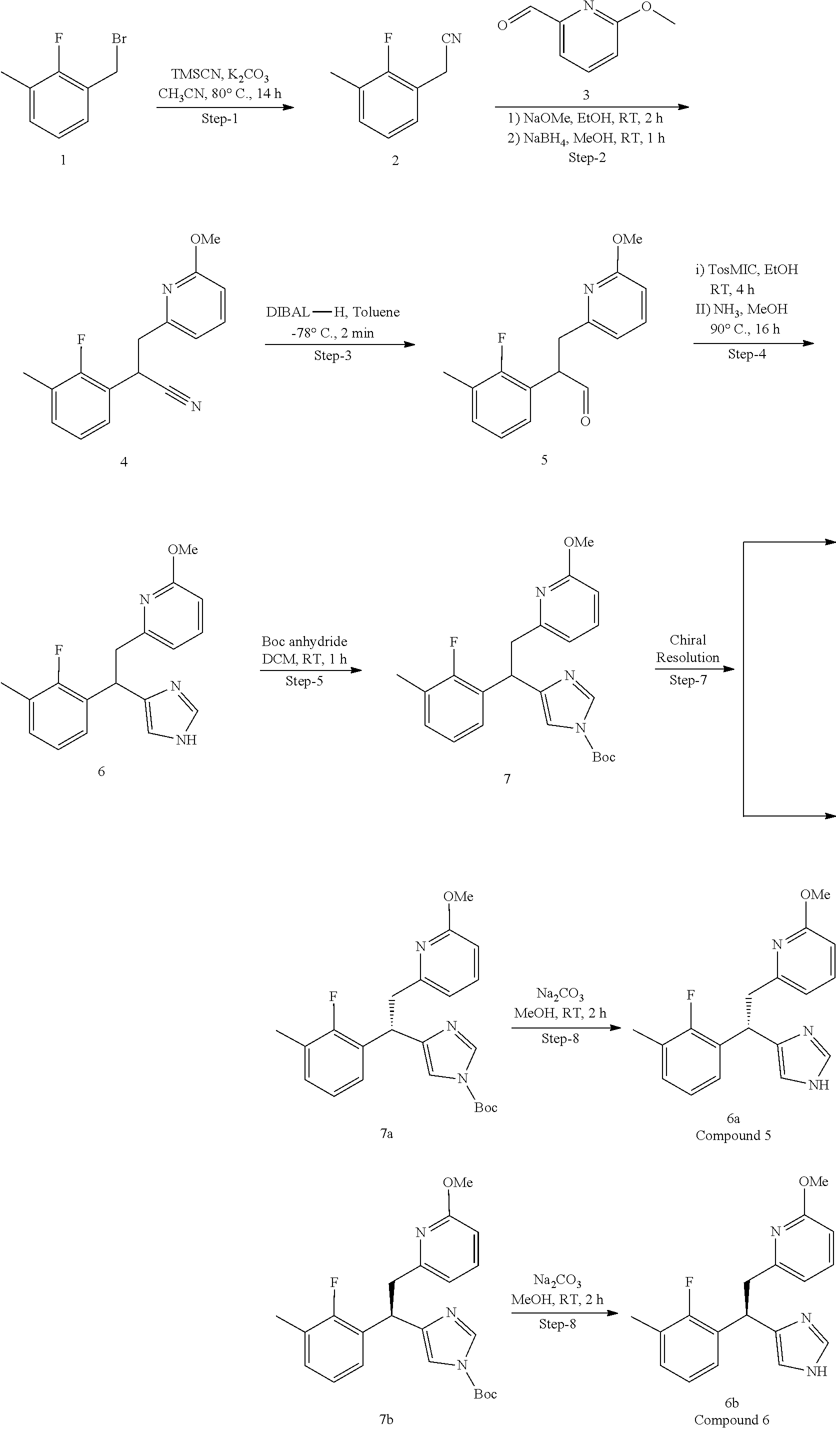

### Step 1: 1-(bromomethyl)-2-fluoro-3-methylbenzene (2)

To a stirred solution of 1-(bromomethyl)-2-fluoro-3-methylbenzene (1) (2.0 g, 9.85 mmol) in anhydrous acetonitrile (20 mL) were added potassium carbonate (2.72 g, 2 eq., 19.7 mmol) and trimethylsilanecarbonitrile (1.85 μL, 1.5 eq., 14.8 mmol) dropwise at room temperature. The reaction mixture was heated to 80° C. and stirred for 16 hours. The progress of the reaction was monitored by TIC. After completion of reaction, the mixture was allowed to cool to room temperature, quenched with water (500 mL), the reaction mixture was extracted with ethyl acetate (2×200 ml). The combined organic layers were washed with brine solution, dried over anhydrous sodium sulphate, filtered and concentrated under reduced pressure to obtain crude, which was purified by flash column chromatography using 4%-3-me acetate in hexane, The appropriate fractions were concentrated to obtain 2-(2-fluoro-3-methylphenyl)acetonitrile (2) (1.3 g, 88.48% yield) as an off-white solid.

¹H NMR (400 MHz, DMSO-d6): δ 7.28-7.23 (m, 2H), 7.12-7.09 (m, 1H), 4.01 (s, 2H), 2.23 (s, 3H).

### Step 2: Synthesis of 2: 2-(2-fluoro-3-methylphenyl)-3-(6-methoxypyridin-2-yl)propane nitrile (4)

To a stirred solution of 2-(2-fluoro-3-methylphenyl)acetonitrile (2) (0.6 g, 4.02 mmol) in ethanol (10 ml) was added 6-methoxypyridine-2-carbaldehyde (552 mg, 4.02 mmol) at room temperature. To the resulting reaction mixture was added sodium methanolate (21.7 mg, 0.1 eq., 402 μmol) at room temperature and stirred for 2 h. The progress of the reaction was monitored by LCMS. After completion of reaction, sodium borohydride (304 mg, 2 eq., 8.04 mmol) was added and stirred for 1 h at room temperature. The reaction mixture was quenched with ice water and concentrated under reduced pressure to remove ethanol. The obtained crude was diluted with water (40 ml) and extracted with ethyl acetate (2×50 ml). The organic layers were washed with brine solution, dried over anhydrous sodium sulphate, filtered & concentrated under reduced pressure to obtain crude, which was purified by flash column chromatography using 25% ethyl acetate in hexane as eluent. The appropriate fractions were concentrated to obtain 2-(2-fluoro-3-methylphenyl)-3-(6-methoxypyridin-2-yl)propanenitrile (4) (0.460 g, 42% yield) as an off-white solid.

MS (ESI): Mass calculated for C₁₆H₁₅FN₂O, 270.3; m/z found, 271.3 [M+H]⁺

### Step 3: Synthesis of 2-(2-fluoro-3-methylphenyl)-3-(5-methoxypyridin-2-yl)propanal (5)

To a stirred solution of 2-(2-fluoro-3-methylphenyl)-3-(6-methoxypyridin-2-yl)propanenitrile (4) (460 mg, 1.7 mmol) in toluene (10 ml) was added diisobutylaluminium hydride (1M solution in cyclohexane) (6.7 ml, 3 eq., 6.76 mmol) at −78° C. and stirred at same temperature for 2 minutes. The reaction mixture was immediately quenched with saturated ammonium chloride (10 ml), the mixture was allowed to warm to room temperature and stirred for 15 min. Then mixture was filtered through celite to remove inorganic precipitate, the filtrate was extracted with ethyl acetate (2×50 ml). The combined organic layers were washed with brine, dried over anhydrous sodium sulphate, filtered & concentrated under reduced pressure to obtain crude, which was purified by flash column chromatography using 2% methanol in dichloromethane. The appropriate fractions were concentrated to obtain 2-(2-fluoro-3-methylphenyl)-3-(6-methoxypyridin-2-yl)propanal (5) (0.3 g, 64% yield) as yellow oil.

MS (ESI): Mass calculated for C₁₆H₁₆FNO₂, 273.31; m/z found, 274.2 [M+H]⁺.

### Step 4: 2-[2-(2-fluoro-3-methylphenyl)-2-(1H-imidazol-4-yl)ethyl]-6-methoxypyridine (6)

To a stirred solution of 2-(2-fluoro-3-methylphenyl)-3-(6-methoxypyridin-2-yl)propanal (5) (0.3 g, 1.1 mmol) in ethanol (10 ml) was added 1-isocyanomethanesulfonyl-4-methylbenzene (0.321 g, 1.5 eq., 1.65 mmol) and stirred at room temperature for 4 hours. The ethanol was concentrated, and the obtained crude was dissolved in methanol (3 ml). Then this mixture was transferred into a high-pressure steel vessel. The freshly prepared solution of ammonia in methanol (20 ml) was added to it. The vessel was closed, and the mixture was stirred at 90° C. for 16 hours. The mixture was allowed to cool to room temperature and solvent was concentrated under vacuum. The obtained crude was dissolved with ethyl acetate, washed with saturated solution of sodium bicarbonate, water, brine, dried over anhydrous sodium sulphate, filtered and concentrated under reduced pressure to obtain crude 2-[2-(2-fluoro-3-methylphenyl)-2-(1H-imidazol-4-yl)ethyl]-6-methoxypyridine (6) (0.350 g, crude) as a brown gummy material. This crude was taken to next step without further purification.

MS (ESI): Mass calculated for C₁₈H₁₈FN₃O, 311.4; m/z found, 312.2 [M+H]⁺.

### Step 5: Synthesis of tert-butyl 4-[1-(2-fluoro-3-methylphenyl)-2-(6-methoxypyridin-2-yl)ethyl]-1H-imidazole-1-carboxylate (7)

To a stirred solution of 2-[2-(2-fluoro-3-methylphenyl)-2-(1H-imidazol-4-yl)ethyl]-6-methoxypyridine (6) (350 mg, 1.12 mmol) in dichloromethane (10 ml) was added triethylamine (228 mg, 2 eq., 2.25 mmol). Then, di-tert-butyl dicarbonate (491 mg, 2 eq., 2.25 mmol) was added at room temperature and stirred for 4 hours. The progress of the reaction was monitored by TLC. After completion of reaction, the mixture was diluted with water and extracted with dichloromethane (2×50 ml). The combined organic layers were washed with brine, dried over anhydrous sodium sulphate, filtered and concentrated under reduced pressure to obtain crude, which was purified by combi flash chromatography using 40% ethyl acetate in n-hexane as an eluent to obtain tert-butyl 4-[1-(2-fluoro-3-methylphenyl)-2-(6-methoxypyridin-2-yl)ethyl]-1H-imidazole-1-carboxylate (7) (0.170 g, 36% yield) as yellow gummy material.

MS (ESI): Mass calculated for C₂₃H₂₆FN₃O₃, 411.5; m/z found, 412.2 [M+H]⁺.

¹H NMR (400 MHz, DMSO-d6): δ 8.07 (s, 1H), 7.49 (t, J=3.6 Hz, 1H), 7.25-7.20 (m, 2H), 7.07-7.03 (m, 1H), 6.96 (t, J=7.6 Hz, 1H), 6.72 (d, J=7.2 Hz, 1H), 6.53 (d, J=8.0 Hz, 1H), 4.84 (t, J=7.6 Hz, 1H), 3.78 (s, 3H), 3.47-3.40 (m, 1H), 3.27-3.21 (m, 1H), 2.10 (s, 3H), 1.57 (s, 9H).

### Step 6: 2-[2-(2-fluoro-3-methylphenyl)-2-(1H-imidazol-4-yl)ethyl]-6-methoxypyridine (8)

To stirred solution of tert-butyl 4-[1-(2-fluoro-3-methylphenyl)-2-(6-methoxypyridin-2-yl)ethyl]-1H-imidazole-1-carboxylate (7) (0.060 g, 146 μmol) in methanol (3 ml) was added sodium carbonate (0.039 g, 2 eq., 292 μmol) at room temperature and stirred for 2 hours. The progress of the reaction was monitored by TLC. After 2 hours, the reaction mixture was evaporated under reduced pressure. The obtained crude was diluted in water (10 ml) and extracted with 5% methanol in dichloromethane (2×20 ml). The combined organic layers were concentrated under reduced pressure to get crude as a yellow solid. The crude was purified by preparative HPLC [Analytical conditions: Column: X-Bridge C-18 (250 mm×4.6 mm×5 mic), mobile phase (A): 0.1% ammonia in water, mobile phase (B): acetonitrile, flow rate: 1.0 ml/min, % of B: 0/2, 18/98, 25/98, 27/2, 30/2] to obtain 2-[2-(2-fluoro-3-methylphenyl)-2-(1H-imidazol-4-yl)ethyl]-6-methoxypyridine (8) (0.015 g, 33% yield) as an off-white solid.

MS (ESI): Mass calculated for C₁₈H₁₈FN₃O, 311.4; m/z found, 312.2 [M+H]⁺.

¹H NMR (400 MHz, DMSO-d6): δ 11.72 (s, 1H), 7.51-7.42 (m, 2H), 7.24-7.21 (m, 1H), 7.07-6.91 (m, 2H), 6.80 (s, 1H), 6.74-6.65 (m, 1H), 6.54-6.49 (m, 1H), 4.82 (t, J=8.0 Hz, 1H), 3.78 (s, 3H), 3.45-3.31 (m, 1H), 3.28-3.15 (m, 1H), 2.12 (s, 3H).

### Step 7: Chiral Resolution

The chiral resolution of compound tert-butyl 4-[1-(2-fluoro-3-methylphenyl)-2-(6-methoxypyridin-2-yl)ethyl]-1H-imidazole-1-carboxylate (7) was done by following method [Analytical conditions: Column: CHIRALPAK IG (100 mm×4.6 mm×3 μm); Mobile phase: n-Hexane: Ethanol (95:05); Flow rate: 1.0 ml/minutes (10 min. run)] and isolated 2 peaks. The isolated peaks were concentrated under reduced pressure and analysed chiral HPLC purity & taken for next step.

Peak 1(7a): (Chiral HPLC—RT: 4.69 min.)

Peak 2 (7b): Chiral HPLC—RT: 5.86 min.) Note: Absolute stereochemistry of 7a and 7b is not known. They are assigned arbitrarily.

### Step 8: Synthesis of (R)-2-(2-(2-fluoro-3-methylphenyl)-2-(1H-imidazol-4-yl)ethyl)-6-methoxypyridine

To stirred solution of tert-butyl 4-[(1R)-1-(2-fluoro-3-methylphenyl)-2-(6-methoxypyridin-2-yl)ethyl]-1H-imidazole-1-carboxylate (6a) (0.055 g, 134 μmol) in methanol (2 ml) was added sodium carbonate (0.028 g, 2 eq., 267 μmol) at room temperature and stirred for 2 hours. After 2 hours, the reaction mixture was concentrated under reduced pressure. The obtained crude was diluted in water (10 ml) and extracted with 5% methanol in dichloromethane (2×20 ml). The combined organic layers were concentrated under reduced pressure the obtain crude, which was purified by combi flash chromatography using 12% methanol in dichloromethane as an eluent to obtain 2-[(2R)-2-(2-fluoro-3-methylphenyl)-2-(1H-imidazol-4-yl)ethyl]-6-methoxypyridine (7a) (0.030 g, 7 2% yield) as an off-white solid.

MS (ESI): Mass calculated for C₁₈H₁₈FN₃O, 311.4; m/z found, 312.3 [M+H]+.

¹H NMR (400 MHz, DMSO-d6): δ 11.72 (s, 1H), 7.51-7.41 (m, 2H), 7.24-7.21 (m, 1H), 7.07-6.92 (m, 2H), 6.80 (s, 1H), 6.74-6.65 (m, 1H), 6.54-6.49 (m, 1H), 4.82 (t, J=8.0 Hz, 1H), 3.78 (s, 3H), 3.45-3.31 (m, 1H), 3.28-3.15 (m, 1H), 2.33-2.12 (m, 3H).

### Step 8: Synthesis of 2-[(2S)-2-(2-fluoro-3-methylphenyl)-2-(1H-imidazol-4-yl)ethyl]-6-methoxypyridine

To stirred solution of tert-butyl 4-[(1S)-1-(2-fluoro-3-methylphenyl)-2-(6-methoxypyridin-2-yl)ethyl]-1H-imidazole-1-carboxylate (6b) (0.055 g, 134 μmol) in methanol (3 ml) was added sodium carbonate (0.028 g, 2 eq., 267 μmol) at room temperature and stirred for 2 hours. After 2 hours, the reaction mixture was concentrated under reduced pressure. The obtained crude was diluted in water (10 ml) and extracted with 5% methanol in dichloromethane (2×20 ml). The combined organic layers were concentrated under reduced pressure the obtain crude, which was purified by combi flash chromatography using 12% methanol in dichloromethane as an eluent to obtain 2-[(2S)-2-(2-fluoro-3-methylphenyl)-2-(1H-imidazol-4-yl)ethyl]-6-methoxypyridine (7b) (0.025 g, 69% yield) as an off white solid. MS (ESI): Mass calculated for C₁₈H₁₈FN₃O, 311.4; m/z found, 312.3 [M+H]+. 1H-NMR (400 MHz, DMSO-d6): δ 11.72 (s, 1H), 7.51-7.42 (m, 2H), 7.24-7.21 (m, 1H), 7.07-6.91 (m, 2H), 6.80 (s, 1H), 6.74-6.65 (m, 1H), 6.54-6.49 (m, 1H), 4.82 (t, J=8.0 Hz, 1H), 3.78 (s, 31H), 3.45-3.31 (m, 1H), 3.28-3.15 (m, 1H), 2.33-2.12 (m, 3H).

| | | | | Molecular | | HPLC | |
|---|---|---|---|---|---|---|---|
| | | | | weight | LCMS | Purity | |
| Procedure | Name | Structure | IUPAC | (g/mol) | [M + H]+ | (%) | 1H NMR |
| A | 1 | 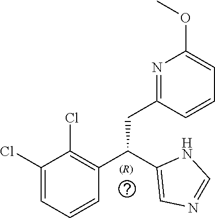 | 2-[(2S)-2-(2,3- dichlorophenyl)- 2-(1H- imidazol-4-yl) ethyl]-6- methoxypyridine | 348.22 | 348.2 | 99.3 | 1H NMR (400 MHz, DMSO-d6): δ 11.85 (s, 1H), 7.53 (s, 1H), 7.49-7.45 (m, 2H), 7.42- 7.40 (m, 1H), 7.26 (t, J = 8.0 Hz, 1H) 6.85 (s, 1H), 6.72 (d, J = 7.2 Hz, 1H), 6.52 (d, J = 8.0 Hz, 1H), 5.15 (t, J = 7.2 Hz, 1H), 3.77 (s, 3H), 3.46-3.41 (m, 1H), 3.31-3.28 (m, 1H). |
| A | 2 | 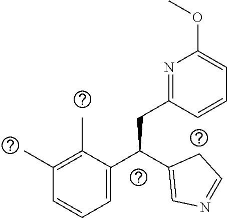 [SMILES: COc1cccc(C[C@@H](C2=CN=CC2)c2cccc(C)c2C)n1] | 2-[(2R)-2-(2,3- dichlorophenyl)- 2-(1H- imidazol-4-yl) ethyl]-6- methoxypyridine | 348.22 | 348.2 | 99.56 | 1H NMR (400 MHz, DMSO-d6): δ 11.79 (s, 1H), 7.52-7.46 (m, 3H), 7.33-7.23 (m, 2H), 6.87 (s, 1H), 6.70 (d, J = 6.8 Hz, 1H), 6.51 (d, J = 8.0 Hz, 1H), 5.12 (t, J = 7.2 Hz, 1H), 3.77 (s, 3H), 3.43-3.40 (m, 1H), 3.28-3.25 (m, 1H). |
| A | 3 | 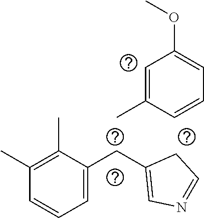 | 2-[(2S)-2-(2,3- dimethylphenyl)- 2-(1H- imidazol-4-yl) ethyl]-6- methoxypyridine | 307.397 | 308.3 | 99.01 | ¹H NMR (400 MHz, DMSO-d6): δ 11.72 (brs, 1H), 7.46-7.43 (m, 2H), 7.10 (s, 1H), 6.97-6.92 (m, 2H), 6.76 (s, 1H), 6.69 (d, J = 7.2 Hz, 1H), 6.52 (d, J = 8.0 Hz, 1H), 4.87 (t, J = 7.2 Hz, 1H), 3.82 (s, 3H), 3.41-3.35 (m, 1H), 3.14-3.12 (m, 1H), 2.23 (s, 3H), 2.19 (s, 3H). |
| A | 4 | 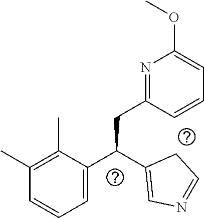 [SMILES: COc1cccc(C[C@@H](C2=CN=CC2)c2cccc(C)c2C)n1] | 2-[(2R)-2-(2,3- dimethylphenyl)- 2-(1H- imidazol-4-yl) ethyl]-6- methoxypyridine | 307.397 | 308.3 | 98.34 | ¹H NMR (400 MHz, DMSO-d6): δ 11.74 (brs, 1H), 7.47-7.43 (m, 2H), 7.09 (s, 1H), 6.97- 6.91 (m, 2H), 6.76 (s, 1H), 6.69 (d, J = 7.2 Hz, 1H), 6.52 (d, J = 8.0 Hz, 1H), 4.87 (t, J = 7.2 Hz, 1H), 3.82 (s, 3H), 3.41-3.35 (m, 1H), 3.14 3.08 (m, 1H), 2.23 (s, 3H), 2.19 (s, 3H). |
| A | 5 | 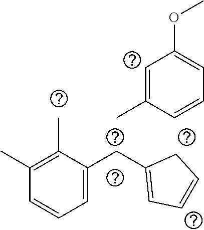 | 2-[(2S)-2-(2- fluoro-3- methylphenyl)- 2-(1H- imidazol-4-yl) ethyl]-6- methoxypyridine | 311.36 | 312.3 | 99.88 | ¹H-NMR (400 MHz, DMSO-d6): δ 11.72 (s, 1H), 7.51-7.42 (m, 2H), 7.24-7.21 (m, 1H), 7.07-6.91 (m, 2H), 6.80 (s, 1H), 6.74-6.65 (m, 1H), 6.54-6.49 (m, 1H), 4.82 (t, J = 8.0 Hz, 1H), 3.78 (s, 3H), 3.45-3.31 (m, 1H), 3.28 3.15 (m, 1H), 2.33- 2.12 (m, 3H). |
| A | 6 | 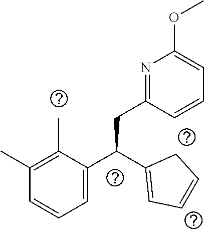 | 2-[(2R)-2-(2- fluoro-3- methylphenyl)- 2-(1H- imidazol-4-yl) ethyl]-6- methoxypyridine | 311.36 | 312.3 | 99.99 | ¹H-NMR (400 MHz, DMSO-d6): δ 11.72 (s, 1H), 7.51-7.41 (m, 2H), 7.24-7.21 (m, 1H), 7.07-6.92 (m, 2H), 6.80 (s, 1H), 6.74-6.65 (m, 1H), 6.54-6.49 (m, 1H), 4.82 (t, J = 8.0 Hz, 1H), 3.78 (s, 3H), 3.45-3.31 (m, 1H), 3.28 3.15 (m, 1H), 2.33-2.12 (m, 3H). |
| A | 7 | 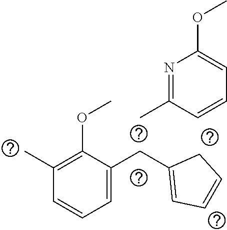 | 2-[(2S)-2-(3- chloro-2- methoxyphenyl)- 2-(1H- imidazol-4-yl) ethyl]-6- methoxypyridine | 343.81 | 344.2 | 99.72 | 1H NMR (400 MHz, DMSO-d6): δ 11.74 (brs, 1H), 7.52-7.43 (m, 3H), 7.24-7.21 (m, 1H), 7.05 (t, J = 7.6 Hz, 1H), 6.83 (s, 1H), 6.66 (d, J = 7.2 Hz, 1H), 6.53 (d, J = 5.6 Hz, 1H), 4.90 (t, J = 7.6 Hz, 1H), 3.80 (s, 3H), 3.69 (s, 3H), 3.43 3.32 (m, 1H), 3.20-3.14 (m, 1H). |
| A | 8 | 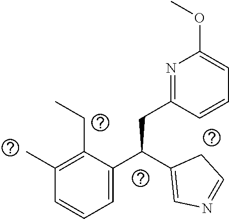 [SMILES: CCc1c(C)cccc1[C@@H](Cc1cccc(OC)n1)C1=CN=CC1] | 2-[(2R)-2-(3- chloro-2- methoxyphenyl)- 2-(1H- imidazol-4-yl) ethyl]-6- methoxypyridine | 343.81 | 344.2 | 97.71 | ¹H NMR (400 MHz, DMSO-d6): δ 11.73 (brs, 1H), 7.52-7.43 (m, 3H), 7.24-7.21 (m, 1H), 7.05 (t, J = 7.6 Hz, 1H), 6.83 (s, 1H), 6.66 (d, J = 7.2 Hz, 1H), 6.53 (d, J = 5.6 Hz, 1H), 4.90 (t, J = 7.6 Hz, 1H), 3.80 (s, 3H), 3.69 (s, 3H), 3.43- 3.32 (m, 1H), 3.20-3.14 (m, 1H). |
| A | 9 | 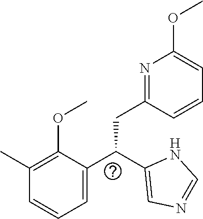 | 2-[(2S)-2-(1H- imidazol- 4-yl)-2-(2- methoxy-3- methylphenyl) ethyl]-6- methoxypyridine | 323.396 | 324.3 | 99.12 | ¹H NMR (400 MHz, DMSO-d6): δ 11.74 (s, 1H), 7.46-7.43 (m, 2H), 7.25 (d, J = 6.8 Hz, 1H), 6.98-6.91 (m, 2H), 6.72 (s, 1H), 6.87 (d, J = 7.2 Hz, 1H), 6.51 (d, J = 8.0 Hz, 1H), 4.92 (t, J = 7.6 Hz, 1H), 3.80 (s, 3H), 3.62 (s, 3H), 3.43-3.39 (m, 1H), 3.16- 3.11 (m, 1H), 2.19 (s, 3H). |
| A | 10 | 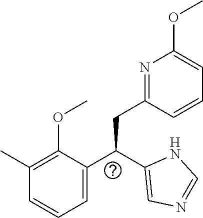 | 2-[(2R)-2-(1H- imidazol- 4-yl)-2-(2- methoxy-3- methylphenyl) ethyl]-6- methoxypyridine | 323.396 | 324.3 | 99.6 | ¹H NMR (400 MHz, DMSO-d6): δ 11.75 (s, 1H), 7.46-7.43 (m, 2H), 7.25 (d, J = 6.8 Hz, 1H), 6.98-6.91 (m, 2H), 6.72 (s, 1H), 6.87 (d, J = 7.2 Hz, 1H), 6.51 (d, J = 8.0 Hz, 1H), 4.92 (t, J = 7.6 Hz, 1H), 3.80 (s, 3H), 3.62 (s, 3H), 3.43-3.39 (m, 1H), 3.16- 3.11 (m, 1H), 2.19 (s, 3H) |
| A | 11 | 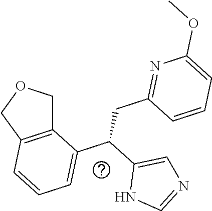 [SMILES: COc1cccc(C[C@H](c2cnc[nH]2)c2cccc3c2COC3)n1] | 2-[(2S)-2-(1,3- dihydro-2- benzofuran-4- yl)-2-(1H- imidazol-4-yl) ethyl]-6- methoxypyridine | 321.38 | 322.3 | 99.8 | ¹H NMR (400 MHz, DMSO-d6): δ 11.76 (s, 1H), 7.49 (s, 1H), 7.47- 7.43 (m, 1H), 7.17- 7.15 (m, 2H), 7.05-7.01 (m, 1H), 6.86-6.84 (s, 1H), 6.67 (d, J = 6.8 Hz, 1H), 6.52 (d, J = 8.4 Hz, 1H), 5.09-5.06 (m, 1H), 4.95-4.92 (m, 2H), 4.76-4.73 (m, 1H), 4.41-4.39 (m, 1H), 3.77 (s, 3H), 3.43- 3.38 (m, 1H), 3.24- 3.18 (m, 1H). |
| A | 12 | 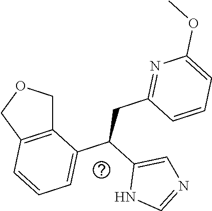 [SMILES: COc1cccc(C[C@@H](c2cnc[nH]2)c2cccc3c2COC3)n1] | 2-[(2R)-2-(1,3- dihydro-2- benzofuran-4- yl)-2-(1H- imidazol-4-yl) ethyl]-6- methoxypyridine | 321.38 | 322.3 | 97.33 | ¹H NMR (400 MHz, DMSO-d6): δ 11.74 (s, 1H), 7.49-7.43 (m, 2H), 7.18-7.04 (m, 3H), 6.86-6.84 (m, 1H), 6.67 (d, J = 6.8 Hz, 1H), 6.52 (d, J = 8.4 Hz, 1H), 5.09-5.06 (m, 1H), 4.92 (s, 2H), 4.76-4.74 (m, 1H), 4.41-4.39 (m, 1H), 3.79 (s, 3H), 3.44-3.38 (m, 1H), 3.24-3.18 (m, 1H). |
| A | 13 | 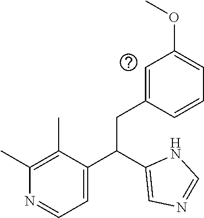 [SMILES: COc1cccc(CC(c2cnc[nH]2)c2ccnc(C)c2C)c1] | 4-[1-(1H- imidazol- 4-yl)- 2-(6- methoxypyridin- 2-yl)ethyl]-2,3- dimethylpyridine | 308.385 | 309.3 | 99.67 | ¹H NMR (400 MHz, DMSO-d6): δ 11.76 (brs, 1H), 8.08 (d, J = 5.2 Hz, 1H), 7.52-7.43 (s, 2 H), 7.14 (d, J = 4.8 Hz, 1H), 6.86 (s, 1H), 6.69 (d, J = 7.2 Hz, 1H), 6.56- 6.51 (m, 1H), 4.83 (t, J = 7.6 Hz, 1H), 3.79 (s, 3H), 3.43-3.34 (m, 1H), 3.20-3.13 (m, 1H), 2.40 (s, 3H), 2.24 (s, 3H). |
| A | 14 | 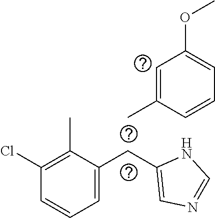 | 2-[(2S)-2- (3-chloro-2- methylphenyl)- 2-(1H- imidazol-4-yl) ethyl]-6- methoxypyridine | 327.81 | 328.1 | 98.82 | ¹H-NMR (400 MHz, DMSO-d6): δ 11.75 (s, 1H), 7.49-7.44 (m, 2H), 7.29 (s, 1H), 7.19- 7.18 (m, 1H), 7.09 (t, J = 7.6 Hz, 1H), 6.84 (s, 1H), 6.70-6.68 (m, 1H), 6.52 (d, J = 8.4 Hz, 1H), 4.89-4.81 (m, 1H), 3.78 (s, 3H), 3.43- 3.38 (m, 1H), 3.19-3.14 (m, 1H), 2.37 (s, 3H). |
| A | 15 | 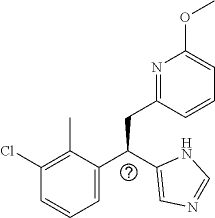 [SMILES: COc1cccc(C[C@@H](c2cnc[nH]2)c2cccc(Cl)c2C)n1] | 2-[(2R)-2- (3-chloro-2- methylphenyl)- 2-(1H- imidazol-4-yl) ethyl]-6- methoxypyridine | 327.81 | 328.2 | 99.76 | ¹H-NMR (400 MHz, DMSO-d6): δ 11.76 (s, 1H), 7.49-7.44 (m, 2H), 7.29 (s, 1H), 7.19- 7.18 (m, 1H), 7.09 (t, J = 7.6 Hz, 1H), 6.84 (s, 1H), 6.70-6.88 (m, 1H), 6.52 (d, J = 8.4 Hz, 1H), 4.89-4.81 (m, 1H), 3.80 (s, 3H), 3.44- 3.38 (m, 1H), 3.19-3.14 (m, 1H), 2.37 (s, 3H). |
| A | 16 | 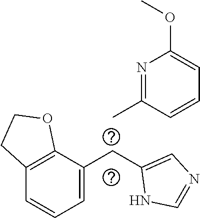 | 2-[(2S)-2- (2,3-dihydro- 1-benzofuran- 7-yl)-2-(1H- imidazol-4-yl) ethyl]-6- methoxypyridine | 321.38 | 322.3 | 99.11 | ¹H NMR (400 MHz, DMSO-d6): δ 11.66 (brs, 1H), 7.49-7.41 (m, 2H), 7.06-6.93 (m, 2H), 6.73-6.47 (m, 4H), 4.65 (t, J = 8.0 Hz, 1H), 4.53-4.35 (m, 2H), 3.80 (s, 3H), 3.40-3.35 (m, 1H), 3.24-3.20 (m, 1H), 3.15-3.08 (m, 2H). |
| A | 17 | 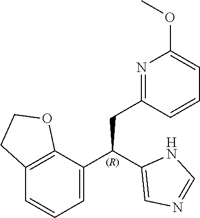 | 2-[(2R)-2- (2,3-dihydro- 1-benzofuran- 7-yl)-2-(1H- imidazol-4-yl) ethyl]-6- methoxypyridine | 321.38 | 322.3 | 98.00 | ¹H NMR (400 MHz, DMSO-d6): δ 11.66 (brs, 1H), 7.49-7.41 (m, 2H), 7.06-6.93 (m, 2H), 6.73-6.47 (m, 4H), 4.65 (t, J = 8.0 Hz, 1H), 4.53-4.35 (m, 2H), 3.80 (s, 3H), 3.40-3.35 (m, 1H), 3.24-3.20 (m, 1H), 3.15-3.08 (m, 2H). |
| A | 18 | 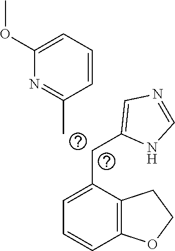 | 2-[(2S)-2- (2,3-dihydro- 1-benzofuran- 4-yl)-2-(1H- imidazol-4- yl)ethyl]-6- methoxypyridine | 321.38 | 322.3 | 99.36 | ¹H NMR (400 MHz, DMSO-d6): δ 11.79 (s, 1H), 7.49-7.44 (m, 2H), 6.95 (t, J = 7.6 Hz, 1H), 6.83 (s, 1H), 6.75 (d, J = 7.6 Hz, 1H), 6.66 (d, J = 7.2 Hz, 1H), 6.51 (t, J = 8.4 Hz, 2H), 4.53-4.35 (m, 3H), 3.80 (s, 3H), 3.47-3.38 (m, 1H), 3.23-3.19 (m, 2H), 2.94-2.90 (m, 1H). |
| A | 19 | 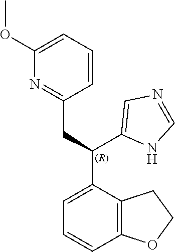 | 2-[(2R)-2- (2,3-dihydro- 1-benzofuran- 4-yl)-2-(1H- imidazol-4- yl)ethyl]-6- methoxypyridine | 321.38 | 322.3 | 99.36 | ¹H NMR (400 MHz, DMSO-d6): δ 11.83 (s, 1H), 7.53-7.49 (m, 2H), 6.96 (t, J = 7.6 Hz, 1H), 6.85 (s, 1H), 6.75 (d, J = 7.6 Hz, 1H), 6.66 (d, J = 7.2 Hz, 1H), 6.52 (t, J = 8.4 Hz, 2H), 4.53-4.35 (m, 3H), 3.80 (s, 3H), 3.49-3.38 (m, 1H), 3.23-3.19 (m, 2H), 2.94-2.90 (m, 1H). |
| A | 20 | 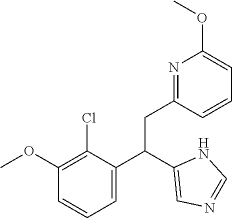 [SMILES: COc1cccc(CC(c2cnc[nH]2)c2cccc(OC)c2Cl)n1] | 2-[2-(2- chloro-3- methoxyphenyl)- 2-(1H- imidazol-4-yl) ethyl]-6- methoxypyridine | 343.81 | 344.1 | 98.08 | ¹H NMR (400 MHz, DMSO-d6): δ 11.70 (s, 1H), 7.47-7.40 (m, 2H), 7.21-7.16 (m, 1H), 7.13 (d, J = 8.0 Hz, 1H), 6.93-6.85 (m, 1H), 6.76-6.72 (m, 1H), 6.65 (d, J = 7.2 Hz, 1H), 6.51-6.46 (m, 1H), 5.17- 5.05 (m, 1H), 3.76 (s, 6H), 3.57-3.34 (m, 1H), 3.24-3.16 (m, 1H). |
| A | 21 | 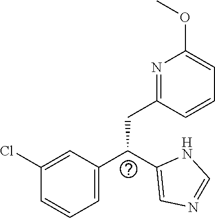 [SMILES: COc1cccc(C[C@@H](c2cccc(Cl)c2)c2cnc[nH]2)n1] | 2-[(2S)-2-(3- chlorophenyl)- 2-(1H- imidazol-4- yl)ethyl]-6- methoxypyridine | 313.79 | 314.2 | 98.44 | ¹H NMR (400 MHz, DMSO-d6): δ 11.81 (s, 1H), 7.49 (s, 1H), 7.45- 7.41 (m, 1H), 7.28 (s, 1H), 7.22-7.07 (m, 3H), 6.84 (s, 1H), 6.65 (d, J = 6.8 Hz, 1H), 6.49 (d, J = 8.4 Hz, 1H), 4.50 (t, J = 7.6 Hz, 1H), 3.77 (s, 3H), 3.41-3.36 (m, 1H), 3.24-3.18 (m, 1H). |
| A | 22 | 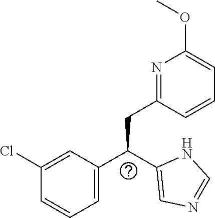 [SMILES: COc1cccc(C[C@H](c2cccc(Cl)c2)c2cnc[nH]2)n1] | 2-[(2R)-2-(3- chlorophenyl)- 2-(1H- imidazol-4-yl) ethyl]-6- methoxypyridine | 313.79 | 314.2 | 99.04 | ¹H NMR (400 MHz, DMSO-d6): δ 11.82 (s, 1H), 7.49 (s, 1H), 7.44- 7.41 (m, 1H), 7.27 (s, 1H), 7.22-7.07 (m, 3H), 6.84 (s, 1H), 6.65 (d, J = 6.8 Hz, 1H), 6.49 (d, J = 8.4 Hz, 1H), 4.50 (t, J = 7.6 Hz, 1H), 3.77 (s, 3H), 3.42-3.36 (m, 1H), 3.25-3.18 (m, 1H). |
| A | 23 | 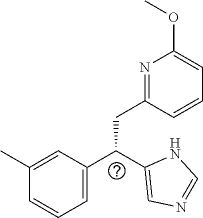 [SMILES: COc1cccc(C[C@@H](c2cccc(C)c2)c2cnc[nH]2)n1] | 2-[(2S)-2- (1H-imidazol- 4-yl)-2-(3- methylphenyl) ethyl]-6- methoxypyridine | 293.37 | 294.3 | 99.36 | ¹H NMR (400 MHz, DMSO-d6): δ 11.84 (brs, 1H), 7.44 (s, 2H), 7.04- 7.02 (m, 3H), 6.88 (s, 1H), 6.77-6.62 (m, 2H), 6.48 (d, J = 8.0 Hz, 1H), 4.41 (s, 1H), 3.78 (s, 3H), 3.48-3.36 (m, 1H), 3.29-3.17 (m, 1H), 2.19 (s, 3H). |
| A | 24 | 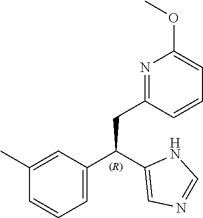 | 2-[(2R)-2- (1H-imidazol- 4-yl)-2-(3- methylphenyl) ethyl]-6- methoxypyridine | 293.37 | 294.3 | 99.13 | ¹H NMR (400 MHz, DMSO-d6): δ 11.75 (brs, 1H), 7.48-7.41 (m, 2H), 7.13-7.12 (m, 3H), 6.90 (d, J = 7.2 Hz, 1H), 6.80-6.70 (m, 1H), 6.63 (d, J = 7.2 Hz, 1H), 6.50 (d, J = 8.0 Hz, 1H), 4.47 (t, J = 7.6 Hz, 1H), 3.81 (s, 3H), 3.44- 3.38 (m, 1H), 3.22-3.15 (m, 1H), 2.22 (s, 3H). |
| A | 25 | 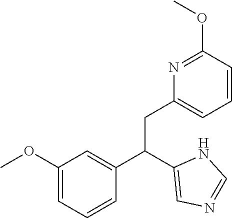 [SMILES: COc1cccc(C(Cc2cccc(OC)n2)c2cnc[nH]2)c1] | 2-[2-(1H- imidazol-4- yl)-2-(3- methoxyphenyl) ethyl]-6- methoxypyridine | 309.369 | 310.3 | 99.92 | ¹H NMR (400 MHz, DMSO-d6): δ 11.70 (s, 1H), 7.48-7.42 (m, 2H), 7.10 (s, 1H), 6.81 (d, J = 7.2 Hz, 3H), 6.66 (s, 2H), 6.50 (d, J = 8.0 Hz, 1H), 4.47 (brs, 1H), 3.81 (s, 3H), 3.67 (s, 3H), 3.43-3.38 (m, 1H), 3.23-3.21(m, 1H). |
| A | 26 | 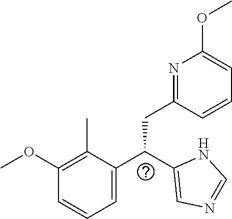 | 2-[(2S)-2- (1H-imidazol- 4-yl)-2-(3- methoxy-2- methylphenyl) ethyl]-6- methoxypyridine | 323.38 | 324.2 | 97.6 | ¹H NMR (400 MHz, DMSO-d6): δ 11.67 (brs, 1H), 7.50-7.41 (m, 2H), 7.09-7.00 (m, 2H), 6.79-6.63 (m, 3H), 6.55- 6.49 (m, 1H), 4.79 (t, J = 7.6 Hz, 1H), 3.81 (s, 3H), 3.68 (s, 3H), 3.43-3.38 (m, 1H), 3.15- 3.10 (m, 1H), 2.15 (s, 3H). |
| A | 27 | 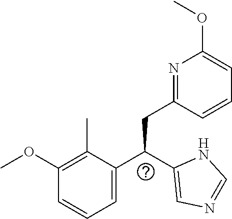 [SMILES: COc1cccc(C[C@@H](c2cnc[nH]2)c2cccc(OC)c2C)n1] | 2-[(2R)-2- (1H-imidazol- 4-yl)-2-(3- methoxy-2- methylphenyl) ethyl]-6- methoxypyridine | 323.396 | 324.2 | 98.25 | ¹H NMR (400 MHz, DMSO-d6): δ 11.71 (brs, 1H), 7.46-7.43 (m, 2H), 7.03 (t, J = 8.0 Hz, 1H), 6.89 (s, 1H), 6.76 (s, 1H), 6.76-6.66 (m, 2H), 6.51 (d, J = 8.0 Hz, 1H), 4.82 (t, J = 5.2 Hz, 1H), 3.81 (s, 3H), 3.71 (s, 3H), 3.41-3.36 (m, 1H), 3.15-3.10 (m, 1H), 2.15 (s, 3H). |
| A | 28 | 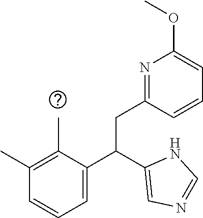 [SMILES: COc1cccc(CC(c2cnc[nH]2)c2cccc(C)c2C)n1] | 2-[2-(2- chloro-3- methylphenyl)- 2-(1H- imidazol-4-yl) ethyl]-6- methoxypyridine | 327.81 | 328.2 | 98.03 | ¹H NMR (400 MHz, DMSO-d6): δ 11.72 (brs, 1H), 7.49-7.45 (m, 2H), 7.33 (s, 1H), 7.11 (s, 2H), 6.79-6.68 (m, 2H), 6.50 (d, J = 8.0 Hz, 1H), 5.11 (s, 1H), 3.78 (s, 3H), 3.43-3.35 (m, 1H), 3.25-3.20 (m, 1H), 2.31 (s, 3H). |
| A | 29 | 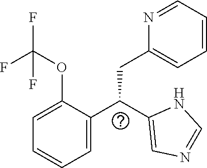 [SMILES: FC(F)(F)Oc1ccccc1[C@H](Cc1ccccn1)c1cnc[nH]1] | 2-[(2S)-2- (1H-imidazol- 4-yl)-2-[2- (trifluoromethoxy) phenyl]ethyl] pyridine | 333.31 | 334.2 | 98.04 | 1H NMR (400 MHz, DMSO-d6): 11.85 (brs, 1H), 8.41 (d, J = 4.0 Hz, 1H), 7.64 (d, J = 6.8 Hz, 1H), 7.55-7.51 (m, 2H), 7.32-7.24 (m, 2H), 7.20-7.18 (m, 1H), 7.10 (m, 1H), 7.02 (d, J = 7.6 Hz, 1H), 6.74 (s, 1H), 4.88 (t, J = 7.6 Hz, 1H), 3.54-3.48 (m, 1H), 3.26-3.21 (m, 1H). |
| A | 30 | 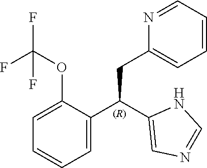 | 2-[(2R)-2-(1H- imidazol- 4-yl)-2-[2- (trifluoromethoxy) phenyl]ethyl] pyridine | 333.31 | 334.2 | 99.78 | 1H NMR (400 MHz, DMSO-d6): 11.81 (brs, 1H), 8.42 (d, J = 4.4 Hz, 1H), 7.64 (d, J = 6.8 Hz, 1H), 7.55-7.52 (m, 2H), 7.32-7.24 (m, 2H), 7.20-7.18 (m, 1H), 7.09 (t, J = 6.8 Hz, 1H), 7.02 (d, J = 7.6 Hz, 1H), 6.75 (s, 1H), 4.88 (t, J = 7.6 Hz, 1H), 3.54- 3.48 (m, 1H), 3.30- 3.21 (m, 1H). |
| A | 31 | 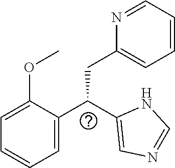 [SMILES: COc1ccccc1[C@H](Cc1ccccn1)c1cnc[nH]1] | 2-[(2S)-2-(1H- imidazol- 4-yl)-2-(2- methoxyphenyl) ethyl]pyridine | 279.343 | 280.2 | 99.94 | ¹H NMR (400 MHz, DMSO-d6): δ 11.70 (brs, 1H), 8.41 (d, J = 4.0 Hz, 1H), 7.55-7.53 (m, 1H), 7.51 (s, 1H), 7.24 (d, J = 7.2 Hz, 1H), 7.19- 7.04 (m, 3H), 6.88-6.85 (m, 2H), 6.82 (s, 1H), 4.94 (t, J = 7.6 Hz, 1H), 3.62 (s, 3H), 3.44-3.38 (m, 1H), 3.23-3.18 (m, 1H). |
| A | 32 | 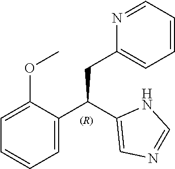 | 2-[(2R)-2- (1H-imidazol- 4-yl)-2-(2- methoxyphenyl) ethyl]pyridine | 279.343 | 280.2 | 99.7 | ¹H NMR (400 MHz, DMSO-d6): δ 11.70 (brs, 1H), 8.41 (d, J = 4.0 Hz, 1H), 7.55-7.52 (m, 1H), 7.51 (s, 1H), 7.24 (d, J = 7.2 Hz, 1H), 7.19- 7.04 (m, 3H), 6.89-6.85 (m, 2H), 6.82 (s, 1H), 4.94 (t, J = 7.6 Hz, 1H), 3.62 (s, 3H), 3.44-3.38 (m, 1H), 3.23-3.18 (m, 1H). |
| A | 33 | 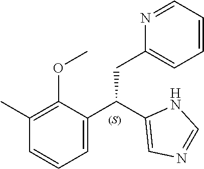 | 2-[(2S)-2- (1H-imidazol- 4-yl)-2-(2- methoxy-3- methylphenyl) ethyl]pyridine | 293.37 | 294.3 | 99.1 | ¹H NMR (400 MHz, DMSO-d6): δ 11.66 (s, 1H), 8.43 (d, J = 4.4 Hz, 1H), 7.55-7.51 (m, 1H), 7.45 (s, 1H), 7.30 (s, 1H), 7.13-7.07 (m, 2H), 6.96-6.92 (m, 2H), 6.68 (s, 1H), 4.90- 4.88 (m, 1H), 3.58 (s, 3H), 3.32-3.28 (m, 2H), 2.18 (s, 3H). |
| A | 34 | 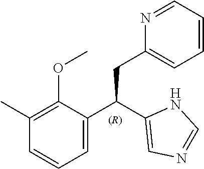 | 2-[(2R)-2- (1H-imidazol- 4-yl)-2-(2- methoxy-3- methylphenyl) ethyl]pyridine | 293.37 | 294.3 | 98.09 | ¹H NMR (400 MHz, DMSO-d6): δ 11.66 (s, 1H), 8.43 (d, J = 4.4 Hz, 1H), 7.55-7.52 (m, 1H), 7.45 (s, 1H), 7.30 (s, 1H), 7.13-7.07 (m, 2H), 6.96-6.92 (m, 2H), 6.67 (s, 1H), 4.90- 4.88 (m, 1H), 3.58 (s, 3H), 3.32-3.28 (m, 2H), 2.18 (s, 3H). |
| A | 35 | 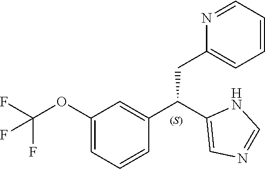 | 2-[(2S)-2- (1H-imidazol- 4-yl)-2-[3- (trifluoromethoxy) phenyl]ethyl] pyridine | 333.314 | 334.2 | 99.07 | ¹H NMR (400 MHz, DMSO-d6): δ 11.81 (s, 1H), 8.44-8.43 (m, 1H), 7.56-7.52 (m, 2H), 7.35-7.26 (m, 2H), 7.19 (s, 1H), 7.11-7.04 (m, 3H), 6.85 (s, 1H), 4.57-4.55 (m, 1H), 3.52-3.47 (m, 1H), 3.31-3.26 (m, 1H) |
| A | 36 | 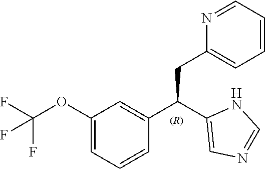 | 2-[(2R)-2- (1H-imidazol- 4-yl)-2-[3- (trifluoromethoxy) phenyl]ethyl] pyridine | 333.314 | 334.2 | 99.79 | ¹H NMR (400 MHz, DMSO-d6): δ 11.76 (s, 1H), 8.44-8.43 (m, 1H), 7.55-7.52 (m, 2H), 7.34-7.26 (m, 2H), 7.19 (s, 1H), 7.11-7.08 (m, 3H), 6.86 (s, 1H), 4.55-4.53 (m, 1H), 3.52-3.47 (m, 1H), 3.31-3.26 (m, 1H). |
| A | 37 | 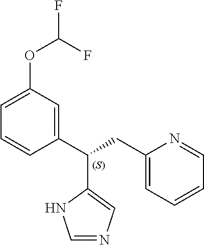 | 2-[(2S)-2-[3- (difluoromethoxy)] phenyl]- 2-(1H-imidazol-4- yl)ethyl]pyridine | 315.324 | 316.3 | 99.52 | ¹H NMR (400 MHz, DMSO-d6): δ 11.73 (brs, 1H), 8.44 (d, J = 3.6 Hz, 1H), 7.55-7.46 (m, 2H), 7.23 (t, J = 7.6 Hz, 1H), 7.14-7.04 (m, 5H), 6.95-6.85 (m, 2H), 4.65-4.47 (m, 1H), 3.52-3.46 (m, 1H), 3.26-3.23 (m, 1H). |
| A | 38 | 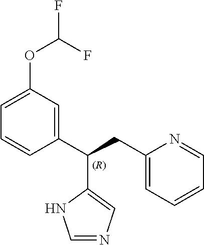 | 2-[(2R)-2-[3- (difluoromethoxy) phenyl]- 2-(1H-imidazol-4- yl)ethyl]pyridine | 315.324 | 316.2 | 99.73 | ¹H NMR (400 MHz, DMSO-d6): δ 11.72 (brs, 1H), 8.43 (d, J = 3.6 Hz, 1H), 7.56-7.53 (m, 2H), 7.23 (t, J = 7.6 Hz, 1H), 7.16-7.09 (m, 5H), 6.97-6.89 (m, 2H), 4.66-4.47 (m, 1H), 3.52-3.46 (m, 1H), 3.26- 3.23 (m, 1H). |
| A | 39 | 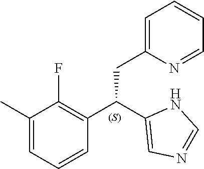 | 2-[(2S)-2- (2-fluoro-3- methylphenyl)- 2-(1H- imidazol-4- yl)ethyl] pyridine | 281.334 | 282.3 | 90.43 | ¹H-NMR (400 MHz, DMSO-d6): δ 11.79 (s, 1H), 8.44-8.42 (m, 1H), 7.57-7.54 (m, 1H), 7.49 (s, 1H), 7.21 (t, J = 8.4 Hz, 1H), 7.11- 7.01 (m, 3H), 6.97-6.94 (m, 1H), 6.77 (s, 1H), 4.84-4.81 (m, 1H), 3.52- 3.46 (m, 1H), 3.27- 3.23 (m, 1H), 2.07 (s, 3H). |
| A | 40 | 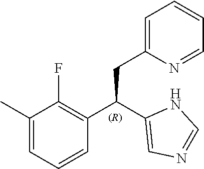 | 2-[(2R)-2-(2- fluoro-3- methylphenyl)- 2-(1H- imidazol-4- yl)ethyl] pyridine | 281.334 | 282.3 | 98.33 | ¹H-NMR (400 MHz, DMSO-d6): δ 11.79 (s, 1H), 8.44-8.42 (m, 1H), 7.57-7.54 (m, 1H), 7.49 (s, 1H), 7.21 (t, J = 8.4 Hz, 1H), 7.11- 7.01 (m, 3H), 6.97-6.94 (m, 1H), 6.77 (s, 1H), 4.84-4.81 (m, 1H), 3.52- 3.46 (m, 1H), 3.27- 3.23 (m, 1H), 2.07 (s, 3H). |
| A | 41 | 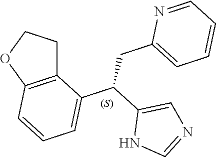 | 2-[(2S)-2- (2,3-dihydro-1- benzofuran-4- yl)-2-(1H- imidazol-4- yl)ethyl]pyridine | 291.354 | 292.3 | 99.44 | ¹H-NMR (400 MHz, DMSO-d6): δ 11.85 (s, 1H), 8.45-8.43 (m, 1H), 7.53-7.48 (m, 2H), 7.11-7.04 (m, 2H), 6.99- 6.95 (m, 1H), 6.81- 6.78 (m, 2H), 6.50 (s, 1H), 4.46-4.34 (m, 3H), 3.50- 3.46 (m, 1H), 3.25-3.20 (m, 2H), 2.92-2.84 (m, 1H). |
| A | 42 | 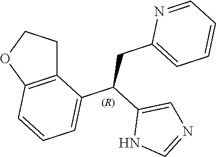 | 2-[(2R)-2-(2,3- dihydro-1- benzofuran-4- yl)-2-(1H- imidazol-4- yl)ethyl] pyridine | 291.354 | 292.3 | 99.7 | ¹H NMR (400 MHz, DMSO-d6): δ 11.85 (s, 1H), 8.45-8.43 (m, 1H), 7.53-7.48 (m, 2H), 7.11-7.01 (m, 2H), 6.99- 6.95 (m, 1H), 6.82- 6.80 (m, 2H), 6.49 (s, 1H), 4.44-4.36 (m, 3H), 3.48- 3.40 (m, 1H), 3.25-3.20 (m, 2H), 2.92-2.84 (m, 1H). |
| A | 43 | 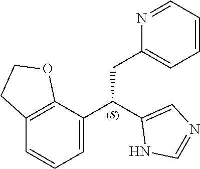 | 2-[(2S)-2- (2,3-dihydro- 1-benzofuran- 7-yl)-2-(1H- imidazol-4- yl)ethyl] pyridine | 291.354 | 292.3 | 98.79 | ¹H NMR (400 MHz, DMSO-d6): δ 11.64 (brs, 1H), 8.42 (s, 1H), 7.54- 7.46 (m, 2H), 7.08- 6.93 (m, 4H), 6.69-6.65 (m, 2H), 4.62 (s, 1H), 4.48-4.41 (m, 2H), 3.47- 3.38 (m, 1H), 3.26-3.21 (m, 1H), 3.12 (t, J = 8.2 Hz, 2H). |
| A | 44 | 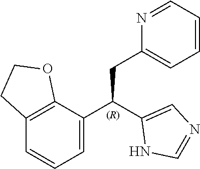 | 2-[(2R)-2- (2,3-dihydro- 1- benzofuran- 7-yl)-2-(1H- imidazol-4- yl)ethyl] pyridine | 291.354 | 292.3 | 99.23 | ¹H NMR (400 MHz, DMSO-d6): δ 11.64 (brs, 1H), 8.42 (s, 1H), 7.57- 7.41 (m, 2H), 7.09- 6.90 (m, 4H), 6.71-6.63 (m, 2H), 4.62 (t, J = 8.0 Hz, 1H), 4.53-4.37 (m, 2H), 3.47-3.38 (m, 1H), 3.26-3.21 (m, 1H), 3.13-3.09 (m, 2H). |
| A | 45 | 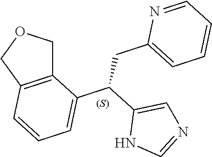 | 2-[(2S)-2- (1,3-dihydro- 2- benzofuran- 4-yl)-2-(1H- imidazol-4- yl)ethyl] pyridine | 291.354 | 292.2 | 99.35 | ¹H NMR (400 MHz, DMSO-d6): δ 11.75 (s, 1H), 8.45-8.43 (m, 1H), 7.74-7.52 (m, 2H), 7.20-7.03 (m, 5H), 6.83 (s, 1H), 5.06-5.03 (m, 1H), 4.95 (s, 2H), 4.72-4.69 (m, 1H), 4.42- 4.39 (m, 1H), 3.52-3.47 (m, 1H), 3.27-3.25 (m, 1H). |
| A | 46 | 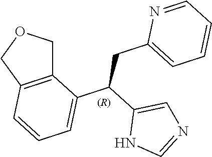 | 2-[(2R)-2- (1,3-dihydro-2- benzofuran-4- yl)-2-(1H- imidazol-4- yl)ethyl] pyridine | 291.354 | 292.3 | 98.25 | ¹H NMR (400 MHz, DMSO-d6): δ 11.77 (s, 1H), 8.45-8.43 (m, 1H), 7.56-7.52 (m, 1H), 7.48-7.47 (m, 1H), 7.22- 7.03 (m, 5H), 6.83 (s, 1H), 5.06-5.03 (m, 1H), 4.91 (s, 2H), 4.72- 4.69 (m, 1H), 4.44-4.41 (m, 1H), 3.52-3.46 (m, 1H), 3.28-3.25 (m, 1H). |
| A | 47 | 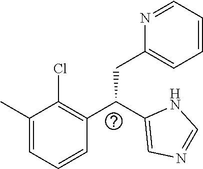 [SMILES: Cc1cccc([C@H](Cc2ccccn2)c2cnc[nH]2)c1Cl] | 2-[(2S)-2- (2-chloro-3- methylphenyl)- 2-(1H- imidazol-4- yl)ethyl] pyridine | 297.79 | 298.3 | 99.86 | ¹H NMR (400 MHz, DMSO-d6): δ 11.70 (brs, 1H), 8.45-8.42 (m, 1H), 7.62-7.58 (m, 1H), 7.55-7.51 (m, 1H), 7.37- 7.34 (m, 1H), 7.18- 7.06 (m, 4H), 6.78-6.67 (m, 1H), 5.05 (t, J = 5.2 Hz, 1H), 3.50-3.39 (m, 1H), 3.27-3.22 (m, 1H), 2.29 (s, 3H). |
| A | 48 | 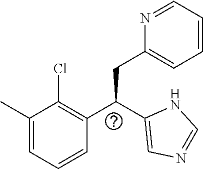 [SMILES: Cc1cccc([C@@H](Cc2ccccn2)c2cnc[nH]2)c1Cl] | 2-[(2R)-2- (2-chloro-3- methylphenyl)- 2-(1H- imidazol-4- yl)ethyl]pyridine | 297.79 | 298.3 | 99.22 | ¹H NMR (400 MHz, DMSO-d6): δ 11.70 (brs, 1H), 8.45-8.42 (m, 1H), 7.62-7.58 (m, 1H), 7.55-7.51 (m, 1H), 7.37- 7.34 (m, 1H), 7.18- 7.06 (m, 4H), 6.78-6.67 (m, 1H), 5.05 (t, J = 5.2 Hz, 1H), 3.50-3.39 (m, 1H), 3.27-3.22 (m, 1H), 2.29 (s, 3H). |
| A | 49 | 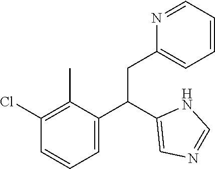 [SMILES: Cc1c(Cl)cccc1C(Cc1ccccn1)c1cnc[nH]1] | 2-[2-(3- chloro-2- methylphenyl)- 2-(1H- imidazol-4- yl)ethyl] pyridine | 297.79 | 298.2 | 99.03 | ¹H NMR (400 MHz, DMSO-d6): δ 11.81 (s, 1H), 8.46-8.45 (m, 1H), 7.58-7.54 (m, 1H), 7.50 (s, 1H), 7.38 (d, J = 9.6 Hz , 1H), 7.20 (d, J = 7.6 Hz, 1H), 7.13- 7.08 (m, 3H), 6.83 (s, 1H), 4.86 (t, J = 7.2 Hz, 1H), 3.51-3.46 (m,1H), 3.26-3.21 (m, 1H), 2.27 (s, 3H). |
| A | 50 | 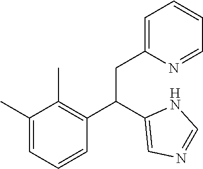 [SMILES: Cc1cccc(C(Cc2ccccn2)c2cnc[nH]2)c1C] | 2-[2-(2,3- dimethylphenyl)- 2-(1H- imidazol-5- yl)ethyl] pyridine | 277.371 | 278.2 | 98.46 | ¹H NMR (400 MHz, DMSO-d6): δ 11.72 (s, 1H), 8.45 (d, J = 4.0 Hz, 1H), 7.56-7.52 (m, 1H), 7.46 (s, 1H), 7.14-7.07 (m, 3H), 7.07-6.99 (m, 2H), 6.74 (s, 1H), 4.85 (t, J = 8.0 Hz, 1H), 3.49- 3.43 (m, 1H), 3.21-3.16 (m, 1H), 2.20 (s, 6H). |
| A | 51 | 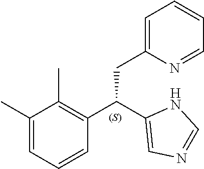 | 2-[(2S)-2-(2,3- dimethylphenyl)- 2-(1H- imidazol-4- yl)ethyl] pyridine | 277.371 | 278.2 | 99.39 | ¹H NMR (400 MHz, DMSO-d6): δ 11.72 (s, 1H), 8.45 (d, J = 4.0 Hz, 1H), 7.56-7.52 (m, 1H), 7.49 (s, 1H), 7.14-7.07 (m, 3H), 7.07- 6.99 (m, 2H), 6.76 (s, 1H), 4.85 (t, J = 8.0 Hz, 1H), 3.49- 3.43 (m, 1H), 3.21-3.16 (m, 1H), 2.20 (s, 6H). |
| A | 52 | 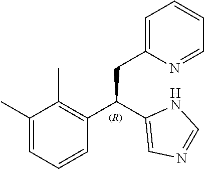 | 2-[(2R)-2-(2,3- dimethylphenyl)- 2-(1H- imidazol-4- yl)ethyl] pyridine | 277.371 | 278.2 | 99.49 | ¹H NMR (400 MHz, DMSO-d6): δ 11.72 (s, 1H), 8.45 (d, J = 4.0 Hz, 1H), 7.56-7.52 (m, 1H), 7.49 (s, 1H), 7.14-7.07 (m, 3H), 7.07-6.99 (m, 2H), 6.76 (s, 1H), 4.85 (t, J = 8.0 Hz, 1H), 3.49- 3.43 (m, 1H), 3.21-3.16 (m, 1H), 2.20 (s, 6H). |
| A | 53 | 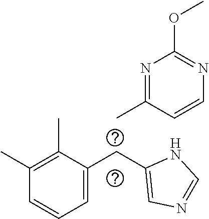 | 4-[(2S)-2- (2,3- dimethylphenyl)- 2-(1H- imidazol-4-yl) ethyl]-2- methoxy- pyrimidine | 308.385 | 309.3 | 99.75 | ¹H NMR (400 MHz, DMSO-d6): δ 11.81 (s, 1H), 8.33 (s, 1H), 7.48 (s, 1H), 7.16-7.15 (m, 1H), 6.98-6.88 (m, 3H), 6.74 (s, 1H), 4.83 (t, J = 7.6 Hz, 1H), 3.82 (s, 3H), 3.47-3.43 (m, 1H), 3.18-3.13 (m, 1H), 2.24 (s, 3H), 2.20 (s, 3H). |
| A | 54 | 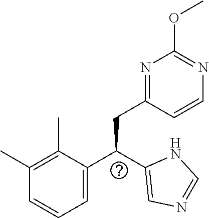 | 4-[(2R)-2- (2,3- dimethylphenyl)- 2-(1H- imidazol-4-yl) ethyl]-2- methoxy- pyrimidine | 308.385 | 309.3 | 99.75 | ¹H NMR (400 MHz, DMSO-d6): δ 11.74 (s, 1H), 8.33 (d, J = 4.8 Hz, 1H), 7.48 (s, 1H), 7.12-.11 (m, 1H), 6.98- 6.88 (m, 3H), 6.74 (s, 1H), 4.85 (d, t = 7.6 Hz, 1H), 3.85 (s, 3H), 3.45-3.40 (m, 1H), 3.18- 3.13 (m, 1H), 2.24 (s, 3H), 2.20 (s, 3H). |
| A | 55 | 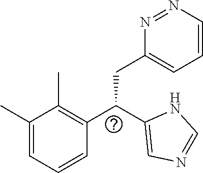 [SMILES: Cc1cccc([C@H](Cc2cccnn2)c2cnc[nH]2)c1C] | 3-[(2S)-2- (2,3- dimethylphenyl)- 2-(1H- imidazol-4- yl)ethyl] pyridazine | 278.359 | 279.3 | 98.73 | ¹H NMR (400 MHz, DMSO-d6): δ 11.71 (brs, 1H), 9.01-8.96 (m, 1H), 7.51-7.48 (m, 1H), 7.44-7.40 (m, 1H), 7.32- 7.29 (m, 1H), 7.24- 7.22 (m, 1H), 7.06-6.91 (m, 2H), 6.77 (s, 1H), 4.79 (t, J = 8.0 Hz, 1H), 3.69-3.61 (m, 1H), 3.43-3.26 (m, 1H), 2.20 (s, 6H). |
| A | 56 | 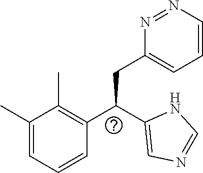 [SMILES: Cc1cccc([C@@H](Cc2cccnn2)c2cnc[nH]2)c1C] | 3-[(2R)-2- (2,3- dimethylphenyl)- 2-(1H- imidazol-4- yl)ethyl] pyridazine | 278.359 | 279.3 | 98.12 | ¹H NMR (400 MHz, DMSO-d6): δ 11.83 (brs, 1H), 9.01 (s, 1H), 7.51- 7.48 (m, 1H), 7.44- 7.40 (m, 1H), 7.32-7.29 (m, 1H), 7.27-7.22 (m, 1H), 7.06-6.91 (m, 2H), 6.77 (s, 1H), 4.79 (t, J = 7.6 Hz, 1H), 3.69- 3.61 (m, 1H), 3.43- 3.26 (m, 1H), 2.20 (s, 6H). |
| A | 57 | 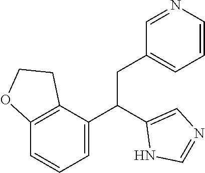 [SMILES: c1cncc(CC(c2cnc[nH]2)c2cccc3c2CCO3)c1] | 3-[2-(2,3- dihydro-1- benzofuran- 4-yl)-2-(1H- imidazol-4- yl)ethyl] pyridine | 291.354 | 292.3 | 98.53 | ¹H NMR (400 MHz, DMSO-d6): δ 11.96 (s, 1H), 8.32-8.30 (m, 1H), 8.28-8.26 (m, 1H), 7.67 (s, 1H), 7.48-7.46 (m, 1H), 7.20-7.17 (m, 1H), 7.00 (t, J = 7.6 Hz, 1H), 6.92 (s, 1H), 6.81 (d, J = 7.6 Hz, 1H), 6.54 (d, J = 7.6 Hz, 1H), 4.46- 4.35 (m, 2H), 4.18 (t, J = 8.0 Hz, 1H), 3.43-3.41 (m, 2H), 3.18-3.10 (m, 2H). |
| A | 58 | 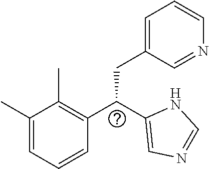 [SMILES: Cc1cccc([C@H](Cc2cccnc2)c2cnc[nH]2)c1C] | 3-[(2S)-2- (2,3- dimethylphenyl)- 2-(1H- imidazol-4- yl)ethyl] pyridine | 277.371 | 278.3 | 99.93 | ¹H NMR (400 MHz, DMSO-d6): δ 11.74 (brs, 1H), 8.30-8.29 (m, 2H), 7.50 (s, 1H), 7.46 (d, J = 8.0 Hz, 1H), 7.23- 7.15 (m, 2H), 7.02- 6.98 (m, 1H), 6.94 (d, J = 7.2 Hz, 1H), 6.76 (s, 1H), 4.49 (t, J = 5.2 Hz, 1H), 3.41-3.31 (m, 1H), 3.09-3.04 (m, 1H), 2.28 (s, 3H), 2.11 (s, 3H). |
| A | 59 | 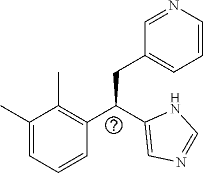 [SMILES: Cc1cccc([C@@H](Cc2cccnc2)c2cnc[nH]2)c1C] | 3-[(2R)-2- (2,3- dimethylphenyl)- 2-(1H- imidazol-4- yl)ethyl] pyridine | 277.371 | 278.3 | 99.58 | ¹H NMR (400 MHz, DMSO-d6): δ 11.75 (brs, 1H), 8.30-8.27 (m, 2H), 7.50 (s, 1H), 7.47 (d, J = 8.0 Hz, 1H), 7.22 (d, J = 7.2 Hz, 1H), 7.19- 7.16 (m, 1H), 7.02-6.93 (m, 2H), 6.77 (s, 1H), 4.49 (t, J = 5.2 Hz, 1H), 3.41-3.31 (m, 1H), 3.09-3.04 (m, 1H), 2.18 (s, 3H), 2.11 (s, 3H). |
| A | 60 | 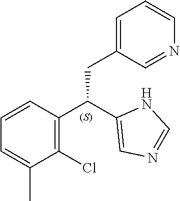 | 3-[(2S)-2- (2-chloro-3- methylphenyl)- 2-(1H- imidazol-4- yl)ethyl] pyridine | 297.79 | 298.2 | 99.86 | ¹H NMR (400 MHz, DMSO-d6): δ 11.83 (s, 1H), 8.30 (s, 2H), 7.56 (s, 1H), 7.50 (d, J = 7.6 (s, 1H), 4.72 (t, J = 7.6 Hz, 1H), 3.40-3.34 (m, 1H), 3.14-3.10 (m, 1H), 2.33 (s, 3H). |
| A | 61 | 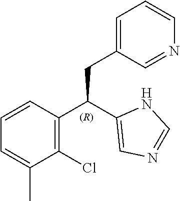 | 3-[(2R)-2-(2- chloro-3- methylphenyl)- 2-(1H- imidazol-4- yl)ethyl]pyridine | 297.79 | 298.2 | 99.77 | ¹H NMR (400 MHz, DMSO-d6): δ 11.85 (s, 1H), 8.26 (s, 2H), 7.56- 7.53 (m, 2H), 7.36 (s, 1H), 7.23-7.15 (m, 3H), 6.80 (s, 1H), 4.74 (t, J = 7.6 Hz, 1H), 3.39- 3.34 (m, 1H), 3.15- 3.10 (m, 1H), 2.26 (s, 3H). |
| A | 62 | 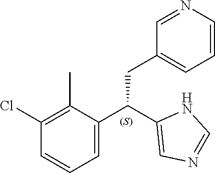 | 3-[(2S)-2- (3-chloro-2- methylphenyl)- 2-(1H- imidazol-4- yl)ethyl]pyridine | 297.79 | 298.2 | 98.53 | ¹H NMR (400 MHz, DMSO-d6): δ 11.82 (brs, 1H), 8.32-8.29 (m, 2H), 7.54 (s, 1H), 7.49 (d, J = 4.4 Hz, 1H), 7.40 (d, J = 7.2 Hz, 1H), 7.23-7.12 (m, 3H), 6.85 (s, 1H), 4.52 (t, J = 7.6 Hz, 1H), 3.44-3.39 (m, 1H), 3.14-3.09 (m, 1H), 2.27 (s, 3H). |
| A | 63 | 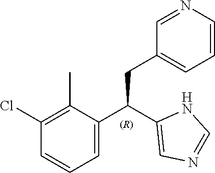 | 3-[(2R)-2-(3- chloro-2- methylphenyl)- 2-(1H- imidazol-4- yl)ethyl]pyridine | 297.79 | 298.2 | 99.49 | ¹H NMR (400 MHz, DMSO-d6): δ 11.80 (brs, 1H), 8.31-8.29 (m, 2H), 7.53 (s, 1H), 7.49 (d, J = 4.4 Hz, 1H), 7.42 (s, 1H), 7.23-7.12 (m, 3H), 6.86 (s, 1H), 4.52 (t, J = 7.6 Hz, 1H), 3.44 3.38 (m, 1H), 3.14-3.08 (m, 1H), 2.27 (s, 3H). |
| A | 64 | 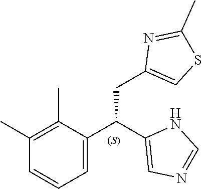 | 4-[(2S)-2- (2,3- dimethylphenyl)- 2-(1H- imidazol-4-yl) ethyl]-2- methyl-1,3- thiazole | 297.4 | 298.3 | 99.88 | 1H NMR (400 MHz, DMSO-d6): 11.68 (brs, 1H), 7.47 (s, 1H), 7.14 (d, J = 7.2 Hz, 1H), 6.99- 6.90 (m, 3H), 6.79 (d, J = 9.6 Hz, 1H), 4.82-4.67 (m, 1H), 3.43-3.37 (m, 1H), 3.13-3.08 (m, 1H), 2.57 (s, 3H), 2.23-2.20 (m, 6H). |
| A | 65 | 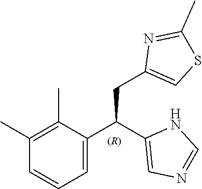 | 4-[(2R)-2- (2,3- dimethylphenyl)- 2-(1H- imidazol-4-yl) ethyl]-2- methyl-1,3- thiazole | 297.4 | 298.3 | 99.28 | 1H NMR (400 MHz, DMSO-d6): 11.67 (brs, 1H), 7.46 (s, 1H), 7.12 (d, J = 6.8 Hz, 1H), 6.98 6.88 (m, 3H), 6.77 (d, J = 10.0 Hz, 1H), 4.80- 4.65 (m, 1H), 3.41-3.37 (m, 1H), 3.11-3.05 (m, 1H), 2.57 (s, 3H), 2.21-2.05 (m, 6H). |
| A | 66 | 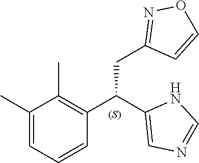 | 3-[(2S)-2- (2,3- dimethylphenyl)- 2-(1H- imidazol-4-yl) ethyl]-1,2- oxazole | 267.33 | 268.3 | 99.44 | 1H NMR (400 MHz, DMSO-d6): δ 11.74 (s, 1H), 8.64 (s, 1H), 7.50 (s, 1H), 7.18-7.15 (m, 1H), 6.98-6.96 (m, 2H), 6.74 (s, 1H), 6.19 (s, 1H), 4.58-4.55 (m, 1H), 3.40-3.35 (m, 1H), 3.20-3.18 (m, 1H), 2.21 (s, 6H). |
| A | 67 | 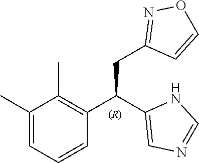 | 3-[(2R)-2- (2,3- dimethylphenyl)- 2-(1H- imidazol-4-yl) ethyl]-1,2- oxazole | 267.33 | 268.3 | 98.41 | 1H NMR (400 MHz, DMSO-d6): δ 11.75 (s, 1H), 8.64 (s, 1H), 7.50 (s, 1H), 7.17-7.15 (m, 1H), 7.00-6.96 (m, 2H), 6.74 (s, 1H), 6.20 (s, 1H), 4.59-4.55 (m, 1H), 3.43-3.38 (m, 1H), 3.20-3.18 (m, 1H), 2.21 (s, 6H). |
| A | 68 | 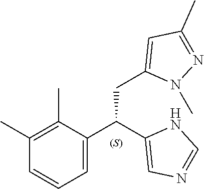 | 5-[(2S)-2- (2,3- dimethylphenyl)- 2-(1H- imidazol-4-yl) ethyl]-1,3- dimethyl-1H- pyrazole | 294.402 | 295.3 | 99.08 | ¹H NMR (400 MHz, DMSO-d6): δ 11.81 (brs, 1H), 7.52 (s, 1H), 7.19 (d, J = 7.2 Hz, 1H), 7.01- 6.95 (m, 2H), 6.76 (s, 1H), 5.60 (s, 1H), 4.49 (t, J = 7.6 Hz, 1H), 3.51 (s, 3H), 3.36-3.30 (m, 1H), 3.06- 3.00 (m, 1H), 2.21 (s, 3H), 2.13 (s, 3H), 1.90 (s, 3H). |
| A | 69 | 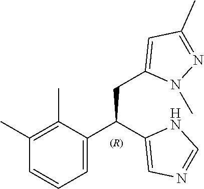 | 5-[(2R)-2- (2,3- dimethylphenyl)- 2-(1H- imidazol-4-yl) ethyl]-1,3- dimethyl-1H- pyrazole | 294.402 | 295.3 | 99.73 | ¹H NMR (400 MHz, DMSO-d6): δ 12.75 (brs, 1H), 7.86 (s, 1H), 7.17 (d, J = 6.8 Hz, 1H), 7.03-6.96 (m, 3H), 5.61 (s, 1H), 4.54 (t, J = 7.6 Hz, 1H), 3.50 (s, 3H), 3.38-3.34 (m, 1H), 3.10-3.02 (m, 1H), 2.22 (s, 3H), 2.15 (s, 3H), 1.98 (s, 3H). |
| A | 70 | 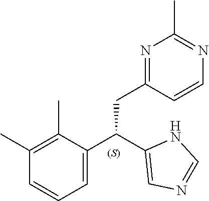 | 4-[(2S)-2- (2,3- dimethylphenyl)- 2-(1H- imidazol-4-yl) ethyl]-2- methyl- pyrimidine | 292.386 | 293.3 | 99.78 | ¹H NMR (400 MHz, DMSO-d6): δ 12.00 (s, 1H), 8.41-8.40 (m, 1H), 7.56 (s, 1H), 7.13 (d, J = 7.2 Hz, 1H), 7.01-6.92 (m, 3H), 6.79 (s, 1H), 4.86 (t, J = 8.0 Hz, 1H), 3.45-3.39 (m, 1H), 3.20- 3.19 (m, 1H), 2.54 (s, 3H), 2.22-2.19 (m, 6H). |
| A | 71 | 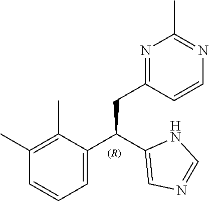 | 4-[(2R)-2- (2,3- dimethylphenyl)- 2-(1H- imidazol-4-yl) ethyl]-2- methyl- pyrimidine | 292.386 | 293.3 | 99.59 | ¹H NMR (400 MHz, DMSO-d6): δ 11.82 (s, 1H), 8.45-8.38 (m, 1H), 7.18 (d, J = 7.6 Hz, 1H), 7.01-6.90 (m, 4H), 6.75-6.70 (m, 1H), 4.81 (t, J = 7.6 Hz, 1H), 3.47-3.42 (m, 1H), 3.19-3.14 (m, 1H), 2.54 (s, 3H), 2.23-2.19 (m, 6H). |
| A | 72 | 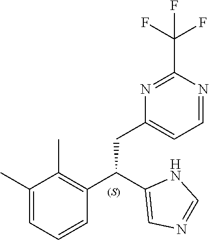 | 4-[(2S)-2- (2,3- dimethylphenyl)- 2-(1H- imidazol-4-yl) ethyl]-2- (trifluoromethyl) pyrimidine | 346.357 | 347.3 | 99.04 | ¹H NMR (400 MHz, DMSO-d6): δ 11.74 (brs, 1H), 8.79 (d, J = 5.2 Hz, 1H), 7.54-7.46 (m, 2H), 7.22 (d, J = 7.6 Hz, 1H), 7.02-6.91 (m, 2H), 6.76 (s, 1H), 4.85 (t, J = 8.0 Hz, 1H), 3.66-3.58 (m, 1H), 3.44-3.37 (m, 1H), 2.20 (s, 6H). |
| A | 73 | 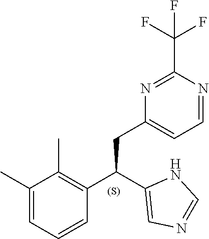 | 4-[(2R)-2- (2,3- dimethylphenyl)- 2-(1H- imidazol-4-yl) ethyl]-2- (trifluoromethyl) pyrimidine | 346.357 | 347.3 | 99.00 | ¹H NMR (400 MHz, DMSO-d6): δ 11.75 (brs, 1H), 8.79 (s, 1H), 7.54- 7.50 (m, 2H), 7.21 (s, 1H), 6.95 (t, J = 7.6 Hz, 2H), 6.75 (s, 1H), 4.85-4.83 (m, 1H), 3.65-3.60 (m, 1H), 3.43-3.38 (m, 1H), 2.20 (s, 6H). |
| A | 74 | 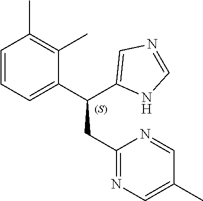 | 2-[(2S)-2- (2,3- dimethylphenyl)- 2-(1H- imidazol-4-yl) ethyl]-5- methyl- pyrimidine | 292.386 | 293.3 | 99.6 | ¹H NMR (400 MHz, DMSO-d6): δ 11.64 (s, 1H), 8.52-8.48 (m, 2H), 7.42 (d, J = 12.0 Hz, 1H), 7.10 (d, J = 8.0 Hz, 1H), 6.97 (s, 1H), 6.94 6.88 (m, 1H), 6.68-6.61 (m, 1H), 5.00 (t, J = 8.0 Hz, 1H), 3.56-3.33 (m, 2H), 2.18 (t, J = 8.0 Hz, 3H), 2.07 (s, 6H). |
| A | 75 | 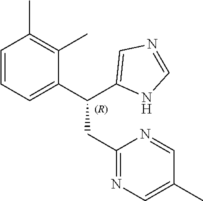 | 2-[(2R)-2- (2,3- dimethylphenyl)- 2-(1H- imidazol-4-yl) ethyl]-5- methylpyrimidine | 292.386 | 293.3 | 99.77 | ¹H NMR (400 MHz, DMSO-d6): δ 11.64 (s, 1H), 8.52-8.48 (m, 2H), 7.41 (d, J = 12.0 Hz, 1H), 7.10 (d, J = 8.0 Hz, 1H), 6.97 (s, 1H), 6.94- 6.88 (m, 1H), 6.68-6.61 (m, 1H), 5.01 (t, J = 8.0 Hz, 1H), 3.57-3.33 (m, 2H), 2.18 (t, J = 8.0 Hz, 3H), 2.07 (s, 6H). |
| A | 76 | 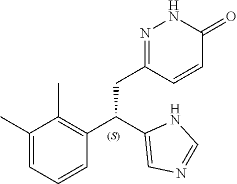 | 6-[(2S)-2- (2,3- dimethylphenyl)- 2-(1H- imidazol-4-yl) ethyl]-2,3- dihydropyridazin- 3-one | 294.358 | 295.3 | 99.51 | ¹H NMR (400 MHz, DMSO-d6): δ 12.69 (s, 1H), 11.75 (s, 1H), 7.49 (s, 1H), 7.16-7.14 (m, 2H), 7.08-6.96 (m, 2H), 6.75-6.65 (m, 2H), 4.60 (s, 1H), 3.30 (s, 1H), 3.05-2.99 (m, 1H), 2.20-2.19 (m, 6H). |
| A | 77 | 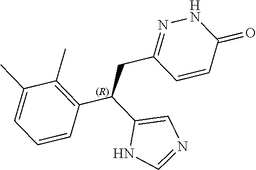 | 6-[(2R)-2- (2,3- dimethylphenyl)- 2-(1H- imidazol-4-yl) ethyl]-2,3- dihydropyridazin- 3-one | 294.358 | 295.3 | 95.74 | ¹H NMR (400 MHz, DMSO-d6): δ 12.68 (s, 1H), 11.73 (s, 1H), 7.49 (s, 1H), 7.16-7.14 (m, 2H), 7.08-6.96 (m, 2H), 6.74-6.65 (m, 2H), 4.61 (s, 1H), 3.30 (s, 1H), 3.05-2.98 (m, 1H), 2.20-2.19 (m, 6H). |
| A | 78 | 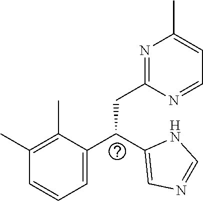 [SMILES: Cc1ccnc(C[C@H](c2cnc[nH]2)c2cccc(C)c2C)n1] | 2-[(2S)-2- (2,3- dimethylphenyl)- 2-(1H- imidazol-4-yl) ethyl]-4- methylpyrimidine | 292.386 | 293.3 | 99.65 | ¹H NMR (400 MHz, DMSO-d6): δ 11.78 (s, 1H), 8.50 (d, J = 4.8 Hz, 1H), 7.42 (d, J = 8.8 Hz, 1H), 7.14-7.08 (m, 2H), 6.99-6.94 (m, 1H), 6.92-6.87 (m, 1H), 6.66 (d, J = 8.4 Hz, 1H), 5.13-5.02 (m, 1H), 3.56-3.51 ( m, 1H), 3.37-3.30 (m, 1H), 2.38 (s, 3H), 2.29-2.18 (m, 6H). |
| A | 79 | 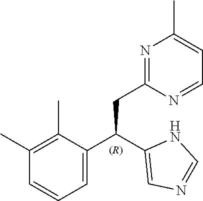 | 2-[(2R)-2- (2,3- dimethylphenyl)- 2-(1H- imidazol-4-yl) ethyl]-4- methylpyrimidine | 292.386 | 293.3 | 98.16 | ¹H NMR (400 MHz, DMSO-d6): δ 11.78 (s, 1H), 8.50 (d, J = 4.8 Hz, 1H), 7.42 (d, J = 8.8 Hz, 1H), 7.14-7.08 (m, 2H), 6.99-6.95 (m, 1H), 6.92-6.87 (m, 1H), 6.67 (d, J = 8.4 Hz, 1H), 5.13-5.02 (m, 1H), 3.56-3.51 ( m, 1H), 3.37-3.30 (m, 1H), 2.38 (s, 3H), 2.29-2.18 (m, 6H). |
| A | 80 | 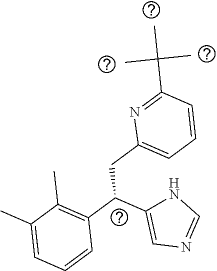 [SMILES: Cc1cccc([C@H](Cc2cccc(C(C)(C)C)n2)c2cnc[nH]2)c1C] | 2-[(2S)-2- (2,3- dimethylphenyl)- 2-(1H- imidazol-4-yl) ethyl]-6- (trifluoromethyl) pyridine | 345.369 | 346.3 | 97.51 | ¹H NMR (400 MHz, DMSO-d6): δ 11.71 (s, 1H), 7.89-7.85 (m, 1H), 7.64-7.59 (m, 1H), 7.44 (s, 1H), 7.36 (d, J = 7.6 Hz, 1H), 7.23 (s, 1H), 6.99-6.88 (m, 2H), 6.72 (s, 1H), 4.84 (t, J = 7.2 Hz, 1H), 3.61- 3.56 (m, 1H), 3.37- 3.35 (m, 1H), 2.15 (s, 6H). |
| A | 81 | 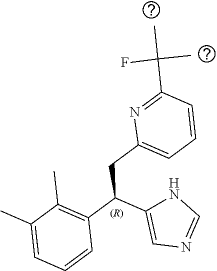 | 2-[(2R)-2- (2,3- dimethylphenyl)- 2-(1H- imidazol-4-yl) ethyl]-6- (trifluoromethyl) pyridine | 345.369 | 346.3 | 99.73 | ¹H NMR (400 MHz, DMSO-d6): δ 11.76 (s, 1H), 7.83 (t, J = 7.6 Hz, 1H), 7.61-7.60 (m, 1H), 7.49 (s, 1H), 7.35 (d, J = 7.6 Hz, 1H), 7.22 (s, 1H), 6.99-6.90 (m, 2H), 6.75 (s, 1H), 4.84 (t, J = 7.2 Hz, 1H), 3.59- 3.54 (m, 1H), 3.37- 3.35 (m, 1H), 2.16 (s, 6H). |
| A | 82 | 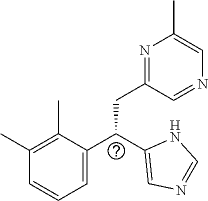 [SMILES: Cc1cncc(C[C@H](c2cnc[nH]2)c2cccc(C)c2C)n1] | 2-[(2S)-2- (2,3- dimethylphenyl)- 2-(1H- imidazol-4-yl) ethyl]-6- methylpyrazine | 292.386 | 293.1 | 99.33 | ¹H NMR (400 MHz, DMSO-d6): δ 11.73 (s, 1H), 8.24 (s, 1H), 8.10 (s, 1H), 7.47 (s, 1H), 7.17 (s, 1H), 6.99-6.92 (m, 2H), 6.76 (s, 1H), 4.79 (s, 1H), 3.49-3.44 (m, 1H), 3.23-3.17 (m, 1H), 2.43 (s, 3H), 2.18 (s, 6H). |
| A | 83 | 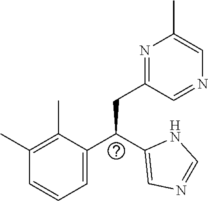 [SMILES: Cc1cncc(C[C@@H](c2cnc[nH]2)c2cccc(C)c2C)n1] | 2-[(2R)-2- (2,3- dimethylphenyl)- 2-(1H- imidazol-4-yl) ethyl]-6- methylpyrazine | 292.386 | 293.1 | 99.87 | ¹H NMR (400 MHz, DMSO-d6): δ 11.71 (s, 1H), 8.24 (s, 1H), 8.09 (s, 1H), 7.48 (s, 1H), 7.20 (s, 1H), 6.99-6.93 (m, 2H), 6.76 (s, 1H), 4.78 (s, 1H), 3.46-3.41 (m, 1H), 3.23-3.17 (m, 1H), 2.43 (s, 3H), 2.18 (s, 6H). |
| A | 84 | 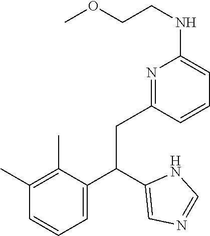 [SMILES: COCCNc1cccc(CC(c2cnc[nH]2)c2cccc(C)c2C)n1] | 6-[2-(2,3- dimethylphenyl)- 2-(1H- imidazol-4-yl) ethyl]-N-(2- methoxyethyl) pyridin-2- amine | 350.466 | 351.3 | 98.9 | ¹H NMR (400 MHz, DMSO-d6): δ 11.75 (s, 1H), 7.45-7.41 (m, 1H), 7.16-7.08 (m, 2H), 6.97-6.87 (m, 2H), 6.74- 6.70 (m, 1H), 6.39- 6.29 (m, 1H), 6.27-6.18 (m, 2H), 4.95-4.80 (m, 1H), 3.46-3.38 (m, 4H), 3.24 (s, 3H), 3.18- 3.13 (m, 1H), 2.98-2.92 (m, 1H), 2.27-2.19 (m, 6H). |
| A | 85 | 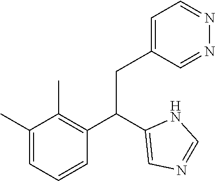 [SMILES: Cc1cccc(C(Cc2ccnnc2)c2cnc[nH]2)c1C] | 4-[2-(2,3- dimethylphenyl)- 2-(1H- imidazol-4- yl)ethyl] pyridazine | 278.4 | 279.3 | 99.04 | 1H NMR (400 MHz, DMSO-d6): δ 11.75 (s, 1H), 8.97-8.86 (m, 2H), 7.51 (s, 1H), 7.39- 7.37 (m, 1H), 7.24 (s, 1H), 7.00-6.96 (m, 2H), 6.76 (s, 1H), 4.59-4.57 (m, 1H), 3.45-3.40 (m, 1H), 3.15-3.10 (m, 1H), 2.19 (s, 3H), 2.15 (s, 3H). |
| A | 86 | 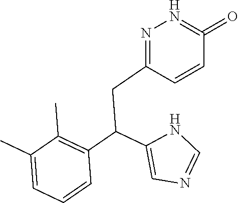 | 6-[2-(2,3- dimethylphenyl)- 2-(1H- imidazol-4-yl) ethyl]-2,3- dihydropyridazin- 3-one | 294.358 | 295.3 | 99.1 | ¹H NMR (400 MHz, DMSO-d6): δ 12.68 (s, 1H), 11.73 (s, 1H), 7.50- 7.45 (m, 1H), 7.23- 7.12 (m, 2H), 7.02-6.92 (m, 2H), 6.76 (s, 1H), 6.72-6.64 (m, 1H), 4.57 (t, J = 7.6 Hz, 1H), 3.24-2.99 (m, 2H), 2.21-2.19 (m, 6H). |
| A | 87 | 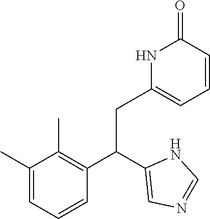 [SMILES: Cc1cccc(C(Cc2cccc(=O)[nH]2)c2cnc[nH]2)c1C] | 6-[2-(2,3- dimethylphenyl)- 2-(1H- imidazol-4- yl)ethyl] pyridin-2-ol | 293.37 | 294.3 | 99.66 | ¹H NMR (400 MHz, DMSO-d6): δ 11.70 (s, 1H), 8.33 (s, 1H), 7.22- 7.17 (m, 2H), 7.18-7.00 (m, 4H), 6.08 (d, J = 9.2 Hz, 1H), 5.77 (d, J = 6.0 Hz, 1H), 4.76 (t, J = 8.0 Hz, 1H), 3.32-3.21 (m, 1H), 3.03-2.97 (m, 1H), 2.21-2.17 (m, 6H). |
| A | 88 | 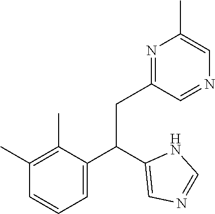 [SMILES: Cc1cncc(CC(c2cnc[nH]2)c2cccc(C)c2C)n1] | 2-[2-(2,3- dimethylphenyl)- 2-(1H- imidazol-4-yl) ethyl]-6- methylpyrazine | 292.386 | 293.1 | 99.79 | ¹H NMR (400 MHz, DMSO-d6): δ 11.74 (s, 1H), 8.24 (s, 1H), 8.10 (s, 1H), 7.48 (s, 1H), 7.17 (s, 1H), 6.99-6.92 (m, 2H), 6.77 (s, 1H), 4.79 (s, 1H), 3.49-3.44 (m, 1H), 3.23-3.17 (m, 1H), 2.43 (s, 3H), 2.19 (s, 6H). |
| A | 89 | 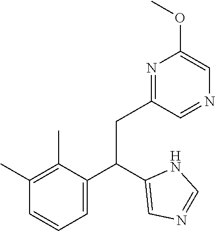 [SMILES: COc1cncc(CC(c2cnc[nH]2)c2cccc(C)c2C)n1] | 2-[2-(2,3- dimethylphenyl)- 2-(1H- imidazol-4-yl) ethyl]-6- methoxy- pyrazine | 308.385 | 309.3 | 99.68 | ¹H NMR (400 MHz, DMSO-d6): δ 11.70 (brs, 1H), 8.00 (s, 1H), 7.91 (s, 1H), 7.48 (s, 1H), 7.18 (s, 1H), 6.97-6.93 (m, 2H), 6.77 (s, 1H), 4.80 (brs, 1H), 3.87 (s, 3H), 3.44- 3.40 (m, 1H), 3.20-3.15 (m, 1H), 2.33-2.19 (m, 6H). |
| A | 90 | 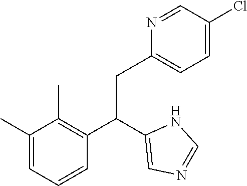 [SMILES: Cc1cccc(C(Cc2ccc(Cl)cn2)c2cnc[nH]2)c1C] | 5-chloro-2- [2-(2,3- dimethylphenyl)- 2-(1H- imidazol-4- yl)ethyl] pyridine | 311.81 | 312.2 | 98.19 | ¹H NMR (400 MHz, DMSO-d6): δ 11.81 (s, 1H), 8.51-8.48 (m, 1H), 7.75-7.70 (m, 1H), 7.48- 7.40 (m, 1H), 7.23-7.20 (m, 1H), 7.13-7.09 (m, 1H), 7.07-6.98 (m, 2H), 6.75-6.71 (m, 1H), 4.79- 4.75 (m, 1H), 3.53-3.49 (m, 1H), 3.23-3.20 (m, 1H), 2.23 (s, 6H). |
| A | 91 | 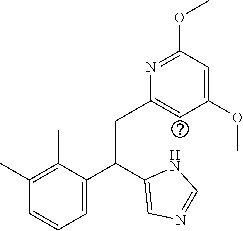 | 2-[2-(2,3- dimethylphenyl)- 2-(1H- imidazol-4- yl)ethyl]-4,6- dimethoxy- pyrimidine | 338.411 | 339.3 | 99.71 | ¹H NMR (400 MHz, DMSO-d6): δ 11.80 (s, 1H), 7.46 (s, 1H), 7.13- 7.11 (m, 1H), 6.89- 6.70 (m, 1H), 6.72-6.69 (s, 2H), 5.99-5.97 (m, 1H), 5.07-5.03 (m, 1H), 3.82-3.80 (m, 6H), 3.46-3.40 (m, 1H), 3.27-3.20 (m, 1H), 2.28-2.19 (m, 6H). |
| A | 92 | 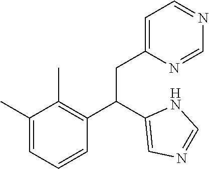 [SMILES: Cc1cccc(C(Cc2ccncn2)c2cnc[nH]2)c1C] | 4-[2-(2,3- dimethylphenyl)- 2-(1H- imidazol- 4-yl)ethyl] pyrimidine | 278.359 | 279.3 | 98.81 | ¹H NMR (400 MHz, DMSO-d6): δ 11.72 (brs, 1H), 9.04 (s, 1H), 8.54 (s, 1H), 7.48 (s, 1H), 7.24- 7.18 (m, 2H), 6.97-6.94 (m, 2H), 6.75 (s, 1H), 4.84 (brs, 1H), 3.51-3.42 (m, 1H), 3.23-3.18 (m, 1H), 2.27 (s, 6H). |
| A | 93 | 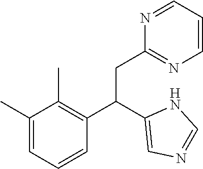 [SMILES: Cc1cccc(C(Cc2ncccn2)c2cnc[nH]2)c1C] | 2-[2-(2,3- dimethylphenyl)- 2-(1H- imidazol-4- yl)ethyl] pyrimidine | 278.359 | 279.2 | 97.1 | ¹H NMR (400 MHz, DMSO-d6): δ 11.74 (s, 1H), 8.66 (d, J = 4.8 Hz, 2H), 7.45 (s, 1H), 7.26- 7.24 (m, 1H), 7.08-7.06 (m, 1H), 6.97-6.94 (m, 2H), 6.68 (s, 1H), 5.06 (t, J = 7.6 Hz, 1H), 3.59- 3.53 (m, 1H), 3.41-3.38 (m, 1H), 2.26 (s, 3H), 2.20 (s, 3H). |
| A | 94 | 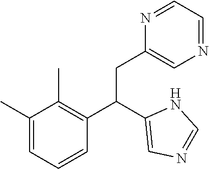 [SMILES: Cc1cccc(C(Cc2cnccn2)c2cnc[nH]2)c1C] | 2-[2-(2,3- dimethylphenyl)- 2-(1H- imidazol-4- yl)ethyl] pyrazine | 278.359 | 279.2 | 99.46 | ¹H NMR (400 MHz, DMSO-d6): δ 11.77 (brs, 1H), 8.53 (s, 1H), 8.37 (d, J = 2.4 Hz, 2H), 7.49 (s, 1H), 7.17 (d, J = 6.4 Hz, 1H), 7.01-6.94 (m, 2H), 6.78 (s, 1H), 4.81 (t, J = 7.6 Hz, 1H), 3.56-3.50 (m, 1H), 3.26-3.21 (m, 1H), 2.20 (s, 6H). |
| A | 95 | 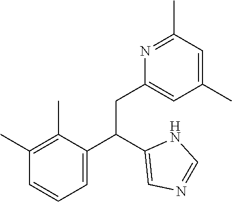 | 2-[2-(2,3- dimethylphenyl)- 2-(1H- imidazol-4-yl) ethyl]-4,6- dimethyl- pyridine | 305.425 | 306.3 | 99.63 | ¹H NMR (400 MHz, DMSO-d6): δ 11.74 (brs, 1H), 7.56 (s, 1H), 7.16- 7.10 (m, 1H), 7.02- 6.91 (m, 2H), 6.80 (s, 1H), 6.75 (s, 1H), 6.70 (s, 1H), 4.87 (t, J = 7.6 Hz, 1H), 3.51 (brs, 1H), 3.18-3.08 (m, 1H), 2.33 (s, 3H), 2.31-2.28 (m, 6H), 2.10 (s, 3H). |
| A | 96 | 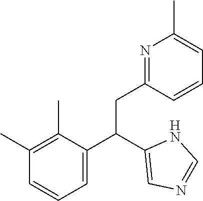 [SMILES: Cc1cccc(CC(c2cnc[nH]2)c2cccc(C)c2C)n1] | 2-[2-(2,3- dimethylphenyl)- 2-(1H- imidazol-4-yl) ethyl]-6- methyl- pyridine | 291.398 | 292.2 | 99.67 | ¹H NMR (400 MHz, DMSO-d6): δ 11.73 (brs, 1H), 7.47 (s, 1H), 7.41 (t, J = 7.6 Hz, 1H), 7.14 (s, 1H), 6.98-6.92 (m, 3H), 6.85-6.83 (m, 1H), 6.75 (s, 1H), 4.84 (t, J = 5.2 Hz, 1H), 3.43-3.36 (m, 1H), 3.19-3.13 (m, 1H), 2.41 (s, 3H), 2.19 (s, 6H). |
| A | 97 | 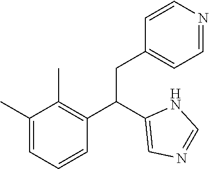 [SMILES: Cc1cccc(C(Cc2ccncc2)c2cnc[nH]2)c1C] | 4-[2-(2,3- dimethylphenyl)- 2-(1H- imidazol-5- yl)ethyl] pyridine | 277.371 | 278.2 | 99.58 | ¹H NMR (400 MHz, DMSO-d6): δ 11.75 (s, 1H), 8.31 (d, J = 4.0 Hz, 2H), 7.48 (s, 1H), 7.17 (d, J = 4.0 Hz, 1H), 7.10-7.04 (m, 2H), 6.99-6.91 (m, 2H), 6.73 (s, 1H), 4.53 (t, J = 8.0 Hz, 1H), 3.44-3.40 (m, 1H), 3.09-3.04 (m, 1H), 2.28 (s, 6H). |
| A | 98 | 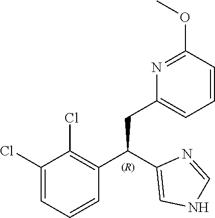 | 4-[(R)-1-(2,3- dichlorophenyl)- 2-(6- methoxy-2- pyridyl)ethyl] imidazole | 348.233 | 348.2 | 99.56 | 1H NMR (400 MHz, DMSO-d6): δ 11.79 (s, 1H), 7.52-7.46 (m, 3H), 7.33-7.23 (m, 2H), 6.87 (s, 1H), 6.70 (d, J = 6.8 Hz, 1H), 6.51 (d, J = 8.0 Hz, 1H), 5.12 (t, J = 7.2 Hz, 1H), 3.77 (s, 3H), 3.43-3.40 (m, 1H), 3.28- 3.25 (m, 1H). |
| A | 99 | 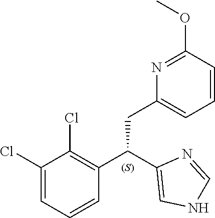 | 4-[(S)-1-(2,3- dichlorophenyl)- 2-(6- methoxy-2- pyridyl)ethyl] imidazole | 348.233 | 348.2 | 99.3 | 1H NMR (400 MHz, DMSO-d6): δ 11.85 (s, 1H), 7.53 (s, 1H), 7.49- 7.45 (m, 2H), 7.42- 7.40 (m, 1H), 7.26 (t, J = 8.0 Hz, 1H) 6.85 (s, 1H), 6.72 (d, J = 7.2 Hz, 1H), 6.51 (d, J = 8.0 Hz, 1H), 5.15 (t, J = 7.2 Hz, 1H), 3.77 (s, 3H), 3.46-3.41 (m, 1H), 3.31- 3.28 (m, 1H). |
| A | 100 | 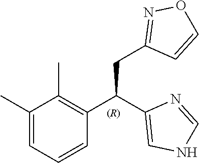 | 3-[(R)-2-(4- imidazolyl)- 2-(2,3- xylyl)ethyl] isoxazole | 267.332 | 268.3 | 98.41 | 1H NMR (400 MHz, DMSO-d6): δ 11.75 (s, 1H), 8.64 (s, 1H), 7.50 (s, 1H), 7.17-7.15 (m, 1H), 7.00-6.96 (m, 2H), 6.74 (s, 1H), 6.20 (s, 1H), 4.59-4.55 (m, 1H), 3.43-3.38 (m, 1H), 3.20-3.18 (m, 1H), 2.21 (s, 6H). |
| A | 101 | 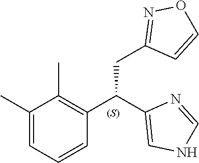 | 3-[(S)-2-(4- imidazolyl)- 2-(2,3- xylyl)ethyl] isoxazole | 267.332 | 268.3 | 99.44 | 1H NMR (400 MHz, DMSO-d6): δ 11.74 (s, 1H), 8.64 (s, 1H), 7.50 (s, 1H), 7.18-7.15 (m, 1H), 6.98-6.96 (m, 2H), 6.74 (s, 1H), 6.19 (s, 1H), 4.58-4.55 (m, 1H), 3.40-3.35 (m, 1H), 3.20-3.18 (m, 1H), 2.21 (s, 6H). |
| A | 102 | 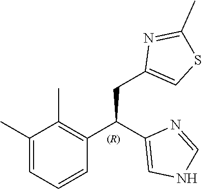 | 4-[(R)-2-(4- imidazolyl)- 2-(2,3-xylyl) ethyl]-2- methyl-1,3- thiazole | 297.427 | 298.3 | 99.28 | 1H NMR (400 MHz, DMSO-d6): δ 11.67 (brs, 1H), 7.46 (s, 1H), 7.12 (d, J = 6.8 Hz, 1H), 6.98 6.88 (m, 3H), 6.77 (d, J = 10.0 Hz, 1H), 4.80- 4.65 (m, 1H), 3.41-3.37 (m, 1H), 3.11-3.05 (m, 1H), 2.57 (s, 3H), 2.21 (s, 3H), 2.20 (s, 3H). |
| A | 103 | 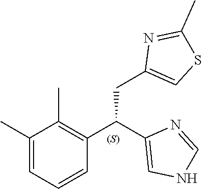 | 4-[(S)-2- (4-imidazolyl)- 2-(2,3-xylyl) ethyl]-2- methyl-1,3- thiazole | 297.427 | 298.3 | 99.88 | 1H NMR (400 MHz, DMSO-d6): δ 11.68 (brs, 1H), 7.47 (brs, 1H), 7.14 (d, J = 7.2 Hz, 1H), 6.99-6.90 (m, 3H), 6.79 (d, J = 9.6 Hz, 1H), 4.82-4.67 (m, 1H), 3.43- 3.37 (m, 1H), 3.13- 3.08 (m, 1H), 2.57 (s, 3H), 2.23-2.20 (m, 6H), |
| A | 104 | 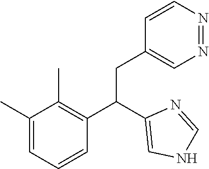 [SMILES: Cc1cccc(C(Cc2ccnnc2)c2c[nH]cn2)c1C] | 4-[2-(4- imidazolyl)- 2-(2,3- xylyl)ethyl] pyridazine | 278.359 | 279.3 | 99.04 | 1H NMR (400 MHz, DMSO-d6): δ 11.75 (s, 1H), 8.97-8.86 (m, 2H), 7.51 (s, 1H), 7.39- 7.37 (m, 1H), 7.24 (s, 1H), 7.00-6.96 (m, 2H), 6.76 (s, 1H), 4.59-4.56 (m, 1H), 3.45-3.40 (m, 1H), 3.15-3.10 (m, 1H), 2.19 (s, 3H), 2.15 (s, 3H) |
| A | 105 | 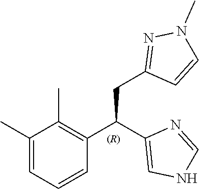 | 3-[(R)-2- (4-imidazolyl)- 2-(2,3-xylyl) ethyl]-1- methylpyrazole | 280.375 | 281.3 | 97.11 | 1H NMR (400 MHz, DMSO-d6): δ 11.67 (s, 1H), 7.47-7.36 (m, 2H), 7.14 (d, J = 7.6 Hz, 1H), 7.00-6.90 (m, 2H), 6.73 (s, 1H), 5.70 (s, 1H), 4.50 (t, J = 7.6 Hz, 1H), 3.71 (s, 3H), 3.31 3.26 (m, 1H), 3.01-2.96 (m, 1H), 2.20 (s, 6H). |
| A | 106 | 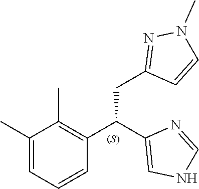 | 3-[(S)-2- (4-imidazolyl)- 2-(2,3-xylyl) ethyl]-1- methylpyrazole | 280.375 | 281.3 | 98.35 | 1H NMR (400 MHz, DMSO-d6): δ 11.71 (s, 1H), 7.46 (s, 1H), 7.38 (s, 1H), 7.09 (s, 1H), 6.98-6.94 (m, 2H), 6.72 (s, 1H), 5.73 (s, 1H), 4.54 (brs, 1H), 3.74 (s, 3H), 3.33-3.29 (m, 1H), 3.02-2.96 (m, 1H), 2.20 (s, 6H). |
| A | 107 | 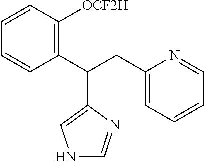 | 4-[1-(o- difluoro- methoxyphenyl) 2-(2- pyridyl)ethyl] imidazole | 315.323 | 316.1 | 99.5 | 1H NMR (400 MHz, DMSO-d6): δ 11.92 (s, 1H), 8.42 (s, 1H), 7.60- 7.55 (m, 3H), 7.26- 7.09 (m, 6H), 6.78 (t, J = 36.0 Hz, 1H), 4.88 (t, J = 10.0 Hz, 1H), 3.53- 3.49 (m, 1H), 3.28- 3.25 (m, 1H). |
| A | 108 | 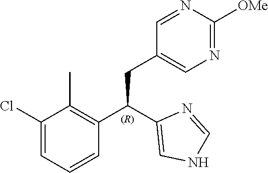 | 5-[(R)-2-(3- chloro-2- tolyl)-2-(4- imidazolyl) ethyl]-2- methoxy- pyrimidine | 328.803 | 329.3 | 99.64 | 1H NMR (400 MHz, DMSO-d6): δ 11.77 (brs, 1H), 8.34 (d, J = 5.2 Hz, 1H), 7.51 (s, 1H), 7.32 (d, J = 7.6 Hz, 1H), 7.20 (d, J = 7.6 Hz, 1H), 7.17-7.08 (m, 1H), 6.90 (d, J = 5.2 Hz, 1H), 6.83 (s, 1H), 4.84 (t, J = 7.6 Hz, 1H), 3.84 (s, 3H), 3.49-3.43 (m, 1H), 3.24-3.16 (m, 1H), 2.40 (s, 3H). |
| A | 109 | 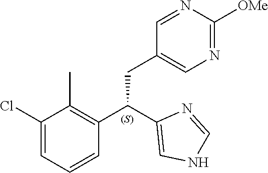 | 5-[(S)-2-(3- chloro-2- tolyl)-2-(4- imidazolyl) ethyl]-2- methoxy- pyrimidine | 328.803 | 329.2 | 99.02 | 1H NMR (400 MHz, DMSO-d6): δ 11.77 (brs, 1H), 8.34 (d, J = 5.2 Hz, 1H), 7.51 (s, 1H), 7.32 (d, J = 7.6 Hz, 1H), 7.20 (d, J = 7.6 Hz, 1H), 7.13-7.08 (m, 1H), 6.89 (d, J = 5.2 Hz, 1H), 6.83 (s, 1H), 4.84 (t, J = 7.6 Hz, 1H), 3.84 (s, 3H), 3.49-3.43 (m, 1H), 3 .24-3.16 (m, 1H), 2.40 (s, 3H). |
| A | 110 | 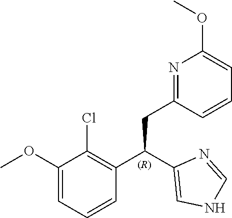 | 4-[(R)-1- (2-chloro-3- methoxyphenyl)- 2-(6- methoxy-2- pyridyl)ethyl] imidazole | 343.814 | 344.3 | 98.09 | 1H NMR (400 MHz, DMSO-d6): δ 11.78 (s, 1H), 7.49-7.44 (m, 2H), 7.20-7.16 (m, 1H), 7.05 (d, J = 7.6 Hz, 1H), 6.90 (d, J = 8.0 Hz, 1H), 6.76 (s, 1H), 6.70 (d, J = 7.2 Hz, 1H), 6.50 (d, J = 8.0 Hz, 1H), 5.14- 5.11 (m, 1H), 3.78 (s, 6H), 3.42-3.36 (m, 1H), 3.26-3.20 (m, 1H). |
| A | 111 | 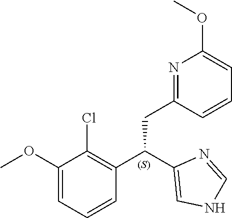 | 4-[(S)-1- (2-chloro-3- methoxyphenyl)- 2-(6- methoxy-2- pyridyl)ethyl] imidazole | 343.814 | 344.3 | 98.69 | 1H NMR (400 MHz, DMSO-d6): δ 11.74 (s, 1H), 7.49-7.44 (m, 2H), 7.20-7.16 (m, 1H), 7.05 (d, J = 7.6 Hz, 1H), 6.90 (d, J = 8.0 Hz, 1H), 6.76 (s, 1H), 6.70 (d, J = 7.2 Hz, 1H), 6.50 (d, J = 8.0 Hz, 1H), 5.12 (s, 1H), 3.78 (s, 6H), 3.42-3.36 (m, 1H), 3.26- 3.20 (m, 1H). |
| A | 112 | 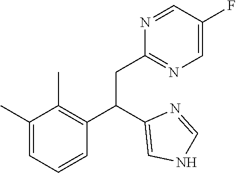 [SMILES: Cc1cccc(C(Cc2ncc(F)cn2)c2c[nH]cn2)c1C] | 5-fluoro-2- [2-(4- imidazolyl)-2- (2,3- xylyl)ethyl] pyrimidine | 296.349 | 297.3 | 96.6 | 1H NMR (400 MHz, DMSO-d6): δ 11.67 (s, 1H), 8.73 (s, 2H), 7.44 (s, 1H), 7.11 (s, 2H), 6.98-6.92 (m, 1H), 6.68 (s, 1H), 4.98 (s, 1H), 3.59-3.41 (m, 2H), 2.33 (s, 6H) |
| A | 113 | 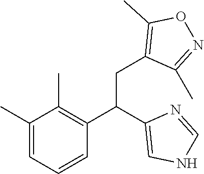 [SMILES: Cc1cccc(C(Cc2c(C)noc2C)c2c[nH]cn2)c1C] | 4-[2-(4- imidazolyl)- 2-(2,3-xylyl) ethyl]-3,5- dimethyl- isoxazole | 295.386 | 296.3 | 99.66 | 1H NMR (400 MHz, DMSO-d6): δ 11.73 (s, 1H), 7.51 (s, 1H), 7.34 (d, J = 7.6 Hz, 1H), 7.18 6.94 (m, 2H), 6.81 (s, 1H), 4.36-4.25 (m, 1H), 3.17-3.04 (m, 1H), 2.82- 2.72 (m, 1H), 2.24 (s, 3H), 2.03-1.87 (m, 9H). |
| A | 114 | 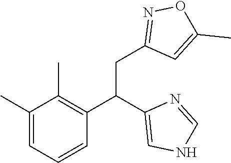 | 3-[2-(4- imidazolyl)-2- (2,3-xylyl) ethyl]-5- methyl- isoxazole | 281.359 | 282.3 | 98.4 | 1H NMR (400 MHz, DMSO-d6): δ 11.73 (s, 1H), 7.49 (s, 1H), 7.14 (s, 1H), 7.00-6.96 (m, 2H), 6.73 (s, 1H), 5.87 (s, 1H), 4.58-4.55 (m, 1H), 3.29-3.26 (m, 1H), 3.10-3.05 (m, 1H), 2.26 (s, 3H), 2.21 (s, 6H) |
| A | 115 | 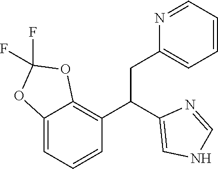 [SMILES: FC1(F)Oc2cccc(C(Cc3ccccn3)c3c[nH]cn3)c2O1] | 2,2-difluoro- 4-[1-(4- imidazolyl)- 2-(2- pyridyl)ethyl]- 2H-1,3- benzodioxole | 329.306 | 330.2 | 98.72 | 1H NMR (400 MHz, DMSO-d6): δ 11.93 (brs, 1H), 8.43 (d, J = 4.0 Hz, 1H), 7.57-7.50 (m, 2H), 7.18-7.13 (m, 3H), 7.08-7.04 (m, 2H), 6.92 (s, 1H), 4.70 (t, J = 8.4 Hz, 1H), 3.56- 3.51 (m, 1H), 3.50-3.45 (m, 1H). |
| A | 116 | 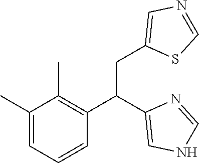 [SMILES: Cc1cccc(C(Cc2cncs2)c2c[nH]cn2)c1C] | 5-[2-(4- imidazolyl)-2- (2,3-xylyl) ethyl]-1,3- thiazole | 283.4 | 284.2 | 98.36 | 1H NMR (400 MHz, DMSO-d6): δ 11.80 (s, 1H), 8.76 (s, 1H), 7.59- 7.49 (m, 2H), 7.21 (d, J = 8.0 Hz, 1H), 7.06- 6.96 (m, 2H), 6.79 (s, 1H), 4.42 (t, J = 8.0 Hz, 1H), 3.65-3.60 (m, 1H), 3.39-3.35 (m, 1H), 2.12 (s, 3H), 2.07 (s, 3H). |
| A | 117 | 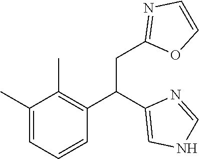 [SMILES: Cc1cccc(C(Cc2ncco2)c2c[nH]cn2)c1C] | 2-[2-(4- imidazolyl)-2- (2,3-xylyl) ethyl]-1,3- oxazole | 267.332 | 268.3 | 99.96 | 1H NMR (400 MHz, DMSO-d6): δ 11.77 (s, 1H), 7.87 (s, 1H), 7.48 (s, 1H), 7.11-7.10 (m, 1H), 6.99-6.94 (m, 3H), 6.73 (s, 1H), 4.79 (t, J = 8.4 Hz, 1H), 3.51-3.46 (m, 1H), 3.30-3.24 (m, 1H), 2.21 (s, 6H) |
| A | 118 | 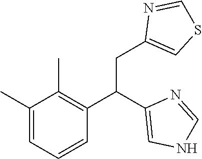 [SMILES: Cc1cccc(C(Cc2cscn2)c2c[nH]cn2)c1C] | 4-[2-(4- imidazolyl)-2- (2,3-xylyl) ethyl]-1,3- thiazole | 283.4 | 284.2 | 99.29 | 1H NMR (400 MHz, DMSO-d6): δ 11.72 (brs, 1H), 8.94 (s, 1H), 7.46 (s, 1H), 7.10 (s, 2H), 7.02-6.92 (m, 2H), 6.76 (s, 1H), 4.77 (t, J = 7.6 Hz, 1H), 3.52-3.46 (m, 1H), 3.24-3.19 (m, 1H), 2.25- 2.15 (m, 6H). |
| A | 119 | 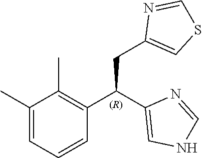 | 4-[(R)-2-(4- imidazolyl)-2- (2,3-xylyl) ethyl]-1,3- thiazole | 283.4 | 284.3 | 97.07 | 1H NMR (400 MHz, DMSO-d6): δ 11.71 (brs, 1H), 8.94 (s, 1H), 7.46 (s, 1H), 7.09 (brs, 2H), 6.98-6.94 (m, 2H), 6.76 (s, 1H), 4.76 (brs, 1H), 3.49 (brs, 1H), 3.24- 3.19 (m, 1H), 2.20 (s, 6H). |
| A | 120 | 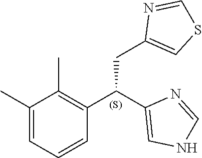 | 4-[(S)-2-(4- imidazolyl)-2- (2,3-xylyl) ethyl]-1,3- thiazole | 283.4 | 284.3 | 99.29 | 1H NMR (400 MHz, DMSO-d6): δ 11.72 (brs, 1H), 8.94 (d, J = 2.0 Hz, 1H), 7.46 (s, 1H), 7.10 (s, 2H), 6.98-6.92 (m, 2H), 6.81 (s, 1H), 4.76 (s, 1H), 3.52-3.46 (m, 1H), 3.24-3.19 (m, 1H), 2.20 (s, 6H). |
| A | 121 | 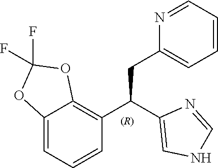 | 4-[(R)-1-(4- imidazolyl)-2- (2-pyridyl) ethyl]-2,2- difluoro-2H- 1,3- benzodioxole | 329.306 | 330.2 | 99.7 | 1H NMR (400 MHz, DMSO-d6): δ 11.81 (brs, 1H), 8.43 (d, J = 4.0 Hz, 1H), 7.58-7.51 (m, 2H), 7.18-7.13 (m, 5H), 6.93 (s, 1H), 4.68 (t, J = 8.4 Hz, 1H), 3.56-3.42 (m, 2H). |
| A | 122 | 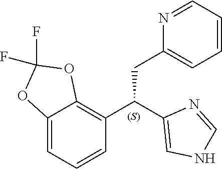 | 4-[(S)-1-(4- imidazolyl)-2- (2-pyridyl) ethyl]-2,2- difluoro-2H- 1,3- benzodioxole | 329.306 | 330.2 | 97.12 | 1H NMR (400 MHz, DMSO-d6): δ 11.81 (brs, 1H), 8.43 (d, J = 4.0 Hz, 1H), 7.60-7.50 (m, 2H), 7.17-7.03 (m, 5H), 6.93 (s, 1H), 4.68 (t, J = 8.4 Hz, 1H), 3.56-3.46 (m, 2H). |
| A | 123 | 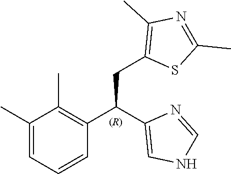 | 5-[(R)-2-(4- imidazolyl)- 2-(2,3-xylyl) ethyl]-2,4- dimethyl-1,3- thiazole | 311.454 | 312.3 | 97.92 | 1H NMR (400 MHz, DMSO-d6): δ 11.77 (brs, 1H), 7.52 (s, 1H), 7.22 (brs, 1H), 7.01-6.95 (m, 2H), 6.76 (s, 1H), 4.34 (brs, 1H), 3.50-3.41 (m, 1H), 3.19- 3.14 (m, 1H), 2.38 (s, 3H), 2.25 (s, 3H), 2.15 (s, 3H), 2.06 (s, 3H). |
| A | 124 | 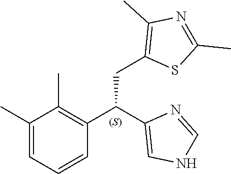 | 5-[(S)-2-(4- imidazolyl)- 2-(2,3-xylyl) ethyl]-2,4- dimethyl-1,3- thiazole | 311.454 | 312.3 | 97.6 | 1H NMR (400 MHz, DMSO-d6): δ 11.81 (brs, 1H), 7.48 (s, 1H), 7.22 (d, J = 7.6 Hz, 1H), 7.01 6.95 (m, 2H), 6.81 (s, 1H), 4.36 (t, J = 7.6 Hz, 1H), 3.46-3.41 (m, 1H), 3.19-3.14 (m, 1H), 2.40 (s, 3H), 2.24 (s, 3H), 2.07 (s, 3H), 2.05 (s, 3H). |
| A | 125 | 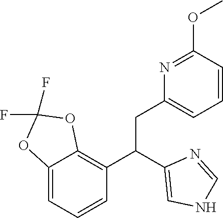 [SMILES: COc1cccc(CC(c2c[nH]cn2)c2cccc3c2OC(F)(F)O3)n1] | 2,2-difluoro- 4-[1-(4- imidazolyl)- 2-(6- methoxy-2- pyridyl)ethyl]- 2H-1,3- benzodioxole | 359.332 | 360.3 | 99.67 | 1H NMR (400 MHz, DMSO-d6): δ 11.83 (s, 1H), 7.54 (s, 1H), 7.45 (t, J = 7.6 Hz, 1H), 7.13- 7.03 (m, 3H), 6.93 (s, 1H), 6.67-6.66 (m, 1H), 6.51 (d, J = 8.0 Hz, 1H), 4.73-4.69 (m, 1H), 3.75 (s, 3H), 3.47-3.43 (m, 1H), 3.31-3.27 (m, 1H) |
| A | 126 | 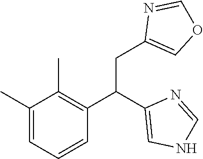 [SMILES: Cc1cccc(C(Cc2cocn2)c2c[nH]cn2)c1C] | 4-[2-(4- imidazolyl)-2- (2,3-xylyl) ethyl]-1,3- oxazole | 267.332 | 268.3 | 99.61 | 1H NMR (400 MHz, DMSO-d6): δ 11.79 (s, 1H), 8.20-8.16 (m, 1H), 7.51-7.44 (m, 2H), 7.15 (d, J = 6.8 Hz, 1H), 6.99-6.91 (m, 2H), 6.76-6.71 (m, 1H), 4.59 (d, J = 7.6 Hz, 1H), 3.27-3.21 (m, 1H), 2.98- 2.93 (m, 1H), 2.22 (s, 6H). |
| A | 127 | 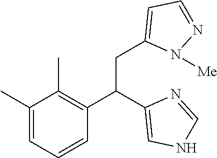 [SMILES: Cc1cccc(C(Cc2ccnn2C)c2c[nH]cn2)c1C] | 5-[2-(4- imidazolyl)-2- (2,3-xylyl) ethyl]-1- methyl- pyrazole | 280.375 | 281.3 | 98.59 | 1H NMR (400 MHz, DMSO-d6): δ 11.79 (brs, 1H), 7.51 (s, 1H), 7.22 (d, J = 7.2 Hz, 1H), 7.14 (s, 1H), 7.01-6.95 (m, 2H), 6.76 (s, 1H), 5.79 (s, 1H), 4.53 (t, J = 7.6 Hz, 1H), 3.61 (s, 3H), 3.43-3.34 (m, 1H), 3.15-3.09 (m, 1H), 2.20 (s, 3H), 2.14 (s, 3H). |
| A | 128 | 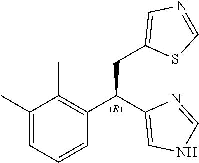 | 5-[(R)-2-(4- imidazolyl)- 2-(2,3-xylyl) ethyl]-1,3- thiazole | 283.4 | 284.2 | 99.64 | 1H NMR (400 MHz, DMSO-d6): δ 11.76 (s, 1H), 8.74 (s, 1H), 7.53 (s, 1H), 7.23 (d, J = 7.2 Hz, 1H), 7.02-6.93 (m, 3H), 6.79 (s, 1H), 4.35 (t, J = 8.0 Hz, 1H), 3.64- 3.59 (m, 1H), 3.39-3.35 (m, 1H), 2.19 (s, 3H), 2.13 (s, 3H). |
| A | 129 | 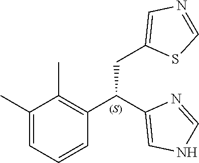 | 5-[(S)-2-(4- imidazolyl)- 2-(2,3-xylyl) ethyl]-1,3- thiazole | 283.4 | 284.2 | 99.53 | 1H NMR (400 MHz, DMSO-d6): δ 12.0 (s, 1H), 8.77 (s, 1H), 7.60- 7.56 (m, 2H), 7.21 (d, J = 4.0 Hz, 1H), 7.03-6.96 (m, 2H), 6.83 ( s, 1H), 4.42 (t, J = 8.0 Hz, 1H), 3.65-3.59 (m, 1H), 3.44-3.35 (m, 1H), 2.21 (s, 3H), 2.17 (s, 3H). |
| A | 130 | 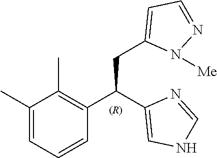 | 5-[(R)-2-(4- imidazolyl)- 2-(2,3-xylyl) ethyl]-1- methyl- pyrazole | 280.375 | 281.3 | 98.1 | 1H NMR (400 MHz, DMSO-d6): δ 11.75 (brs, 1H), 7.50 (s, 1H), 7.22 (brs, 1H), 7.14 (brs, 1H), 7.01-6.95 (m, 2H), 6.76 (s, 1H), 5.79 (m, 1H), 4.52 (brs, 1H), 3.61 (s, 3H), 3.43-3.37 (m, 1H), 3.14-3.09 (m, 1H), 2.20 (s, 3H), 2.14 (s, 3H). |
| A | 131 | 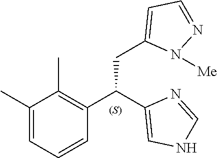 | 5-[(S)-2-(4- imidazolyl)- 2-(2,3-xylyl) ethyl]-1- methyl- pyrazole | 280.375 | 281.3 | 99.37 | 1H NMR (400 MHz, DMSO-d6): δ 11.78 (brs, 1H), 7.51 (s, 1H), 7.22 (d, J = 7.2Hz, 1H), 7.14 (d, J = 1.6 Hz, 1H), 7.01- 6.95 (m, 2H), 6.76 (s, 1H), 5.79 (d, J = 2.0 Hz, 1H), 4.53 (t, J = 7.6 Hz, 1H), 3.66 (s, 3H), 3.43- 3.37 (m, 1H), 3.15- 3.09 (m, 1H), 2.25 (s, 3H), 2.18 (s, 3H). |
| A | 132 | 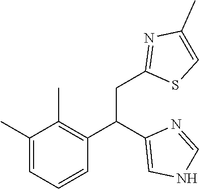 [SMILES: Cc1csc(CC(c2c[nH]cn2)c2cccc(C)c2C)n1] | 2-[2-(4- imidazolyl)-2- (2,3-xylyl) ethyl]-4- methyl-1,3- thiazole | 297.427 | 298.3 | 97.67 | 1H NMR (400 MHz, DMSO-d6): δ 11.75 (s, 1H), 7.52 (s, 1H), 7.20 (d, J = 7.2 Hz, 1H), 7.04 6.93 (m, 3H), 6.79 (s, 1H), 4.65 (t, J = 7.6 Hz, 1H), 3.72-3.67 (m, 1H), 3.44- 3.38 (m, 1H), 2.27 (s, 3H), 2.22 (s, 6H) |
| A | 133 | 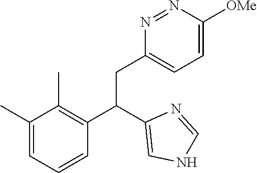 [SMILES: COc1ccc(CC(c2c[nH]cn2)c2cccc(C)c2C)nn1] | 3-[2-(4- imidazolyl)-2- (2,3-xylyl) ethyl]-6- methoxy- pyridazine | 308.385 | 309.3 | 99.88 | 1H NMR (400 MHz, DMSO-d6): δ 11.69 (s, 1H), 7.47-7.41 (m, 1H), 7.24-7.17 (m, 2H), 7.00-6.91 (m, 3H), 6.74- 6.70 (m, 1H), 4.69 (s, 1H), 3.92 (s, 3H), 3.57-3.30 (m, 2H), 2.17 (s, 6H). |
| A | 134 | 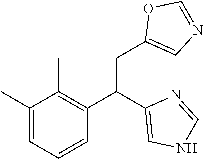 [SMILES: Cc1cccc(C(Cc2cnco2)c2c[nH]cn2)c1C] | 5-[2-(4- imidazolyl)-2- (2,3-xylyl) ethyl]-1,3- oxazole | 267.332 | 268.3 | 98.93 | 1H NMR (400 MHz, DMSO-d6): δ 11.78 (s, 1H), 8.11 (s, 1H), 7.50 (s, 1H), 7.14 (d, J = 7.2 Hz, 1H), 7.01-6.95 (m, 2H), 6.77 (s, 1H), 6.62 (s, 1H), 4.58 (t, J = 7.6 Hz, 1H), 3.50-3.41 (m, 1H), 3.20-3.15 (m, 1H), 2.21 (s, 3H), 2.19 (s, 3H) |
| A | 135 | 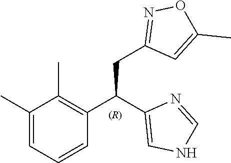 | 3-[(R)-2-(4- imidazolyl)- 2-(2,3-xylyl) ethyl]-5- methyl- isoxazole | 281.359 | 282.3 | 97.04 | 1H NMR (400 MHz, DMSO-d6): δ 11.73 (s, 1H), 7.50 (s, 1H), 7.16 (d, J = 6.8 Hz, 1 H), 7.03- 6.93 (m, 2H), 6.74 (s, 1H), 5.85 (s, 1H), 4.53 (t, J = 7.6 Hz, 1H), 3.35-3.24 (m, 1H), 3.10-3.04 (m, 1H), 2.26 (s, 3H), 2.19 (s, 6H) |
| A | 136 | 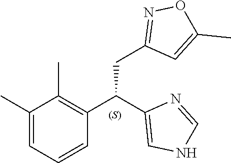 | 3-[(S)-2-(4- imidazolyl)- 2-(2,3-xylyl) ethyl]-5- methyl- isoxazole | 281.359 | 282.3 | 99.36 | 1H NMR (400 MHz, DMSO-d6): δ 11.72 (s, 1H), 7.50 (s, 1H), 7.16 (d, J = 6.8 Hz, 1 H), 6.99- 6.93 (m, 2H), 6.69 (s, 1H), 5.85 (s, 1H), 4.53 (t, J = 7.6 Hz, 1H), 3.35-3.26 (m, 1H), 3.10-3.04 (m, 1H), 2.26 (s, 3H), 2.21 (s, 6H) |

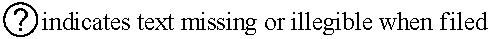

### Procedure B

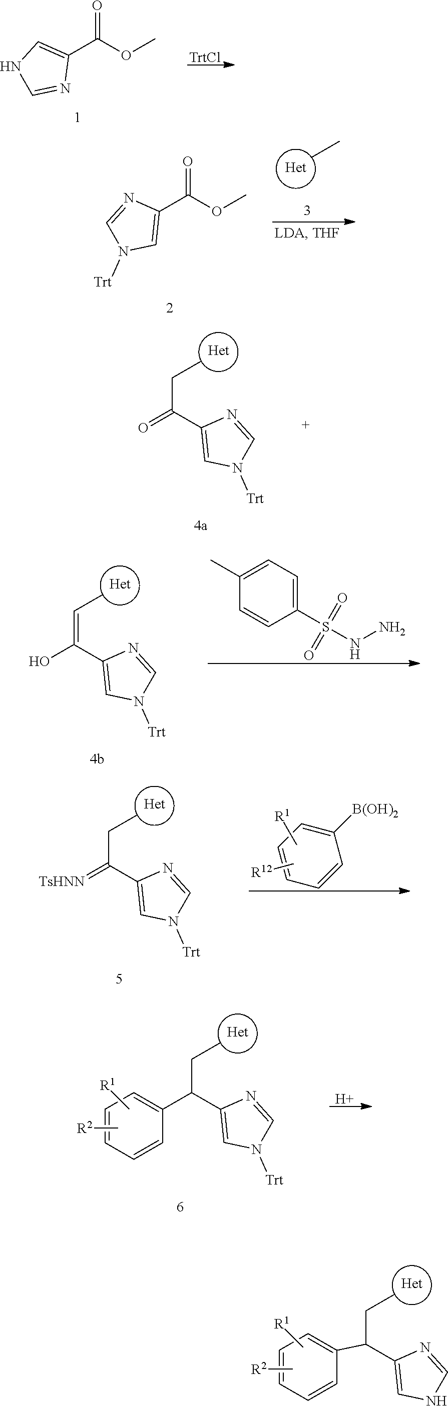

### Procedure B Example: Synthesis of Compound 226

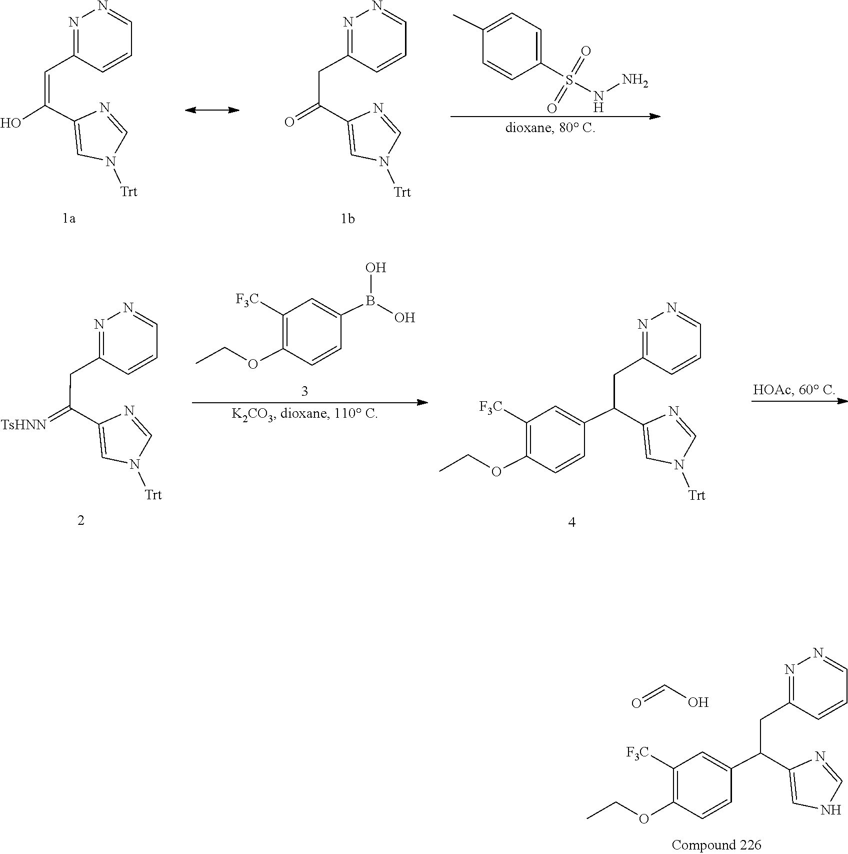

### Experimental Details

### 1. Synthesis of 2

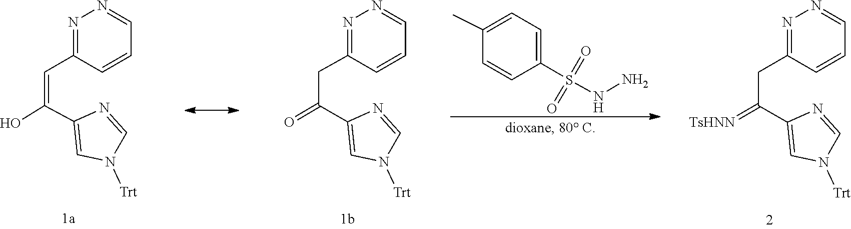

### (Z)-4-methyl-N′-(2-(pyridazin-3-yl)-1-(1-trityl-1H-imidazol-4-yl)ethylidene)benzenesulfonohydrazide

A solution of 2-(pyridazin-3-yl)-1-[1-(triphenylmethyl)imidazol-4-yl]ethanone (50 g, 116 mmol, 1 equiv) and 4-toluenesulfonyl hydrazide (32.4 g, 174. mmol, 1.5 equiv) in 1,4-dioxane (500 ml) was stirred for 3 h at 80° C. under nitrogen atmosphere. The resulting mixture was concentrated under reduced pressure. The crude product was re-crystallized from MeOH to afford 4-methyl-N′-[(1E)-2-(pyridazin-3-yl)-1-[1-(triphenylmethyl)imidazol-4-yl]ethylidene]benzenesulfonohydrazide (35 g, 50%) as a white solid. LCMS (ESI) [M+H]⁺: 599. ¹H NMR (400 MHz, DMSO-d6) δ 12.99 (s, 1H), 9.08 (dd, J=4.9, 1.6 Hz, 1H), 7.79 (d, J=1.3 Hz, 1H), 7.66-7.60 (m, 2H), 7.51 (dd, J=8.4, 4.9 Hz, 1H), 7.47 (d, J=1.4 Hz, 1H), 7.43-7.35 (m, 12H), 7.21-7.17 (m, 1H), 7.10-7.01 (m, 6H), 3.18 (s, 2H), 2.39 (s, 3H).

### 2. Synthesis of 4

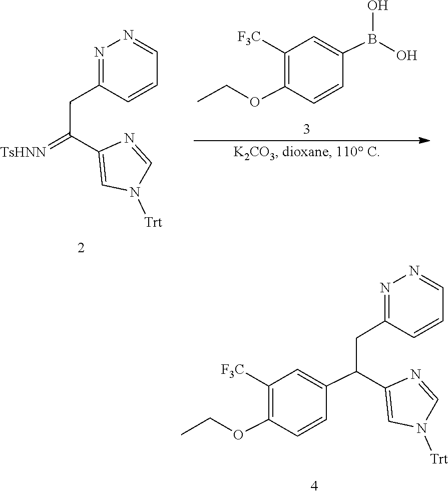

### 3-(2-(4-ethoxy-3-(trifluoromethyl)phenyl)-2-(1-trityl-1H-imidazol-4-yl)ethyl)pyridazine

A solution of 4-methyl-N′-[2-(pyridazin-3-yl)-1-[1-(triphenylmethyl)imidazol-4-yl]ethylidene]benzenesulfonohydrazide (600 mg, 1.0 mmol, 1 equiv), 4-ethoxy-3-(trifluoromethyl)phenylboronic acid (352 mg, 1.51 mmol, 1.50 equiv) and K₂CO₃ (415 mg, 3.0 mmol, 3 equiv) in 1,4-dioxane (5 ml) was stirred for 2 h at 110° C. under air atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 3-{2-[4-ethoxy-3-(trifluoromethyl)phenyl]-2-[1-(triphenylmethyl)imidazol-4-yl]ethyl}pyridazine (136 mg, 22%) as a yellow oil. LCMS (ESI) [M+H]⁺: 605.

### 3. Synthesis of Compound 226

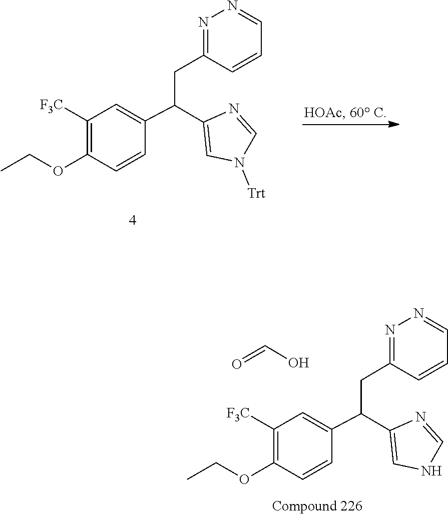

### 3-(2-(4-ethoxy-3-(trifluoromethyl)phenyl)-2-(1H-imidazo-4-yl)ethyl)pyridazine Formic Acid

A solution of 3-{2-[4-ethoxy-3-(trifluoromethyl)phenyl]-2-[1-(triphenylmethyl) imidazol-4-yl]ethyl}pyridazine (131 mg, 0.21 mmol, 1 equiv) in AcOH (5 ml) was stirred for 1 h at 60° C. under air atmosphere. The mixture was allowed to cool down to room temperature. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 3-{2-[4-ethoxy-3-(trifluoromethyl)phenyl]-2-(1H-imidazol-4-yl)ethyl}pyridazine (26.2 mg, 32.41%) as a yellow solid. LCMS (ESI) [M+H]⁺: 363. ¹H NMR (400 MHz, DMSO-d6) δ 11.89 (s, 1H), 8.98 (dd, J=4.8, 1.7 Hz, 1H), 8.14 (s, 1H), 7.55 (s, 1H), 7.53-7.44 (m, 31H), 7.40 (dd, J=8.4, 1.8 Hz, 1H), 7.10 (d, J=8.4 Hz, 1H), 6.85 (s, 1H), 4.56 (t, J=8.2 Hz, 1H), 4.09 (q, J=7.0 Hz, 2H), 3.72-3.62 (m, 1H), 3.52-3.42 (m, 1H), 1.29 (t, J=7.0 Hz, 3H).

TABLE B

| | | | | Molecular | | | |
|---|---|---|---|---|---|---|---|
| | | | | weight | LCMS | Purity | |
| Procedure | Name | Structure | IUPAC | (g/mol) | [M + H]+ | (%) | 1H NMR |
| B | 137 | 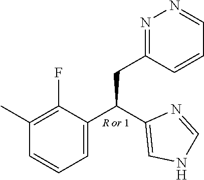 [SMILES: Cc1cccc([C@@H](Cc2cccnn2)c2c[nH]cn2)c1F] | 3-[(R)-2-(2-fluoro-3- tolyl)-2-(4- imidazolyl)ethyl] pyridazine | 282.322 | 283.15 | 99.4 | ¹H NMR (400 MHz, DMSO-d₆) δ 11.98-11.52 (m, 1H), 8.98- 8.84 (m, 1H), 7.45 (s, 1H), 7.43-7.35 (m, 1H), 7.32-7.25 (m, 1H), 7.25-7.15 (m, 1H), 7.08-6.94 (m, 1H), 6.90 (t, J = 7.6 Hz, 1H), 6.81-6.64 (m, 1H), 4.93-4.67 (m, 1H), 3.62 (dd, J = 13.7, 7.9 Hz, 1H), 3.39 (dd, J = 13.7, 8.0 Hz, 1H), 2.17-2.02 (m, 3H). |
| B | 138 | 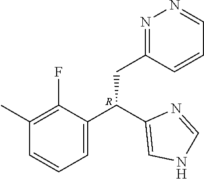 | 3-[(S)-2-(2-fluoro-3- tolyl)-2-(4- imidazolyl)ethyl] pyridazine | 282.322 | 283.15 | 97.5 | ¹H NMR (400 MHz, DMSO-d₆) δ 12.13-11.60 (m, 1H), 9.09- 8.86 (m, 1H), 7.52 (s, 1H), 7.49-7.42 (m, 1H), 7.40-7.33 (m, 1H), 7.28 (t, J = 7.5 Hz, 1H), 7.19-6.93 (m, 2H), 6.88- 6.69 (m, 1H), 5.00-4.71 (m, 1H), 3.76-3.58 (m, 1H), 3.57- 3.40 (m, 1H), 2.24-2.04 (m, 3H). |
| B | 139 | 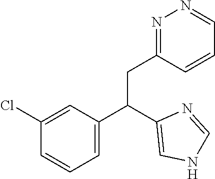 [SMILES: Clc1cccc(C(Cc2cccnn2)c2c[nH]cn2)c1] | 3-[2-(m- chlorophenyl)-2-(4- imidazolyl)ethyl] pyridazine | 284.75 | 285 | 99.6 | ¹H NMR (300 MHz, DMSO-d₆) δ 12.07-11.70 (m, 1H), 8.99 (dd, J = 4.9, 1.7 Hz, 1H), 7.58-7.43 (m, 2H), 7.43-7.29 (m, 2H), 7.29-7.15 (m, 3H), 6.93-6.76 (m, 1H), 4.74-4.46 (m, 1H), 3.68 (dd, J = 13.7, 8.0 Hz, 1 H), 3.61-3.41 (m, 1H). |
| B | 140 | 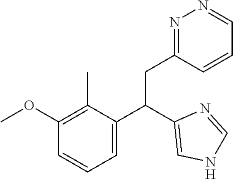 [SMILES: COc1cccc(C(Cc2cccnn2)c2c[nH]cn2)c1C] | 3-[2-(4-imidazolyl)-2 (3-methoxy-2- tolyl)ethyl]pyridazine | 294.358 | 295.15 | 98.5 | ¹H NMR (400 MHz, DMSO-d₆) δ 11.90-11.66 (m, 1H), 9.07- 8.91 (m, 1H), 7.55-7.47 (m, 1H), 7.47-7.27 (m, 2H), 7.12-6.70 (m, 4H), 4.94-4.68 (m, 1H), 3.77-3.70 (m, 3H), 3.70-3.55 (m, 1H), 3.40 (dd, J = 13.7, 7.6 Hz, 1H), 2.12 (s, 3 H). |
| B | 141 | 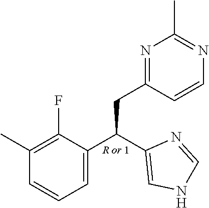 [SMILES: Cc1nccc(C[C@@H](c2c[nH]cn2)c2cccc(C)c2F)n1] | 4-[(R)-2-(2-fluoro-3- tolyl)-2-(4- imidazolyl)ethyl]-2- methylpyrimidine | 296.349 | 297.1 | 99.7 | ¹H NMR (400 MHz, DMSO-d₆) δ 12.06-11.64 (m, 1H), 8.56- 8.31 (m, 1H), 7.50 (s, 1H), 7.24 (s, 1H), 7.12-6.89 (m, 3H), 6.82 (s, 1H), 4.80 (s, 1H), 3.51-3.43 (m, 1H), 3.26- 3.12 (m, 1H), 2.60-2.52 (m, 3H), 2.17 (s, 3H). |
| B | 142 | 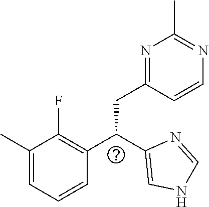 [SMILES: Cc1nccc(C[C@H](c2c[nH]cn2)c2cccc(C)c2F)n1] | 4-[(S)-2-(2-fluoro-3- tolyl)-2-(4- imidazolyl)ethyl]-2- methylpyrimidine | 296.349 | 297.1 | 99.7 | ¹H NMR (400 MHz, DMSO-d₆) δ 12.05-11.63 (m, 1H), 8.57- 8.30 (m, 1H), 7.50 (s, 1H), 7.24 (s, 1H), 7.16-6.91 (m, 3H), 6.82 (s, 1H), 5.03-4.72 (m, 1H), 3.47 (dd, J = 13.8, 8.0) Hz, 1H), 3.21 (dd, J = 13.9, 7.9 Hz, 1 H), 2.53 (s, 3H), 2.17 (s, 3H). |
| B | 143 | 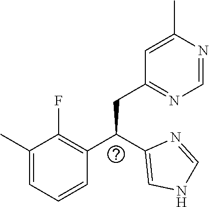 [SMILES: Cc1cc(C[C@@H](c2c[nH]cn2)c2cccc(C)c2F)ncn1] | 4-[(R)-2-(2-fluoro-3- tolyl)-2-(4- imidazolyl)ethyl]-6- methylpyrimidine | 296.349 | 297.15 | 99.2 | ¹H NMR (400 MHz, DMSO-d₆) δ 11.87 (d, J = 72.0 Hz, 1H), 8.88-8.86 (m, 1H), 7.51 (s, 1H), 7.28-6.94 (m, 4H), 6.81 (s, 1H), 4.85-4.81 (m, 1H), 3.49-3.44 (m, 1H), 3.29-3.12 (m, 1H), 2.35-2.32 (m, 3H), 2.19-2.17 (m, 3H). |
| B | 144 | 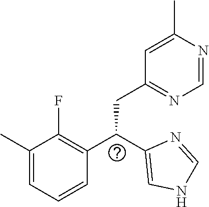 [SMILES: Cc1cc(C[C@H](c2c[nH]cn2)c2cccc(C)c2F)ncn1] | 4-[(S)-2-(2-fluoro-3- tolyl)-2-(4- imidazolyl)ethyl]-6- methylpyrimidine | 296.349 | 297.1 | 99.3 | ¹H NMR (400 MHz, DMSO-d₆) δ 11.87 (d, J = 72.0 Hz, 1H), 8.88 (s, 1H), 7.51 (s, 1H), 7.28-6.94 (m, 4H), 6.81 (s, 1H), 4.99-4.85 (m, 1H), 3.48-3.39 (m, 1H), 3.29-3.12 (m, 1H), 2.36-2.33 (m, 3H), 2.17 (s, 3H). |
| B | 145 | 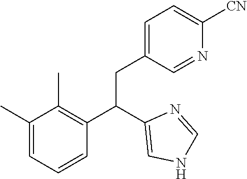 [SMILES: Cc1cccc(C(Cc2ccc(C#N)nc2)c2c[nH]cn2)c1C] | 5-[2-(4-imidazolyl)-2 (2,3-xylyl)ethyl]-2- pyridinecarbonitrile | 302.381 | 303.1 | 98.0 | ¹H NMR (400 MHz, DMSO-d₆) δ 11.78 (s, 1H), 8.47 (d, J = 2.1 Hz, 1H), 7.85 (d, J = 8.0 Hz, 1H), 7.75 (dd, J = 8.0, 2.2 Hz, 1H), 7.52 (s, 1H), 7.32-7.15 (m, 1H), 7.05-6.89 (m, 2H), 6.77 (s, 1H), 4.61-4.47 (m, 1H), 3.50 (dd, J = 13.6, 8.6 Hz, 1H), 3.20 (dd, J = 13.7, 7.2 Hz, 1H), 2.19 (s, 3H), 2.14 (s, 3H) |
| B | 146 | 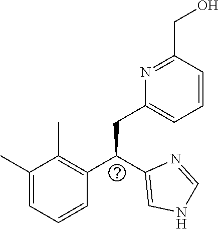 [SMILES: Cc1cccc([C@@H](Cc2cccc(CO)n2)c2c[nH]cn2)c1C] | {6-[(R)-2-(4- imidazolyl)-2-(2,3- xylyl)ethyl]-2- pyridyl}methanol | 307.397 | 308.15 | 99.6 | ¹H NMR (400 MHz, DMSO-d₆) δ 11.73 (d, J = 43.2 Hz, 1H), 7.50 (d, J = 28.1 Hz, 2H), 7.19 (d, J = 7.7 Hz, 2H), 6.93 (d, J = 19.9 Hz, 3H), 6.74 (s, 1H), 5.37-5.25 (m, 1H), 4.82 (s, 1H), 4.51 (d, J = 5.8 Hz, 2H), 3.21-3.11 (m, 1H), 3.81-3.16 (m, 1H), 2.18 (s, 6H). |
| B | 147 | 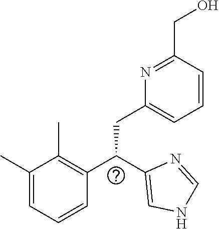 [SMILES: Cc1cccc([C@H](Cc2cccc(CO)n2)c2c[nH]cn2)c1C] | {6-[(S)-2-(4- imidazolyl)-2-(2,3- xylyl)ethyl]-2- pyridyl}methanol | 307.397 | 308.15 | 99.8 | ¹H NMR (400 MHz, DMSO-d₆) δ 11.73 (d, J = 43.2 Hz, 1H), 7.50 (d, J = 28.1 Hz, 2H), 7.19 (d, J = 7.7 Hz, 2H), 6.93 (d, J = 19.9 Hz, 3H), 6.74 (s, 1H), 5.37-5.25 (m, 1H), 4.82 (s, 1H), 4.51 (d, J =5.8 Hz, 2H), 3.21-3.11 (m, 1H), 3.81-3.16 (m, 1H), 2.18 (s, 6H). |
| B | 148 | 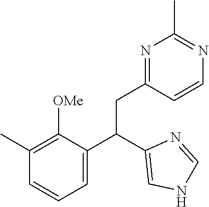 [SMILES: COc1c(C)cccc1C(Cc1ccnc(C)n1)c1c[nH]cn1] | 4-[2-(4-imidazolyl)-2- (2-methoxy-3- tolyl)ethyl]-2- methylpyrimidine | 308.385 | 309.2 | 99.4 | ¹H NMR (400 MHz, DMSO-d₆) δ 11.99-11.61 (m, 1H), 8.49- 8.33 (m, 1H), 7.55-7.41 (m, 1H), 7.36-7.14 (m, 1H), 7.10-6.86 (m, 3H), 6.79-6.56 (m, 1H), 5.04-4.79 (m, 1 H), 3.63 (s, 3H), 3.45 (dd, J = 13.8, 7.8 Hz, 1H), 3.18 (dd, J = 13.8, 8.0 Hz, 1H), 2.53 (s, 3H), 2.25-2.12 (m, 3H). |
| B | 149 | 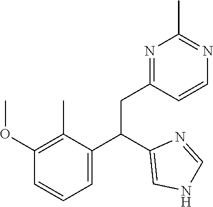 [SMILES: COc1cccc(C(Cc2ccnc(C)n2)c2c[nH]cn2)c1C] | 4-[2-(4-imidazolyl)-2- (3-methoxy-2- tolyl)ethyl]-2- methylpyrimidine | 308.385 | 309.15 | 99.6 | ¹H NMR (400 MHz, DMSO-d₆) δ 11.89-11.64 (m, 1H), 8.51- 8.31 (m, 1H), 7.52-7.43 (m, 1H), 7.15-6.91 (m, 3H), 6.83-6.68 (m, 2H), 4.94-4.71 (m, 1H), 3.72 (d, J = 6.2 Hz, 3 H), 3.44 (dd, J = 14.0, 7.6 Hz, 1H), 3.19 (dd, J = 14.0, 8.0 Hz, 1H), 2.54 (s, 3H), 2.21-2.12 (m, 3H). |
| B | 150 | 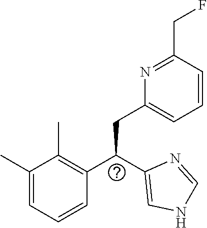 [SMILES: Cc1cccc([C@@H](Cc2cccc(CF)n2)c2c[nH]cn2)c1C] | 4-{(R)-2-[6- (fluoromethyl)-2- pyridyl]-1-(2,3- xylyl)ethyl}imidazole | 309.388 | 310.15 | 99.7 | ¹H NMR (400 MHz, DMSO-d₆) δ 11.70 (s, 1H), 7.63 (t, J = 7.7 Hz, 1H), 7.47 (s, 1H), 7.30-7.09 (m, 2H), 7.05 (d, J = 7.6 Hz, 1 H), 6.95 (dt, J = 16.8, 7.4 Hz, 2 H), 6.74 (s, 1H), 5.47 (s, 1H), 5.35 (s, 1H), 4.83 (s, 1H), 3.61-3.42 (m, 1H), 3.24-3.15 (m, 1H), 2.18 (s, 6H). |
| B | 151 | 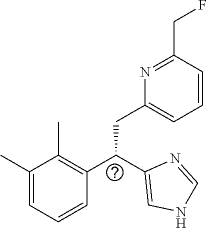 [SMILES: Cc1cccc([C@H](Cc2cccc(CF)n2)c2c[nH]cn2)c1C] | 4-{(S)-2-[6- (fluoromethyl)-2- pyridyl]-1-(2,3- xylyl)ethyl}imidazole | 309.388 | 310.15 | 99.4 | ¹H NMR (400 MHz, DMSO-d₆) δ 11.70 (s, 1H), 7.63 (t, J = 7.7 Hz, 1H), 7.47 (s, 1H), 7.30-7.09 (m, 2H), 7.05 (d, J = 7.6 Hz, 1H), 6.95 (dt, J = 16.8, 7.4 Hz, 2 H), 6.74 (s, 1H), 5.47 (s, 1H), 5.35 (s, 1H), 4.83 (s, 1H), 3.61-3.42 (m, 1H), 3.24-3.15 (m, 1H), 2.18 (s, 6H). |
| B | 152 | 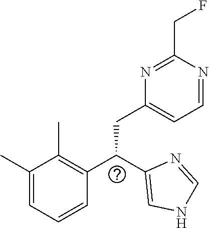 [SMILES: Cc1cccc([C@H](Cc2ccnc(CF)n2)c2c[nH]cn2)c1C] | 4-[(S)-2-(4- imidazolyl)-2-(2,3- xylyl)ethyl]-2- (fluoromethyl) pyrimidine | 310.38 | 311.3 | 98.81 | ¹H NMR (400 MHz, DMSO-d6): δ 11.72 (s, 1H), 8.58-8.57 (m, 1H), 7.48 (s, 1H), 7.18 (s, 2H), 6.99-6.92 (m, 2H), 6.74 (s, 1H), 5.50 (s, 1H), 5.38 (s, 1H), 4.88-4.86 (m, 1H), 3.54- 3.48 (m, 1H), 3.27-3.24 (m, 1H), 2.21 (s, 3H), 2.19 (s, 3H). |
| B | 153 | 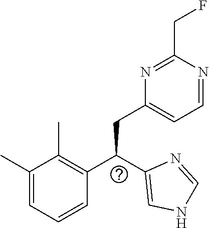 [SMILES: Cc1cccc([C@@H](Cc2ccnc(CF)n2)c2c[nH]cn2)c1C] | 4-[(R)-2-(4- imidazolyl)-2-(2,3- xylyl)ethyl]-2- (fluoromethyl) pyrimidine | 310.38 | 311.3 | 99.1 | ¹H NMR (400 MHz, DMSO-d6): δ 11.71 (s, 1H), 8.58-8.57 (m, 1H), 7.48 (s, 1H), 7.18 (s, 2H), 6.99-6.94 (m, 2H), 6.74 (s, 1H), 5.50 (s, 1H), 5.38 (s, 1H), 4.88-4.86 (m, 1H), 3.50- 3.44 (m, 1H), 3.26-3.16 (m, 1H), 2.21 (s, 3H), 2.19 (s, 3H). |
| B | 154 | 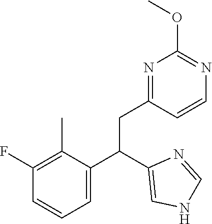 [SMILES: COc1nccc(CC(c2c[nH]cn2)c2cccc(F)c2C)n1] | 4-[2-(3-fluoro-2- tolyl)-2-(4- imidazolyl)ethyl]-2- methoxypyrimidine | 312.348 | 313.1 | 99.5 | ¹H NMR (400 MHz, DMSO-d₆) δ 11.93-11.69 (m, 1H), 8.43- 8.28 (m, 1H), 7.58-7.39 (m, 1H), 7.23-7.06 (m, 2H), 7.05-6.74 (m, 3H), 4.95-4.74 (m, 1H), 3.85 (s, 3H), 3.53- 3.36 (m, 1H), 3.22 (dd, J = 14.2, 7.7 Hz, 1H), 2.24 (d, J = 2.2 Hz, 3H). |
| B | 155 | 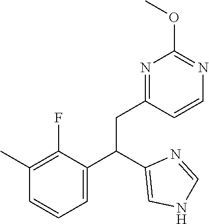 [SMILES: COc1nccc(CC(c2c[nH]cn2)c2cccc(C)c2F)n1] | 4-[2-(2-fluoro-3- tolyl)-2-(4- imidazolyl)ethyl]-2- methoxypyrimidine | 312.348 | 313.05 | 99.7 | ¹H NMR (400 MHz, DMSO-d₆) δ 8.35 (d, J = 5.0 Hz, 1H), 8.19 (s, 1H), 7.51 (s, 1H), 7.21 (t, J = 7.4 Hz, 1H), 7.06 (t, J = 7.4 Hz, 1H), 6.97 (t, J = 7.6 Hz, 1H), 6.89 (d, J = 5.0 Hz, 1H), 6.80 (s, 1H), 4.84 (t, J = 7.8 Hz, 1H), 3.84 (s, 3H), 3.49-3.45 (m, 1 H), 3.24 (dd, J = 14.1, 8.1 Hz, 1H), 2.17 (s, 3H). |
| B | 156 | 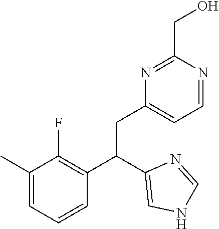 [SMILES: Cc1cccc(C(Cc2ccnc(CO)n2)c2c[nH]cn2)c1F] | {4-[2-(2-fluoro-3- tolyl)-2-(4- imidazolyl)ethyl]-2- pyrimidinyl}methanol | 312.348 | 313.15 | 97.2 | ¹H NMR (400 MHz, DMSO-d₆) δ 11.87 (d, J = 81.3 Hz, 1H), 8.68-8.34 (m, 1H), 7.49 (d, J = 16.8 Hz, 1H), 7.30-7.16 (m, 1H), 7.15-6.93 (m, 3H), 6.78 (d, J = 40.8 Hz, 1H), 5.19- 5.06 (m, 1H), 4.97-4.76 (m, 1H), 4.53 (d, J = 6.2 Hz, 2H), 3.58-3.44 (m, 1H), 3.30-3.22 (m, 1H), 2.17 (d, J = 2.1 Hz, 3 H). |
| B | 157 | 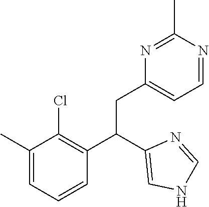 [SMILES: Cc1nccc(CC(c2c[nH]cn2)c2cccc(C)c2Cl)n1] | 4-[2-(2-chloro-3- tolyl)-2-(4- imidazolyl)ethyl]-2- methylpyrimidine | 312.804 | 313 | 98.9 | ¹H NMR (400 MHz, DMSO-d₆) δ 12.02-11.62 (m, 1H), 8.53- 8.32 (m, 1H), 7.57-7.41 (m, 1H), 7.36 (t, J = 4.8 Hz, 1H), 7.23-6.95 (m, 3H), 6.84-6.51 (m, 1H), 5.24-4.98 (m, 1H), 3.45 (dd, J = 14.0, 8.3 Hz, 1H), 3.22 (dd, J = 14.0, 7.4 Hz, 1H), 2.53 (s, 3H), 2.35-2.27 (m, 3H). |
| B | 158 | 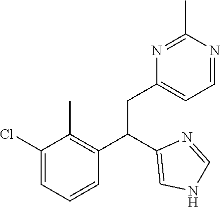 [SMILES: Cc1nccc(CC(c2c[nH]cn2)c2cccc(Cl)c2C)n1] | 4-[2-(3-chloro-2- tolyl)-2-(4- imidazolyl)ethyl]-2- methylpyrimidine | 312.804 | 313 | 99.1 | ¹H NMR (300 MHz, DMSO-d₆) δ 11.98-11.63 (m, 1H), 8.60- 8.18 (m, 1H), 7.58-7.46 (m, 1H), 7.35 (dd, J = 7.7, 1.4 Hz, 1H), 7.29-6.96 (m, 3H), 6.80 (d, J = 24.7 Hz, 1H), 5.04- 4.72 (m, 1H), 3.46 (dd, J = 14.2, 7.6 Hz, 1H), 3.24 (dd, J 14.1, 7.8 Hz, 1H), 2.59-2.53 (m, 3H), 2.43-2.30 (m, 3H). |
| B | 159 | 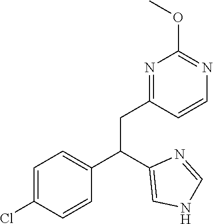 [SMILES: COc1nccc(CC(c2ccc(Cl)cc2)c2c[nH]cn2)n1] | 4-[2-(p- chlorophenyl)-2-(4- imidazolyl)ethyl]-2- methoxypyrimidine | 314.776 | 314.95 | 99.7 | ¹H NMR (400 MHz, DMSO-d₆) δ 12.02-11.60 (m, 1H), 8.41- 8.14 (m, 1H), 7.46 (s, 1H), 7.33-7.01 (m, 4H), 6.97-6.63 (m, 2H), 4.68-4.36 (m, 1H), 3.78 (s, 3H), 3.38 (dd, J = 14.1, 7.5 Hz, 1 H), 3.24-3.07 (m, 1H). |
| B | 160 | 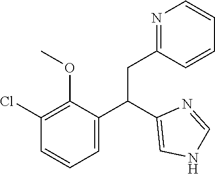 [SMILES: COc1c(Cl)cccc1C(Cc1ccccn1)c1c[nH]cn1] | 3-[2-(3-chloro-2- methoxyphenyl)-2- (4- imidazolyl)ethyl] pyridazine | 314.776 | 314.9 | 90.7 | ¹H NMR (400 MHz, DMSO-d₆) δ 12.04-11.69 (m, 1H), 9.03- 8.94 (m, 1H), 7.56-7.41 (m, 3H), 7.38-7.23 (m, 2H), 7.14-7.04 (m, 1H), 6.87-6.68 (m, 1H), 5.06-4.87 (m, 1H), 3.76-3.57 (m, 4H), 3.53-3.41 (m, 1H). |
| B | 161 | 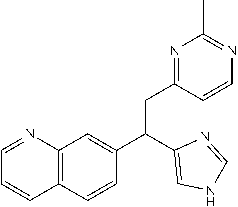 [SMILES: Cc1nccc(CC(c2ccc3cccnc3c2)c2c[nH]cn2)n1] | 4-[2-(4-imidazolyl)-2- (7-quinolyl)ethyl]-2- methylpyrimidine | 315.38 | 316.15 | 99.3 | ¹H NMR (400 MHz, DMSO-d6) δ 11.81 (s, 1H), 8.82 (d, J = 4.3 Hz, 1 H), 8.42 (d, J = 21.1, 5.1 Hz, 1 H), 8.27 (t, J = 8.7 Hz, 1H), 7.92-7.80 (m, 2H), 7.61 (d, J = 8.5 Hz, 1H), 7.55 (s, 1H), 7.52-7.40 (m, 1H), 7.05 (d, J = 5.1 Hz, 1H), 6.94 (s, 1H), 4.73 (t, J = 7.9 Hz, 1H), 3.58 (d, J = 14.0, 7.5 Hz, 1H), 3.55 (d, J = 14.0, 7.5 Hz, 1H), 2.54 (s, 3H). |
| B | 162 | 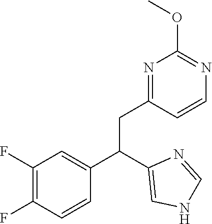 [SMILES: COc1nccc(CC(c2ccc(F)c(F)c2)c2c[nH]cn2)n1] | 4-[2-(3,4- difluorophenyl)-2-(4- imidazolyl)ethyl]-2- methoxypyrimidine | 316.311 | 317 | 99.0 | ¹H NMR (400 MHz, DMSO-d₆) δ 12.02-11.74 (m, 1H), 8.45- 8.28 (m, 1H), 7.55 (s, 1H), 7.42-7.19 (m, 2H), 7.15-6.93 (m, 1H), 6.91-6.68 (m, 2H), 4.74-4.44 (m, 1H), 3.86 (s, 3H), 3.44 (dd, J = 14.1, 7.5 Hz, 1H), 3.25 (dd, J = 14.1, 8.5 Hz, 1H). |
| B | 163 | 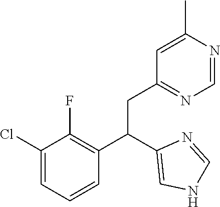 [SMILES: Cc1cc(CC(c2c[nH]cn2)c2cccc(Cl)c2F)ncn1] | 4-[2-(3-chloro-2- fluorophenyl)-2-(4- imidazolyl)ethyl]-6- methylpyrimidine | 316.767 | 317.1 | 96.6 | 1H NMR (400 MHz, DMSO-d6) δ 11.84 (s, 1H), 8.87 (d, J = 1.3 Hz, 1H), 7.52 (d, J = 1.1 Hz, 1H), 7.44-7.31 (m, 2H), 7.20-7.09 (m, 2H), 6.87 (s, 1H), 4.89 (s, 1H), 3.54-3.42 (m, 1H), 3.30-3.23 (m, 1H), 2.34 (s, 3H). |
| B | 164 | 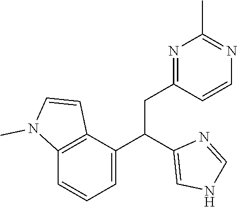 [SMILES: Cc1nccc(CC(c2c[nH]cn2)c2cccc3c2ccn3C)n1] | 4-[2-(4-imidazolyl)-2 (1-methyl-4- indolyl)ethyl]-2- methylpyrimidine | 317.396 | 318.15 | 98.6 | ¹H NMR (400 MHz, DMSO-d₆) δ 8.35 (s, 1H), 7.51 (s, 1H), 7.31-7.17 (m, 2H), 7.13-6.90 (m, 3H), 6.81 (s, 1H), 6.58 (dd, J = 3.1, 0.9 Hz, 1H), 5.16-4.80 (m, 1H), 3.73 (s, 3H), 3.64-3.50 (m, 1H), 3.47-3.30 (m, 1H), 2.55 (s, 3H). |
| B | 165 | 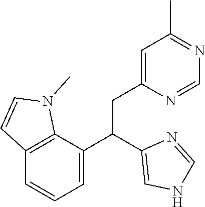 [SMILES: Cc1cc(CC(c2c[nH]cn2)c2cccc3ccn(C)c23)ncn1] | 4-[2-(4-imidazolyl)-2- (1-methyl-7- indolyl)ethyl]-6- methylpyrimidine | 317.396 | 318.2 | 99.0 | 1H NMR (400 MHz, DMSO-d6) δ 11.82 (d, J = 65.0 Hz, 1H), 8.88 (d, J = 12.5 Hz, 1H), 7.49 (d, J = 16.0 Hz, 1H), 7.38- 7.14 (m, 4H), 6.95-6.86 (m, 1H), 6.74 (s, 1H), 6.39-6.31 (m, 1H), 5.77-5.57 (m, 1H), 4.19 (d, J = 8.2 Hz, 3H), 3.60- 3.52 (m, 1H), 3.46-3.37 (m, 1H), 2.32 (d, J = 15.8 Hz, 3H). |
| B | 166 | 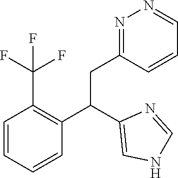 [SMILES: FC(F)(F)c1ccccc1C(Cc1cccnn1)c1c[nH]cn1] | 3-{2-(4-imidazolyl)-2- [o- (trifluoromethyl) phenyl]ethyl}pyridazine | 318.302 | 319 | 98.7 | ¹H NMR (400 MHz, Chloroform-d) δ 9.03 (dd, J = 4.1, 2.4 Hz, 1H), 7.88 (s, 1H), 7.79 (d, J = 7.9 Hz, 1H), 7.64 (d, J = 7.9 Hz, 1H), 7.57 (t, J = 7.6 Hz, 1H), 7.47-7.29 (m, 3H), 6.83 (s, 1 H), 5.05 (dd, J = 9.4, 5.1 Hz, 1 H), 4.03 (dd, J = 14.4, 9.4 Hz, 1 H), 3.58 (dd, J = 14.5, 5.2 Hz, 1H). |
| B | 167 | 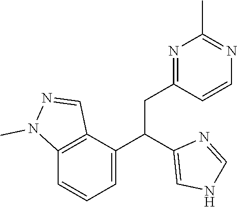 [SMILES: Cc1nccc(CC(c2c[nH]cn2)c2cccc3c2cnn3C)n1] | 4-[1-(4-imidazolyl)-2- (2-methyl-4- pyrimidinyl)ethyl]-1- methyl-1H-indazole | 318.384 | 319 | 97.1 | ¹H NMR (400 MHz, DMSO-d₆) δ 8.37 (d, J = 5.1 Hz, 1H), 8.21 (s, 1H), 8.12 (d, J = 0.8 Hz, 1H), 7.49 (d, J = 1.1 Hz, 1H), 7.39 (d, J = 8.4 Hz, 1H), 7.26 (dd, J = 8.4, 7.0 Hz, 1H), 7.02 (dd, J = 10.2, 6.1 Hz, 2H), 6.91 (s, 1H), 4.95 (t, J = 7.8 Hz, 1H), 3.98 (s, 3H), 3.64-3.58 (m, 1H), 3.44-3.39 (m, 1H), 2.54 (s, 3 H). |
| B | 168 | 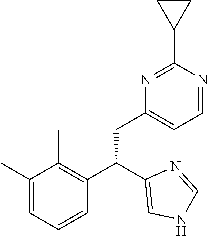 [SMILES: Cc1cccc([C@H](Cc2ccnc(C3CC3)n2)c2c[nH]cn2)c1C] | 4-[(S)-2-(4- imidazolyl)-2-(2,3- xylyl)ethyl]-2- cyclopropylpyrimidine | 318.42 | 319.3 | 98.5 | ¹H NMR (400 MHz, DMSO-d6): δ 11.74 (s, 1H), 8.34 (d, J = 5.2 Hz, 1 H), 7.47 (s, 1H), 7.10 (s, 1H), 7.01-6.89 (m, 3H), 6.73 (s, 1H), 4.83 (s, 1H), 3.42-3.36 (m, 1H), 3.17-3.11 (m, 1H), 2.22-2.17 (m, 6H), 2.12-2.04 (m, 1H), 0.98-0.83 (m, 4H). |
| B | 169 | 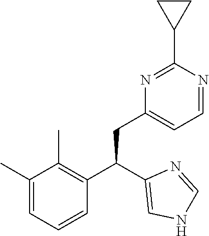 [SMILES: Cc1cccc([C@@H](Cc2ccnc(C3CC3)n2)c2c[nH]cn2)c1C] | 4-[(R)-2-(4- imidazolyl)-2-(2,3- xylyl)ethyl]-2- cyclopropylpyrimidine | 318.42 | 319.3 | 99.56 | ¹H NMR (400 MHz, DMSO-d6): δ 11.47 (s, 1H), 8.30 (s, 1H), 7.41 (s, 1H), 7.14 (s, 1H), 6.94-6.87 (m, 3H), 6.65 (s, 1H), 4.81 (s, 1H), 3.42 (s, 1H), 3.18-3.12 (m, 1H), 2.14-2.07 (m, 6H), 2.13-2.02 (m, 1H), 0.95-0.84 (m, 4H). |
| B | 170 | 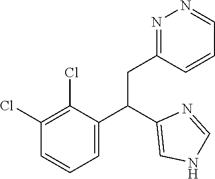 [SMILES: Clc1cccc(C(Cc2cccnn2)c2c[nH]cn2)c1Cl] | 3-[2-(2,3- dichlorophenyl)-2-(4- imidazolyl)ethyl] pyridazine | 319.195 | 319.05 | 98.9 | ¹H NMR (400 MHz, DMSO-d₆) δ 12.21-11.41 (m, 1H), 8.93 (d, J = 4.5 Hz, 1H), 7.55-7.28 (m, 5H), 7.23 (t, J = 7.9 Hz, 1 H), 6.82 (s, 1H), 5.02 (t, J = 7.9 Hz, 1H), 3.61 (dd, J = 13.9, 8.3 Hz, 1H), 3.46-3.41 (m, 1H). |
| B | 171 | 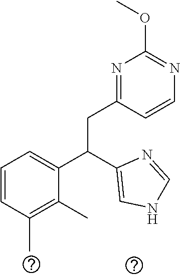 [SMILES: COc1nccc(CC(c2c[nH]cn2)c2cccc(C)c2C)n1] | 6-[1-(4-imidazolyl)-2- (2-methoxy-4- pyrimidinyl)ethyl]-2- toluonitrile | 319.368 | 320.1 | 99.5 | ¹H NMR (400 MHz, DMSO-d₆) δ 11.94-11.72 (m, 1H), 8.44- 8.31 (m, 1H), 7.75-7.58 (m, 1H), 7.59-7.44 (m, 2H), 7.37-7.22 (m, 1H), 7.03-6.76 (m, 2H), 5.00-4.79 (m, 1 H), 3.84 (s, 3H), 3.53-3.36 (m, 1H), 3.26 (dd, J = 14.4, 7.8 Hz, 1H), 2.55 (s, 3H). |
| B | 172 | 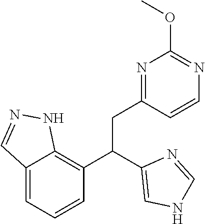 [SMILES: COc1nccc(CC(c2c[nH]cn2)c2cccc3cn[nH]c23)n1] | 7-[1-(4-imidazolyl)-2- (2-methoxy-4- pyrimidinyl)ethyl]- 1H-indazole | 320.356 | 321.15 | 99.3 | ¹H NMR (400 MHz, DMSO-d₆) δ 13.22 (s, 1H), 12.03 (s, 1H), 8.32 (dd, J = 5.0, 1.2 Hz, 1H), 8.16 (s, 1H), 8.03 (d, J = 1.2 Hz, 1H), 7.59-7.51 (m, 2H), 7.28 (d, J = 7.1 Hz, 1H), 7.01 (t, J = 7.4 Hz, 1H), 6.89 (d, J = 6.1 Hz, 2H), 5.04 (t, J = 8.0 Hz, 1H), 3.80 (s, 3H), 3.59 (dd, J = 14.3, 7.8 Hz, 1H), 3.47 (dd, J = 14.3, 8.2 Hz, 1H). |
| B | 173 | 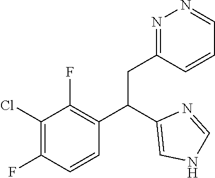 [SMILES: Fc1ccc(C(Cc2cccnn2)c2c[nH]cn2)c(F)c1Cl] | 3-[2-(3-chloro-2,4- difluorophenyl)-2-(4- imidazolyl)ethyl] pyridazine | 320.73 | 320.9 | 98.6 | ¹H NMR (400 MHz, DMSO-d₆) δ 12.18-11.63 (m, 1H), 9.12- 8.89 (m, 1H), 7.62-7.46 (m, 3H), 7.43 (dd, J = 8.5, 1.7 Hz, 1H), 7.37-7.19 (m, 1H), 7.02-6.75 (m, 1H), 5.03-4.77 (m, 1H), 3.70 (dd, J = 13.7, 7.5 Hz, 1H), 3.59-3.45 (m, 1H). |
| B | 174 | 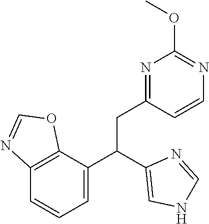 [SMILES: COc1nccc(CC(c2c[nH]cn2)c2cccc3ncoc23)n1] | 7-[1-(4-imidazolyl)-2- (2-methoxy-4- pyrimidinyl)ethyl]- 1,3-benzoxazole | 321.34 | 322.1 | 98.3 | ¹H NMR (400 MHz, DMSO-d₆) δ 12.09-11.65 (m, 1H), 8.80- 8.61 (m, 1H), 8.41-8.23 (m, 1H), 7.67-7.54 (m, 1H), 7.54-7.46 (m, 1H), 7.37 (d, J = 7.5 Hz, 1H), 7.27 (t, J = 7.7 Hz, 1H), 6.97-6.74 (m, 2H), 5.20-4.93 (m, 1H), 3.79 (s, 3H), 3.60 (dd, J = 14.1, 7.3 Hz, 1H), 3.43 (dd, J = 14.1, 8.5 Hz, 1H). |
| B | 175 | 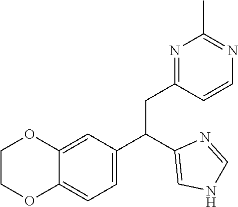 [SMILES: Cc1nccc(CC(c2ccc3c(c2)OCCO3)c2c[nH]cn2)n1] | 4-[2-(2,3-dihydro- 1,4-benzodioxin-6- yl)-2-(4 imidazolyl)ethyl]-2- methylpyrimidine | 322.368 | 322.95 | 99.7 | ¹H NMR (400 MHz, DMSO-d6) δ 11.88-11.72 (m, 1H), 8.48- 8.38 (m, 1H), 7.49-7.44 (m, 1H), 7.07-6.97 (m, 1H), 6.82-6.64 (m, 4H), 4.49 (s, 1H), 4.16 (s, 4H), 3.40 (dd, J = 13.8, 7.7 Hz, 1H), 3.71-3.33 (m, 1H), 2.54 (s, 3H), |
| B | 176 | 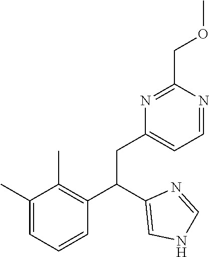 | 4-[2-(4-imidazolyl)-2- (2,3-xylyl)ethyl]-2- (methoxymethyl) pyrimidine | 322.41 | 323.3 | 94.25 | ¹H NMR (400 MHz, DMSO-d6): δ 11.89 (s, 1H), 8.52-8.50 (m, 1H), 7.69 (s, 1H), 7.15-7.12 (m, 2H), 6.98-6.93 (m, 2H), 6.75-6.73 (m, 1H), 4.87 (d, J = 7.6 Hz, 1H), 4.49 (s, 2H), 3.50-3.45 (m, 1H), 3.25-3.21 (m, 4H), 2.21-2.18 (m, 6H). |
| B | 177 | 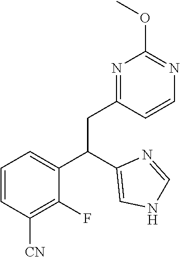 [SMILES: COc1nccc(CC(c2c[nH]cn2)c2cccc(C#N)c2F)n1] | 2-fluoro-3-[1-(4- imidazolyl)-2-(2- methoxy-4- pyrimidinyl)ethyl] benzonitrile | 323.331 | 324.15 | 97.7 | ¹H NMR (400 MHz, DMSO-d₆) δ 11.98-11.73 (m, 1H), 8.40- 8.25 (m, 1H), 7.83-7.58 (m, 2H), 7.52-7.36 (m, 1H), 7.31-7.17 (m, 1H), 6.96-6.70 (m, 2H), 4.96-4.77 (m, 1H), 3.75 (s, 3H), 3.44 (dd, J = 14.3, 7.1 Hz, 1H), 3.26-3.19 (m, 1H). |
| B | 178 | 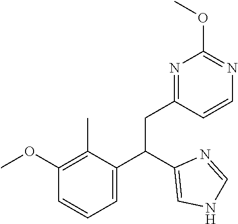 [SMILES: COc1nccc(CC(c2c[nH]cn2)c2cccc(OC)c2C)n1] | 4-[2-(4-imidazolyl)-2- (3-methoxy-2- tolyl)ethyl]-2- methoxypyrimidine | 324.384 | 325.15 | 99.7 | ¹H NMR (400 MHz, DMSO-d₆) δ 11.93-11.53 (m, 1H), 8.33 (s, 1H), 7.48 (s, 1H), 7.11-6.99 (m, 1H), 6.99-6.81 (m, 2H), 6.81-6.66 (m, 2H), 4.97-4.70 (m, 1H), 3.85 (s, 3H), 3.72 (s, 3H), 3.50-3.39 (m, 1H), 3.17 (dd, J = 14.1, 7.5 Hz, 1H), 2.18 (s, 3H). |
| B | 179 | 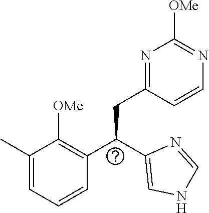 [SMILES: COc1nccc(C[C@@H](c2c[nH]cn2)c2cccc(C)c2OC)n1] | 4-[(R)-2-(4- imidazolyl)-2-(2- methoxy-3- tolyl)ethyl]-2- methoxypyrimidine | 324.384 | 325.15 | 99.7 | ¹H NMR (400 MHz, DMSO-d6) δ 11.74 (s, 1H), 8.34 (d, J = 4.9 Hz, 1H), 7.48 (s, 1H), 7.26 (s, 1H), 7.00-6.98 (m, 2H), 6.96- 6.74 (m, 2H), 4.91 (s, 1H), 3.85 (s, 3H), 3.66 (s, 3H), 3.48- 3.38 (m, 1H), 3.21-3.11 (m, 1H), 2.20 (s, 3H). |
| B | 180 | 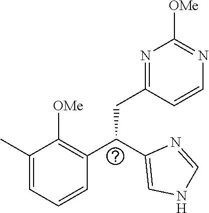 [SMILES: COc1nccc(C[C@H](c2c[nH]cn2)c2cccc(C)c2OC)n1] | 4-[(S)-2-(4- imidazolyl)-2-(2- methoxy-3- tolyl)ethyl]-2- methoxypyrimidine | 324.384 | 325.15 | 99.8 | ¹H NMR (400 MHz, DMSO-d6) δ 11.74 (s, 1H), 8.34 (d, J = 4.9 Hz, 1H), 7.48 (s, 1H), 7.26 (s, 1H), 7.00-6.98 (m, 2H), 6.96- 6.74 (m, 2H), 4.91 (s, 1H), 3.85 (s, 3H), 3.66 (s, 3H), 3.48- 3.38 (m, 1H), 3.21-3.11 (m, 1H), 2.20 (s, 3H). |
| B | 181 | 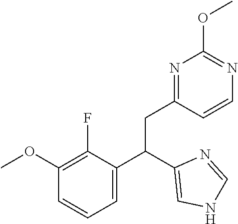 | 4-[2-(2-fluoro-3- methoxyphenyl)-2- (4-imidazolyl)ethyl]- 2- methoxypyrimidine | 328.347 | 329.1 | 99.6 | ¹H NMR (300 MHz, DMSO-d₆) δ 12.05-11.67 (m, 1H), 8.48- 8.27 (m, 1H), 7.59-7.42 (m, 1H), 7.13-6.67 (m, 5H), 5.01-4.70 (m, 1H), 3.84 (s, 3H), 3.77 (s, 3H), 3.47 (dd, J 14.1, 7.2 Hz, 1H), 3.24 (dd, J = 14.1, 8.6 Hz, 1H). |
| B | 182 | 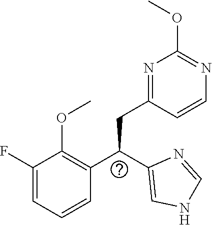 [SMILES: COc1nccc(C[C@@H](c2c[nH]cn2)c2cccc(F)c2OC)n1] | 4-[(R)-2-(3-fluoro-2- methoxyphenyl)-2- (4-imidazolyl)ethyl]- 2- methoxypyrimidine | 328.347 | 329.1 | 99.1 | 1H NMR (400 MHz, DMSO-d6) δ 11.77 (s, 1H), 8.35 (d, J = 5.0 Hz, 1H), 7.52-7.47 (m, 1H), 7.20 (s, 1H), 7.01 (td, J = 9.7, 8.1, 5.2 Hz, 2H), 6.83 (d, J = 20.1 Hz, 2H), 4.93 (s, 1H), 3.84 (s, 3H), 3.74 (d, J = 1.3 Hz, 3H), 3.44 (d, J = 6.3 Hz, 1H), 3.22-3.17 (m, 1H). |
| B | 183 | 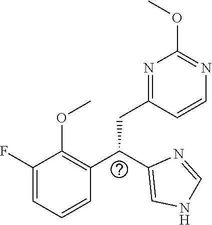 [SMILES: COc1nccc(C[C@H](c2c[nH]cn2)c2cccc(F)c2OC)n1] | 4-[(S)-2-(3-fluoro-2- methoxyphenyl)-2- (4-imidazolyl)ethyl]- 2- methoxypyrimidine | 328.347 | 329.15 | 99.3 | 1H NMR (400 MHz, DMSO-d6) δ 11.77 (s, 1H), 8.35 (d, J = 5.0 Hz, 1H), 7.52-7.47 (m, 1H), 7.20 (s, 1H), 7.01 (td, J = 9.7, 8.1, 5.2 Hz, 2H), 6.83 (d, J = 20.1 Hz, 2H), 4.93 (s, 1H), 3.84 (s, 3H), 3.74 (d, J = 1.3 Hz, 3H), 3.44 (d, J = 6.3 Hz, 1H), 3.22-3.17 (m, 1H). |
| B | 184 | 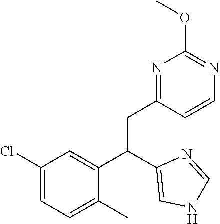 [SMILES: COc1nccc(CC(c2c[nH]cn2)c2cc(Cl)ccc2C)n1] | 4-[2-(5-chloro-2- tolyl)-2-(4- imidazolyl)ethyl]-2- methoxypyrimidine | 328.803 | 328.95 | 99.6 | ¹H NMR (400 MHz, DMSO-d₆) δ 12.03-11.72 (m, 1H), 8.43- 8.31 (m, 1H), 7.53 (s, 1H), 7.44-7.35 (m, 1H), 7.19-6.98 (m, 2H), 7.00-6.75 (m, 2H), 4.90-4.70 (m, 1H), 3.85 (s, 3H), 3.44 (dd, J = 14.1, 8.0 Hz, 1H), 3.21 (dd, J = 14.1, 7.5 Hz, 1H), 2.32 (s, 3H). |
| B | 185 | 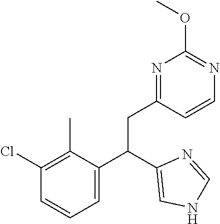 [SMILES: COc1nccc(CC(c2c[nH]cn2)c2cccc(Cl)c2C)n1] | 4-[2-(3-chloro-2- tolyl)-2-(4- imidazolyl)ethyl]-2- methoxypyrimidine | 328.803 | 329.05 | 99.6 | ¹H NMR (400 MHz, DMSO-d₆) δ 11.92-11.73 (m, 1H), 8.48- 8.28 (m, 1H), 7.55-7.45 (m, 1H), 7.37-7.24 (m, 1H), 7.21 (d, J = 7.9 Hz, 1H), 7.15-7.06 (m, 1H), 7.02-6.87 (m, 1H), 6.87-6.74 (m, 1H), 4.99-4.81 (m, 1H), 3.85 (s, 3H), 3.46 (dd, J = 14.2, 7.8 Hz, 1H), 3.22 (dd, J = 14.1, 7.6 Hz, 1H), 2.48-2.34 (m, 3H). |
| B | 186 | 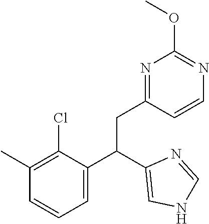 [SMILES: COc1nccc(CC(c2c[nH]cn2)c2cccc(C)c2Cl)n1] | 4-[2-(2-chloro-3- tolyl)-2-(4- imidazolyl)ethyl]-2- methoxypyrimidine | 328.803 | 329.15 | 99.5 | ¹H NMR (400 MHz, DMSO-d₆) δ 12.01-11.71 (m, 1H), 8.42- 8.32 (m, 1H), 7.53-7.46 (m, 1H), 7.38-7.29 (m, 1H), 7.23-7.09 (m, 2H), 6.98-6.85 (m, 1H), 6.83-6.68 (m, 1H), 5.23-5.04 (m, 1H), 3.84 (s, 3H), 3.44 (dd, J = 14.1, 8.2 Hz, 1H), 3.23 (dd, J = 14.1, 7.3 Hz, 1H), 2.38-2.26 (m, 3H). |
| B | 187 | 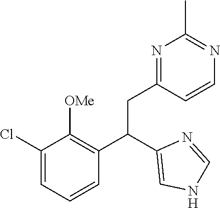 [SMILES: COc1c(Cl)cccc1C(Cc1ccnc(C)n1)c1c[nH]cn1] | 4-[2-(3-chloro-2- methoxyphenyl)-2- (4-imidazolyl)ethyl]- 2-methylpyrimidine | 328.803 | 329.15 | 99.5 | ¹H NMR (400 MHz, DMSO-d₆) δ 8.36 (d, J = 5.1 Hz, 1H), 8.10 (s, 1H), 7.46 (s, 1H), 7.37 (dd, J = 7.9, 1.6 Hz, 1H), 7.20 (dd, J = 8.0, 1.6 Hz, 1H), 7.01 (t, J = 7.9 Hz, 1H), 6.94 (d, J = 5.1 Hz, 1H), 6.73 (s, 1H), 4.86 (t, J = 7.9 Hz, 1H), 3.65 (s, 3H), 3.40-3.37 (m, 1H), 3.20-3.14 (m, 1H), 2.46 (s, 3H). |
| B | 188 | 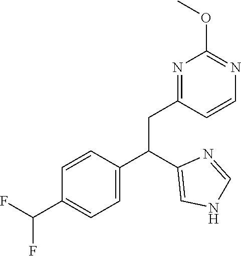 [SMILES: COc1nccc(CC(c2ccc(C(F)F)cc2)c2c[nH]cn2)n1] | 4-{2-[p- (difluoromethyl) phenyl]-2-(4- imidazolyl)ethyl}-2- methoxypyrimidine | 330.338 | 331 | 97.1 | ¹H NMR (400 MHz, DMSO-d₆) δ 12.02-11.67 (m, 1H), 8.41- 8.24 (m, 1H), 7.58-7.34 (m, 5H), 7.14-6.69 (m, 3H), 4.77-4.47 (m, 1H), 3.85 (s, 3H), 3.48 (dd, J = 14.1, 7.4 Hz, 1H), 3.27 (dd, J = 14.1, 8.4 Hz, 1H). |
| B | 189 | 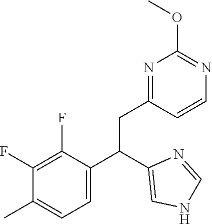 [SMILES: COc1nccc(CC(c2c[nH]cn2)c2ccc(C)c(F)c2F)n1] | 4-[2-(2,3-difluoro-4- tolyl)-2-(4- imidazolyl)ethyl]-2- methoxypyrimidine | 330.338 | 331.15 | 99.6 | ¹H NMR (400 MHz, DMSO-d₆) δ 11.90 (s, 1H), 8.36 (d, J = 5.0 Hz, 1H), 7.54 (s, 1H), 7.10 (t, J = 7.4 Hz, 1H), 6.97 (t, J 7.6 Hz, 1H), 6.93-6.81 (m, 2H), 4.83 (t, J = 7.9 Hz, 1H), 3.84 (s, 3H), 3.47 (dd, J = 14.2, 7.2 Hz, 1H), 3.29-3.23 (m, 1H), 2.19 (d, J = 2.0 Hz, 3H). |
| B | 190 | 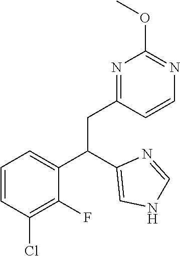 [SMILES: COc1nccc(CC(c2c[nH]cn2)c2cccc(Cl)c2F)n1] | 4-[2-(3-chloro-2- fluorophenyl)-2-(4- imidazolyl)ethyl]-2- methoxypyrimidine | 332.766 | 333.05 | 99.5 | ¹H NMR (400 MHz, DMSO-d₆) δ 12.10-11.67 (m, 1H), 8.49 8.23 (m, 1H), 7.58-7.47 (m, 1H), 7.46-7.23 (m, 2H), 7.20-7.06 (m, 1H), 7.01-6.72 (m, 2H), 5.03-4.76 (m, 1H), 3.83 (s, 3H), 3.49 (dd, J = 14.2, 7.3 Hz, 1H), 3.30-3.26 (m, 1H). |
| B | 191 | 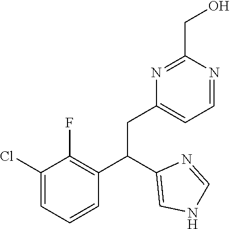 [SMILES: OCc1nccc(CC(c2c[nH]cn2)c2cccc(Cl)c2F)n1] | {4-[2-(3-chloro-2- fluorophenyl)-2-(4- imidazolyl)ethyl]-2- pyrimidinyl}methanol | 332.766 | 333.05 | 99.8 | ¹H NMR (400 MHz, DMSO-d₆) δ 11.93 (d, J = 75.5 Hz, 1H), 8.54 (d, J = 5.1 Hz, 1H), 7.53 (s, 1H), 7.47-7.37 (m, 1H), 7.35 (d, J = 6.9 Hz, 1H), 7.12 (t, J = 7.4 Hz, 2H), 6.91 (s, 1H), 5.14 (t, J = 6.2 Hz, 1H), 4.86 (t, J = 7.9 Hz, 1H), 4.52 (d, J = 6.0 Hz, 2H), 3.74-3.49 (m, 1H), 3.30 (d, J = 10.1 Hz, 1H). |
| B | 192 | 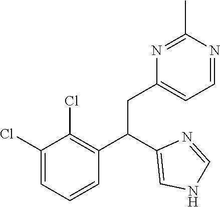 [SMILES: Cc1nccc(CC(c2c[nH]cn2)c2cccc(Cl)c2Cl)n1] | 4-[2-(2,3- dichlorophenyl)-2-(4- imidazolyl)ethyl]-2- methylpyrimidine | 333.222 | 332.9 | 97.0 | ¹H NMR (400 MHz, DMSO-d₆) δ 12.04-11.72 (m, 1H), 8.52- 8.38 (m, 1H), 7.57-7.46 (m, 2H), 7.47-7.23 (m, 2H), 7.14-6.98 (m, 1H), 6.92-6.72 (m, 1H), 5.22-5.02 (m, 1H), 3.52-3.41 (m, 1H), 3.30-3.22 (m, 1H), 2.52 (s, 3H). |
| B | 193 | 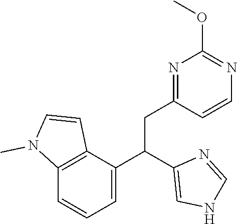 [SMILES: COc1nccc(CC(c2c[nH]cn2)c2cccc3c2ccn3C)n1] | 4-[2-(4-imidazolyl)-2- (1-methyl-4- indolyl)ethyl]-2- methoxypyrimidine | 333.395 | 334.15 | 98.6 | ¹H NMR (400 MHz, DMSO-d₆) δ 8.21 (s, 1H), 7.44 (s, 1H), 7.24-7.08 (m, 2H), 6.99 (t, J = 7.7 Hz, 1H), 6.94-6.76 (m, 2H), 6.74 (s, 1H), 6.51 (d, J = 3.1 Hz, 1H), 5.05-4.79 (m, 1H), 3.79 (s, 3H), 3.66 (s, 3H), 3.56-3.40 (m, 1H), 3.33 (dd, J = 14.1, 7.8 Hz, 1H). |
| B | 194 | 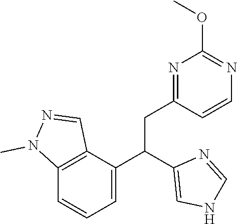 [SMILES: COc1nccc(CC(c2c[nH]cn2)c2cccc3c2cnn3C)n1] | 4-[1-(4-imidazolyl)-2- (2-methoxy-4- pyrimidinyl)ethyl]-1- methyl-1H-indazole | 334.383 | 335.15 | 99.0 | ¹H NMR (400 MHz, DMSO-d₆) δ 8.31 (d, J = 5.0 Hz, 1H), 8.14 (s, 1H), 7.50 (s, 1H), 7.40 (d, J = 8.4 Hz, 1H), 7.26 (dd, J = 8.4, 7.1 Hz, 1H), 7.28-7.24 (m, 1H), 7.03 (d, J = 7.0 Hz, 1H), 6.96-6.82 (m, 2H), 4.97 (t, J = 7.8 Hz, 1H), 3.98 (s, 3H), 3.84 (s, 3H), 3.61 (dd, J = 14.1, 7.7 Hz, 1H), 3.42 (dd, J = 14.1, 8.0 Hz, 1H). |
| B | 195 | 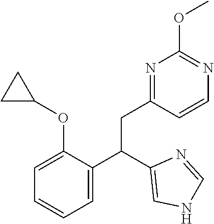 [SMILES: COc1nccc(CC(c2c[nH]cn2)c2ccccc2OC2CC2)n1] | 4-[2-(o- cyclopropoxyphenyl)- 2-(4- imidazolyl)ethyl]-2- methoxypyrimidine | 336.395 | 337.1 | 99.8 | ¹H NMR (300 MHz, DMSO-d₆) δ 11.90-11.55 (m, 1H), 8.31 (s, 1H), 7.49 (s, 1H), 7.32-7.02 (m, 3H), 6.95-6.63 (m, 3H), 4.79 (s, 1H), 3.83 (s, 3H), 3.77 (s, 1H), 3.30 (s, 1H), 3.21- 2.99 (m, 1H), 0.80-0.64 (m, 2H), 0.62-0.51 (m, 1H), 0.49-0.33 (m, 1H). |
| B | 196 | 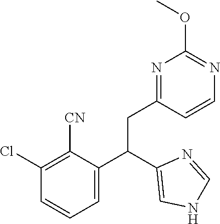 [SMILES: COc1nccc(CC(c2c[nH]cn2)c2cccc(Cl)c2C#N)n1] | 6-chloro-2-[1-(4- imidazolyl)-2-(2- methoxy-4- pyrimidinyl)ethyl] benzonitrile | 339.786 | 340.15 | 98.9 | ¹H NMR (400 MHz, DMSO-d₆) δ 12.03-11.76 (m, 1H), 8.41- 8.24 (m, 1H), 7.66-7.27 (m, 4H), 6.96-6.68 (m, 2H), 5.05-4.75 (m, 1H), 3.77 (s, 3H), 3.49 (dd, J = 14.5, 6.9 Hz, 1H), 3.35 (dd, J = 14.6, 8.7 Hz, 1 H). |
| B | 197 | 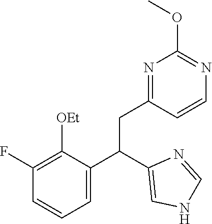 [SMILES: CCOc1c(F)cccc1C(Cc1ccnc(OC)n1)c1c[nH]cn1] | 4-[2-(2-ethoxy-3- fluorophenyl)-2-(4- imidazolyl)ethyl]-2- methoxypyrimidine | 342.374 | 343.15 | 99.7 | ¹H NMR (400 MHz, DMSO-d₆) δ 11.98-11.66 (m, 1H), 8.44- 8.26 (m, 1H), 7.49 (d, J = 14.6 Hz, 1H), 7.28-7.15 (m, 1H), 7.15-6.93 (m, 2H), 6.93-6.61 (m, 2H), 5.08-4.90 (m, 1H), 4.10-3.96 (m, 1H), 3.95-3.78 (m, 4H), 3.50- 3.35 (m, 1H), 3.27-3.12 (m, 1H), 1.30 (t, J = 7.0 Hz, 3H). |
| B | 198 | 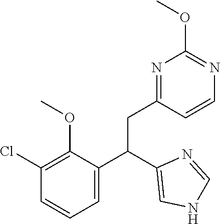 [SMILES: COc1nccc(CC(c2c[nH]cn2)c2cccc(Cl)c2OC)n1] | 4-[2-(3-chloro-2- methoxyphenyl)-2- (4-imidazolyl)ethyl]- 2- methoxypyrimidine | 344.802 | 345.15 | 98.1 | ¹H NMR (400 MHz, DMSO-d₆) δ 12.02-11.69 (m, 1H), 8.48- 8.23 (m, 1H), 7.55-7.47 (m, 1H), 7.44 (dd, J = 7.8, 1.6 Hz, 1H), 7.36-7.21 (m, 1H), 7.15-6.93 (m, 1H), 6.91-6.66 (m, 2H), 5.08-4.85 (m, 1H), 3.84 (s, 3H), 3.75 (s, 3H), 3.46 (dd, J = 14.0, 7.8 Hz, 1H), 3.20 (dd, J = 13.9, 7.9 Hz, 1 H). |
| B | 199 | 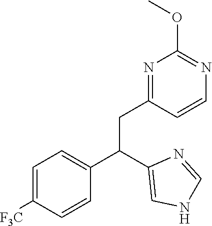 [SMILES: COc1nccc(CC(c2ccc(C(F)(F)F)cc2)c2c[nH]cn2)n1] | 4-{2-(4-imidazolyl)-2- [p- (trifluoromethyl) phenyl]ethyl}-2- methoxypyrimidine | 348.328 | 348.95 | 99.1 | ¹H NMR (400 MHz, DMSO-d₆) δ 12.06-11.62 (m, 1H), 8.45- 8.19 (m, 1H), 7.79-7.37 (m, 5H), 7.06-6.84 (m, 2H), 4.87-4.50 (m, 1H), 3.84 (s, 3H), 3.49 (dd, J = 14.2, 7.6 Hz, 1H), 3.32-3.24 (m, 1H). |
| B | 200 | 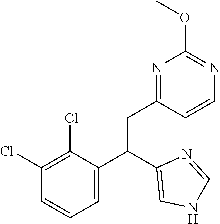 [SMILES: COc1nccc(CC(c2c[nH]cn2)c2cccc(Cl)c2Cl)n1] | 4-[2-(2,3- dichlorophenyl)-2-(4- imidazolyl)ethyl ]-2- methoxypyrimidine | 349.221 | 349.05 | 97.7 | ¹H NMR (400 MHz, DMSO-d₆) δ 11.93 (s, 1H), 8.37 (d, J = 5.0 Hz, 1H), 8.17 (s, 1H), 7.53 (s, 1H), 7.45 (d, J = 7.5 Hz, 2H), 7.28 (t, J = 7.9 Hz, 1H), 6.91 (d, J = 5.0 Hz, 1H), 6.86 (s, 1H), 5.11 (t, J = 7.8 Hz, 1H), 3.83 (s, 3H), 3.46 (dd, J = 14.3, 7.9 Hz, 1H), 3.29 (d, J = 6.7 Hz, 1H). |
| B | 201 | 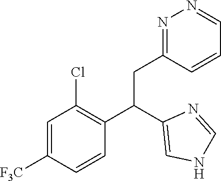 [SMILES: FC(F)(F)c1ccc(C(Cc2cccnn2)c2c[nH]cn2)c(Cl)c1] | 3-{2-[2-chloro-4- (trifluoromethyl) phenyl]-2-(4- imidazolyl)ethyl} pyridazine | 352.747 | 353 | 96.4 | ¹H NMR (400 MHz, Chloroform-d) δ 9.04 (s, 1H), 8.09 7.96 (m, 1H), 7.75-7.59 (m, 2H), 7.51 (t, J = 6.8 Hz, 1H), 7.42-7.29 (m, 2H), 7.03-6.93 (m, 1H), 5.30 (dd, J = 9.7, 5.1 Hz, 1H), 4.01 (dd, J = 14.5, 9.5 Hz, 1H), 3.65 (dd, J = 14.4, 5.4 Hz, 1H). |
| B | 202 | 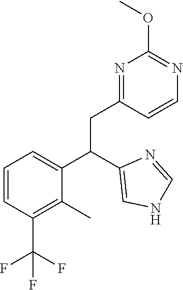 [SMILES: COc1nccc(CC(c2c[nH]cn2)c2cccc(C(F)(F)F)c2C)n1] | 4-{2-(4-imidazolyl)-2 [3-(trifluoromethyl)- 2-tolyl]ethyl}-2- methoxypyrimidine | 362.355 | 363 | 99.8 | ¹H NMR (400 MHz, DMSO-d₆) δ 12.09-11.67 (m, 1H), 8.45- 8.28 (m, 1H), 7.69 (d, J = 7.7 Hz, 1H), 7.59-7.40 (m, 2H), 7.30 (t, J = 7.9 Hz, 1H), 7.06-6.78 (m, 2H), 5.12-4.87 (m, 1H), 3.84 (s, 3H), 3.51 (dd, J = 14.3, 7.8 Hz, 1H), 3.27 (dd, J = 14.2, 7.7 Hz, 1H), 2.47 (s, 3H). |
| B | 203 | 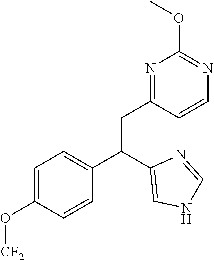 | 4-[2-(4-imidazolyl)-2 (p- trifluoromethoxyphenyl) ethyl]-2- methoxypyrimidine | 364.327 | 365 | 99.5 | ¹H NMR (400 MHz, DMSO-d₆) δ 8.36 (d, J = 5.0 Hz, 1H), 7.57 (s, 1H), 7.42 (d, J = 8.6 Hz, 2H), 7.23 (d, J = 8.2 Hz, 2H), 6.92- 6.85 (m, 2H), 4.60 (t, J = 7.9 Hz, 1H), 3.85 (s, 3H), 3.48 (dd, J = 14.1, 7.7 Hz, 1H), 3.27 (dd, J = 14.1, 8.1 Hz, 1H). |
| B | 204 | 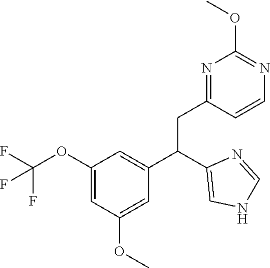 [SMILES: COc1cc(OC(F)(F)F)cc(C(Cc2ccnc(OC)n2)c2c[nH]cn2)c1] | 4-[2-(4-imidazolyl)-2- (3-methoxy-5- trifluoromethoxyphenyl) ethyl]-2- methoxypyrimidine | 394.353 | 395.05 | 99.6 | ¹H NMR (300 MHz, DMSO-d₆) δ 12.06-11.69 (m, 1H), 8.37 (dd, J = 14.8, 5.0 Hz, 1H), 7.61-7.46 (m, 1H), 7.03-6.61 (m, 5H), 4.72-4.46 (m, 1H), 3.85 (s, 3H), 3.77-3.64 (m, 3H), 3.43 (dd, J = 14.1, 7.6 Hz, 1H), 3.25 (dd, J = 14.1, 8.3 Hz, 1H). |
| B | 205 | 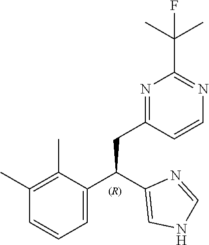 | 4-[(R)-2-(4- imidazolyl)-2-(2,3- xylyl)ethyl]-2-(1- fluoro-1- methylethyl)pyrimidine | 338.43 | 339.15 | 99.70% | ¹H NMR (400 MHz, DMSO-d₆) δ 11.79 (s, 1H), 8.57 (d, J = 5.1 Hz, 1H), 7.50 (d, J = 1.1 Hz, 1H), 7.17 (m, 2H), 7.01- 6.88 (m, 2H), 6.76 (s, 1H), 4.87 (t, J = 7.7 Hz, 1H), 3.52-3.40 (m, 1H), 3.29-3.17 (m, 1H), 2.19 (d, J = 5.6 Hz, 6H), 1.66 (d, J = 21.6 Hz, 6H). |
| B | 206 | 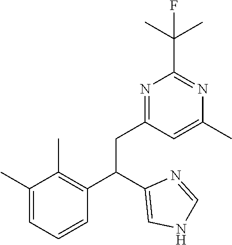 | 2-(1-fluoro-1- methylethyl)-4-[2-(4 imidazolyl)-2-(2,3- xylyl)ethyl]-6- methylpyrimidine | 352.457 | 353.15 | 98.5 | ¹H NMR (400 MHz, DMSO-d6) δ 11.77 (d, J = 45.8 Hz, 1H), 7.49 (s, 1H), 7.30-6.85 (m, 4H), 6.74 (s, 1H), 4.90 (d, J = 32.4 Hz, 1H), 3.45 (s, 1H), 3.22-3.20 (m, 1H), 2.34 (s, 3H), 2.19 (d, J = 8.4 Hz, 6H), 1.64 (d, J = 21.7 Hz, 6H). |
| B | 207 | 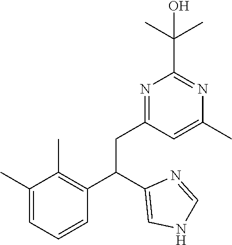 | 2-{4-[2-(4- imidazolyl)-2-(2,3- xylyl)ethyl]-6-methyl 2-pyrimidinyl}-2- propanol | 350.466 | 351.15 | 98.80% | ¹H NMR (400 MHz, DMSO-d₆) δ 11.96-11.47 (m, 1H), 7.50 (s, 1H), 7.23-6.85 (m, 4H), 6.76 (s, 1H), 5.08-4.74 (m, 2H), 3.51-3.41 (m, 1H), 3.22 (dd, J = 14.1, 7.7 Hz, 1H), 2.36 (s, 3H), 2.28-2.04 (m, 6H), 1.51-1.32 (m, 6H). |
| B | 208 | 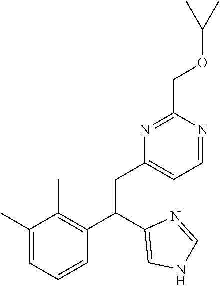 [SMILES: Cc1cccc(C(Cc2ccnc(COC(C)C)n2)c2c[nH]cn2)c1C] | 2- (cyclopropoxymethyl)- 4-[2-(4-imidazolyl) 2-(2,3- xylyl)ethyl]pyrimidine | 348.45 | 349.15 | 99.10% | ¹H NMR (400 MHz, DMSO-d₆) δ 11.95-11.67 (m, 1H), 8.62- 8.45 (m, 1H), 7.53-7.41 (m, 1H), 7.29-6.85 (m, 4H), 6.80-6.65 (m, 1H), 5.01-4.79 (m, 1H), 4.68-4.45 (m, 2H), 3.58-3.38 (m, 2H), 3.30-3.20 (m, 1H), 2.28-2.10 (m, 6H), 0.60-0.35 (m, 4H). |
| B | 209 | 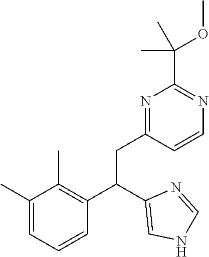 | 4-[2-(4-imidazolyl)-2- (2,3-xylyl)ethyl]-2-(1- methoxy-1- methylethyl)pyrimidine | 350.466 | 351.15 | 98.80% | ¹H NMR (400 MHz, DMSO-d₆) δ 11.90-11.56 (m, 1H), 8.66- 8.43 (m, 1H), 7.57-7.38 (m, 1H), 7.24-7.00 (m, 2H), 7.00-6.83 (m, 2H), 6.78-6.62 (m, 1H), 5.01-4.78 (m, 1H), 3.56-3.39 (m, 1H), 3.27 (dd, J = 14.0, 7.9 Hz, 1H), 2.87 (s, 3H), 2.31-2.08 (m, 6H), 1.56-1.34 (m, 6H). |
| B | 210 | 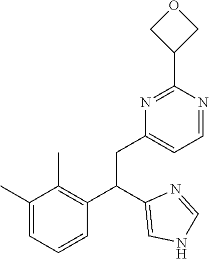 [SMILES: Cc1cccc(C(Cc2ccnc(C3COC3)n2)c2c[nH]cn2)c1C] | 4-[2-(4-imidazolyl)-2- (2,3-xylyl)ethyl]-2-(3- oxetanyl)pyrimidine | 334.423 | 335.1 | 97.10% | ¹H NMR (400 MHz, Chloroform-d6) δ 8.55-8.43 (m, 1H), 8.01 (s, 1H), 7.22-7.09 (m, 1H), 7.09-7.01 (m, 2H), 7.02- 6.97 (m, 1H), 6.69 (s, 1H), 5.16-4.97 (m, 3H), 4.97-4.89 (m, 1H), 4.87 (t, J = 5.9 Hz, 1H), 4.49-4.35 (m, 1H), 3.76- 3.64 (m, 1H), 3.47-3.31 (m, 1H), 2.31-2.13 (m, 6H). |
| B | 211 | 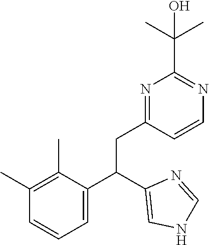 [SMILES: Cc1cccc(C(Cc2ccnc(C(C)(C)O)n2)c2c[nH]cn2)c1C] | 2-{4-[(S)-2-(4- imidazolyl)-2-(2,3- xylyl)ethyl]-2- pyrimidinyl}-2- propanol | 336.439 | 337.1 | 99.10% | ¹H NMR (400 MHz, DMSO-d₆) δ 11.77 (s, 1H), 8.54 (d, J = 5.1 Hz, 1H), 7.49 (s, 1H), 7.24-7.04 (m, 2H), 7.04-6.86 (m, 2H), 6.76 (s, 1H), 4.93 (s, 1H), 4.88 (s, 1H), 3.51 (dd, J 14.1, 7.6 Hz, 1H), 3.27 (dd, J = 14.0, 7.8 Hz, 1H), 2.31 2.04 (m, 6H), 1.42 (s, 6H). |
| B | 212 | 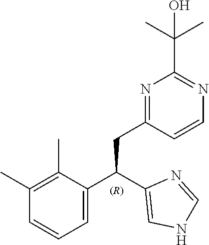 | 2-{4-[(R)-2-(4- imidazolyl)-2-(2,3- xylyl)ethyl]-2- pyrimidinyl}-2- propanol | 336.439 | 337.1 | 99.40% | ¹H NMR (400 MHz, DMSO-d₆) δ 11.96-11.63 (m, 1H), 8.64- 8.44 (m, 1H), 7.49 (s, 1H), 7.28-7.03 (m, 2H), 7.03-6.83 (m, 2H), 6.76 (s, 1H), 5.04-4.70 (m, 2H), 3.60-3.41 (m, 1H), 3.27 (dd, J = 14.0, 7.8 Hz, 1H), 2.29-2.04 (m, 6H), 1.42 (s, 6H). |
| B | 213 | 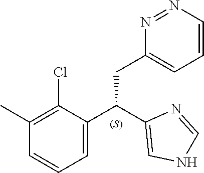 | 3-[(S)-2-(2-chloro-3- tolyl)-2-(4- imidazolyl)ethyl] pyridazine | 298.8 | 299 | 98.1 | 1H NMR (400 MHz, Methanol-d4) δ 8.95 (dd, J = 4.9, 1.6 Hz, 1H), 7.59 (s, 1H), 7.54-7.46 (m, 1H), 7.46-7.39 (m, 1H), 7.31-7.22 (m, 1H), 7.18-7.10 (m, 2H), 6.90 (s, 1H), 5.19 (t, J = 8.0 Hz, 1H), 3.79-3.69 (m, 1H), 3.65-3.52 (m, 2H), 2.32 (s, 3H). |
| B | 214 | 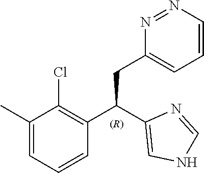 | 3-[(R)-2-(2-chloro-3- tolyl)-2-(4- imidazolyl)ethyl] pyridazine | 298.8 | 299 | 98.5 | 1H NMR (400 MHz, Methanol-d4) δ 8.96 (dd, J = 4.9, 1.7 Hz, 1H), 7.73 (s, 1H), 7.55-7.47 (m, 1H), 7.47-7.40 (m, 1H), 7.30-7.22 (m, 1H), 7.19-7.12 (m, 2H), 6.97 (s, 1H), 5.21 (t, J = 8.0 Hz, 1H), 3.80-3.70 (m, 1H), 3.63-3.53 (m, 1H), 2.32 (s, 3H). |
| B | 215 | 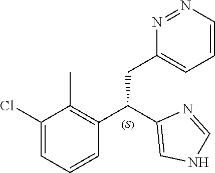 | 3-[(S)-2-(3-chloro-2- tolyl)-2-(4- imidazolyl)ethyl] pyridazine | 298.8 | 299 | 96.9 | 1H NMR (400 MHz, Methanol-d4) δ 8.96 (dd, J = 4.9, 1.6 Hz, 1H), 7.59 (d, J = 1.2 Hz, 1H), 7.54-7.46 (m, 1H), 7.41- 7.34 (m, 1H), 7.33-7.26 (m, 1H), 7.24-7.17 (m, 1H), 7.10 (t, J = 7.8 Hz, 1H), 6.86 (s, 1H), 4.94 (t, J = 8.0 Hz, 1H), 3.82- 3.72 (m, 1H), 3.57-3.47 (m, 1H), 2.31 (s, 3H). |
| B | 216 | 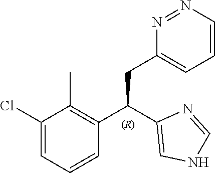 | 3-[(R)-2-(3-chloro-2- tolyl)-2-(4- imidazolyl)ethyl] pyridazine | 298.8 | 299 | 97.6 | 1H NMR (400 MHz, Methanol-d4) δ 8.96 (dd, J = 5.0, 1.6 Hz, 1H), 7.62 (s, 1H), 7.54-7.46 (m, 1H), 7.41-7.34 (m, 1H), 7.29 (d, J = 7.7 Hz, 1H), 7.21 (d, J = 7.8 Hz, 1H), 7.11 (t, J = 7.8 Hz, 1H), 6.87 (s, 1H), 4.95 (t, J = 8.0 Hz, 1H), 3.82-3.73 (m, 1H), 3.58-3.48 (m, 1H), 2.31 (s, 3H). |
| B | 217 | 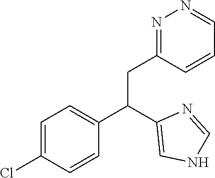 [SMILES: Clc1ccc(C(Cc2cccnn2)c2c[nH]cn2)cc1] | 3-[2-(p- chlorophenyl)-2-(4- imidazolyl)ethyl] pyridazine | 284.8 | 285 | 98.7 | 1H NMR (400 MHz, DMSO-d6) δ 11.94 (s, 1H), 8.98 (dd, J = 4.9, 1.7 Hz, 1H), 8.14 (s, 1H), 7.54 (s, 1H), 7.51-7.44 (m, 1H), 7.43-7.36 (m, 1H), 7.34-7.24 (m, 4H), 6.85 (s, 1H), 4.54 (t, J = 8.1 Hz, 1H), 3.72-3.63 (m, 1H), 3.52-3.42 (m, 1H). |
| B | 218 | 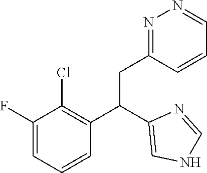 [SMILES: Fc1cccc(C(Cc2cccnn2)c2c[nH]cn2)c1Cl] | 3-[2-(2-chloro-3- fluorophenyl)-2-(4- imidazolyl)ethyl] pyridazine | 302.7 | 303 | 98.7 | 1H NMR (400 MHz, Methanol-d4) δ 8.97 (dd, J = 4.9, 1.7 Hz, 1H), 7.60 (d, J = 1.2 Hz, 1H), 7.57-7.49 (m, 1H), 7.49- 7.42 (m, 1H), 7.32-7.22 (m, 2H), 7.13-7.03 (m, 1H), 6.94 (s, 1H), 5.15 (t, J = 8.0 Hz, 1H), 3.83-3.73 (m, 1H), 3.64- 3.54 (m, 1H). |
| B | 219 | 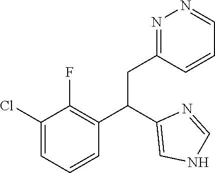 [SMILES: Fc1c(Cl)cccc1C(Cc1cccnn1)c1c[nH]cn1] | 3-[2-(3-chloro-2- fluorophenyl)-2-(4- imidazolyl)ethyl] pyridazine | 302.7 | 303 | 95.6 | 1H NMR (400 MHz, Methanol-d4) δ 8.98 (dd, J = 4.9, 1.7 Hz, 1H), 8.31 (s, 1H), 7.78 (s, 1H), 7.59-7.44 (m, 2H), 7.30 (dd, J = 8.0, 6.8 Hz, 2H), 7.13-7.03 (m, 2H), 4.97 (t, J = 8.1 Hz, 1H), 3.86-3.76 (m, 1H), 3.65-3.55 (m, 1H). |
| B | 220 | 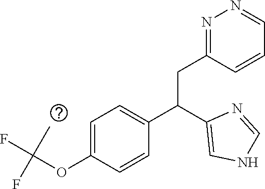 [SMILES: CC(F)(F)Oc1ccc(C(Cc2cccnn2)c2c[nH]cn2)cc1] | 3-[2-(4-imidazolyl)-2- (p- trifluoromethoxyphenyl) ethyl]pyridazine | 334.3 | 335 | 98.1 | 1H NMR (400 MHz, Methanol-d4) δ 9.03-8.97 (m, 1H), 8.47 (s, 1H), 7.67-7.47 (m, 2H), 7.42-7.33 (m, 3H), 7.23- 7.16 (m, 2H), 4.83 (t, J = 8.1 Hz, 1H), 3.88-3.78 (m, 1H), 3.69-3.59 (m, 1H). |
| B | 221 | 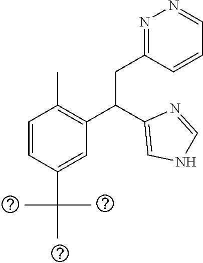 [SMILES: Cc1ccc(C(C)(C)C)cc1C(Cc1cccnn1)c1c[nH]cn1] | 3-{2-(4-imidazolyl)-2- [5-(trifluoromethyl)- 2- tolyl]ethyl}pyridazine | 332.3 | 333 | 98.2 | 1H NMR (400 MHz, Methanol-d4) δ 8.97 (dd, J = 4.9, 1.6 Hz, 1H), 8.23 (s, 1H), 7.85 (d, J = 1.3 Hz, 1H), 7.62-7.57 (m, 1H), 7.51 (dd, J = 8.5, 4.9 Hz, 1H), 7.44-7.34 (m, 2H), 7.29 (d, J = 8.0 Hz, 1H), 7.03 (t, J = 1.0 Hz, 1H), 5.01 (t, J = 8.0 Hz, 1H), 3.85-3.75 (m, 1H), 3.61-3.51 (m, 1H), 2.37 (s, 3H). |
| B | 222 | 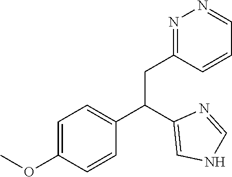 [SMILES: COc1ccc(C(Cc2cccnn2)c2c[nH]cn2)cc1] | 3-[2-(4-imidazolyl)-2- (p- methoxyphenyl)ethyl] pyridazine | 280.3 | 281 | 99.5 | 1H NMR (400 MHz, DMSO-d6) δ 11.74 (s, 1H), 8.97 (d, J = 6.0 Hz, 1H), 7.53-7.41 (m, 2H), 7.34 (s, 1H), 7.18 (d, J = 8.1 Hz, 2H), 6.78 (d, J = 9.0 Hz, 3H), 4.63-4.34 (m, 1H), 3.71- 3.60 (m, 4H), 3.48-3.38 (m, 1H). |
| B | 223 | 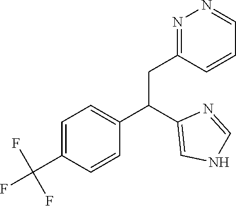 [SMILES: FC(F)(F)c1ccc(C(Cc2cccnn2)c2c[nH]cn2)cc1] | 3-{2-(4-imidazolyl)-2- [p- (trifluoromethyl) phenyl]ethyl}pyridazine | 318.3 | 319 | 99.5 | 1H NMR (400 MHz, Methanol-d4) δ 8.95 (dd, J = 4.7, 1.7 Hz, 1H), 7.62 (s, 1H), 7.57-7.41 (m, 6H), 6.96 (s, 1H), 4.70 (t, J = 8.1 Hz, 1H), 3.86-3.76 (m, 1H), 3.66-3.56 (m, 1H). |
| B | 224 | 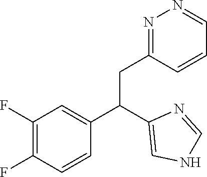 [SMILES: Fc1ccc(C(Cc2cccnn2)c2c[nH]cn2)cc1F] | 3-[2-(3,4- difluorophenyl)-2-(4- imidazolyl)ethyl] pyridazine | 286.3 | 287 | 95.5 | 1H NMR (400 MHz, Methanol-d4) δ 8.96 (dd, J = 4.9, 1.7 Hz, 1H), 7.61 (d, J = 1.2 Hz, 1H), 7.52 (dd, J = 8.5, 4.9 Hz, 1H), 7.45 (dd, J = 8.5, 1.7 Hz, 1H), 7.23-7.07 (m, 2H), 7.06- 6.99 (m, 1H), 6.93 (t, J = 0.9 Hz, 1H), 4.58 (t, J = 8.2 Hz, 1H), 3.79-3.69 (m, 1H), 3.60-3.50 (m, 1H). |
| B | 225 | 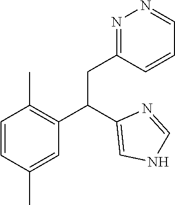 [SMILES: Cc1ccc(C)c(C(Cc2cccnn2)c2c[nH]cn2)c1] [symbol] | 3-[2-(5-chloro-2- tolyl)-2-(4- imidazolyl)ethyl] pyridazine | 298.8 | 299 | 98.5 | 1H NMR (400 MHz, Methanol-d4) δ 8.96 (dd, J = 4.9, 1.6 Hz, 1H), 7.60 (d, J = 1.2 Hz, 1H), 7.49 (dd, J = 8.5, 4.9 Hz, 1H), 7.41-7.33 (m, 2H), 7.05 (d, J = 1.4 Hz, 2H), 6.88 (t, J = 1.0 Hz, 1H), 4.83 (d, J = 8.0 Hz, 1H), 3.75 (dd, J = 13.8, 8.0 Hz, 1H), 3.50 (dd, J = 13.8, 8.0 Hz, 1H), 2.23 (s, 3H). |
| B | 226 | 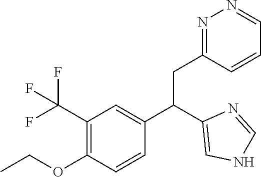 [SMILES: CCOc1ccc(C(Cc2cccnn2)c2c[nH]cn2)cc1C(F)(F)F] | 3-(2-[4-ethoxy-3- (trifluoromethyl) phenyl]-2-(4- imidazolyl)ethyl] pyridazine | 362.4 | 363 | 97.1 | 1H NMR (400 MHz, DMSO-d6) δ 11.89 (s, 1H), 8.98 (dd, J = 4.8, 1.7 Hz, 1H), 8.14 (s, 1H), 7.55 (s, 1H), 7.53-7.44 (m, 3H), 7.40 (dd, J = 8.4, 1.8 Hz, 1H), 7.10 (d, J = 8.4 Hz, 1H), 6.85 (s, 1H), 4.56 (t, J = 8.2 Hz, 1H), 4.09 (q, J = 7.0 Hz, 2H), 3.72-3.62 (m, 1H), 3.52-3.42 (m, 1H), 1.29 (t, J = 7.0 Hz, 3H). |
| B | 227 | 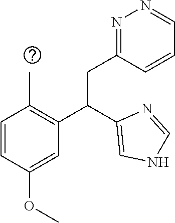 [SMILES: COc1ccc(C)c(C(Cc2cccnn2)c2c[nH]cn2)c1] | 3-[2-(2-chloro-5- methoxyphenyl)-2- (4- imidazolyl)ethyl] pyridazine | 314.8 | 315 | 97.8 | 1H NMR (400 MHz, DMSO-d6) δ 12.13-11.68 (m, 1H), 9.09-8.95 (m, 1H), 7.62-7.49 (m, 1H), 7.49-7.42 (m, 1H), 7.38-7.28 (m, 1H), 7.27-7.18 (m, 1H), 7.16-6.94 (m, 1H), 6.91-6.81 (m, 1H), 6.79-6.72 (m, 1H), 5.13- 4.85 (m, 1H), 3.84-3.59 (m, 4H), 3.57-3.41 (m, 1H). |
| B | 228 | 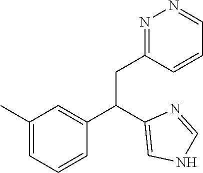 [SMILES: Cc1cccc(C(Cc2cccnn2)c2c[nH]cn2)c1] | 3-[2-(4-imidazolyl)-2- (m- tolyl)ethyl]pyridazine | 264.3 | 265 | 97.5 | 1H NMR (400 MHz, DMSO-d6) δ 12.34-11.19 (m, 1H), 8.98 (d, J = 4.9 Hz, 1H), 7.69-7.27 (m, 3H), 7.23-7.03 (m, 3H), 7.01-6.66 (m, 2H), 4.61-4.36 (m, 1H), 3.74-3.64 (m, 1H), 3.52-3.42 (m, 1H), 2.23 (s, 3H). |
| B | 229 | 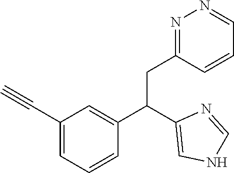 [SMILES: C#Cc1cccc(C(Cc2cccnn2)c2c[nH]cn2)c1] | 3-[2-(m- ethynylphenyl)-2-(4- imidazolyl)ethyl] pyridazine | 274.3 | 275 | 97.7 | 1H NMR (400 MHz, DMSO-d6) δ 12.11-11.54 (m, 1H), 8.98 (dd, J = 4.8, 1.7 Hz, 1H), 7.63-7.15 (m, 7H), 6.98- 6.73 (m, 1H), 4.79-4.43 (m, 1H), 4.13 (d, J = 3.8 Hz, 1H), 3.73-3.63 (m, 1H), 3.53-3.43 (m, 1H). |
| B | 230 | 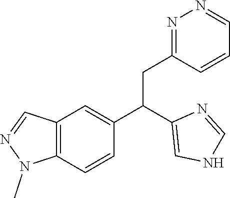 [SMILES: Cn1ncc2cc(C(Cc3cccnn3)c3c[nH]cn3)ccc21] | 5-[1-(4-imidazolyl)-2- (3-pyridazinyl)ethyl]- 1-methyl-1H- indazole | 304.4 | 305 | 98.3 | 1H NMR (400 MHz, DMSO-d6) δ 12.45 (s, 1H), 8.96 (dd, J = 4.8, 1.7 Hz, 1H), 7.92 (s, 1H), 7.72-7.67 (m, 1H), 7.57 (s, 1H), 7.52-7.42 (m, 2H), 7.41-7.33 (m, 2H), 6.92 (d, J = 3.2 Hz, 1H), 4.66 (t, J = 8.1 Hz, 1H), 3.98 (s, 3H), 3.80-3.70 (m, 1H), 3.61-3.51 (m, 1H). |
| B | 231 | 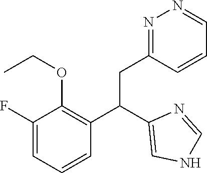 [SMILES: CCOc1c(F)cccc1C(Cc1cccnn1)c1c[nH]cn1] | 3-[2-(2-ethoxy-3- fluorophenyl)-2-(4- imidazolyl)ethyl] pyridazine | 312.4 | 313 | 96.8 | 1H NMR (400 MHz, DMSO-d6) δ 12.04-11.65 (m, 1H), 8.98 (d, J = 4.9, 1.6 Hz, 1H), 7.60-7.39 (m, 2H), 7.36-7.21 (m, 2H), 7.17-6.96 (m, 2H), 6.89-6.66 (m, 1H), 5.11- 4.86 (m, 1H), 4.06-3.94 (m, 1H), 3.87-3.75 (m, 1H), 3.73- 3.59 (m, 1H), 3.54-3.45 (m, 1H), 1.28 (t, J = 7.0 Hz, 3H). |

[symbol]

indicates data missing or illegible when filed

### Procedure C

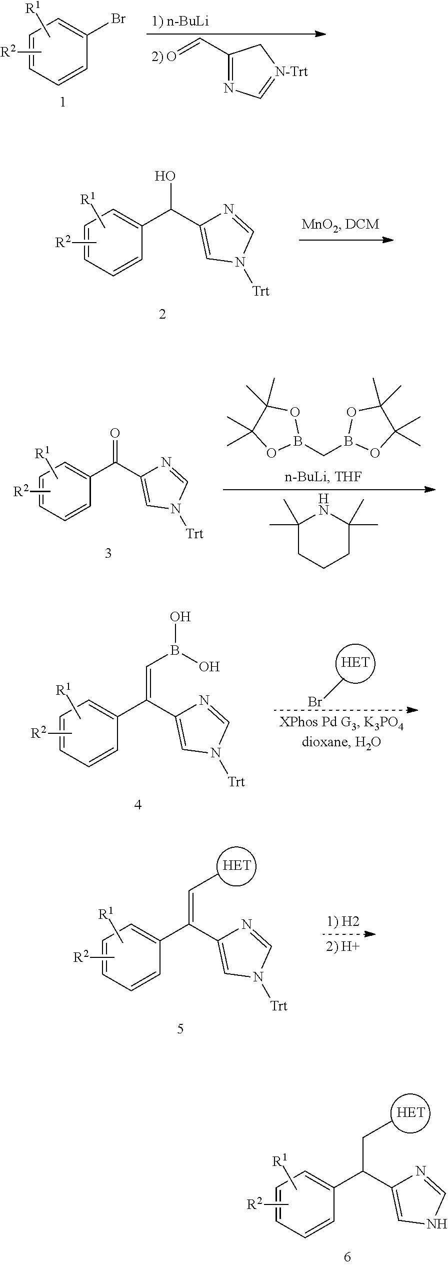

### Procedure C Example: Synthesis of 335

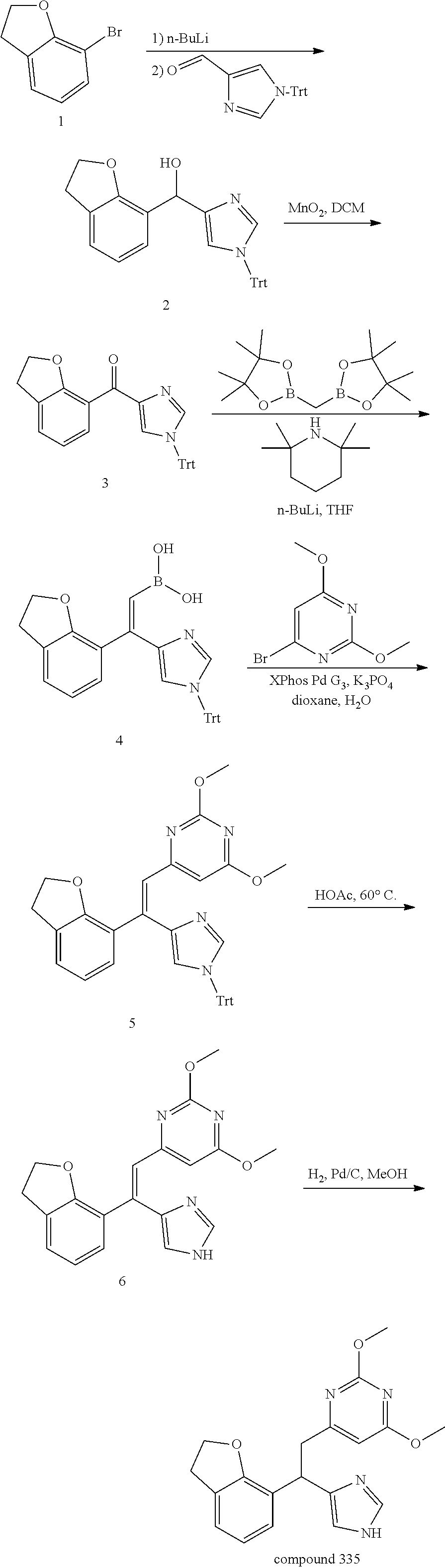

### Experimental Details

### 1. Synthesis of 2

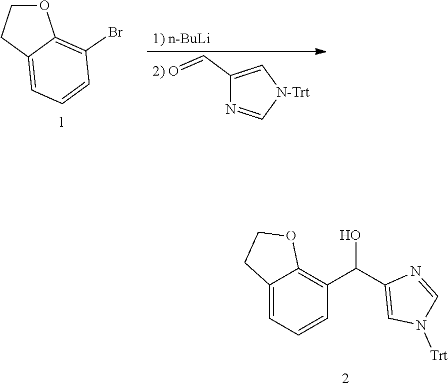

### (2,3-dihydrobenzofuran-7-yl)-trityl-1H-imidazol-4-yl)methanol

To a stirred solution of 7-bromo-2,3-dihydrobenzofuran (15.0 g, 75.77 mmol, 1.0 equiv) in THF (150 ml) was added n-BuLi (45.5 ml, 113.66 mmol, 1.5 equiv) dropwise at −78° C. The solution was stirred at this temperature for 30 min to give solution A. To a stirred solution of 1-trityl-1H-imidazole-4-carbaldehyde (25.6 g, 75.77 mmol, 1.0 equiv) in THF (200 ml) was added solution A dropwise at −78° C. The mixture was stirred at this temperature for 1 h. The reaction mixture was quenched by the addition of 100 mL saturated NH₄Cl aqueous. The aqueous solution was extracted with EtOAc (100 mL×3). The combined organic solution was dried over Na₂SO₄ and concentrated to give the residue which was triturated with MeOH to afford (2,3-dihydrobenzofuran-7-yl)(1-trityl-1H-imidazol-4-yl)methanol (14 g, 40% yield) as white solid. LCMS (ESI) [M+H]⁺: 459. ¹H NMR (400 MHz, Chloroform-d) δ 7.50 (s, 1H), 7.35-7.28 (m, 10H), 7.15-7.03 (m, 7H), 6.94 (d, J=7.7 Hz, 1H), 6.68 (d, J=7.9 Hz, 1H), 6.51 (s, 1H), 5.78 (s, 1H), 4.43 (t, J=8.7 Hz, 2H), 2.98 (t, J=8.7 Hz, 2H).

### 2. Synthesis of 3

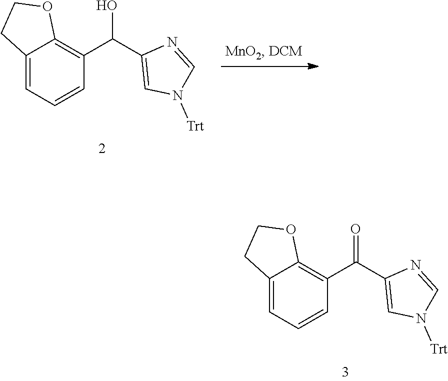

### (2,3-dihydrobenzofuran-7-yl)(1-trityl-1H-imidazol-4-yl)methanone

A mixture of (2,3-dihydrobenzofuran-7-yl)(1-trityl-1H-imidazol-4-yl)methanol (14 g, 30.55 mmol, 1.0 equiv) and MnO₂ (26.6 g, 305.54 mmol, 10.0 equiv) in DCM (300 ml) was stirred at 40° C. overnight. The mixture was filtered. The filtrate was concentrated to give the residue which was purified by flash column with 0-30% EtOAc in petroleum ether to afford (2,3-dihydrobenzofuran-7-yl)(1-trityl-1H-imidazol-4-yl)methanone (12.5 g, 90% yield) as white solid. LCMS (ESI) [M+H]⁺: 457. ¹H NMR (400 MHz, Chloroform-d) δ 7.72 (d, J=7.8 Hz, 1H), 7.67 (s, 1H), 7.53 (s, 1H), 7.38-7.32 (m, 9H), 7.20-7.11 (m, 7H), 6.93 (d, J=7.9 Hz, 1H), 4.58 (t, J=8.8 Hz, 2H), 3.48 (t, J=8.8 Hz, 2H).

### 3. Synthesis of 4

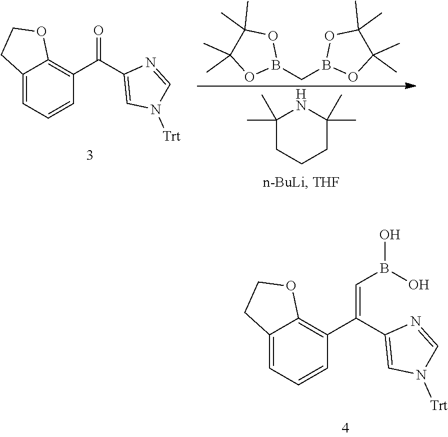

### (Z)-(2-(2,3-dihydrobenzofuran-7-yl)-2-(1-trityl-1H-imidazol-4-yl)vinyl)boronic Acid

To a stirred solution of 2,2,6,6-tetramethylpiperidine (5.8 g, 41.10 mmol, 1.5 equiv) in THF (50 ml) was added n-BuLi (16.4 ml, 41.10 mmol, 1.5 equiv) dropwise at −78° C. The solution was stirred at this temperature for 30 min to give solution A. To a stirred solution of bis(4,4,5,5-tetramethyl-1,3,2-dioxaborolan-2-yl)methane (11.0 g, 41.10 mmol, 1.5 equiv) in THF (50 ml) was added solution A dropwise at 0° C. The solution was stirred at this temperature for 30 min to give solution B. To a solution of (2,3-dihydrobenzofuran-7-yl)(1-trityl-1H-imidazol-4-yl)methanone (12.5 g, 27.40 mmol, 1.0 equiv) in THF (80 ml) was added solution B dropwise at 0° C. The mixture was stirred at this temperature for 1h. The reaction was quenched by the addition of 100 mL saturated NH4Cl aqueous. The aqueous solution was extracted with EtOAc (100 mL×3). The combined organic solution was dried over Na₂SO₄ and concentrated to give the residue which was purified by flash column with 0-60% EtOAc in petroleum ether to afford (Z)-(2-(2,3-dihydrobenzofuran-7-yl)-2-(1-trityl-1H-imidazol-4-yl)vinyl)boronic acid (7.5 g, 37% yield) as white solid. LCMS (ESI) [M+H]⁺: 499. ¹H NMR (400 MHz, Chloroform-d) δ 7.50 (d, J=14.1 Hz, 1H), 7.38-7.29 (m, 10H), 7.15-7.02 (m, 7H), 6.81-6.68 (m, 1H), 6.51 (d, J=7.7 Hz, 1H), 5.57 (d, J=19.7 Hz, 1H), 4.34 (t, J=8.6 Hz, 2H), 3.09 (t, J=9.1 Hz, 2H).

### 4. Synthesis of 5

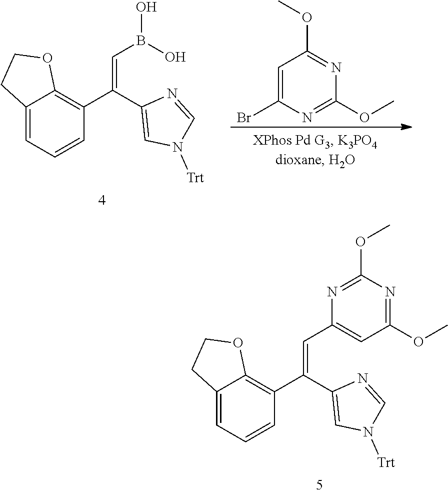

### (Z)-4-(2-(2,3-dihydrobenzofuran-7-yl)-2-(1-trityl-1H-imidazol-4-yl)vinyl)-2,6-dimethoxypyrimidine

To a mixture of (Z)-(2-(2,3-dihydrobenzofuran-7-yl)-2-(1-trityl-1H-imidazol-4-yl)vinyl)boronic acid (600 mg, 1.20 mmol, 1.0 equiv), 4-bromo-2,6-dimethoxypyrimidine (523 mg, 2.40 mmol, 2.0 equiv) and K₃PO₄ (764 mg, 3.60 mmol, 3.0 eq) in dioxane (10 ml) and H₂O (2 ml) was added XPhos Pd G₃ (102 mg, 0.12 mmol, 0.1 equiv). The mixture was stirred at 90° C. for 2 h under nitrogen atmosphere. The mixture was filtered through Celite pad. The filtrate was concentrated to give the residue which was purified by reverse phase flash column with 20-60% acetonitrile in water to afford (Z)-4-(2-(2,3-dihydrobenzofuran-7-yl)-2-(1-trityl-1H-imidazol-4-yl)vinyl)-2,6-dimethoxypyrimidine (250 mg, 35% yield) as yellow solid. LCMS (ESI) [M+H]⁺: 593.

### 5. Synthesis of 6

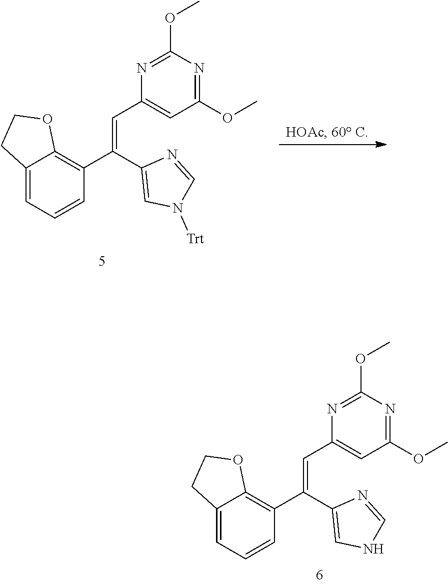

### (Z)-4(2-(2,3-dihydrobenzofuran-7-yl)-2-(H-imidazol-4-yl)viny)-2,6-dimethoxypyrimidine

A solution of (Z)-4-(2-(2,3-dihydrobenzofuran-7-yl)-2-(1-trityl-1H-imidazol-4-yl)vinyl)-2,6-dimethoxypyrimidine (250 mg, 0.42 mmol, 1.0 equiv) in HOAc (2 ml) was stirred at 60° C. for 1.5 h. The solution was concentrated to give the residue which was purified by reverse phase flash column with 10-50% acetonitrile in water to afford (Z)-4-(2-(2,3-dihydrobenzofuran-7-yl)-2-(1H-imidazol-4-yl)vinyl)-2,6-dimethoxypyrimidine (115 mg, 33% yield) as yellow solid. LCMS (ESI) [M+H]⁺: 351.

### 5. Synthesis of 335

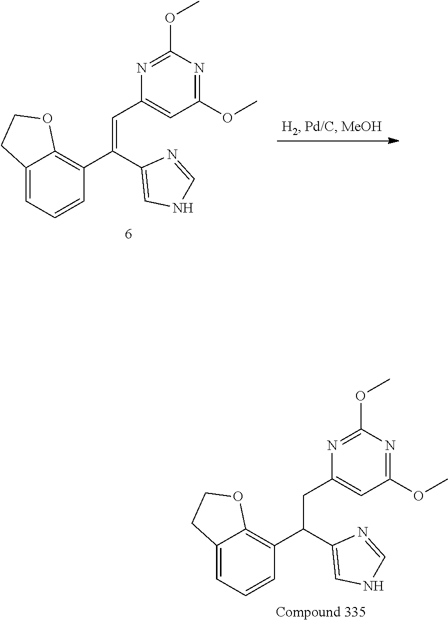 [SMILES: COc1cc(/C=C(\c2c[nH]cn2)c2cccc3c2OCC3)nc(OC)n1 ; COc1cc(CC(c2c[nH]cn2)c2cccc3c2OCC3)nc(OC)n1]

### 4-(2-(2,3-dihydrobenzofuran-7-yl)-2-(1H-imidazol-4-yl)ethyl)-2,6-dimethoxypyrimidine

To a solution of (Z)-4-(2-(2,3-dihydrobenzofuran-7-yl)-2-(1H-imidazol-4-yl)vinyl)-2,6-dimethoxypyrimidine (110 mg, 0.31 mmol, 1.0 equiv) in MeOH (3 ml) was added Pd/C (40 mg). The mixture was stirred at room temperature for 1 h. The mixture was filtered through Celite pad to give the residue which was purified by prep-HPLC (Column: YMC-Actus Triart C18 ExRS 30*150 mm, 5m; Mobile Phase A: Water (10 mmol/L NH4HCO3), Mobile Phase B: ACN; Flow rate: 60 ml/min ml/min; Gradient: 25% B to 50% B in 7 min; Wave Length: 254 nm/220 nm; RT1 (min): 5.77) to afford 4-(2-(2,3-dihydrobenzofuran-7-yl)-2-(1H-imidazol-4-yl)ethyl)-2,6-dimethoxypyrimidine (38.7 mg, 35% yield) as white solid. LCMS (ESI) [M+H]⁺: 353.00. ¹H NMR (400 MHz, Chloroform-d) δ 7.51 (s, 1H), 7.07-6.92 (m, 2H), 6.78-6.70 (m, 2H), 6.11 (s, 1H), 4.73 (t, J=7.6 Hz, 1H), 4.56 (t, J=8.7 Hz, 2H), 3.95 (s, 3H), 3.88 (s, 3H), 3.45-3.35 (m, 1H), 3.33-3.24 (m, 1H), 3.18 (t, J=8.7 Hz, 2H).

TABLE C

| | | | | Molecular | | | |
|---|---|---|---|---|---|---|---|
| | | | | weight | LCMS | Purity | |
| Procedure | Name | Structure | IUPAC | (g/mol) | [M + H]+ | (%) | 1H NMR |
| C | 232 | 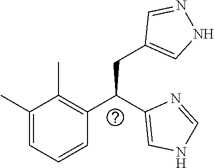 [SMILES: Cc1cccc([C@@H](Cc2cn[nH]c2)c2c[nH]cn2)c1C] | 4-[(R)-2-(4-imidazolyl)-2- (2,3-xylyl)ethyl]pyrazole | 266.348 | 267.15 | 99.3 | 1H NMR (400 MHz, DMSO-d6) δ 12.34 (s, 1H), 11.76 (s, 1H), 7.49 (s, 1H), 7.15 (d, J = 7.3 Hz, 3H), 6.97 (dt, J = 14.1, 7.3 Hz, 2H), 6.74 (s, 1H), 4.36 (t, J = 7.6 Hz, 1H), 3.18 (dd, J = 14.5, 7.9 Hz, 1H), 2.92 (dd, J = 14.5, 7.4 Hz, 1H), 2.18 (d, J = 19.5 Hz, 6H). |
| C | 233 | 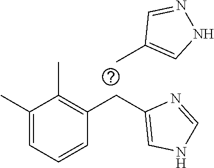 | 4-[(S)-2-(4-imidazolyl)-2- (2,3-xylyl)ethyl]pyrazole | 266.348 | 267.15 | 99.4 | 1H NMR (400 MHz, DMSO-d6) δ 12.34 (s, 1H), 11.76 (s, 1H), 7.49 (s, 1H), 7.15 (d, J = 7.3 Hz, 3H), 6.97 (dt, J = 14.1, 7.3 Hz, 2H), 6.74 (s, 1H), 4.36 (t, J = 7.6 Hz, 1H), 3.18 (dd, J = 14.5, 7.9 Hz, 1H), 2.92 (dd, J = 14.5, 7.4 Hz, 1H), 2.18 (d, J = 19.5 Hz, 6H). |
| C | 234 | 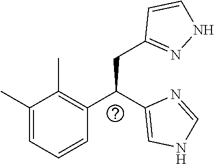 [SMILES: Cc1cccc([C@@H](Cc2cc[nH]n2)c2c[nH]cn2)c1C] | 3-[(R)-2-(4-imidazolyl)-2- (2,3-xylyl)ethyl]pyrazole | 266.348 | 267.15 | 99.6 | 1H NMR (400 MHz, DMSO-d6) δ 12.11 (s, 2H), 7.48 (s, 1H), 7.31 (s, 1H), 7.12 (d, J = 7.4 Hz, 1H), 7.03- 6.90 (m, 2H), 6.72 (s, 1H), 5.84-5.73 (m, 1H), 4.56 (t, J = 7.7 Hz, 1H), 3.34 (s, 1H), 3.15-3.02 (m, 1H), 2.19 (d, J = 8.5 Hz, 6H). |
| C | 235 | 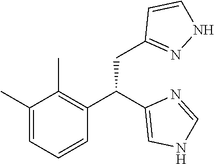 [SMILES: Cc1cccc([C@H](Cc2cc[nH]n2)c2c[nH]cn2)c1C] | 3-[(S)-2-(4-imidazolyl)-2- (2,3-xylyl)ethyl]pyrazole | 266.348 | 267.15 | 99.3 | 1H NMR (400 MHz, DMSO-d6) δ 12.11 (s, 2H), 7.48 (s, 1H), 7.31 (s, 1H), 7.12 (d, J = 7.4 Hz, 1H), 7.03- 6.90 (m, 2H), 6.72 (s, 1H), 5.84-5.73 (m, 1H), 4.56 (t, J = 7.7 Hz, 1H), 3.34 (s, 1H), 3.15-3.02 (m, 1H), 2.19 (d, J = 8.5 Hz, 6H). |
| C | 236 | 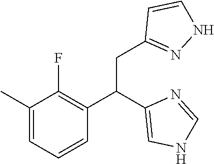 [SMILES: Cc1cccc(C(Cc2cc[nH]n2)c2c[nH]cn2)c1F] | 3-[2-(2-fluoro-3-tolyl)-2- (4- imidazolyl)ethyl]pyrazole | 270.311 | 271.1 | 99.6 | ¹H NMR (400 MHz, DMSO-d6) δ 12.40 (d, J = 80.3 Hz, 1H), 11.83 (s, 1H), 7.51 (s, 1H), 7.40 (s, 2H), 7.20 (td, J = 7.4, 1.8 Hz, 1H), 7.05 (t, J = 7.3 Hz, 1H), 6.97 (t, J = 7.5 Hz, 1H), 6.78 (s, 1H), 5.77 (s, 1H), 4.56 (t, J = 7.9 Hz, 1H), 3.20-3.13 (m, 1H), 2.18 (d, J = 2.0 Hz, 3H) |
| C | 237 | 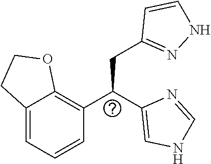 [SMILES: c1cc2c(c([C@@H](Cc3cc[nH]n3)c3c[nH]cn3)c1)OCC2] | 3-[(R)-2-(2,3-dihydro-1- benzofuran-7-yl)-2-(4- imidazolyl)ethyl]pyrazole | 280.331 | 281.15 | 97.4 | ¹H NMR (400 MHz, DMSO-d6) δ 12.98-11.02 (m, 2H), 7.57-7.41 (m, 1H), 7.31 (s, 1H), 7.10-6.94 (m, 2H), 6.79-6.63 (m, 2H), 5.82-5.67 (m, 1H), 4.58- 4.33 (m, 3H), 3.31-3.25 (m, 1H), 3.21-3.09 (m, 3H). |
| C | 238 | 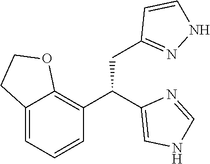 [SMILES: c1cc2c(c([C@H](Cc3cc[nH]n3)c3c[nH]cn3)c1)OCC2] | 3-[(S)-2-(2,3-dihydro-1- benzofuran-7-yl)-2-(4- imidazolyl)ethyl]pyrazole | 280.331 | 281.1 | 95.0 | ¹H NMR (400 MHz, DMSO-d6) δ 12.98-11.02 (m, 1H), 7.57-7.41 (m, 1H), 7.31 (s, 1H), 7.10-6.94 (m, 2H), 6.79-6.63 (m, 2H), 5.82-5.67 (m, 1H), 4.58- 4.33 (m, 3H), 3.31-3.25 (m, 1H), 3.21-3.09 (m, 3H). |
| C | 239 | 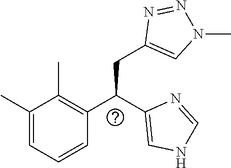 | 4-[(R)-2-(4-imidazolyl)-2- (2,3-xylyl)ethyl]-1- methyl-1H-1,2,3-triazole | 281.363 | 282.15 | 99.5 | ¹H NMR (400 MHz, DMSO-d6) δ 11.73 (s, 1H), 7.48 (s, 2H), 7.12 (s, 1H), 6.97 (d, J = 8.3 Hz, 2H), 6.74 (s, 1H), 4.59 (s, 1H), 3.89 (s, 3H), 3.41-3.31 (m, 1H), 3.15-3.05 (m, 1H), 2.21 (s, 6H). |
| C | 240 | 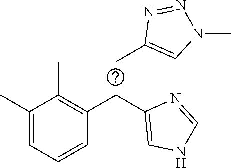 | 4-[(S)-2-(4-imidazolyl)-2- (2,3-xylyl)ethyl]-1- methyl-1H-1,2,3-triazole | 281.363 | 282.15 | 99.6 | ¹H NMR (400 MHz, DMSO-d6) δ 11.73 (s, 1H), 7.48 (s, 2H), 7.12 (s, 1H), 6.97 (d, J = 8.3 Hz, 2H), 6.74 (s, 1H), 4.59 (s, 1H), 3.89 (s, 3H), 3.41-3.31 (m, 1H), 3.15-3.05 (m, 1H), 2.21 (s, 6H). |
| C | 241 | 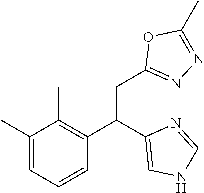 | 2-[2-(4-imidazolyl)- 2-(2,3-xylyl)ethyl]-5- methyl-1,3,4- oxadiazole | 282.347 | 283.15 | 99.1 | ¹H NMR (400 MHz, DMSO-d₆) δ 8.15 (s, 1H), 7.52 (s, 1H), 7.19-7.09 (m, 1H), 6.99 (d, J = 6.8 Hz, 2H), 6.77 (s, 1H), 4.76 (t, J = 7.8 Hz, 1H), 3.57 (dd, J = 15.5, 8.1 Hz, 1H), 3.34 (dd, J = 15.5, 7.6 Hz, 1H), 2.38 (d, J 1.3 Hz, 3H), 2.22 (s, 6H). |
| C | 242 | 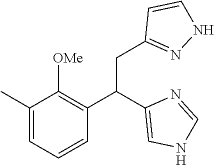 [SMILES: COc1c(C)cccc1C(Cc1cc[nH]n1)c1c[nH]cn1] | 3-[2-(4-imidazolyl)- 2-(2-methoxy-3- tolyl)ethyl]pyrazole | 282.347 | 283.05 | 99.5 | 1H NMR (400 MHz, DMSO-d6) δ 12.49-12.27 (m, 1H), 11.87-11.72 (m, 1H), 7.49 (s, 1H), 7.26 (s, 1H), 6.96 (d, J = 14.2 Hz, 2H), 6.75 (s, 1H), 5.70 (s, 1H), 4.61 (s, 1H), 3.57 (s, 3H), 3.39 (s, 1H), 3.10-3.08 (m, 1H), 2.20 (s, 3H). |
| C | 243 | 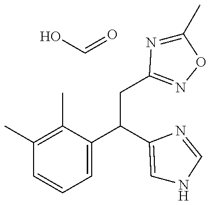 | 3-[2-(4-imidazolyl)- 2-(2,3-xylyl)ethyl]-5- methyl-1,2,4- oxadiazole-formic acid (1/1) | 282.35 | 283.05 | 99.0 | 1H NMR (400 MHz, DMSO-d6) δ 13.8-10.21 (m, 1H), 8.14 (s, 1H), 7.50 (d, J = 1.2 Hz, 1H), 7.11 (dd, J = 7.0, 2.3 Hz, 1H), 7.03-6.92 (m, 2H), 6.74 (t, J = 0.9 Hz, 1H), 4.77 (t, J = 7.7 Hz, 1H), 3.41 (dd, J = 15.0, 8.1 Hz, 1H), 3.16 (dd, J = 15.0, 7.3 Hz, 1H), 2.66 (s, 3H), 2.23 (d, J = 8.7 Hz, 6H). |
| C | 244 | 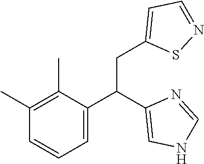 [SMILES: Cc1cccc(C(Cc2ccns2)c2c[nH]cn2)c1C] | 5-[2-(4-imidazolyl)- 2-(2,3- xylyl)ethyl]isothiazole | 283.4 | 284.05 | 98.3 | 1H NMR (400 MHz, DMSO-d6) δ 11.87 (d, J = 38.6 Hz, 1H), 8.36-8.27 (m, 1H), 7.54 (d, J = 20.7 Hz, 1H), 7.28-7.21 (m, 1H), 7.18-6.92 (m, 3H), 6.80 (d, J = 26.1 Hz, 1H), 4.65-4.37 (m, 1H), 3.84-3.63 (m, 1H), 3.50 (s, 1H), 2.21 (d, J = 8.2 Hz, 6H). |
| C | 245 | 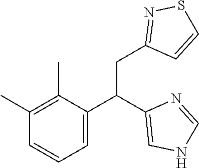 [SMILES: Cc1cccc(C(Cc2ccsn2)c2c[nH]cn2)c1C] | 3-[2-(4-imidazolyl)- 2-(2,3- xylyl)ethyl]isothiazole | 283.4 | 284.1 | 99.1 | 1H NMR (400 MHz, DMSO-d6) δ 11.74 (s, 1H), 8.82 (d, J = 4.6 Hz, 1H), 7.49 (s, 1H), 7.13 (d, J = 7.4 Hz, 1H), 7.06-6.89 (m, 3H), 6.74 (s, 1H), 4.84-4.71 (m, 1H), 3.58-3.54 (m, 1H), 3.33-3.28 (m, 1H), 2.20 (d, J = 4.4 Hz, 6H). |
| C | 246 | 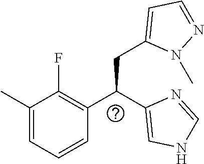 [SMILES: Cc1cccc([C@@H](Cc2ccnn2C)c2c[nH]cn2)c1F] | 5-[(R)-2-(2-fluoro-3- tolyl)-2-(4- imidazolyl)ethyl]-1- methylpyrazole | 284.338 | 285.1 | 98.1 | 1H NMR (400 MHz, DMSO-d6) δ 7.94 (s, 1H), 7.23 (t, J = 7.2 Hz, 1H), 7.18-7.05 (m, 3H), 7.02 (t, J = 7.5 Hz, 1H), 5.80 (d, J = 1.8 Hz, 1H), 4.58 (t, J = 7.8 Hz, 1H), 3.69 (s, 3H), 3.43 (dd, J = 15.3, 7.9 Hz, 1H), 3.22 (dd, J = 15.3, 7.9 Hz, 1H), 2.20 (d, J = 2.1 Hz, 3H). |
| C | 247 | 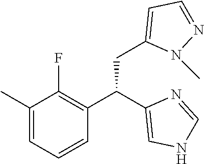 | 5-[(S)-2-(2-fluoro-3- tolyl)-2-(4- imidazolyl)ethyl]-1- methylpyrazole | 284.338 | 285.1 | 98.6 | 1H NMR (400 MHz, DMSO-d6) δ 7.94 (s, 1H), 7.23 (t, J = 7.2 Hz, 1H), 7.18-7.05 (m, 3H), 7.02 (t, J = 7.5 Hz, 1H), 5.80 (d, J = 1.8 Hz, 1H), 4.58 (t, J = 7.8 Hz, 1H), 3.69 (s, 3H), 3.43 (dd, J = 15.3, 7.9 Hz, 1H), 3.22 (dd, J = 15.3, 7.9 Hz, 1H), 2.20 (d, J = 2.1 Hz, 3H). |
| C | 248 | 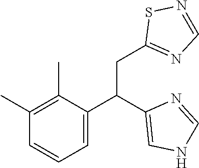 [SMILES: Cc1cccc(C(Cc2ncns2)c2c[nH]cn2)c1C] | 5-[2-(4-imidazolyl)- 2-(2,3-xylyl)ethyl]- 1,2,4-thiadiazole | 284.388 | 285.15 | 97.5 | 1H NMR (400 MHz, DMSO-d6) δ 8.70 (s, 1H), 7.59 (s, 1H), 7.25-7.17 (m, 1H), 7.07-6.93 (m, 2H), 6.86 (s, 1H), 4.77-4.66 (m, 1H), 4.01-3.94 (m, 1H), 3.70- 3.64 (m, 1H), 2.23 (d, J = 3.5 Hz, 6H). |
| C | 249 | 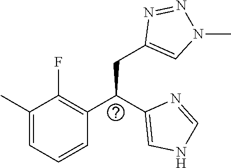 | 4-[(R)-2-(2-fluoro-3- tolyl)-2-(4- imidazolyl)ethyl]-1- methyl-1H-1,2,3- triazole | 285.326 | 286.25 | 99.3 | 1H NMR (400 MHz, DMSO-d6) δ 11.86 (d, J = 69.5 Hz, 1H), 7.51 (s, 2H), 7.21 (s, 1H), 7.05 (d, J = 8.2 Hz, 1H), 6.98 (t, J = 7.4 Hz, 1H), 6.81 (s, 1H), 4.57 (s, 1H), 3.90 (s, 3H), 3.40 (s 1H),3.18 (dd, J = 14.6, 7.9 Hz, 1H), 2.19 (d, J = 2.2 Hz, 3H). |
| C | 250 | 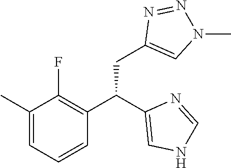 | 4-[(S)-2-(2-fluoro-3- tolyl)-2-(4- imidazolyl)ethyl]-1- methyl-1H-1,2,3- triazole | 285.326 | 286.25 | 99.7 | 1H NMR (400 MHz, DMSO-d6) δ 11.86 (d, J = 69.5 Hz, 1H), 7.58-7.48 (m, 2H), 7.24-7.20 (m, 1H), 7.11-6.05 (m, 2H), 6.81 (s, 1H), 4.67-4.54 (m, 1H), 3.90 (s, 3H), 3.40 (s 1H), 3.18 (dd, J = 14.6, 7.9 Hz, 1H), 2.19 (d, J = 2.2 Hz, 3H). |
| C | 251 | 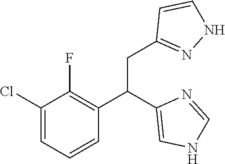 [SMILES: Fc1c(Cl)cccc1C(Cc1cc[nH]n1)c1c[nH]cn1] | 3-[2-(3-chloro-2- fluorophenyl)-2-(4- imidazolyl)ethyl]py- razole | 290.729 | 291 | 99.1 | 1H NMR (400 MHz, DMSO-d6) δ 7.55 (s, 1H), 7.40- 7.34 (m, 3H), 7.13 (t, J = 7.9 Hz, 1H), 6.88 (s, 1H), 5.83 (d, J = 2.0 Hz, 1H), 4.61 (t, J = 8.0 Hz, 1H), 3.37 (dd, J = 14.6, 6.9 Hz, 1H), 3.23 (dd, J = 14.6, 9.1 Hz, 1H). |
| C | 252 | 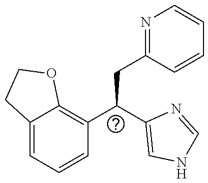 [SMILES: c1ccc(C[C@@H](c2c[nH]cn2)c2cccc3c2OCC3)nc1] | 4-[(R)-1-(2,3- dihydro-1-benzofuran-7-yl)-2- (2- pyridyl)ethyl]imida- [symbol] | 291.354 | 292.1 | 99.6 | 1H NMR (400 MHz, DMSO-d6) δ 12.02-11.43 (m, 1H), 8.42 (s, 1H), 7.49 (d, J = 32.0 Hz, 2H), 7.18- 6.95 (m, 4H), 6.69 (s, 2H), 4.75-4.35 (m, 3H), 3.45 (s, 1H), 3.25 (s, 1H), 3.12 (t, J = 9.1 Hz, 2H). |
| C | 253 | 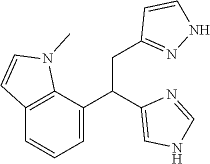 [SMILES: Cn1ccc2cccc(C(Cc3cc[nH]n3)c3c[nH]cn3)c21] | 3-[2-(4-imidazolyl)- 2-(1-methyl-7- indolyl)ethyl]pyrazole | 291.358 | 292.15 | 99.3 | 1H NMR (400 MHz, DMSO-d6) δ 12.46-12.28 (m, 1H), 11.88-11.75 (m, 1H), 7.52 (s, 1H), 7.48 (s, 2H), 7.36 (s, 2H), 6.95-6.86 (m, 1H), 6.74 (s, 1H), 6.33 (d, J = 3.1 Hz, 1H), 5.82-5.77 (m, 1H), 5.22 (s, 1H), 4.08 (s, 3H), 3.46 (s, 1H), 3.42 (s, 1 H). |
| C | 254 | 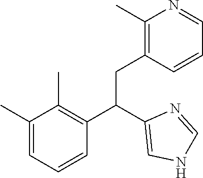 [SMILES: Cc1cccc(C(Cc2cccnc2C)c2c[nH]cn2)c1C] | 4-[2-(2-methyl-3- pyridyl)-1-(2,3- xylyl)ethyl]imidazole | 291.398 | 292.15 | 98.0 | ¹H NMR (400 MHz, DMSO-d₆) δ 11.75 (s, 1H), 8.17 (s, 1H), 7.50 (s, 1H), 7.29 (d, J = 7.7 Hz, 1H), 7.19 (d, J = 7.6 Hz, 1H), 7.08-6.85 (m, 3H), 6.75 (s, 1H), 4.51 (t, J = 7.6 Hz, 1H), 3.64-3.53 (m, 1H), 3.09- 3.02 (m, 1H), 2.40 (s, 3H), 2.17 (s, 3H), 2.01 (s, 3H). |
| C | 255 | 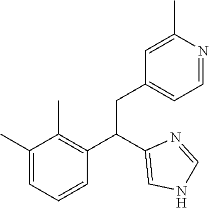 [SMILES: Cc1cc(CC(c2c[nH]cn2)c2cccc(C)c2C)ccn1] | 4-[2-(2-methyl-4- pyridyl)-1-(2,3- xylyl)ethyl]imidazole | 291.398 | 292.15 | 98.7 | ¹H NMR (400 MHz, Chloroform-d ) δ 8.29-7.99 (m, 1H), 7.61 (d, J = 1.2 Hz, 1H), 7.17 (d, J = 7.6 Hz, 1H), 7.11-7.04 (m, 1H), 7.04-6.95 (m, 1H), 6.87 (s, 1H), 6.79 (dd, J = 4.8, 1.5 Hz, 1H), 6.67 (s, 1H), 4.65- 4.50 (m, 1H), 3.48 (dd, J = 13.7, 7.5 Hz, 1H), 3.12 (dd, J = 13.6, 7.8 Hz, 1H), 2.44 (s, 3H), 2.22 (s, 3H), 2.05 (s, 3H). |
| C | 256 | 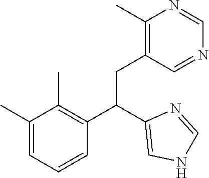 [SMILES: Cc1cccc(C(Cc2cncnc2C)c2c[nH]cn2)c1C] | 5-[2-(4-imidazolyl)- 2-(2,3-xylyl)ethyl]-4- methylpyrimidine | 292.386 | 293.25 | 99.7 | ¹H NMR (400 MHz, DMSO-d₆) δ 11.87-11.66 (m, 1H), 8.75 (s, 1H), 8.15 (s, 1H), 7.57-7.45 (m, 1H), 7.41-7.15 (m, 1H), 7.10-6.92 (m, 2H), 6.82-6.70 (m, 1H), 4.65-4.43 (m, 1H), 3.44 (dd, J = 14.1, 8.0 Hz, 1H), 3.09 (dd, J = 14.1, 7.5 Hz, 1H), 2.45-2.31 (m, 3H), 2.17 (s, 3H), 2.09-1.94 (m, 3H). |
| C | 257 | 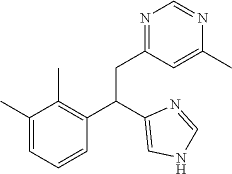 | 4-[2-(4-imidazolyl)- 2-(2,3-xylyl)ethyl]-6- methylpyrimidine | 292.386 | 293 | 99.5 | ¹H NMR (400 MHz, DMSO-d₆) δ 11.85-11.59 (m, 1H), 8.94-8.83 (m, 1H), 7.51-7.41 (m, 1H), 7.25- 6.89 (m, 4H), 6.79-6.68 (m, 1H), 4.99-4.79 (m, 1H), 3.44 (dd, J = 14.0, 8.4 Hz, 1H), 3.19-3.07 (m, 1H), 2.40-2.28 (m, 3H), 2.28-2.15 (m, 6H). |
| C | 258 | 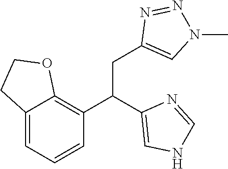 [SMILES: Cn1cc(CC(c2c[nH]cn2)c2cccc3c2OCC3)nn1] | 4-[2-(2,3-dihydro-1- benzofuran-7-yl)-2- (4-imidazolyl)ethyl] 1-methyl-1H-1,2,3- triazole | 295.346 | 296.15 | 99.2 | ¹H NMR (300 MHz, DMSO-d₆) δ 11.98-11.59 (m, 1H), 7.60-7.34 (m, 2H), 7.15-6.85 (m, 2H), 6.82- 6.60 (m, 2H), 4.61-4.30 (m, 3H), 3.89 (s, 3H), 3.32- 3.26 (m, 1H), 3.15 (t, J = 8.7 Hz, 3H). |
| C | 259 | 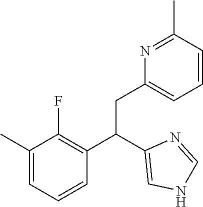 [SMILES: Cc1cccc(CC(c2c[nH]cn2)c2cccc(C)c2F)n1] | 4-[1-(2-fluoro-3- tolyl)-2-(6-methyl-2- pyridyl)ethyl]imida- zole | 295.361 | 296.1 | 99.5 | 1H NMR (400 MHz, Chloroform-d) δ 7.89 (d, J = 1.2 Hz, 1H), 7.50 (t, J = 7.7 Hz, 1H), 7.06 (dd, J = 8.8, 6.5 Hz, 2H), 6.93 (dt, J = 11.6, 6.9 Hz, 3H), 6.72 (s, 1H), 4.79 (dd, J = 8.9, 4.2 Hz, 1H), 3.67 (dd, J = 14.4, 8.8 Hz, 1H), 3.36 (dd, J = 14.4, 4.2 Hz, 1H), 2.61 (s, 3H), 2.25 (d, J = 2.2 Hz, 3H). |
| C | 260 | 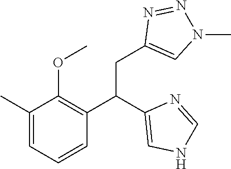 | 4-[2-(4-imidazolyl)- 2-(2-methoxy-3- tolyl)ethyl]-1- methyl-1H-1,2,3- triazole | 297.362 | 298.15 | 99.6 | 1H NMR (400 MHz, DMSO-d6) δ 11.81 (d, J = 72.3 Hz, 1H), 7.57-7.36 (m, 2H), 7.32-7.12 (m, 1H), 7.09-6.88 (m, 2H), 6.72 (d, J = 60.2 Hz, 1H), 4.90- 4.49 (m, 1H), 3.89 (d, J = 9.0 Hz, 3H), 3.62 (d, J = 6.9 Hz, 3H), 3.47-3.33 (m, 1H), 3.23-3.00 (m, 1H), 2.21 (d, J = 6.8 Hz, 3H). |
| C | 261 | 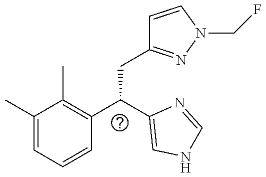 [SMILES: Cc1cccc([C@H](Cc2ccn(CF)n2)c2c[nH]cn2)c1C] | 3-[(S)-2-(4- imidazolyl)-2-(2,3- xylyl)ethyl]-1- (fluoromethyl)pyra- zole | 298.365 | 299.2 | 92.2 | ¹H NMR (400 MHz, DMSO-d₆) δ 11.95-11.68 (m, 1H), 7.57-7.46 (m, 1H), 7.40 (s, 1H), 7.31-7.10 (m, 1H), 7.08-6.87 (m, 2H), 6.83-6.64 (m, 1H), 6.27-5.98 (m, 2H), 5.90 (s, 1H), 4.69-4.51 (m, 1H), 3.57-3.48 (m, 1H), 3.25-3.18 (m, 1H), 2.28- 2.06 (m, 6H). |
| C | 262 | 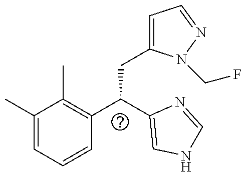 [SMILES: Cc1cccc([C@H](Cc2ccnn2CF)c2c[nH]cn2)c1C] | 5-[(S)-2-(4- imidazolyl)-2-(2,3- xylyl)ethyl]-1- (fluoromethyl)pyra- zole | 298.365 | 299.15 | 93.8 | ¹H NMR (400 MHz, DMSO-d₆) δ 7.78 (d, J = 2.5 Hz, 1H), 7.49 (d, J = 1.2 Hz, 1H), 7.12 (d, J = 7.4 Hz, 1H), 7.01-6.84 (m, 2H), 6.75 (s, 1H), 6.07 (5, 1H), 5.99- 5.80 (m, 2H), 4.59 (t, J = 7.7 Hz, 1H), 3.33-3.19 (m, 1H), 3.15-2.91 (m, 1H), 2.20 (d, J = 4.5 Hz, 6H). |
| C | 263 | 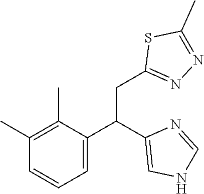 [SMILES: Cc1nnc(CC(c2c[nH]cn2)c2cccc(C)c2C)s1] | 2-[2-(4-imidazolyl)- 2-(2,3-xylyl)ethyl]-5- methyl-1,3,4- thiadiazole | 298.415 | 299.1 | 95.4 | 1H NMR (400 MHz, DMSO-d6) δ 11.90 (d, J = 46.7 Hz, 1H), 7.55 (d, J = 20.7 Hz, 1H), 7.22 (d, J = 7.0 Hz, 1H), 7.12-6.93 (m, 2H), 6.80 (d, J = 25.7 Hz, 1H), 4.79-4.53 (m, 1H), 3.82 (dd, J = 15.0, 8.4 Hz, 1H), 3.56 (s, 1H), 2.58-2.50 (m, 3H), 2.23 (s, 6H). |
| C | 264 | 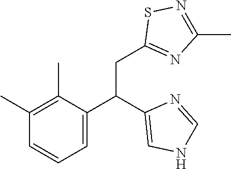 | 5-[2-(4-imidazolyl)- 2-(2,3-xylyl)ethyl]-3- methyl-1,2,4- thiadiazole | 298.415 | 299.1 | 97.0 | 1H NMR (400 MHZ, DMSO-d6) δ 11.86 (s, 1H), 7.68- 7.49 (m, 1H), 7.29-7.15 (m, 1H), 7.07-6.93 (m, 2H), 6.85 (s, 1H), 4.76-4.60 (m, 1H), 3.98-3.82 (m, 1H), 3.62-3.52 (m, 1H), 2.51 (d, J = 1.9 Hz, 3H), 2.24 (d, J = 3.9 Hz, 6H). |
| C | 265 | 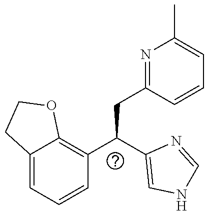 [SMILES: Cc1cccc(C[C@@H](c2c[nH]cn2)c2cccc3c2OCC3)n1] | 4-[(R)-1-(2,3- dihydro-1- benzofuran-7-yl)-2- (6-methyl-2- pyridyl)ethyl]imida- zole | 305.381 | 305.95 | 98.4 | ¹H NMR (300 MHz, DMSO-d₆) δ 12.00-11.38 (m, 1H), 7.53-7.32 (m, 2H), 7.11-6.90 (m, 3H), 6.84 (d, J = 7.7 Hz, 1H), 6.78-6.61 (m, 2H), 4.65 (t, J = 7.9 Hz, 1H), 4.59-4.32 (m, 2H), 3.43-3.37 (m, 1H), 3.27-3.07 (m, 3H), 2.40 (s, 3H). |
| C | 266 | 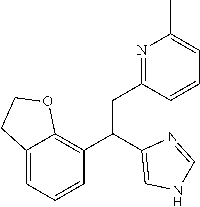 [SMILES: Cc1cccc(CC(c2c[nH]cn2)c2cccc3c2OCC3)n1] | 4-[(S)-1-(2,3- dihydro-1- benzofuran-7-yl)-2- (6-methyl-2- pyridyl)ethyl]imida- [symbol] | 305.381 | 306.05 | 97.9 | ¹H NMR (300 MHz, DMSO-d₆) δ 7.49-7.34 (m, 2H), 7.01 (dd, J = 7.4, 4.8 Hz, 2H), 6.89 (dd, J = 31.8, 7.6 Hz, 2H), 6.74-6.66 (m, 2H), 4.65 (t, J = 7.8 Hz, 1H), 4.55-4.35 (m, 2H), 3.24-3.18 (m, 2H), 3.12 (t, J = 8.7 Hz, 2H), 2.40 (s, 3H). |
| C | 267 | 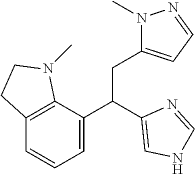 [SMILES: CN1CCc2cccc(C(Cc3ccnn3C)c3c[nH]cn3)c21] | 5-[2-(4-imidazolyl)- 2-(1-methyl-7- indolyl)ethyl]-1- methylpyrazole | 305.385 | [M − H]− 304.2 | 99.5 | 1H NMR (400 MHz, DMSO-d₆) δ 12.04 (s, 1H), 7.61 (s, 1H), 7.35 (dd, J = 7.8, 1.1 Hz, 1H), 7.23-7.15 (m, 2H), 7.13 (d, J = 1.8 Hz, 1H), 6.94 (t, J = 7.6 Hz, 1H), 6.82 (s, 1H), 6.35 (d, J = 3.1 Hz, 1H), 5.84 (d, J = 1.9 Hz, 1H), 5.21 (t, J = 7.6 Hz, 1H), 4.02 (s, 3H), 3.64 (s, 3H), 3.52 (dd, J = 15.5, 7.6 Hz, 1H), 3.41- 3.32 (m, 1H). |
| C | 268 | 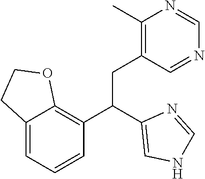 [SMILES: Cc1ncncc1CC(c1c[nH]cn1)c1cccc2c1OCC2] | 5-[2-(2,3-dihydro-1- benzofuran-7-yl)-2- (4-imidazolyl)ethyl]- 4-methylpyrimidine | 306.369 | 307 | 96.2 | ¹H NMR (400 MHz, Chloroform-d) δ 8.83 (s, 1H), 8.12 (s, 1H), 7.69 (s, 1H), 7.04 (t, J = 7.6 Hz, 2H), 6.91-6.64 (m, 2H), 4.51 (t, J = 8.6 Hz, 1H), 4.45- 4.21 (m, 2H), 3.47 (dd, J = 13.8, 7.2 Hz, 1H), 3.25 (dd, J = 13.9, 8.5 Hz, 1H), 3.16 (t, J = 8.7 Hz, 2H), 2.51 (s, 3H). |
| C | 269 | 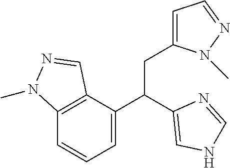 | 4-[1-(4-imidazolyl)- 2-(1-methyl-5- pyrazolyl)ethyl]-1- methyl-1H-indazole | 306.373 | 307 | 98.7 | 1H NMR (400 MHz, DMSO-d6) δ 11.82 (s, 1H), 8.16 (s, 1H), 7.52 (s, 1H), 7.42 (d, J = 8.4 Hz, 1H), 7.29 (t, J = 7.7 Hz, 1H), 7.10 (dd, J = 4.2, 2.6 Hz, 2H), 6.90 (s, 1H), 5.84 (d, J = 1.8 Hz, 1H), 4.67 (s, 1H), 3.99 (s, 3H), 3.61 (s, 3H), 3.64-3.50 (m, 1H), 3.43-3.35 (m, 1H). |
| C | 270 | 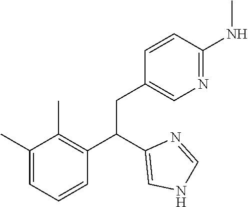 | N-methyl{5-[2-(4- imidazolyl)-2-(2,3- xylyl)ethyl]-2- pyridyl}amine | 306.413 | 307.1 | 97.7 | ¹H NMR (400 MHz, DMSO-d₆) δ 11.90-11.60 (m, 1H), 7.68 (s, 1H), 7.49 (s, 1H), 7.28-7.16 (m, 1H), 7.10 (d, J = 8.6 Hz, 1H), 7.04-6.86 (m, 2H), 6.75 (s, 1H), 6.25 (d, J = 8.5 Hz, 1H), 6.12 (s, 1H), 4.38 (s, 1H), 3.20-3.10 (m, 1H), 2.87 (dd, J = 13.9, 7.5 Hz, 1H), 2.67 (d, J = 4.7 Hz, 3H), 2.29-2.07 (m, 6H). |
| C | 271 | 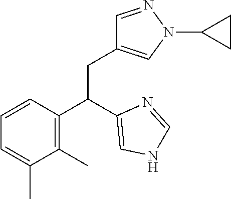 [SMILES: Cc1cccc(C(Cc2cnn(C3CC3)c2)c2c[nH]cn2)c1C] | 1-cyclopropyl-4-[2- (4-imidazolyl)-2- (2,3- xylyl)ethyl]pyrazole | 306.413 | 307.2 | 99.6 | ¹H NMR (400 MHz, DMSO-d₆) δ 11.86-11.64 (m, 1H), 7.57-7.41 (m, 1H), 7.33-7.23 (m, 1H), 7.18 (d, J = 7.5 Hz, 1H), 7.06-6.86 (m, 3H), 6.83-6.64 (m, 1H), 4.43-4.26 (m, 1H), 3.61-3.47 (m, 1H), 3.21-3.03 (m, 1H), 2.91-2.78 (m, 1H), 2.24-2.20 (m, 3H), 2.19-2.09 (m, 3H), 1.04-0.70 (m, 4H). |
| C | 272 | 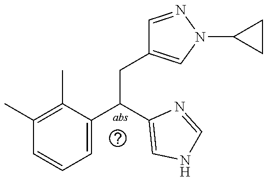 [SMILES: Cc1cccc(C(Cc2cnn(C3CC3)c2)c2c[nH]cn2)c1C] | 3-[(S)-2-(4- imidazolyl)-2-(2,3- xylyl)ethyl]-1- cyclopropylpyrazole | 306.413 | 307.15 | 99.5 | ¹H NMR (400 MHz, Chloroform-d) δ 7.58 (s, 1H), 7.23 (d, J = 1.8 Hz, 1H), 7.20 (dd, J = 7.3, 1.8 Hz, 1H), 7.13-7.06 (m, 1H), 7.06-7.01 (m, 1H), 6.68 (s, 1H), 5.81 (d, J = 1.8 Hz, 1H), 4.67 (t, J = 7.5 Hz, 1H), 3.70-3.64 (m, 1H), 3.37-3.30 (m, 1H), 3.23- 3.09 (m, 1H), 2.32-2.22 (m, 3H), 2.21-2.10 (m, 3H), 1.17-1.05 (m, 2H), 1.04-0.92 (m, 2H). |
| C | 273 | 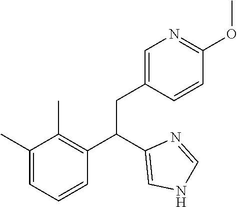 | 4-[2-(6-methoxy-3- pyridyl)-1-(2,3- xylyl)ethyl]imidazole | 307.397 | 308.15 | 96.9 | ¹H NMR (400 MHz, DMSO-d₆) δ 11.89-11.40 (m, 1H), 7.77 (d, J = 2.6 Hz, 1H), 7.43 (s, 1H), 7.33 (dd, J = 8.5, 2.5 Hz, 1H), 7.22-7.05 (m, 1H), 6.99-6.81 (m, 2H), 6.69 (s, 1H), 6.55 (d, J = 8.5 Hz, 1H), 4.36 (t, J = 7.7 Hz, 1H), 3.68 (s, 3H), 3.24-3.17 (m, 1H), 2.92 (dd, J = 13.8, 7.3 Hz, 1H), 2.19-1.97 (m, 6H). |
| C | 274 | 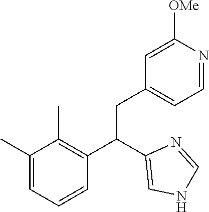 [SMILES: COc1cc(CC(c2c[nH]cn2)c2cccc(C)c2C)ccn1] | 4-[2-(2-methoxy-4- pyridyl)-1-(2,3- xylyl)ethyl]imidazole | 307.397 | 308.15 | 99.7 | ¹H NMR (400 MHz, DMSO-d₆) δ 11.77 (d, J = 44.9 Hz, 1H), 8.04-7.79 (m, 1H), 7.48 (d, J = 18.3 Hz, 1H), 7.28-6.87 (m, 3H), 6.81-6.34 (m, 3H), 4.81- 4.31 (m, 1H), 3.75 (d, J = 5.1 Hz, 3H), 3.45-3.35 (m, 1H), 3.11-2.99 (m, 1H), 2.24-2.09 (m, 6H). |
| C | 275 | 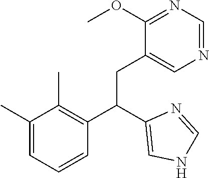 [SMILES: COc1ncncc1CC(c1c[nH]cn1)c1cccc(C)c1C] | 5-[2-(4-imidazolyl)- 2-(2,3-xylyl)ethyl]-4- methoxypyrimidine | 308.385 | 309.1 | 99.0 | ¹H NMR (400 MHz, DMSO-d₆) δ 11.90-11.61 (m, 1H), 8.63-8.47 (m, 1H), 8.14-7.94 (m, 1H), 7.55- 7.40 (m, 1H), 7.24 (d, J = 7.6 Hz, 1H), 7.10-6.89 (m, 2H), 6.86-6.76 (m, 1H), 4.74-4.52 (m, 1H), 3.95 (s, 3H), 3.29-3.21 (m, 1H), 2.98 (dd, J = 13.7, 7.0 Hz, 1H), 2.19 (s, 3H), 2.16 (s, 3H). |
| C | 276 | 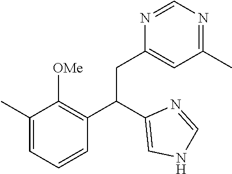 | 4-[2-(4-imidazolyl)- 2-(2-methoxy-3- tolyl)ethyl]-6- methylpyrimidine | 308.385 | 309.15 | 99.2 | 1H NMR (400 MHz, DMSO-d6) δ 11.73 (s, 1H), 8.87 (s, 1H), 7.48 (s, 1H), 7.26 (d, J = 7.5 Hz, 1H), 7.11 (s, 1H), 6.97 (dt, J = 15.0, 7.3 Hz, 2H), 6.72 (s, 1H), 4.95 (t, J = 7.8 Hz, 1H), 3.65 (s, 3H),3.45 (s, 1H), 3.17 (dd, J = 13.9, 7.4 Hz, 1H), 2.32 (s, 3H), 2.20 (s, 3H). |
| C | 277 | 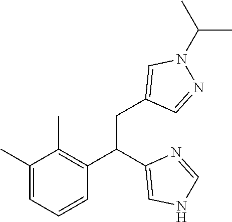 [SMILES: Cc1cccc(C(Cc2cnn(C(C)C)c2)c2c[nH]cn2)c1C] | 4-[2-(4-imidazolyl)-2- (2,3-xylyl)ethyl]-1- isopropylpyrazole | 308.429 | 309.1 | 99.4 | ¹H NMR (400 MHz, DMSO-d₆) δ 12.67-11.80 (m, 1H), 8.15 (s, 1H), 7.51 (d, J = 1.1 Hz, 1H), 7.20 (s, 1H), 7.12 (dd, J = 7.5, 1.7 Hz, 1H), 7.04-6.90 (m, 3H), 6.78 (s, 1H), 4.38-4.22 (m, 2H), 3.15 (dd, J 14.4, 7.9 Hz, 1H), 2.86 (dd, J = 14.4, 7.4 Hz, 1H), 2.21 (s, 3H), 2.13 (s, 3H), 1.28 (dd, J = 6.7, 1.5 Hz, 6H). |
| C | 278 | 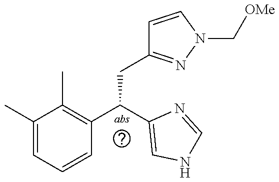 [SMILES: COCn1ccc(C[C@H](c2c[nH]cn2)c2cccc(C)c2C)n1] | 3-[(S)-2-(4-imidazolyl)-2- (2,3-xylyl)ethyl]-1- (methoxymethyl)pyrazole | 310.401 | 311.15 | 90.2 | ¹H NMR (400 MHz, DMSO-d₆) δ 12.07-11.44 (m, 1H), 7.60 (d, J = 2.3 Hz, 1H), 7.50 (d, J = 1.2 Hz, 1H), 7.12 (dd, J = 7.6, 1.7 Hz, 1H), 7.00-6.89 (m, 2H), 6.75 (s, 1H), 5.86 (d, J = 2.3 Hz, 1H), 5.22 (s, 2H), 4.58 (t, J = 7.7 Hz, 1H), 3.30-3.25 (m, 1H), 3.09 (s, 3H), 3.08-3.01 (m, 1H), 2.22-2.16 (m, 6H). |
| C | 279 | 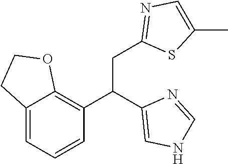 [SMILES: Cc1cnc(CC(c2c[nH]cn2)c2cccc3c2OCC3)s1] | 2-[2-(2,3-dihydro-1- benzofuran-7-yl)-2-(4- imidazolyl)ethyl]-5- methyl-1,3-thiazole | 311.41 | 312.1 | 98.6 | ¹H NMR (400 MHz, DMSO-d₆) δ 12.04-11.63 (m, 1H), 7.51 (d, J = 21.9 Hz, 1H), 7.36-7.22 (m, 1H), 7.13-6.85 (m, 2H), 6.87-6.47 (m, 2H), 4.57-4.44 (m, 3H), 3.62 (dd, J = 14.7, 8.3 Hz, 1H), 3.53-3.38 (m, 1H), 3.15 (t, J = 8.7 Hz, 2H), 2.39-2.16 (m, 3 H). |
| C | 280 | 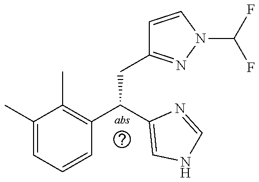 [SMILES: Cc1cccc([C@H](Cc2ccn(C(F)F)n2)c2c[nH]cn2)c1C] | 3-[(S)-2-(4-imidazolyl)-2- (2,3-xylyl)ethyl]-1- (difluoromethyl)pyrazole | 316.355 | 317.1 | 99.4 | ¹H NMR (400 MHz, DMSO-d₆) δ 12.01-11.35 (m, 1H), 7.72 (t, J = 57.9 Hz, 1H), 7.52-7.38 (m, 2H), 7.16 (d, J = 7.4 Hz, 1H), 6.98-6.86 (m, 2H), 6.70 (s, 1H), 5.91 (d, J = 1.7 Hz, 1H), 4.52 (t, J = 7.7 Hz, 1H), 3.53 (dd, J = 15.7, 8.0 Hz, 2H), 2.23-2.04 (m, 6H). |
| C | 281 | 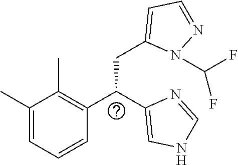 [SMILES: Cc1cccc([C@H](Cc2ccnn2C(F)F)c2c[nH]cn2)c1C] | 5-[(S)-2-(4-imidazolyl)-2- (2,3-xylyl)ethyl]-1- (difluoromethyl)pyrazole | 316.355 | 317.05 | 99.6 | ¹H NMR (400 MHz, DMSO-d₆) δ 8.21 (s, 1H), 7.95 (d, J = 2.6 Hz, 1H), 7.81-7.41 (m, 2H), 7.14 (d, J = 7.4 Hz, 1H), 7.03-6.88 (m, 2H), 6.75 (s, 1H), 6.05 (d, J = 2.6 Hz, 1H), 4.60 (t, J = 7.7 Hz, 1H), 3.16-3.03 (m, 2H), 2.20 (d, J = 6.5 Hz, 6H). |
| C | 282 | 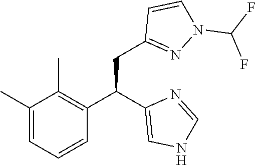 [SMILES: Cc1cccc([C@@H](Cc2ccn(C(F)F)n2)c2c[nH]cn2)c1C] | 3-[(R)-2-(4-imidazolyl)-2- (2,3-xylyl)ethyl]-1- (difluoromethyl)pyrazole | 316.355 | 317.15 | 99.2 | ¹H NMR (400 MHz, Methanol-d₄) δ 7.60 (d, J = 1.2 Hz, 1H), 7.58-7.27 (m, 2H), 7.17-7.11 (m, 1H), 7.07-6.97 (m, 2H), 6.74 (s, 1H), 5.91 (d, J = 1.6 Hz, 1H), 4.75-4.66 (m, 1H), 4.59 (s, 1H), 3.70-3.59 (m, 1H), 3.45-3.34 (m, 1H), 2.25 (s, 3H), 2.13 (s, 3H) |
| C | 283 | 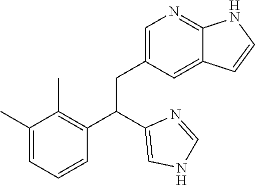 [SMILES: Cc1cccc(C(Cc2cnc3[nH]ccc3c2)c2c[nH]cn2)c1C] | 4-[2-(1H-1,7-diazainden- 5-yl)-1-(2,3- xylyl)ethyl]imidazole | 316.408 | 317.2 | 99.8 | ¹H NMR (400 MHz, DMSO-d₆) δ 11.54-11.21 (m, 1H), 8.18 (s, 2H), 7.97 (d, J = 2.0 Hz, 1H), 7.67 (d, J = 2.2 Hz, 2H), 7.36 (t, J = 2.8 Hz, 1H), 7.25 (d, J = 7.7 Hz, 1H), 7.02 (t, J = 7.6 Hz, 1H), 6.95 (d, J = 7.4 Hz, 1H), 6.87 (s, 1H), 6.31 (d, J = 3.3 Hz, 1H), 4.59- 4.56 (m, 1H), 3.45 (dd, J = 13.8, 8.2 Hz, 1H), 3.16 (dd, J = 13.8, 7.2 Hz, 1H), 2.26-2.06 (m, 6H). |
| C | 284 | 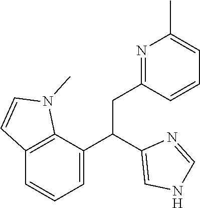 [SMILES: Cc1cccc(CC(c2c[nH]cn2)c2cccc3ccn(C)c23)n1] | 4-[1-(1-methyl-7-indolyl)- 2-(6-methyl-2- pyridyl)ethyl]imidazole | 316.408 | 317.15 | 96.8 | 1H NMR (400 MHz, DMSO-d6) δ 8.15 (s, 2H), 7.53 (s, 1H), 7.40 (t, J = 7.6 Hz, 1H), 7.28 (dd, J = 7.8, 1.2 Hz, 1H), 7.16 (d, J = 3.1 Hz, 1H), 7.11 (d, J = 7.3 Hz, 1H), 6.98-6.86 (m, 3H), 6.81 (s, 1H), 6.31 (d, J = 3.1 Hz, 1H), 5.62 (t, J = 7.6 Hz, 1H), 4.21 (s, 3H), 3.50 (dd, J = 14.0, 7.4 Hz, 1H), 3.41 (dd, J = 14.0, 7.9 Hz, 1H), 2.39 (s, 3H). |
| C | 285 | 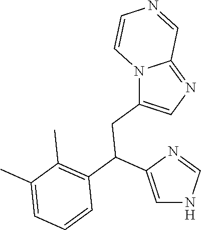 [SMILES: Cc1cccc(C(Cc2cnc3cnccn23)c2c[nH]cn2)c1C] | 3-[2-(4-imidazolyl)-2- (2,3-xylyl)ethyl]-1,3a,6- triazaindene | 317.396 | 318.15 | 98.4 | ¹H NMR (400 MHz, DMSO-d₆) δ 12.09-11.65 (m, 1H), 8.93 (s, 1H), 8.33 (d, J = 4.5 Hz, 1H), 7.80 (d, J = 4.7 Hz, 1H), 7.52 (s, 1H), 7.36 (d, J = 18.3 Hz, 2H), 7.07-6.87 (m, 2H), 6.81 (s, 1H), 4.86-4.59 (m, 1H), 3.74 (dd, J = 15.5, 8.1 Hz, 1H), 3.53-3.44 (m, 1H), 2.17 (s, 3H), 2.11 (s, 3H). |
| C | 286 | 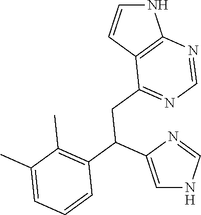 [SMILES: Cc1cccc(C(Cc2ncnc3[nH]ccc23)c2c[nH]cn2)c1C] | 4-[2-(4-imidazolyl)-2- (2,3-xylyl)ethyl]-1H-1,5,7- triazaindene | 317.396 | 318.05 | 99.1 | ¹H NMR (400 MHz, DMSO-d₆) δ 12.05-11.53 (m, 2H), 8.68-8.51 (m, 1H), 7.50-7.34 (m, 2H), 7.29- 7.05 (m, 1H), 7.03-6.83 (m, 2H), 6.77-6.55 (m, 2H), 5.26-5.04 (m, 1H), 3.78-3.61 (m, 1H), 3.50- 3.39 (m, 1H), 2.31-2.03 (m, 6H). |
| C | 287 | 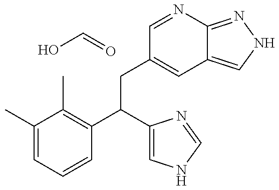 | 5-[2-(4-imidazolyl)-2- (2,3-xylyl)ethyl]-2H-1,2,7- triazaindene-formic acid (1/1) | 317.4 | 318.2 | 97.2 | ¹H NMR (400 MHz, DMSO-d₆) δ 13.43 (s, 1H), 12.33 (s, 1H), 8.25 (d, J = 2.0 Hz, 1H), 8.13 (s, 1H), 8.00 (s, 1H), 7.89 (d, J = 2.0 Hz, 1H), 7.66 (s, 1H), 7.28- 7.21 (m, 1H), 7.02 (t, J = 7.6 Hz, 1H), 6.98-6.93 (m, 1H), 6.87 (s, 1H), 4.56 (t, J = 7.8 Hz, 1H), 3.50 (dd, J = 13.8, 8.4 Hz, 1H), 3.21-3.16 (m, 1H), 2.17 (s, 3H), 2.12 (s, 3H). |
| C | 288 | 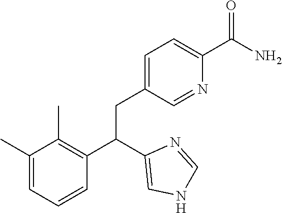 [SMILES: Cc1cccc(C(Cc2ccc(C(N)=O)nc2)c2c[nH]cn2)c1C] | 5-[2-(4-imidazolyl)-2- (2,3-xylyl)ethyl]-2- pyridinecarboxamide | 320.396 | 321.2 | 99.4 | ¹H NMR (400 MHz, DMSO-d₆) δ 11.90-11.65 (m, 1H), 8.36-8.24 (m, 1H), 7.99 (s, 1H), 7.88-7.79 (m, 1H), 7.76-7.63 (m, 1H), 7.58-7.41 (m, 2H), 7.32-7.10 (m, 1H), 7.08-6.87 (m, 2H), 6.82-6.70 (m, 1H), 4.67-4.42 (m, 1H), 3.55-3.36 (m, 1H), 3.23-3.10 (m, 1H), 2.28-2.12 (m, 5H), 2.11-2.04 (m, 1H). |
| C | 289 | 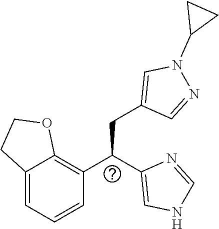 [SMILES: c1cc2c(c([C@@H](Cc3cnn(C4CC4)c3)c3c[nH]cn3)c1)OCC2] | 4-[(R)-2-12,3- dihydro-1- benzofuran-7-yl)-2- (4-imidazolyl)ethyl]- 1- [symbol] | 320.396 | 321.1 | 94.0 | ¹H NMR (400 MHz, DMSO-d₆) δ 11.76 (s, 1H), 7.50 (s, 1H), 7.26 (s, 1H), 7.02 (t, J = 6.7 Hz, 2H), 6.95 (s, 1H), 6.79-6.64 (m, 2H), 4.59-4.37 (m, 2H), 4.21 (t, J = 7.8 Hz, 1H), 3.64-3.47 (m, 1H), 3.22-3.01 (m, 3H), 2.93 (dd, J = 14.5, 7.6 Hz, 1H), 1.04-0.71 (m, 4H) |
| C | 290 | 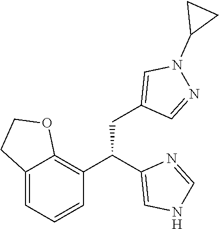 [SMILES: c1cc2c(c([C@H](Cc3cnn(C4CC4)c3)c3c[nH]cn3)c1)OCC2] | 4-[(S)-2-(2,3- dihydro-1- benzofuran-7-yl)-2- (4-imidazolyl)ethyl]- 1- [symbol] | 320.396 | 321.05 | 97.1 | ¹H NMR (400 MHz, DMSO-d₆) δ 11.75 (s, 1H), 7.50 (s, 1H), 7.26 (s, 1H), 7.02 (t, J = 6.7 Hz, 2H), 6.95 (s, 1H), 6.78-6.65 (m, 2H), 4.57-4.39 (m, 2H), 4.21 (t, J = 7.8 Hz, 1H), 3.62-3.47 (m, 1H), 3.19-3.05 (m, 3H), 2.93 (dd, J = 14.5, 7.6 Hz, 1H), 0.96-0.82 (m, 4H). |
| C | 291 | 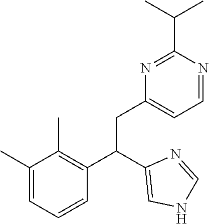 [SMILES: Cc1cccc(C(Cc2ccnc(C(C)C)n2)c2c[nH]cn2)c1C] | 4-[2-(4-imidazolyl)- 2-(2,3-xylyl)ethyl]-2- isopropylpyrimidine | 320.4 | 321.3 | 96.89 | ¹H NMR (400 MHz, DMSO-d₆): 11.64 (s, 1H), 8.45 (d, J = 4.8 Hz, 1H), 7.49 (s, 1H), 7.13 (d, J = 7.2 Hz, 1H), 7.01-6.90 (m, 3H), 6.74 (s, 1H), 4.88 (t, J = 7.6 Hz, 1H), 3.51-3.42 (m, 1H), 3.30-3.18 (m, 1H), 3.09-2.99 (m, 1H), 2.19 (d, J = 8.4 Hz, 6H), 1.22 (s, 3H), 1.20 (s, 3H). |
| C | 292 | 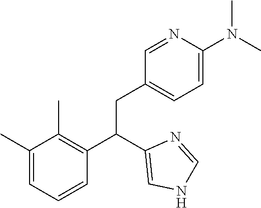 | N,N-dimethyl{5-[2- (4-imidazolyl)-2- (2,3-xylyl)ethyl]-2- pyridyl}amine | 320.44 | 321.2 | 98.7 | ¹H NMR (400 MHz, DMSO-d₆) δ 11.86-11.63 (m, 1H), 7.86-7.73 (m, 1H), 7.55-7.39 (m, 1H), 7.29- 7.06 (m, 2H), 7.05-6.87 (m, 2H), 6.80-6.66 (m, 1H), 6.44 (d, J = 8.7 Hz, 1H), 4.50-4.30 (m, 1H), 3.28-3.10 (m, 1H), 3.00-2.77 (m, 7H), 2.30-2.00 (m, 6H). |
| C | 293 | 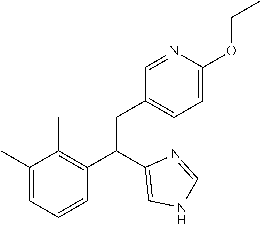 [SMILES: CCOc1ccc(CC(c2c[nH]cn2)c2cccc(C)c2C)cn1] | 4-[2-(6-ethoxy-3- pyridyl)-1-(2,3- xylyl)ethyl]imidazole | 321.424 | 322.1 | 99.6 | ¹H NMR (300 MHz, DMSO-d₆) δ 11.94-11.55 (m, 1H), 7.82 (d, J = 2.4 Hz, 1H), 7.50 (s, 1H), 7.38 (dd, J = 8.5, 2.5 Hz, 1H), 7.30-7.11 (m, 1H), 7.08-6.88 (m, 2H), 6.77 (s, 1H), 6.58 (d, J = 8.5 Hz, 1H), 4.58- 4.35 (m, 1H), 4.19 (q, J = 7.0 Hz, 2H), 3.32-3.23 (m, 1H), 2.98 (dd, J = 13.8, 7.3 Hz, 1H), 2.30-2.00 (m, 6H), 1.25 (t, J = 7.0 Hz, 3H). |
| C | 294 | 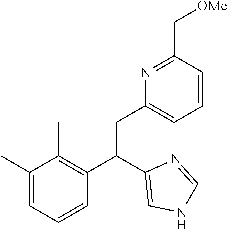 [SMILES: COCc1cccc(CC(c2c[nH]cn2)c2cccc(C)c2C)n1] | 4-{2-[6- (methoxymethyl)-2- pyridyl]-1-(2,3- xylyl)ethyl}imidazole | 321.424 | 322.2 | 98.8 | ¹H NMR (400 MHz, DMSO-d₆) δ 8.21 (s, 1H), 7.55 (t, J = 7.7 Hz, 1H), 7.50 (s, 1H), 7.14 (t, J = 7.0 Hz, 2H), 7.03-6.94 (m, 2H), 6.93-6.87 (m, 1H), 6.75 (s, 1H), 4.84 (t, J = 7.7 Hz, 1H), 4.50-4.33 (m, 2H), 3.46-3.43 (m, 1H), 3.32 (s, 3H), 3.21 (dd, J = 13.8, 7.7 Hz, 1H), 2.17 (s, 6H). |
| C | 295 | 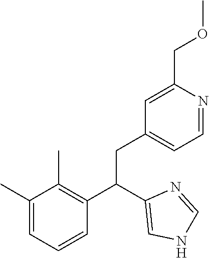 | 4-{2-[2- (methoxymethyl)-4- pyridyl]-1-(2,3- xylyl)ethyl}imidazole | 321.424 | 322.15 | 99.7 | ¹H NMR (400 MHz, DMSO-d₆) δ 11.78 (d, J = 50.1 Hz, 1H), 8.42-8.19 (m, 1H), 7.59-7.40 (m, 1H), 7.32-7.05 (m, 2H), 7.07-6.79 (m, 3H), 6.83 -6.55 (m, 1H), 4.69-4.47 (m, 1H), 4.38 (d, J = 6.5 Hz, 2H), 3.52-3.37 (m, 1H), 3.25 (d, J =5.4 Hz, 3H), 3.20- 2.99 (m, 1H), 2.27-2.07 (m, 6H). |
| C | 296 | 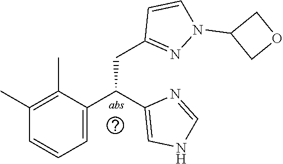 [SMILES: Cc1cccc([C@H](Cc2ccn(C3COC3)n2)c2c[nH]cn2)c1C] | 3-[(S)-2-(4- imidazolyl)-2-(2,3- xylyl)ethyl]-1-(3- oxetanyl)pyrazole | 322.412 | 323 | 98.2 | ¹H NMR (400 MHz, Chloroform-d) δ 8.36-7.99 (m, 2H), 7.47-7.32 (m, 1H), 7.07 (s, 3H), 6.70 (s, 1H), 6.05-5.83 (m, 1H), 5.46-5.28 (m, 1H), 5.14-5.02 (m, 2H), 5.02-4.93 (m, 2H), 4.73 (dd, J = 9.2, 5.2 Hz, 1H), 3.53-3.39 (m, 1H), 3.25 (dd, J = 15.1, 5.0 Hz, 1H), 2.31-2.25 (m, 3H), 2.23-2.15 (m, 3H). |
| C | 297 | 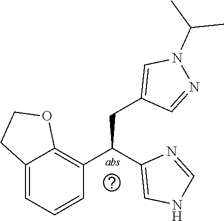 [SMILES: CC(C)n1cc(C[C@@H](c2c[nH]cn2)c2cccc3c2OCC3)cn1] | 4-[(R)-2-(2,3- dihydro-1- benzofuran-7-yl)-2- (4-imidazolyl)ethyl] 1-isopropylpyrazole | 322.412 | 323.2 | 95.6 | 1H NMR (400 MHz, DMSO-d6) δ 11.75 (s, 1H), 7.50 (s, 1H), 7.23 (s, 1H), 7.04-7.01 (m, 2H), 6.95 (s, 1H) 6.79-6.65 (m, 2H), 4.47 (dq, J = 20.7, 8.6 Hz, 2H), 4.30 (h, J = 6.7 Hz, 1H), 4.22 (t, J = 7.7 Hz, 1H), 3.19- 3.04 (m, 3H), 2.94 (dd, J = 14.4, 7.7 Hz, 1H), 1.29 (d, J = 6.6 Hz, 6H). |
| C | 298 | 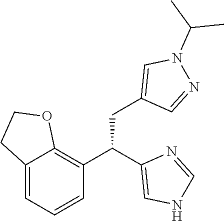 [SMILES: CC(C)n1cc(C[C@H](c2c[nH]cn2)c2cccc3c2OCC3)cn1] | 4-[(S)-2-(2,3- dihydro-1- benzofuran-7-yl)-2- (4-imidazolyl)ethyl]- 1-isopropylpyrazole | 322.412 | 323.15 | 99.2 | 1H NMR (400 MHz, DMSO-d6) δ 11.75 (s, 1H), 7.50 (s, 1 H), 7.23 (s, 1H), 7.06-6.92 (m, 3H), 6.79-6.65 (m, 2H), 4.47 (dq, J = 20.7, 8.6 Hz, 2H), 4.30 (h, J = 6.7 Hz, 1H), 4.22 (t, J = 7.7 Hz, 1H), 3.19-3.04 (m, 3H), 2.94 (dd, J = 14.4, 7.7 Hz, 1H), 1.29 (d, J = 6.6 Hz, 6H). |
| C | 299 | 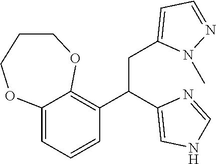 [SMILES: Cn1nccc1CC(c1c[nH]cn1)c1cccc2c1OCCCO2] | 5-[2-(3,4-dihydro- 2H-1,5- benzodioxepin-6-yl)- 2-(4- imidazolyl)ethyl]-1- [symbol] | 324.384 | 325.2 | 99.3 | 1H NMR (400 MHz, DMSO-d6) δ 11.78 (s, 1H), 7.53 (s, 1H), 7.14 (d, J = 1.9 Hz, 1H), 7.03 (s, 1H), 6.82 (dt, J = 18.7, 7.7 Hz, 3H), 5.75 (d, J = 1.9 Hz, 1H), 4.68 (s, 1H), 4.02 (ddt, J = 17.1, 11.3, 5.8 Hz, 3H), 3.81 (dt, J = 11.6, 5.5 Hz, 1H), 3.69 (s, 3H), 3.32 (s, 1H), 3.13 (d, J = 10.6 Hz, 1H), 2.05 (p, J = 5.0 Hz, 2H). |
| C | 300 | 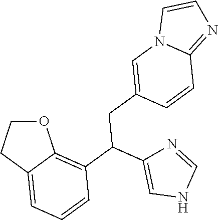 [SMILES: c1cc2c(c(C(Cc3ccc4nccn4c3)c3c[nH]cn3)c1)OCC2] | 4-[2-(1,3a-diaza-5- indenyl)-1-(2,3- dihydro-1- benzofuran-7- ylethyl]imidazole | 330.391 | 331 | 97.4 | ¹H NMR (400 MHz, DMSO-d₆) δ 12.65-11.86 (m, 1H), 8.21 (s, 1H), 7.81 (d, J = 1.0 Hz, 1H), 7.70 (s, 1H), 7.48 (d, J = 1.2 Hz, 1H), 7.40 (d, J = 9.2 Hz, 1H), 7.10-6.92 (m, 3H), 6.88 (s, 1H), 6.73 (t, J = 7.5 Hz, 1H), 4.57-4.45 (m, 1H), 4.45-4.31 (m, 2H), 3.29- 3.27 (m, 1H), 3.17-3.05 (m, 3H). |
| C | 301 | 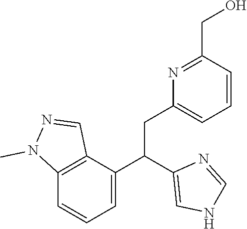 [SMILES: Cn1ncc2c(C(Cc3cccc(CO)n3)c3c[nH]cn3)cccc21] | {6-[2-(4-imidazolyl)-2-(1- methyl-1H-indazol-4- yl)ethyl]-2- pyridyl}methanol | 333.395 | 334.15 | 98.8 | ¹H NMR (400 MHz, DMSO-d6) δ 11.75 (d, J = 74.5 Hz, 1H), 8.02 (d, J = 12.0 Hz, 1H), 7.50-7.33 (m, 2H), 7.31-7.05 (m, 3H), 7.00-6.74 (m, 3H), 5.35- 5.22 (m, 1H), 5.02-4.77 (m, 1H), 4.44 (d, J = 5.7 Hz, 2H), 3.90 (d, J = 4.5 Hz, 3H), 3.61-3.48 (m, 2H). |
| C | 302 | 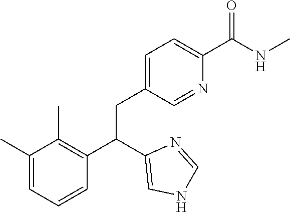 | N-methyl-5-[2-(4- imidazolyl)-2-(2,3- xylyl)ethyl]-2- pyridinecarboxamide | 334.423 | 335.15 | 99.3 | ¹H NMR (400 MHz, DMSO-d₆) δ 11.91-11.62 (m, 1H), 8.64 (d, J = 5.2 Hz, 1H), 8.26 (s, 1H), 7.82 (d, J = 7.9 Hz, 1H), 7.68 (dd, J = 8.2, 2.1 Hz, 1H), 7.59- 7.44 (m, 1H), 7.37-7.11 (m, 1H), 7.09-6.90 (m, 2H), 6.85-6.65 (m, 1H), 4.66-4.41 (m, 1H), 3.49 (dd, J = 13.5, 8.3 Hz, 1H), 3.16 (dd, J = 13.5, 7.4 Hz, 1H), 2.77 (d, J = 4.8 Hz, 3H), 2.18 (s, 3H), 2.15- [symbol] |
| C | 303 | 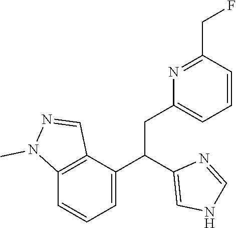 [SMILES: Cn1ncc2c(C(Cc3cccc(CF)n3)c3c[nH]cn3)cccc21] | 4-{2-[6-(fluoromethyl)-2- pyridyl]-1-(4- imidazolyl)ethyl}-1- methyl-1H-indazole | 335.386 | 336.15 | 94.5 | 1H NMR (400 MHz, Chloroform-d) δ 7.89-7.84 (m, 1H), 7.64 (s, 1H), 7.50-7.44 (m, 1H), 7.28-7.16 (m, 3H), 6.97-6.93 (m, 1H), 6.82 (d, J = 7.7 Hz, 1H), 6.75 (s, 1H), 5.46 (d, J = 46.9 Hz, 2H), 5.02-4.94 (m, 1H), 4.01 (s, 3H), 3.78-3.71 (m, 1H), 3.57- 3.48 (m, 1H). |
| C | 304 | 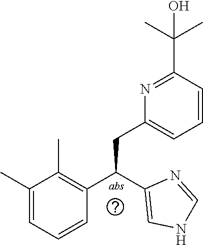 [SMILES: Cc1cccc([C@@H](Cc2cccc(C(C)(C)O)n2)c2c[nH]cn2)c1C] | 2-{6-[(R)-2-(4-imidazolyl) 2-(2,3-xylyl)ethyl]-2- pyridyl}-2-propanol | 335.451 | 336.2 | 98.9 | ¹H NMR (400 MHz, DMSO-d₆) δ 11.89-11.56 (m, 1H), 7.67-7.27 (m, 3H), 7.23-6.81 (m, 4H), 6.76 (s, 1H), 5.11 (s, 1H), 4.95-4.75 (m, 1H), 3.53-3.41 (m, 1H), 3.19 (dd, J = 13.7, 7.6 Hz, 1H), 2.17 (s, 6H), 1.39 (d, J = 3.9 Hz, 6H). |
| C | 305 | 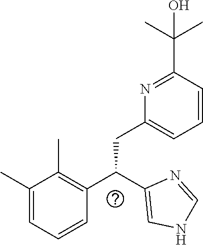 [SMILES: Cc1cccc([C@H](Cc2cccc(C(C)(C)O)n2)c2c[nH]cn2)c1C] | 2-{6-[(S)-2-(4-imidazolyl) 2-(2,3-xylyl)ethyl]-2- pyridyl}-2-propanol | 335.451 | 336.25 | 99.4 | ¹H NMR (400 MHz, DMSO-d₆) δ 11.81-11.62 (m, 1H), 7.57-7.32 (m, 3H), 7.17-6.86 (m, 4H), 6.79- 6.69 (m, 1H), 5.15-5.08 (m, 1H), 4.96-4.77 (m, 1H), 3.49-3.36 (m, 1H), 3.23-3.14 (m, 1H), 2.21- 2.12 (m, 6H), 1.44-1.33 (m, 6H). |
| C | 306 | 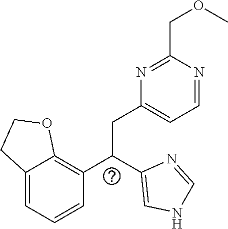 [SMILES: COCc1nccc(CC(c2c[nH]cn2)c2cccc3c2OCC3)n1] | 4-[2-(2,3-dihydro-1- benzofuran-7-yl)-2-(4- imidazolyl)ethyl]-2- (methoxymethyl)pyrimidine | 336.395 | 337.1 | 98.5 | ¹H NMR (400 MHz, DMSO-d₆) δ 8.52 (d, J = 5.1 Hz, 1H), 8.18 (s, 1H), 7.52 (s, 1H), 7.09 (d, J = 5.1 Hz, 1H), 7.03 (dd, J = 7.5, 4.5 Hz, 2H), 6.72 (dd, J = 13.2, 5.8 Hz, 2H), 4.68 (t, J = 7.9 Hz, 1H), 4.54- 4.45 (m, 3H), 4.40 (q, J = 8.7 Hz, 1H), 3.46 (dd, J [symbol] 13.8, 8.3 Hz, 1H), 3.32 (s, 3H), 3.30 -3.23 (m, 1H), 3.12 (t, J = 8.7 Hz, 2H). |
| C | 307 | 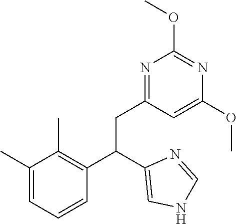 | 4-[2-(4-imidazolyl)-2- (2,3-xylyl)ethyl]-2,6- dimethoxypyrimidine | 338.411 | 339.15 | 99.4 | ¹H NMR (400 MHz, DMSO-d₆) δ 11.73 (s, 1H), 7.48 (s, 1H), 7.11 (s, 1H), 7.01-6.88 (m, 2H), 6.75 (s, 1H), 6.30 (5, 1H), 4.85 (s, 1H), 3.85 (s, 3H), 3.80 (s, 3H), 3.39-3.33 (m, 1H), 3.06 (dd, J = 14.0, 7.1 Hz, 1H), 2.23 (d, J = 19.0 Hz, 6H). |
| C | 308 | 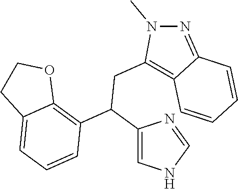 [SMILES: Cn1nc2ccccc2c1CC(c1c[nH]cn1)c1cccc2c1OCC2] | 3-[2-(2,3-dihydro-1- benzofuran-7-yl)-2-(4- imidazolyl)ethyl]-2- methyl-2H-indazole | 344.418 | 345 | 98.1 | ¹H NMR (400 MHz, DMSO-d₆) δ 8.19 (5, 1H), 7.58 (s, 1H), 7.47 (d, J = 8.4 Hz, 1H), 7.41 (d, J = 8.6 Hz, 1H), 7.12 (t, J = 7.8 Hz, 2H), 7.02 (d, J = 7.2 Hz, 1H), 6.91- 6.82 (m, 1H), 6.79 (s, 1H), 6.72 (t, J = 7.5 Hz, 1H), 4.50 (dt, J = 27.0, 8.2 Hz, 2H), 4.30 (q, J = 8.7 Hz, 1H), 3.96 (s, 3H), 3.74 (dd, J = 14.6, 8.2 Hz, 2H), 3.16-2.99 (m, 2H). |
| C | 309 | 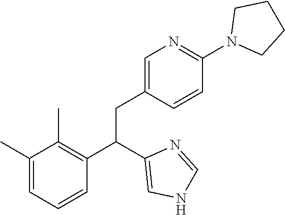 [SMILES: Cc1cccc(C(Cc2ccc(N3CCCC3)nc2)c2c[nH]cn2)c1C] | 4-{2-[6-(1-pyrrolidinyl)-3- pyridyl]-1-(2,3- xylyl)ethyl}imidazole | 346.478 | 347.3 | 99.1 | ¹H NMR (400 MHz, DMSO-d₆) δ 11.75 (s, 1H), 7.77 (d, J = 2.4 Hz, 1H), 7.55-7.44 (m, 1H), 7.19 (dd, J = 8.6, 2.4 Hz, 2H), 7.05-6.88 (m, 2H), 6.74 (s, 1H), 6.24 (d, J = 8.6 Hz, 1H), 4.39 (t, J = 7.7 Hz, 1H), 3.30- 3.23 (m, 4H), 3.19 (dd, J = 14.1, 8.4 Hz, 1H), 2.90 (dd, J = 13.9, 7.3 Hz, 1H), 2.25-2.09 (m, 6H), 1.95- 1.80 (m, 4H). |
| C | 310 | 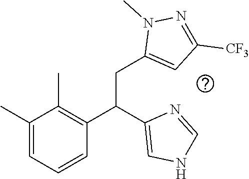 [SMILES: Cc1cccc(C(Cc2cc(C(F)(F)F)nn2C)c2c[nH]cn2)c1C] | 5-[2-(4-imidazolyl)-2- (2,3-xylyl)ethyl]-1- methyl-3- (trifluoromethyl)pyrazole | 348.372 | 349.15 | 99.8 | ¹H NMR (400 MHz, DMSO-d₆) δ 11.89-11.71 (m, 1H), 7.57-7.46 (m, 1H), 7.27 (d, J = 7.4 Hz, 1H), 7.15-6.91 (m, 2H), 6.85-6.74 (m, 1H), 6.25 (s, 1H), 4.69-4.50 (m, 1H), 3.79-3.62 (m, 3H), 3.49 (dd, J = 15.3, 8.5 Hz, 1H), 3.13 (dd, J = 15.3, 6.8 Hz, 1H), 2.31-2.01 (m, 6H). |
| C | 311 | 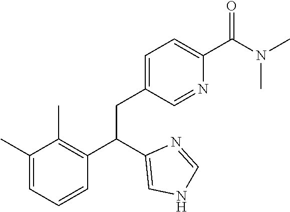 | N,N-dimethyl-5-[2-(4- imidazolyl)-2-(2,3- xylyl)ethyl]-2- pyridinecarboxamide | 348.45 | 349.2 | 99.5 | ¹H NMR (400 MHz, DMSO-d₆) δ 11.90-11.63 (m, 1H), 8.29-8.15 (m, 1H), 7.62-7.55 (m, 1H), 7.54- 7.45 (m, 1H), 7.39-7.31 (m, 1H), 7.30-7.09 (m, 1H), 7.08-6.97 (m, 1H), 6.96-6.89 (m, 1H), 6.84- 6.71 (m, 1H), 4.71-4.37 (m, 1H), 3.53-3.34 (m, 1H), 3.20-3.04 (m, 1H), 2.96 (s, 3H), 2.87 (s, 3H), 2.17 (s, 3H), 2.14-2.03 (m, 3H). |
| C | 312 | 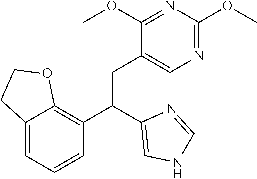 [SMILES: COc1ncc(CC(c2c[nH]cn2)c2cccc3c2OCC3)c(OC)n1] | 5-[2-(2,3-dihydro-1- benzofuran-7-yl)-2-(4- imidazolyl)ethyl]-2,4- dimethoxypyrimidine | 352.394 | 353.05 | 99.4 | ¹H NMR (400 MHz, DMSO-d₆) δ 11.88-11.46 (m, 1H), 7.85-7.53 (m, 1H), 7.53-7.19 (m, 1H), 7.06- 6.45 (m, 4H), 4.46-4.27 (m, 2H), 4.19 (q, J = 8.6 Hz, 1H), 3.82 (s, 3H), 3.73 (d, J = 4.5 Hz, 3H), 3.17- 2.81 (m, 4H). |
| C | 313 | 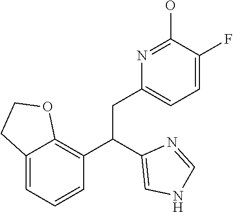 [SMILES: Oc1nc(CC(c2c[nH]cn2)c2cccc3c2OCC3)ccc1F] | 4-[1-(2,3-dihydro-1- benzofuran-7-yl)-2-(5- fluoro-6-methoxy-2- pyridyl)ethyl]imidazole | 339.37 | 340 | 99.40% | ¹H NMR (400 MHz, DMSO-d₆) δ 11.73 (s, 1H), 7.51- 7.43 (m, 1H), 7.40 (m, 1H), 7.01 (t, J = 9.1 Hz, 2H), 6.75-6.62 (m, 3H), 4.73-4.58 (m, 1H), 4.44 (s, 2H), 3.87 (s, 3H), 3.41-3.34 (m, 1H), 3.22-3.20 (m, 1H), 3.11 (t, J = 8.7 Hz, 2H). |
| C | 314 | 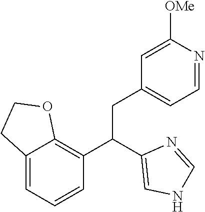 [SMILES: COc1cc(CC(c2c[nH]cn2)c2cccc3c2OCC3)ccn1] | 4-[1-(2,3-dihydro-1- benzofuran-7-yl)-2-(2- methoxy-4- pyridyl)ethyl]imidazole | 321.38 | 322.1 | 99.10% | ¹H NMR (400 MHz, DMSO-d₆) δ 11.81 (d, J = 53.8 Hz, 1H), 7.93 (d, J = 5.3 Hz, 1H), 7.52 (s, 1H), 7.20- 6.86 (m, 2H), 6.86-6.61 (m, 3H), 6.49 (s, 1H), 4.44- 4.12 (m, 3H), 3.76 (s, 3H), 3.30-3.28 (m, 1H), 3.14 (t, J = 8.7 Hz, 3H). |
| C | 315 | 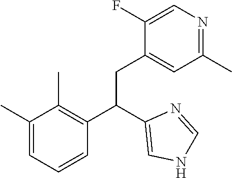 | 4-[2-(5-fluoro-2-methyl- 4-pyridyl)-1-(2,3- xylyl)ethyl]imidazole | 309.388 | 310.1 | 99.30% | ¹H NMR (400 MHz, DMSO-d₆) δ 11.76 (s, 1H), 8.22 (d, J = 1.7 Hz, 1H), 7.59-7.43 (m, 1H), 7.22 (s, 1H), 7.09-6.88 (m, 3H), 6.78 (s, 1H), 4.60 (t, J = 7.8 Hz, 1H), 3.36 (d, J = 13.7 Hz, 1H), 3.11-3.10 (m, 1H), 2.30 (s, 3H), 2.18 (d, J = 12.5 Hz, 6H). |
| C | 316 | 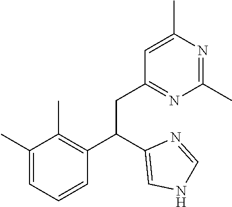 | 4-[2-(4-imidazolyl)-2- (2,3-xylyl)ethyl]-2,6- dimethylpyrimidine | 306.413 | 307.1 | 98.30% | ¹H NMR (400 MHz, DMSO-d₆) δ 7.58 (s, 1H), 7.11 (d, J = 7.5 Hz, 1H), 7.01-6.87 (m, 3H), 6.78 (s, 1H), 4.87 (t, J = 7.8 Hz, 1H), 3.35 (dd, J = 14.1, 8.0 Hz, 1H), 3.14 (dd, J = 14.1, 7.6 Hz, 1H), 2.48 (s, 3H), 2.25 (s, 3H), 2.22-2.12 (m, 6H). |
| C | 317 | 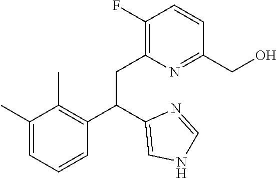 [SMILES: Cc1cccc(C(Cc2nc(CO)ccc2F)c2c[nH]cn2)c1C] | {5-fluoro-6-[2-(4- imidazolyl)-2-(2,3- xylyl)ethyl]-2- pyridyl}methanol | 325.387 | 326.1 | 99.70% | ¹H NMR (400 MHz, DMSO-d₆) δ 11.93-11.49 (m, 1H), 7.60-7.37 (m, 2H), 7.31-7.12 (m, 2H), 7.10- 6.86 (m, 2H), 6.79-6.62 (m, 1H), 5.48-5.30 (m, 1H), 5.04-4.80 (m, 1H), 4.57-4.36 (m, 2H), 3.48- 3.32 (m, 2H), 2.27-2.11 (m, 6H). |
| C | 318 | 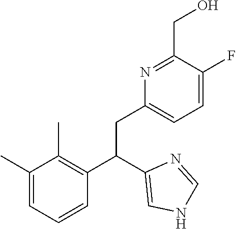 [SMILES: Cc1cccc(C(Cc2ccc(F)c(CO)n2)c2c[nH]cn2)c1C] | {3-fluoro-6-[2-(4- imidazolyl)-2-(2,3- xylyl)ethyl]-2- pyridyl}methanol | 325.387 | 326.15 | 99.60% | ¹H NMR (400 MHz, DMSO-d₆) δ 11.88-11.57 (m, 1H), 7.52-7.34 (m, 2H), 7.26-7.05 (m, 1H), 7.05- 6.85 (m, 3H), 6.82-6.67 (m, 1H), 5.29-5.13 (m, 1H), 4.95-4.68 (m, 1H), 4.62-4.47 (m, 2H), 3.55- 3.36 (m, 1H), 3.20 (dd, J = 13.8, 7.3 Hz, 1H), 2.19 (d, J = 9.0 Hz, 6H). |
| C | 319 | 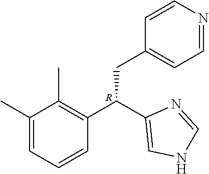 | 4-[(S)-2-(4-pyridyl)-1- (2,3- xylyl)ethyl]imidazole | 277.371 | 278.15 | 96.10% | ¹H NMR (400 MHz, DMSO-d₆) δ 11.90-11.59 (m, 1H), 8.43-8.20 (m, 2H), 7.58-7.39 (m, 1H), 7.28- 7.06 (m, 3H), 7.05-6.87 (m, 2H), 6.82-6.64 (m, 1H), 4.69-4.45 (m, 1H), 3.53-3.36 (m, 1H), 3.08 (dd, J = 13.7, 7.4 Hz, 1H), 2.25-2.07 (m, 6H). |
| C | 320 | 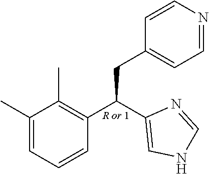 [SMILES: Cc1cccc([C@@H](Cc2ccncc2)c2c[nH]cn2)c1C] | 4-[(R)-2-(4-pyridyl)-1- (2,3- xylyl)ethyl]imidazole | 277.371 | 278.1 | 98.50% | ¹H NMR (400 MHz, DMSO-d₆) δ 11.96-11.60 (m, 1H), 8.33 (d, J = 5.0 Hz, 2H), 7.50 (s, 1H), 7.23 (s, 1H), 7.12 (d, J = 5.0 Hz, 2H), 7.05-6.88 (m, 2H), 6.75 (s, 1H), 4.75-4.39 (m, 1H), 3.50-3.35 (m, 1H), 3.09 (dd, J = 14.0, 7.3 Hz, 1H), 2.29-2.04 (m, 6H). |
| C | 321 | 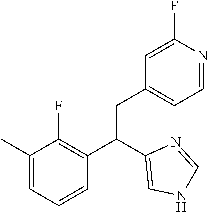 [SMILES: Cc1cccc(C(Cc2ccnc(F)c2)c2c[nH]cn2)c1F] | 4-[2-(2-fluoro-4-pyridyl)- 1-(2-fluoro-3- tolyl)ethyl]imidazole | 299.324 | 300.1 | 99.60% | ¹H NMR (400 MHz, DMSO-d₆) δ 12.13-11.63 (m, 1H), 8.02 (d, J = 5.2 Hz, 1H), 7.63-7.41 (m, 1H), 7.28 (t, J = 7.5 Hz, 1H), 7.17-7.02 (m, 2H), 7.02- 6.94 (m, 1H), 6.94-6.71 (m, 2H), 4.73-4.46 (m, 1H), 3.49 (dd, J = 13.7, 8.0 Hz, 1H), 3.21 (dd, J = 13.7, 7.9 Hz, 1H), 2.28-2.10 (m, 3H). |
| C | 322 | 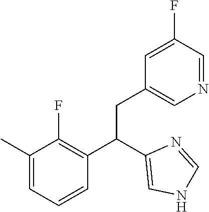 [SMILES: Cc1cccc(C(Cc2cncc(F)c2)c2c[nH]cn2)c1F] | 4-[2-(5-fluoro-3-pyridyl)- 1-(2-fluoro-3- tolyl)ethyl]imidazole | 299.324 | 300.05 | 99.60% | ¹H NMR (400 MHz, DMSO-d₆) δ 12.08-11.62 (m, 1H), 8.40-8.26 (m, 1H), 8.18-8.08 (m, 1H), 7.62- 7.37 (m, 2H), 7.35-7.22 (m, 1H), 7.20-6.94 (m, 2H), 6.92-6.73 (m, 1H), 4.68-4.42 (m, 1H), 3.46 (dd, J = 13.6, 8.0 Hz, 1H), 3.18 (dd, J = 13.6, 7.9 Hz, 1H), 2.28-2.10 (m, 3H). |
| C | 323 | 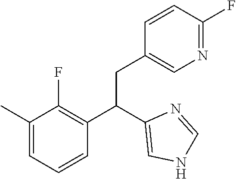 [SMILES: Cc1cccc(C(Cc2ccc(F)nc2)c2c[nH]cn2)c1F] | 4-[2-(6-fluoro-3-pyridyl)- 1-(2-fluoro-3- tolyl)ethyl]imidazole | 299.324 | 300.05 | 99.70% | ¹H NMR (400 MHz, DMSO-d₆) δ 12.04-11.60 (m, 1H), 7.93-7.83 (m, 1H), 7.75-7.62 (m, 1H), 7.59- 7.48 (m, 1H), 7.28 (dt, J = 7.5, 4.1 Hz, 1H), 7.10- 7.03 (m, 1H), 7.03-6.95 (m, 2H), 6.91-6.75 (m, 1H), 4.59-4.39 (m, 1H), 3.48-3.37 (m, 1H), 3.22- 3.06 (m, 1H), 2.21-2.10 (m, 3H). |
| C | 324 | 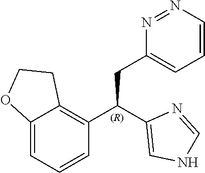 | 3-[(R)-2-(2,3-dihydro-1- benzofuran-4-yl)-2-(4- imidazolyl)ethyl]pyridazine | 292.3 | 293 | 99.3 | 1H NMR (400 MHz, Methanol-d4) δ 8.96 (dd, J = 4.9, 1.7 Hz, 1H), 7.58 (d, J = 1.2 Hz, 1H), 7.52-7.44 (m, 1H), 7.33 (dd, J = 8.5, 1.6 Hz, 1H), 7.01 (t, J = 7.9 Hz, 1H), 6.91 (t, J = 1.0 Hz, 1H), 6.81-6.75 (m, 1H), 6.53 (dd, J = 7.9, 0.9 Hz, 1H), 4.61-4.52 (m, 1H), 4.49-4.29 (m, 2H), 3.83-3.74 (m, 1H), 3.59- 3.49 (m, 1H), 3.21-3.09 (m, 1H), 2.83 -2.70 (m, |

[symbol]

indicates data missing or illegible when filed

### Procedure D

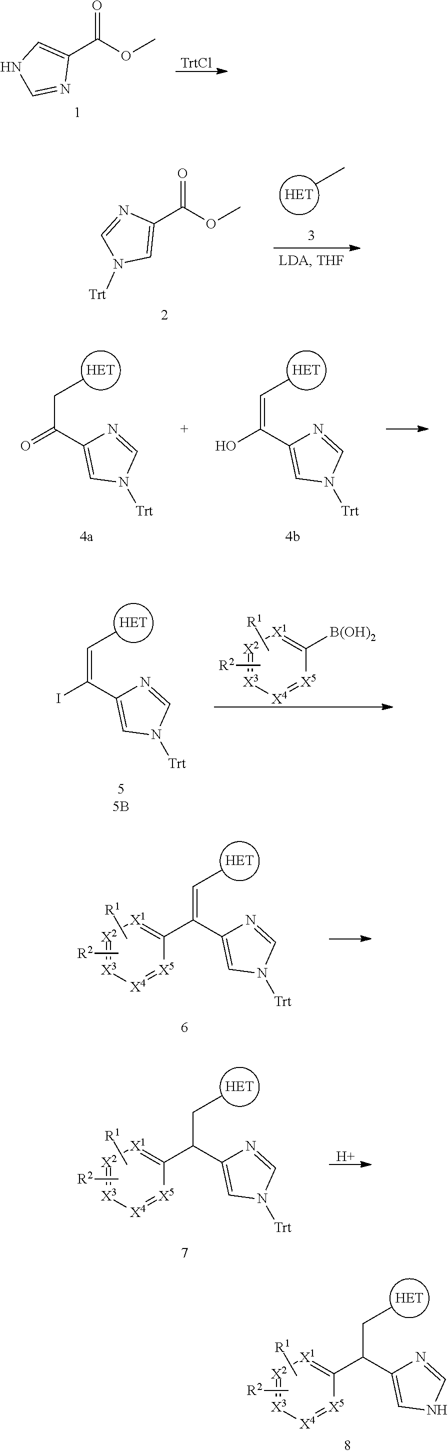

### Example of Procedure D: Synthesis of Compounds 362 & 363

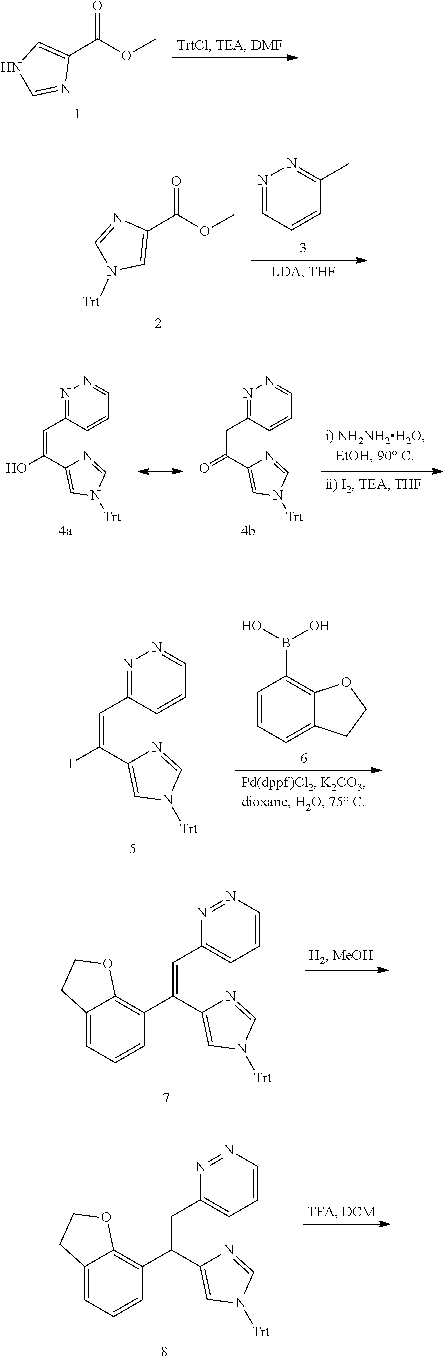

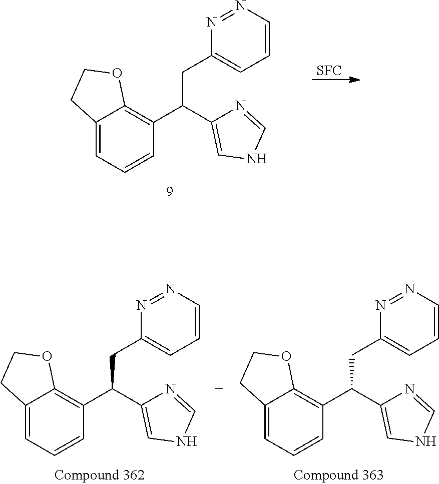

### Experimental Details

### 1. Synthesis of 2

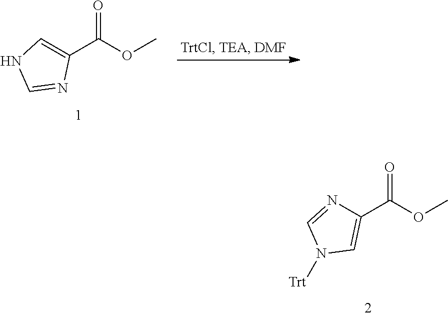

### methyl 1-tri-1H-imidazole-4-carboxylate

To a stirred solution of methyl 1H-imidazole-4-carboxylate (200 g, 1.59 mol, 1.0 equiv) and TEA (482.0 g, 4.77 mol, 3.0 equiv) in DMF (3.0 L) was added TrtCl (530.0 g, 1.90 mol, 1.2 equiv). The mixture was stirred at room temperature overnight. The reaction was quenched by the addition of 3 L water. The aqueous solution was extracted with EtOAc (1.5 L×3). The combined organic solution was dried over Na₂SO₄ and concentrated to give the residue which was triturated with EtOAc to afford methyl 1-trityl-1H-imidazole-4-carboxylate (450 g, 77% yield) as white solid. LCMS (ESI) [M+H]⁺: 369. ¹H NMR (400 MHz, Chloroform-d) δ 7.60 (d, J=1.4 Hz, 1H), 7.48 (d, J=1.5 Hz, 1H), 7.37-7.32 (m, 9H), 7.14-7.11 (m, 6H), 3.87 (s, 3H).

### 2. Synthesis of 4a,4b

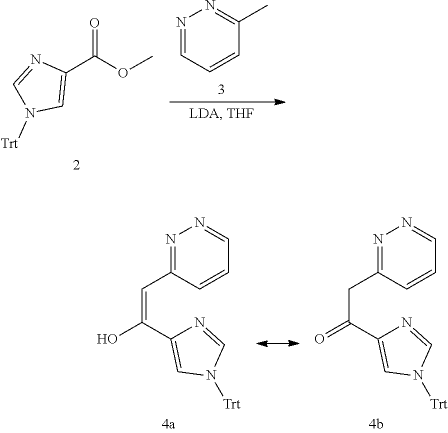

### (2,3-dihydrobenzofuran-7-yl)(1-trityl-1H-imidazol-4-yl)methanone & (E)-2-(pyridazin-3-yl)-1-(1-trityl-1H-imidazol-4-yl)ethen-1-ol

To a stirred solution of 3-methylpyridazine (42.9 g, 456.34 mmol, 1.2 equiv) in THF (1.5 L) was added LDA (380.3 ml, 760.56 mmol, 2.0 equiv) dropwise at −78° C. The solution was stirred at this temperature for 30 min. A solution of methyl 1-trityl-1H-imidazole-4-carboxylate (140 g, 380.28 mmol, 1.0 equiv) in THF (200 ml) was then added dropwise. The mixture was stirred at −78° C.—room temperature for 3 h. The reaction was quenched by the addition of 2 L water. The aqueous solution was extracted with EtOAc (1 L×3). The combined organic solution was dried over Na₂SO₄ and concentrated to give the residue which was purified by flash column with 0-80% EtOAc in petroleum ether to afford a mixture of (2,3-dihydrobenzofuran-7-yl)(1-trityl-1H-imidazol-4-yl)methanone and (E)-2-(pyridazin-3-yl)-1-(1-trityl-1H-imidazol-4-yl)ethen-1-ol (50 g, 31% yield) as yellow solid. LCMS (ESI) [M+H]⁺: 431. ¹H NMR (400 MHz, Chloroform-d) δ 9.10 (dd, J=4.9, 1.7 Hz, 0.7H), 8.13 (dd, J=4.2, 1.7 Hz, 0.31H), 7.67 (d, J=1.3 Hz, 0.7H), 7.59-7.53 (dd, J=8.5, 1.7 Hz, 0.7H), 7.49 (d, J=1.4 Hz, 1H), 7.46-7.41 (m, 1H), 7.40-7.29 (m, 9.3H), 7.17-7.04 (m, 6.3H), 6.19 (s, 0.3H), 4.74 (s, 1.4H).

### 3. Synthesis of 5

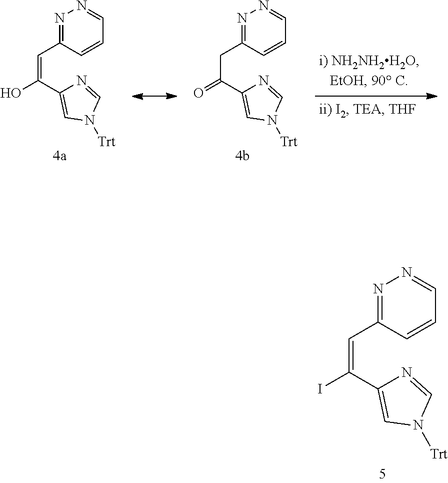

### (E)-3-(2-iodo-2-(1-trityl-1H-imidazol-4-yl)vinyl)pyridazine

To a solution of a mixture of (2,3-dihydrobenzofuran-7-yl)(1-trityl-1H-imidazol-4-yl)methanone and (E)-2-(pyridazin-3-yl)-1-(1-trityl-1H-imidazol-4-yl)ethen-1-ol (15 g, 34.87 mmol, 1.0 equiv) in EtOH (150 ml) was added hydrazine hydrate (11.2 g, 278.95 mmol, 8.0 equiv). The solution was stirred at 90° C. for 1 h. The mixture was concentrated, quenched by the addition of 150 mL water. The aqueous solution was extracted with DCM (150 mL×3). The combined organic solution was dried over Na₂SO₄ and concentrated to give the residue which was directly used. To the above residue in TEA (75 ml) was added a solution of I₂ (17.7 g, 69.74 mmol, 2.0 equiv) in THF (75 ml). The mixture was stirred at room temperature for 1 h. The reaction was quenched by the addition of saturated Na2S2O3 aqueous (100 ml). The aqueous solution was extracted with EtOAc (100 mL×3). The combined organic solution was dried over Na2SO4 and concentrated to give the residue which was purified by flash column with 0-100% EtOAc in petroleum ether to afford (E)-3-(2-iodo-2-(1-trityl-1H-imidazol-4-yl)vinyl)pyridazine (4.8 g, 26% yield) as red solid. LCMS (ESI) [M+H]⁺: 541. ¹H NMR (400 MHz, Chloroform-d) δ 9.08 (dd, J=5.0, 1.7 Hz, 1H), 8.10 (s, 1H), 7.99 (dd, J=8.6, 1.7 Hz, 1H), 7.57 (d, J=1.5 Hz, 1H), 7.50-7.42 (m, 1H), 7.41-7.30 (m, 12H), 7.25 (d, J=1.4 Hz, 1H), 7.22-7.12 (m, 7H), 7.10-7.04 (m, 1H).

### 4. Synthesis of 7

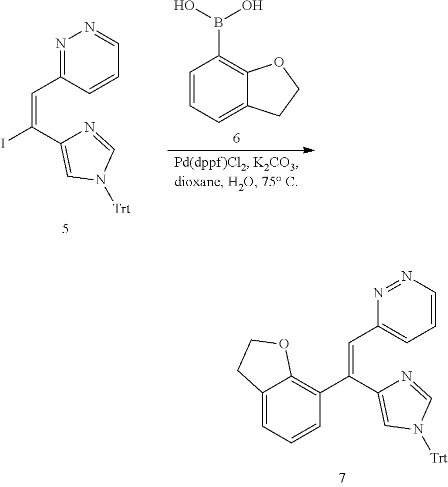

### (Z)-3-(2-(2,3-dihydrobenzofuran-7-yl)-2-(1-trityl-1H-imidazol-4-yl)vinyl)pyridazine

To a mixture of (E)-3-(2-iodo-2-(1-trityl-1H-imidazol-4-yl)vinyl)pyridazine (600 mg, 1.11 mmol, 1.0 equiv), (2,3-dihydrobenzofuran-7-yl)boronic acid (219 mg, 1.33 mmol, 1.2 equiv) and K₂C₃ (460 mg, 3.33 mmol, 3.0 equiv) in dioxane (10 ml) and H₂O (2 ml) was added Pd(dppf)Cl₂ (80 gm, 0.11 mmol, 0.1 equiv). The mixture was stirred at 75° C. for 1 h. The mixture was filtered through Celite pad. The filtrate was concentrated to give the residue which was purified by reverse phase flash column with 5-80% acetonitrile in water to afford (Z)-3-(2-(2,3-dihydrobenzofuran-7-yl)-2-(1-trityl-1H-imidazol-4-yl)vinyl)pyridazine (282 mg, 48% yield) as yellow solid. LCMS (ESI) [M+H]⁺: 533.

### 5. Synthesis of 8

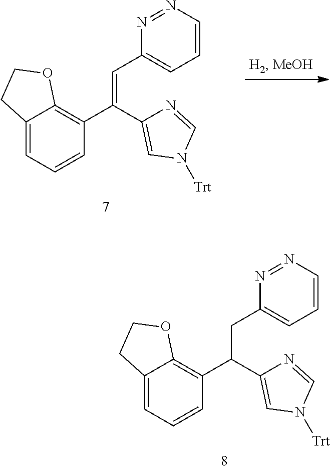

### 3-(2-(2,3-dihydrobenzofuran-7-yl)-2-(1-trityl-1H-imidazol-4-yl)ethyl)pyridazine

To a solution of (Z)-3-(2-(2,3-dihydrobenzofuran-7-yl)-2-(1-trityl-1H-imidazol-4-yl)vinyl)pyridazine (282 mg, 0.53 mmol, 1.0 equiv) in MeOH (3 ml) was added Pd/C (40 mg). The mixture was stirred at room temperature for 4 h under H₂ atmosphere. The mixture was filtered through Celite pad. The filtrate was concentrated to give the residue which was purified by reverse phase flash column with 5-80% acetonitrile in water to afford 3-(2-(2,3-dihydrobenzofuran-7-yl)-2-(1-trityl-1H-imidazol-4-yl)ethyl)pyridazine (92 mg, 32% yield) as yellow solid. LCMS (ESI) [M+H]⁺: 535.

### 5. Synthesis of 9

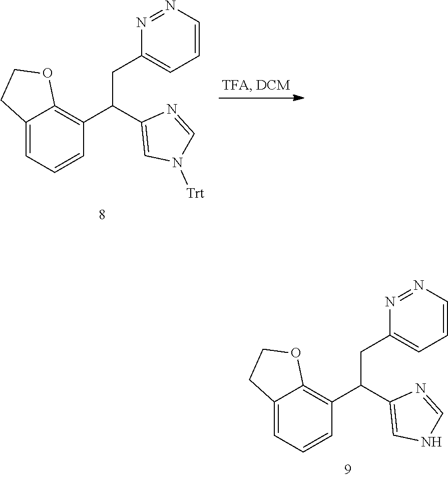

### 3-(2-(2,3-dihydrobenzofuran-7-yl)-2-(1H-imidazol-4-yl)ethyl)pyridazine

To a solution of 3-(2-(2,3-dihydrobenzofuran-7-yl)-2-(1-trityl-1H-imidazol-4-yl)ethyl)pyridazine (92 mg, 0.17 mmol, 1.0 equiv) in DCM (1 ml) was added TFA (0.5 ml). The solution was stirred at room temperature for 1 h. The solution was concentrated to give the residue which was adjusted to pH10 with saturated K2CO3 aqueous. The mixture was purified by prep-HPLC (Column: YMC-Actus Triart C18 ExRS 30*150 mm, 5m; Mobile Phase A: Water (10 mmol/L NH₄HCO₃), Mobile Phase B: ACN; Flow rate: 60 ml/min ml/min; Gradient: 10% B to 32% B in 7 min; Wave Length: 254 nm/220 nm; RT1 (min): 6.62) to afford 3-(2-(2,3-dihydrobenzofuran-7-yl)-2-(1H-imidazol-4-yl)ethyl)pyridazine (35 mg, 70% yield) as white solid. LCMS (ESI) [M+H]⁺: 293. ¹H NMR (400 MHz, DMSO-d6) δ 12.03-11.62 (m, 1H), 9.17-8.78 (m, 1H), 7.56-7.34 (m, 3H), 7.33-7.23 (m, 1H), 7.14-6.89 (m, 2H), 6.83-6.60 (m, 3H), 4.86-4.57 (m, 1H), 4.55-4.30 (m, 1H), 3.71-3.57 (m, 1H), 3.54-3.41 (m, 1H), 3.20-3.04 (m, 3H).

### 6. Isolation of Compounds 362 and 363

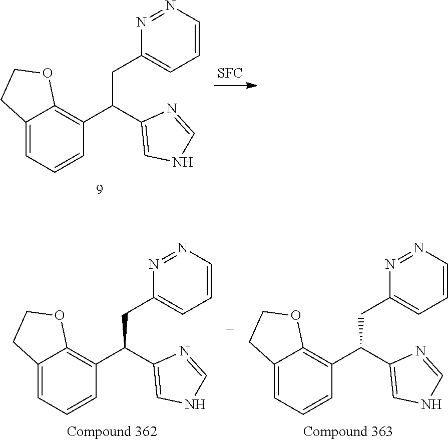

### (R)-3-(2-(2,3-dihydrobenzofuran-7-yl)-2-(1H-imidazol-4-yl)ethyl)pyridazine & (S)-3-(2-(2,3-dihydrobenzofuran-7-yl)-2-(1H-imidazol-4-yl)ethyl)pyridazine

3-(2-(2,3-dihydrobenzofuran-7-yl)-2-(1H-imidazol-4-yl)ethyl)pyridazine (100 mg) was separated by prep-Chiral-HPLC (Column: CHIRALPAK IG, 3*25 cm, 5 μm; Mobile Phase A: Hex (10 mM NH3-MeOH), Mobile Phase B: EtOH; Flow rate: 40 ml/min; Gradient: isocratic 50; Wave Length: 201/283 nm; RT1 (min): 5.93; RT2 (min): 10.49; Sample Solvent: ETOH:DCM=1: 1; Sample concentration: mg/ml; Injection Volume: 1 ml; Number Of Runs: 3) to afford (R)-3-(2-(2,3-dihydrobenzofuran-7-yl)-2-(1H-imidazol-4-yl)ethyl)pyridazine (31.5 mg, 100% e.e.). LCMS (ESI) [M+H]⁺: 293. ¹H NMR (300 MHz, DMSO-d6) δ 11.78 (s, 1H), 8.97 (dd, J=4.9, 1.7 Hz, 1H), 7.56-7.41 (m, 2H), 7.37-7.27 (m, 1H), 7.03 (t, J=7.3 Hz, 2H), 6.84-6.65 (m, 2H), 4.65 (t, J=8.0 Hz, 1H), 4.55-4.30 (m, 2H), 3.70-3.55 (m, 1H), 3.52-3.40 (m, 1H), 3.12 (t, J=8.7 Hz, 2H).

(S)-3-(2-(2,3-dihydrobenzofuran-7-yl)-2-(1H-imidazol-4-yl)ethyl)pyridazine (32.3 mg, 100% e.e.). LCMS (ESI) [M+H]⁺: 293. ¹H NMR (300 MHz, DMSO-d6) δ 11.79 (s, 1H), 8.97 (dd, J=4.9, 1.7 Hz, 1H), 7.56-7.43 (m, 2H), 7.31 (dd, J=8.4, 1.7 Hz, 1H), 7.03 (t, J=7.2 Hz, 2H), 6.83-6.65 (m, 2H), 4.65 (t, J=8.0 Hz, 1H), 4.56-4.34 (m, 2H), 3.68-3.56 (m, 1H), 3.51-3.40 (m, 1H), 3.12 (t, J=8.7 Hz, 2H).

TABLE D

| | | | | Molecular | | | |
|---|---|---|---|---|---|---|---|
| | | | | weight | LCMS | | |
| Procedure | Name | Structure | IUPAC | (g/mol) | [M + H]+ | Purity (%) | 1H NMR |
| D | 337 | 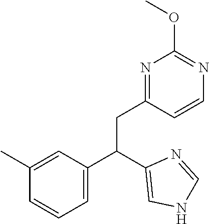 [SMILES: COc1nccc(CC(c2cccc(C)c2)c2c[nH]cn2)n1] | 4-[2-(4-imidazolyl)-2-(m- tolyl)ethyl]-2- methoxypyrimidine | 294.358 | 295.05 | 99.5 | ¹H NMR (400 MHz, DMSO-d₆) δ 11.98-11.63 (m, 1H), 8.44-8.23 (m, 1H), 7.56-7.37 (m, 1H), 7.19-7.01 (m, 3H), 7.01-6.69 (m, 3H), 4.64- 4.37 (m, 1H), 3.85 (s, 3H), 3.50-3.38 (m, 1H), 3.30-3.15 (m, 1H), 2.23 (s, 3H). |
| D | 338 | 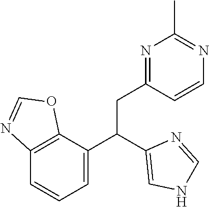 [SMILES: Cc1nccc(CC(c2c[nH]cn2)c2cccc3ncoc23)n1] | 7-[1-(4-imidazolyl)-2-(2- methyl-4-pyrimidinyl)ethyl]- 1,3-benzoxazole | 305.341 | 306.15 | 99.5 | ¹H NMR (400 MHz, DMSO-d₆) δ 12.05-11.76 (m, 1H), 8.76-8.61 (m, 1H), 8.45-8.34 (m, 1H), 7.68-7.55 (m, 1H), 7.55-7.44 (m, 1H), 7.38 (d, J = 7.8 Hz, 1H), 7.34-7.23 (m, 1H), 7.10-6.96 (m, 1H), 6.91-6.76 (m, 1H), 5.14-4.91 (m, 1H), 3.60 (dd, J = 13.9, 7.7 Hz, 1H), 3.40 (dd, J = 13.9, 8.2 Hz, 1H), 2.49 (s, 3H). |
| D | 339 | 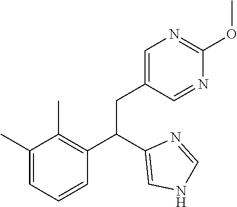 | 5-[2-(4-imidazolyl)-2-(2,3- xylyl)ethyl]-2- methoxypyrimidine | 308.385 | 309.15 | 98.0 | ¹H NMR (400 MHz, DMSO-d₆) δ 11.93-11.59 (m, 1H), 8.25 (s, 2H), 7.52 (s, 1H), 7.34-7.07 (m, 1H), 7.09-6.84 (m, 2H), 6.78 (s, 1H), 4.60- 4.29 (m, 1H), 3.82 (s, 3H), 3.31-3.24 (m, 1H), 3.01 (dd, J = 13.9, 7.2 Hz, 1H), 2.19 (s, 3H), 2.14 (s, 3H). |
| D | 340 | 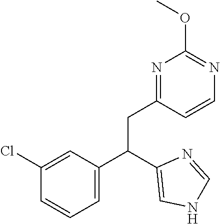 [SMILES: COc1nccc(CC(c2cccc(Cl)c2)c2c[nH]cn2)n1] | 4-[2-(m-chlorophenyl)-2-(4- imidazolyl)ethyl]-2- methoxypyrimidine | 314.776 | 315 | 95.7 | ¹H NMR (400 MHz, DMSO-d₆) δ 12.01-11.70 (m, 1H), 8.45-8.26 (m, 1H), 7.58-7.46 (m, 1H), 7.40-7.13 (m, 4H), 7.01-6.74 (m, 2H), 4.72- 4.41 (m, 1H), 3.85 (s, 3H), 3.45 (dd, J = 14.1, 7.7 Hz, 1H), 3.25 (dd, J = 14.2, 8.3 Hz, 1H). |
| D | 341 | 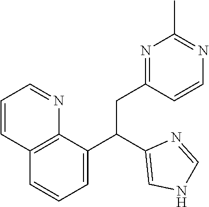 [SMILES: Cc1nccc(CC(c2c[nH]cn2)c2cccc3cccnc23)n1] | 4-[2-(4-imidazolyl)-2-(8- quinolyl)ethyl]-2- methylpyrimidine | 315.38 | 316.15 | 96.5 | ¹H NMR (400 MHz, Chloroform-d) δ 9.00-8.95 (m, 1H), 8.28 (d, J = 5.2 Hz, 1H), 8.19-8.13 (m, 1H), 7.71-7.61 (m, 2H), 7.53-7.41 (m, 3H), 7.27 (s, 1H), 6.85 (s, 1H), 6.76 (d, J = 5.2 Hz, 1H), 5.84-5.72 (m, 1H), 3.84-3.73 (m, 1H), 3.66-3.57 (m, 1H), 2.66 (s, 3H). |
| D | 342 | 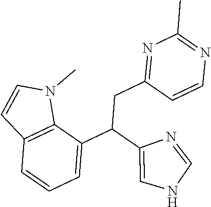 [SMILES: Cc1nccc(CC(c2c[nH]cn2)c2cccc3ccn(C)c23)n1] | 4-[2-(4-imidazolyl)-2-(1- methyl-7-indolyl)ethyl]-2- methylpyrimidine | 317.396 | 318.2 | 99.7 | ¹H NMR (400 MHz, DMSO-d₆) δ 8.40 (d, J = 5.1 Hz, 1H), 8.20 (s, 1H), 7.54 (s, 1H), 7.31 (dd, J = 7.8, 1.1 Hz, 1H), 7.19 (d, J = 3.1 Hz, 1H), 7.16-7.11 (m, 1H), 7.08 (d, J = 5.1 Hz, 1H), 6.91 (t, J = 7.6 Hz, 1H), 6.79 (s, 1H), 6.33 (d, J = 3.1 Hz, 1H), 5.63 (t, J =7.7 Hz, 1H), 4.21 (s, 3H), 3.54 (dd, J = 14.3, 7.5 Hz, 1H), 3.44 (dd, J = 14.3, 7.9 Hz, 1H), 2.53 (s, 3H). |
| D | 343 | 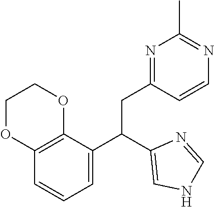 [SMILES: Cc1nccc(CC(c2c[nH]cn2)c2cccc3c2OCCO3)n1] | 4-[2-(2,3-dihydro-1,4- benzodioxin-5-yl)-2-(4- imidazolyl)ethyl]-2- methylpyrimidine | 322.368 | 323.15 | 99.5 | ¹H NMR (400 MHz, DMSO-d₆) δ 8.42 (d, J = 5.1 Hz, 1H), 8.15 (s, 1H), 7.55 (s, 1H), 6.95 (d, J = 5.1 Hz, 1H), 6.84-6.77 (m, 2H), 6.71 (t, J = 7.8 Hz, 1H), 6.67-6.62 (m, 1H), 4.86 (t, J = 7.9 Hz, 1H), 4.47-3.87 (m, 4H), 3.47-3.27 (m, 1H), 3.27-3.10 (m, 1H), 2.54 (s, 3H). |
| D | 344 | 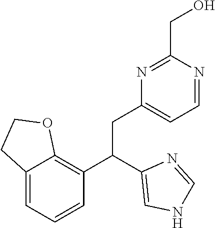 [SMILES: OCc1nccc(CC(c2c[nH]cn2)c2cccc3c2OCC3)n1] | {4-[2-(2,3-dihydro-1- benzofuran-7-yl)-2-(4- imidazolyl)ethyl]-2- pyrimidinyl}methanol | 322.368 | 323.1 | 99.5 | ¹H NMR (400 MHz, DMSO-d₆) δ 8.51 (d, J = 5.8 Hz, 1H), 8.17 (s, 1H), 7.51 (s, 1H), 7.10-6.96 (m, 3H), 6.72 (dd, J = 13.3, 5.8 Hz, 2H), 4.69 (t, J = 7.9 Hz, 1H), 4.53 (s, 2H), 2.51-2.40 (m, 2H), 3.46 (dd, J 13.9, 8.4 Hz, 1H), 3.27 (dd, J = 13.9, 8.4 Hz, 1H), 3.18-3.08 (m, 2H). |
| D | 345 | 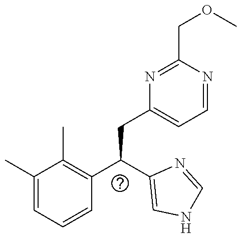 | 4-[(R)-2-(4-imidazolyl)-2- (2,3-xylyl)ethyl]-2- (methoxymethyl)pyrimidine | 322.412 | 323.15 | 99.6 | ¹H NMR (400 MHz, DMSO-d₆) δ 11.77 (s, 1H), 8.52 (d, J =5.1 Hz, 1H), 7.49 (s, 1H), 7.26-7.11 (m, 2H), 7.00-6.87 (m, 2H), 6.74 (s, 1H), 4.87 (t, J = 7.7 Hz, 1H), 4.49 (s, 2H), 3.59-3.41 (m, 1H), 3.32 (s, 3H), 3.26- 3.14 (m, 1H), 2.20 (d, J = 10.0 Hz, 6H). |
| D | 346 | 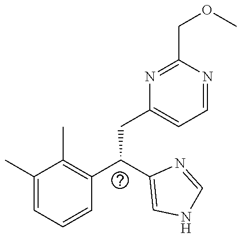 | 4-[(S)-2-(4-imidazolyl)-2- (2,3-xylyl)ethyl]-2- (methoxymethyl)pyrimidine | 322.412 | 323.15 | 99.7 | ¹H NMR (400 MHz, DMSO-d₆) δ 11.77 (s, 1H), 8.52 (d, J = 5.1 Hz, 1H), 7.49 (s, 1H), 7.26-7.11 (m, 2H), 7.00-6.87 (m, 2H), 6.74 (s, 1H), 4.87 (t, J = 7.7 Hz, 1H), 4.49 (s, 2H), 3.59-3.41 (m, 1H), 3.32 (s, 3H), 3.26- 3.14 (m, 1H), 2.20 (d, J = 10.0 Hz, 6H). |
| D | 347 | 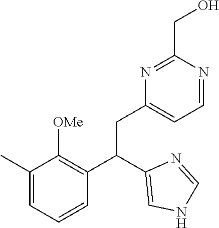 [SMILES: COc1c(C)cccc1C(Cc1ccnc(CO)n1)c1c[nH]cn1] | {4-[2-(4-imidazolyl)-2-(2- methoxy-3-tolyl)ethyl]-2- pyrimidinyl}methanol | 324.384 | 325.1 | 97.5 | ¹H NMR (400 MHz, DMSO-d₆) δ 12.18-11.61 (m, 1H), 8.52 (d, J = 5.1 Hz, 1H), 7.53 (s, 1H), 7.29 (dd, J = 7.5, 2.0 Hz, 1H), 7.07 (d, J = 5.1 Hz, 1H), 7.03-6.91 (m, 2H), 6.74 (s, 1H), 5.15 (s, 1H), 4.92 (t, J = 7.9 Hz, 1H), 4.53 (s, 2H), 3.63 (s, 3H), 3.48 (dd, J = 14.0, 7.9 Hz, 1H), 3.26 (dd, J = 14.0, 8.0 Hz, 1H), 2.19 (s, 3H). |
| D | 348 | 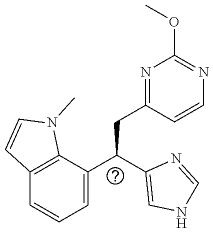 [SMILES: COc1nccc(C[C@@H](c2c[nH]cn2)c2cccc3ccn(C)c23)n1] | 4-[(R)-2-(4-imidazolyl)-2-(1- methyl-7-indolyl)ethyl]-2- methoxypyrimidine | 333.395 | 334.15 | 99.4 | 1H NMR (400 MHz, DMSO-d6) δ 11.79 (s, 1H), 8.34 (d, J = 5.0 Hz, 1H), 7.51 (s, 1H), 7.31 (d, J = 7.8 Hz, 1H), 7.20 (d, J = 3.1 Hz, 1H), 7.11 (s, 1H), 6.98 (d, J = 5.0 Hz, 1H), 6.89 (t, J = 7.5 Hz, 1H), 6.77 (s, 1H), 6.34 (d, J = 3.1 Hz, 1H), 5.62 (s, 1H), 4.21 (s, 3H), 3.83 (s, 3H), 3.53 (s, 1H), 3.45-3.37 (m, 1H) |
| D | 349 | 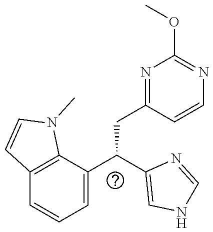 [SMILES: COc1nccc(C[C@H](c2c[nH]cn2)c2cccc3ccn(C)c23)n1] | 4-[(S)-2-(4-imidazolyl)-2-(1- methyl-7-indolyl)ethyl]-2- methoxypyrimidine | 333.395 | 334.15 | 99.8 | 1H NMR (400 MHz, DMSO-d6) δ 11.79 (s, 1H), 8.34 (d, J = 5.0 Hz, 1H), 7.51 (s, 1H), 7.31 (d, J = 7.8 Hz, 1H), 7.20 (d, J = 3.1 Hz, 1H), 7.11 (s, 1H), 6.98 (d, J = 5.0 Hz, 1H), 6.89 (t, J = 7.5 Hz, 1H), 6.77 (s, 1H), 6.34 (d, J = 3.1 Hz, 1H), 5.62 (s, 1H), 4.21 (s, 3H), 3.83 (s, 3H), 3.53 (s, 1H), 3.45-3.37 (m, 1H) |
| D | 350 | 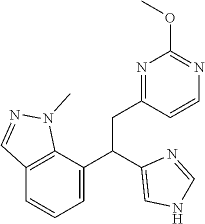 [SMILES: COc1nccc(CC(c2c[nH]cn2)c2cccc3cnn(C)c23)n1] | 7-[1-(4-imidazolyl)-2-(2- methoxy-4- pyrimidinyl)ethyl]-1-methyl- 1H-indazole | 334.383 | 335.1 | 97.7 | 1H NMR (400 MHz, DMSO-d6) δ 11.87 (d, J =50.7 Hz, 1H), 8.34 (d, J = 4.9 Hz, 1H), 7.96 (s, 1H), 7.53 (d, J = 7.3 Hz, 2H), 7.36 (d, J = 7.2 Hz, 1H), 7.15-6.96 (m, 2H), 6.83 (s, 1H), 5.60 (d, J = 47.7 Hz, 1H), 4.44 (s, 3H), 3.81 (s, 3H), 3.58 (t, J = 11.3 Hz, 1H), 3.46 (dd, J = 14.5, 7.7 Hz, 1H). |
| D | 351 | 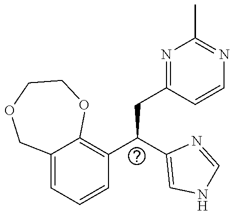 [SMILES: Cc1nccc(C[C@@H](c2c[nH]cn2)c2cccc3c2OCCOC3)n1] | 4-[(R)-2-(3,4-dihydro-2H-1,5- benzodioxepin-6-yl)-2-(4- imidazolyl)ethyl]-2- methylpyrimidine | 336.395 | 337.1 | 99.5 | ¹H NMR (400 MHz, DMSO-d₆) δ 11.71 (s, 1H), 8.42 (d, J = 5.1 Hz, 1H), 7.50 (s, 1H), 7.04-6.92 (m, 2H), 6.89-6.81 (m, 1H), 6.80-6.71 (m, 2H), 5.03-4.85 (m, 1H), 4.11-3.90 (m, 3H), 3.81-3.70 (m, 1H), 3.44 (s, 1H), 3.26-3.16 (m, 1H), 2.54-2.51 (m, 3H), 2.12-1.96 (m, 2H). |
| D | 352 | 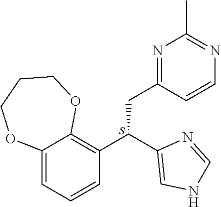 | 4-[(S)-2-(3,4-dihydro-2H-1,5- benzodioxepin-6-yl)-2-(4- imidazolyl)ethyl]-2- methylpyrimidine | 336.395 | 337.1 | 99.6 | ¹H NMR (400 MHz, DMSO-d₆) δ 11.71 (s, 1H), 8.42 (d, J = 5.1 Hz, 1H), 7.50 (s, 1H), 7.04-6.92 (m, 2H), 6.89-6.81 (m, 1H), 6.80-6.71 (m, 2H), 5.03-4.85 (m, 1H), 4.11-3.90 (m, 3H), 3.81-3.70 (m, 1H), 3.44 (s, 1H), 3.26-3.16 (m, 1H), 2.55-2.50 (m, 3H), 2.12-1.96 (m, 2H). |
| D | 353 | 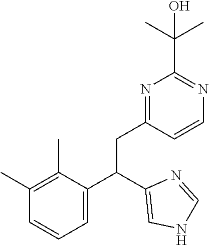 [SMILES: Cc1cccc(C(Cc2ccnc(C(C)(C)O)n2)c2c[nH]cn2)c1C] | 2-{4-[2-(4-imidazolyl)-2-(2,3- xylyl)ethyl]-2-pyrimidinyl}-2 propanol | 336.439 | 337.15 | 99.2 | ¹H NMR (400 MHz, DMSO-d₆) δ 11.79 (d, J = 47.1 Hz, 1H), 8.55 (dd, J = 21.6, 5.0 Hz, 1H), 7.47 (d, J = 19.5 Hz, 1H), 7.23-7.13 (m, 1H), 7.11- 6.87 (m, 3H), 6.74 (d, J = 16.9 Hz, 1H), 5.01-4.80 (m, 2H), 3.60-3.40 (m, 1H), 3.27 (dd, J = 14.0, 8.0 Hz, 1H), 2.19 (s, 6H), 1.41 (s, 6H). |
| D | 354 | 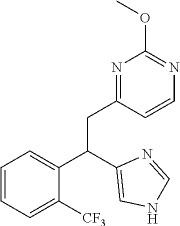 [SMILES: COc1nccc(CC(c2c[nH]cn2)c2ccccc2C(F)(F)F)n1] | 4-{2-(4-imidazolyl)-2-[o- (trifluoromethyl)phenyl]eth- yl}-2-methoxypyrimidine | 348.328 | 349.1 | 99.7 | ¹H NMR (400 MHz, DMSO-d₆) δ 11.78 (s, 1H), 8.33 (d, J = 5.0 Hz, 1H), 7.89 (d, J = 7.9 Hz, 1H), 7.60 (d, J = 7.7 Hz, 2H), 7.52 (s, 1H), 7.37 (t, J = 7.6 Hz, 1H), 6.84 - 6.73 (m, 2H), 4.86 (d, J = 6.0 Hz, 1H), 3.81 (s, 3H), 3.54 (dd, J = 13.7, 8.5 Hz, 1H), 3.20 (dd, J = 13.9, 6.8 Hz, 1H). |
| D | 355 | 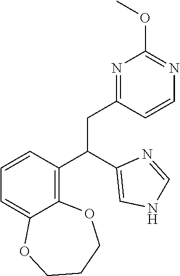 [SMILES: COc1nccc(CC(c2c[nH]cn2)c2cccc3c2OCCCO3)n1] | 4-[2-(3,4-dihydro-2H-1,5- benzodioxepin-6-yl)-2-(4- imidazolyl)ethyl]-2- methoxypyrimidine | 352.394 | 353.15 | 98.2 | ¹H NMR (400 MHz, DMSO-d₆) δ 11.87-11.64 (m, 1H), 8.42-8.25 (m, 1H), 7.53-7.39 (m, 1H), 7.04-6.62 (m, 5H), 5.05-4.85 (m, 1H), 4.09- 3.92 (m, 3H), 3.93-3.70 (m, 4H), 3.49-3.34 (m, 1H), 3.26-3.11 (m, 1H), 2.16-1.91 (m, 2H). |
| D | 356 | 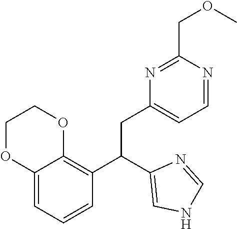 [SMILES: COCc1nccc(CC(c2c[nH]cn2)c2cccc3c2OCCO3)n1] | 4-[2-(2,3-dihydro-1,4- benzodioxin-5-yl)-2-(4- imidazolyl)ethyl]-2- (methoxymethyl)pyrimidine | 352.394 | 353.05 | 99.1 | ¹H NMR (400 MHz, DMSO-d₆) δ 11.72 (s, 1H), 8.70-8.40 (m, 1H), 7.68- 7.41 (m, 1H), 7.23-6.96 (m, 1H), 6.90-6.47 (m, 4H), 5.17-4.66 (m, 1H), 4.49 (d, J = 2.4 Hz, 2H), 4.37-3.80 (m, 4H), 3.73-3.37 (m, 1H), 3.32 (s, 3H), 3.24-3.13 (m, 1H). |
| D | 357 | 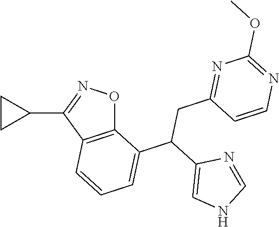 [SMILES: COc1nccc(CC(c2c[nH]cn2)c2cccc3c(C4CC4)noc23)n1] | 3-cyclopropyl-7-[1-(4- imidazolyl)-2-(2-methoxy-4- pyrimidinyl)ethyl]-1,2- benzisoxazole | 361.405 | 362.15 | 99.5 | 1H NMR (400 MHz, DMSO-d6) δ 11.89 (d, J = 85.4 Hz, 1H), 8.34 (dd, J = 20.0, 5.0 Hz, 1H), 7.67 (dd, J = 23.4, 7.8 Hz, 1H), 7.59-7.43 (m, 2H), 7.27 (dt, J = 15.2, 7.6 Hz, 1H), 6.99-6.72 (m, 2H), 5.05 (dt, J =50.2, 7.9 Hz, 1H), 3.79 (s, 3H), 3.58 (dd, J = 14.2, 7.3 Hz, 1H), 3.46 (td, J = 13.8, 8.2 Hz, 1H), 2.32 (ddd, J = 13.5, 8.2, 5.1 Hz, 1H), 1.18-1.02 (m, 4H). |
| D | 358 | 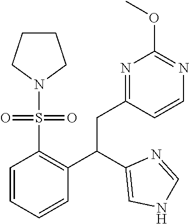 [SMILES: COc1nccc(CC(c2c[nH]cn2)c2ccccc2S(=O)(=O)N2CCCC2)n1] | 4-{2-(4-imidazolyl)-2-[o-(1- pyrrolidinylsulfonyl)phenyl] ethyl}-2-methoxypyrimidine | 413.503 | 414.2 | 97.0 | ¹H NMR (400 MHz, DMSO-d₆) δ 11.89-11.65 (m, 1H), 8.44-8.29 (m, 1H), 7.89-7.71 (m, 2H), 7.70-7.57 (m, 1H), 7.50 (s, 1H), 7.49-7.35 (m, 1H), 6.99-6.84 (m, 1H), 6.72 (s, 1H), 5.70-5.44 (m, 1H), 3.83 (s, 3H), 3.50 (dd, J = 14.0, 9.7 Hz, 1H), 3.29-3.09 (m, 5H), 1.82-1.67 (m, 4H). |
| D | 359 | 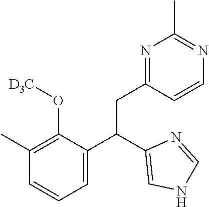 [SMILES: [2H]C([2H])([2H])Oc1c(C)cccc1C(Cc1ccnc(C)n1)c1c[nH]cn1] | 4-{2-(4-imidazolyl)-2-[2- (²H₃)methoxy-3-tolyl]ethyl}- 2-methylpyrimidine | 311.403 | 312.1 | 98.60 | ¹H NMR (400 MHz, DMSO-d₆) δ 11.82 (d, J = 76.6 Hz, 1H), 8.49-8.34 (m, 1H), 7.49 (s, 1H), 7.35-7.15 (m, 1H), 6.96 (q, J = 7.7 Hz, 3H), 6.67 (d, J = 56.3 Hz, 1H), 4.87 (t, J = 7.9 Hz, 1H), 3.45-3.40 (m, 1H), 3.18- 3.02 (m, 1H), 2.53 (s, 3H), 2.18 (s, 3H). |
| D | 360 | 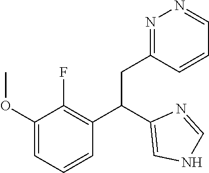 [SMILES: COc1cccc(C(Cc2cccnn2)c2c[nH]cn2)c1F] | 3-[2-(2-fluoro-3- methoxyphenyl)-2-(4- imidazolyl)ethyl]pyridazine | 298.3 | 299 | 98.6 | 1H NMR (400 MHz, Chloroform-d) δ 9.02-8.96 (m, 1H), 7.56 (d, J = 1.1 Hz, 1H), 7.32-7.27 (m, 1H), 7.25-7.20 (m, 1H), 7.02-6.93 (m, 1H), 6.93-6.86 (m, 1H), 6.85-6.76 (m, 2H), 5.02-4.94 (m, 1H), 3.91- 3.78 (m, 4H), 3.68-3.58 (m, 1H). |
| D | 361 | 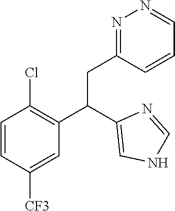 [SMILES: FC(F)(F)c1ccc(Cl)c(C(Cc2cccnn2)c2c[nH]cn2)c1] | 3-{2-[2-chloro-5- (trifluoromethyl)phenyl]-2- (4- imidazolyl)ethyl}pyridazine | 352.8 | 353 | 98.1 | 1H NMR (400 MHz, DMSO-d6) δ 12.16-11.78 (m, 1H), 8.99 (d, J =4.9, 1.7 Hz, 1H), 7.94 (d, J = 2.3 Hz, 1H), 7.72-7.52 (m, 3H), 7.52-7.46 (m, 1H), 7.43-7.35 (m, 1H), 6.89 (d, J = 55.2 Hz, 1H), 5.28-5.04 (m, 1H), 3.78-3.64 (m, 1H), 3.62-3.46 (m, 1H). |
| D | 362 | 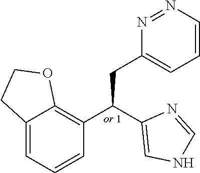 [SMILES: c1cnnc(C[C@@H](c2c[nH]cn2)c2cccc3c2OCC3)c1] | 3-[(R)-2-(2,3-dihydro-1- benzofuran-7-yl)-2-(4- imidazolyl)ethyl]pyridazine | 292.3 | 293 | 98.9 | 1H NMR (300 MHz, DMSO-d6) δ 11.78 (s, 1H), 8.97 (dd, J = 4.9, 1.7 Hz, 1H), 7.56-7.41 (m, 2H), 7.37-7.27 (m, 1H), 7.03 (t, J = 7.3 Hz, 2H), 6.84-6.65 (m, 2H), 4.65 (t, J = 8.0 Hz, 1H), 4.55-4.30 (m, 2H), 3.70- 3.55 (m, 1H), 3.52-3.40 (m, 1H), 3.12 (t, J = 8.7 Hz, 2H). |
| D | 363 | 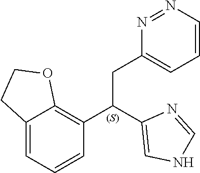 | 3-[(S)-2-(2,3-dihydro-1- benzofuran-7-yl)-2-(4- imidazolyl)ethyl]pyridazine | 292.3 | 293 | 97.1 | 1H NMR (300 MHz, DMSO-d6) δ 11.79 (s, 1H), 8.97 (dd, J = 4.9, 1.7 Hz, 1H), 7.56-7.43 (m, 2H), 7.31 (dd, J =8.4, 1.7 Hz, 1H), 7.03 (t, J = 7.2 Hz, 2H), 6.83-6.65 (m, 2H), 4.65 (t, J = 8.0 Hz, 1H), 4.56-4.34 (m, 2H), 3.68-3.56 (m, 1H), 3.51-3.40 (m, 1H), 3.12 (t, J = 8.7 Hz, 2H). |

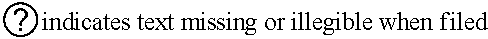

### Procedure E

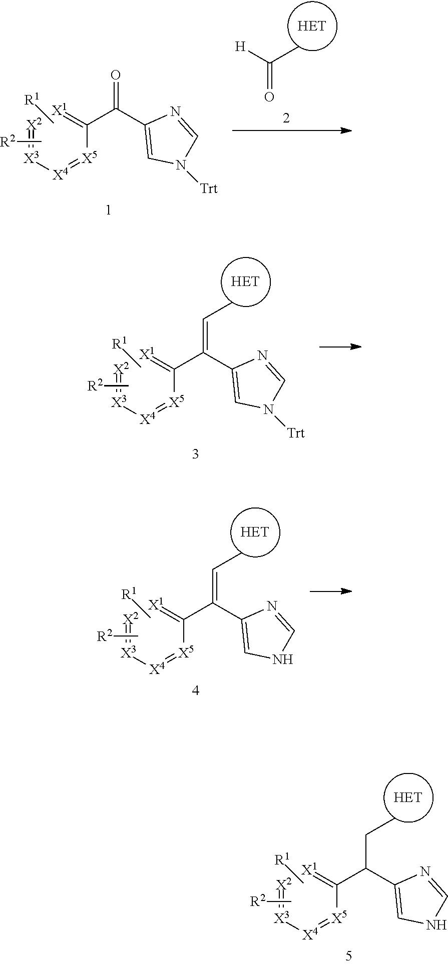

### Example of Procedure E: Synthesis of Compounds 391 & 392

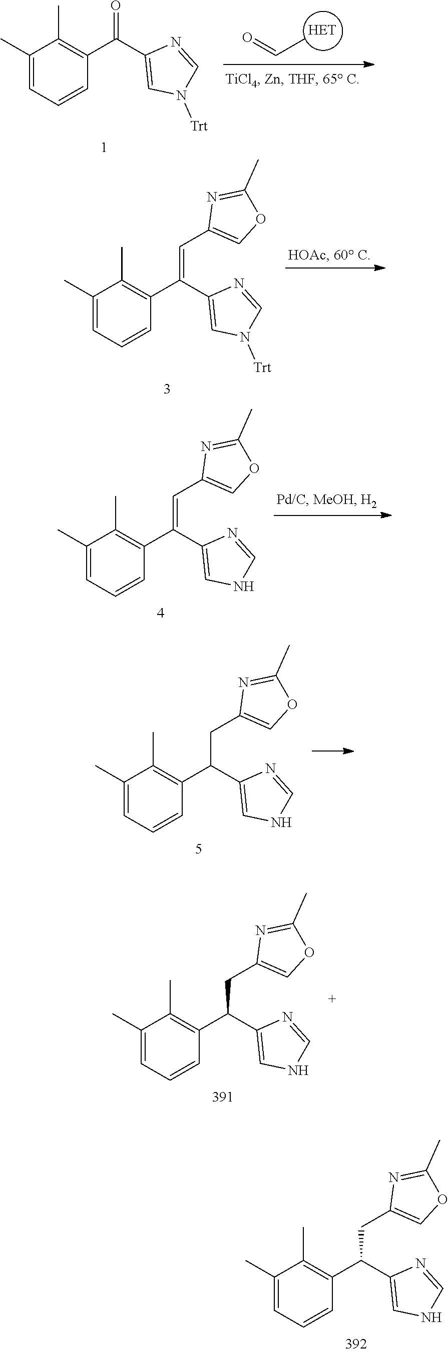

### Experimental Details

### 1. Synthesis of 3

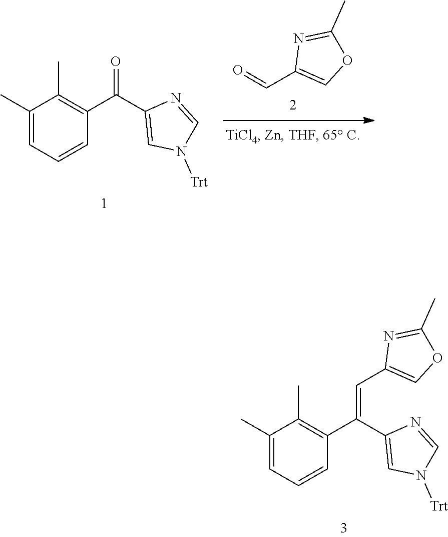

### 4-[(Z)-2-(2,3-dimethylphenyl)-2-[1-(triphenylmethyl)imidazol-4-yl]ethenyl]-2-methyl-1,3-oxazole

A mixture of Zinc (1.06 g, 16.20 mmol, 6.0 equiv) in THF (20.00 mL) was treated with titanium tetrachloride (1.5 g, 8.10 mmol, 3.0 equiv) at 0° C. for 10 min under nitrogen atmosphere followed by the addition of 2-methyl-1,3-oxazole-4-carbaldehyde (300 mg, 2.70 mmol, 1.0 equiv) and 4-(2,3-dimethylbenzoyl)-1-(triphenylmethyl)imidazole (1.2 g, 2.70 mmol, 1.0 equiv) in portions. The mixture was stirred at 65° C. for 35 min. The reaction was quenched by the addition of water. The resulting mixture was extracted with ethyl acetate (3×30 mL). The combined organic layers were washed with water (30 mL), dried over anhydrous sodium sulfate. After filtration, the filtrate was concentrated under reduced pressure. The residue was purified by silica gel column chromatography, eluted with petroleum ether/ethyl acetate (1:1) to afford 4-[(Z)-2-(2,3-dimethylphenyl)-2-[1-(triphenylmethyl)imidazol-4-yl]ethenyl]-2-methyl-1,3-oxazole (720 mg, 51.15% yield) as a yellow solid. LCMS (ESI) [M+H]⁺: 522.

### 2. Synthesis of 4

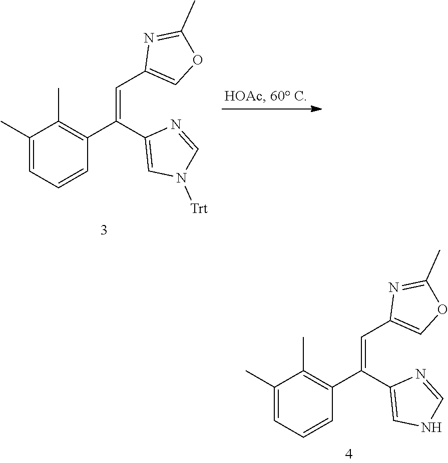

### 4-[(Z)-2-(2,3-dimethylphenyl)-2-(1H-imidazol-4-yl)ethenyl]-2-methyl-1,3-oxazole

A mixture of 4-[(Z)-2-(2,3-dimethylphenyl)-2-[1-(triphenylmethyl)imidazol-4-yl]ethenyl]-2-methyl-1,3-oxazole (720 mg, 1.38 mmol, 1.0 equiv) in acetic acid (20 mL) was stirred at 60° C. for 1h under nitrogen atmosphere. The resulting mixture was concentrated under vacuum to give the residue which was purified by reversed-phase flash chromatography with the following conditions: column, C18 silica gel; mobile phase, acetonitrile in water (10 mmol/L NH₄HCO₃), 5% to 100% gradient in 25 min; detector, UV 254 nm. This resulted in 4-[(Z)-2-(2,3-dimethylphenyl)-2-(1H-imidazol-4-yl)ethenyl]-2-methyl-1,3-oxazole (330 mg, 85.58% yield) as a yellow solid. LCMS (ESI) [M+H]⁺: 280.

### 3. Synthesis of 5

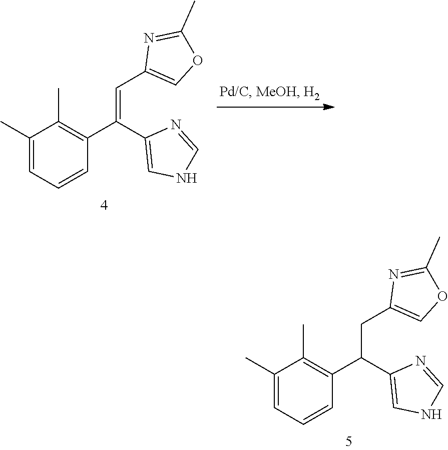 [SMILES: Cc1nc(/C=C(\c2c[nH]cn2)c2cccc(C)c2C)co1 ; Cc1nc(CC(c2c[nH]cn2)c2cccc(C)c2C)co1]

### 4-[2-(2,3-dimethylphenyl)-2-(1H-imidazol-4-yl)ethyl]-2-methyl-1,3-oxazole

A mixture of 4-[(Z)-2-(2,3-dimethylphenyl)-2-(1H-imidazol-4-yl)ethenyl]-2-methyl-1,3-oxazole (310 mg, 1.11 mmol, 1.0 equiv) and Pd/C (50 mg) in methanol (20 mL) was stirred at room temperature for 1h under hydrogen atmosphere. The resulting mixture was filtered, the filter cake was washed with methanol (3×10 mL). The filtrate was concentrated under reduced pressure. The crude product was purified by Prep-HPLC with the following conditions (Column: YMC-Actus Triart C18 ExRS 30*150 mm, 5m; Mobile Phase A: Water (10 mmol/L NH4HCO₃), Mobile Phase B: ACN; Flow rate: 60 mL/min; Gradient: 22% B to 47% B in 7 min; Wave Length: 254 nm/220 nm; RT1 (min): 5.97) to afford 4-[2-(2,3-dimethylphenyl)-2-(1H-imidazol-4-yl)ethyl]-2-methyl-1,3-oxazole (11.4 mg, 3.65% yield) as a light yellow solid. LCMS (ESI) [M+H]⁺: 282.20. ¹H NMR (300 MHz, DMSO-d₆) δ 11.71 (s, 1H), 7.42 (d, J=39.2 Hz, 2H), 7.18-6.87 (m, 3H), 6.77 (s, 1H), 4.62 (d, J=25.8 Hz, 1H), 3.24-3.05 (m, 1H), 2.92-2.81 (m, 1H), 2.32 (s, 3H), 2.23 (d, J=4.3 Hz, 6H).

### Isolation of Compounds 391& 392

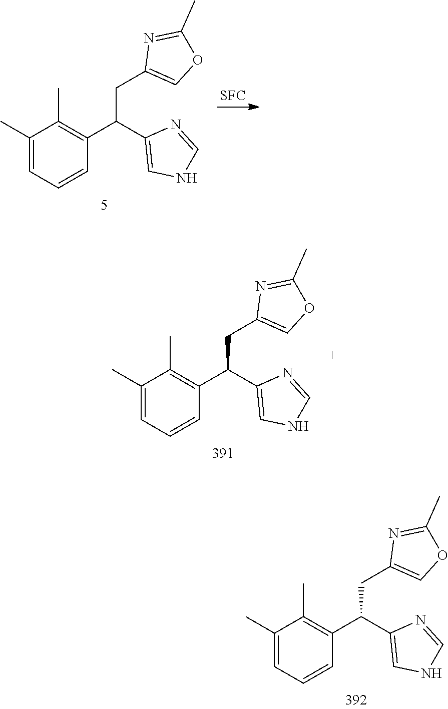

### (R) 4-[2-(2,3-dimethylphenyl)-2-(1H-imidazol-4-yl)ethyl]-2-methyl-1,3-oxazole & (S) 4-[2-(2,3-dimethylphenyl)-2-(1H-imidazol-4-yl)ethyl]-2-methyl-1,3-oxazole

4-[2-(2,3-dimethylphenyl)-2-(1H-imidazol-4-yl)ethyl]-2-methyl-1,3-oxazole (205.4 mg, 0.73 mmol, 1 equiv) was separated by prep-Chiral-HPLC (Column: CHIRALCEL AY-H, 2*25 cm, 5 μm; Mobile Phase A: Hex (10 mM NH₃-MeOH), Mobile Phase B: EtOH-HPLC; Flow rate: 20 mL/min; Gradient: isocratic 20; Wave Length: 214/230 nm; RT1 (min): 4.285; RT2 (min): 11.35; Sample Solvent: MeOH:EtOH=1:1—HPLC; Injection Volume: 1.5 mL; Number Of Runs: 20). The resulted in (R) 4-[2-(2,3-dimethylphenyl)-2-(1H-imidazol-4-yl)ethyl]-2-methyl-1,3-oxazole (82.5 mg, 100% e.e.) as a white solid. LCMS (ESI) [M+H]⁺: 282.10. ¹H NMR (400 MHz, DMSO-d₆) δ 11.72 (s, 1H), δ 7.47 (s, 1H), δ 7.37 (s, 1H), 7.17-6.85 (m, 3H), 6.75 (s, 1H), 4.59 (s, 1H), 3.17 (d, J=14.9 Hz, 1H), 2.86 (dd, J=14.7, 6.8 Hz, 1H), 2.31 (s, 3H), 2.22 (d, J=5.5 Hz, 6H).

(S) 4-[2-(2,3-dimethylphenyl)-2-(1H-imidazol-4-yl)ethyl]-2-methyl-1,3-oxazole (82.4 mg, 100% e.e.) as a white solid. LCMS (ESI) [M+H]⁺: 282.15. ¹H NMR (400 MHz, DMSO-d₆) δ 11.72 (s, 1H), δ 7.47 (s, 1H), δ 7.37 (s, 1H), 7.17-6.85 (m, 3H), 6.75 (s, 1H), 4.59 (s, 1H), 3.17 (d, J=14.9 Hz, 1H), 2.86 (dd, J=14.7, 6.8 Hz, 1H), 2.31 (s, 3H), 2.22 (d, J=5.5 Hz, 6H).

TABLE E

| | | | | Molecular | | | |
|---|---|---|---|---|---|---|---|
| | | | | weight | LCMS | Purity | |
| Procedure | Name | Structure | IUPAC | (g/mol) | [M + H]+ | (%) | 1H NMR |
| E | 364 | 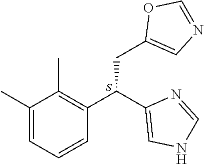 | 5-[(S)-2-(4- imidazolyl)-2-(2,3- xylyl)ethyl]-1,3- oxazole | 267.3 | 268.3 | 98.8 | ¹H NMR (400 MHz, DMSO-d6): δ 11.74 (s, 1H), 8.11 (s, 1H), 7.50 (s, 1H), 7.18 (d, J = 7.2 Hz, 1H), 7.03-6.61 (m, 2H), 6.79-6.61 (m, 2H), 4.55 (t, J = 7.6 Hz, 1H), 3.48-3.40 (m, 1H), 3.20-3.14 (m, 1H), 2.21 (s, 3H), 2.19 (s, 3H). |
| E | 365 | 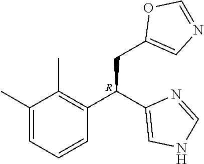 | 5-[(R)-2-(4- imidazolyl)-2-(2,3- xylyl)ethyl]-1,3- oxazole | 267.3 | 268.3 | 99.38 | ¹H NMR (400 MHz, DMSO-d6): δ 11.74 (s, 1H), 8.11 (s, 1H), 7.49 (s, 1H), 7.17-6.61 (m, 5H), 4.58-4.54 (m, 1H), 3.47-3.41 (m, 1H), 3.20- 3.14 (m, 1H), 2.21 (s, 3H), 2.19 (s, 3H). |
| E | 366 | 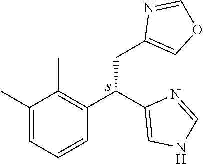 | 4-[(S)-2-(4- imidazolyl)-2-(2,3- xylyl)ethyl]-1,3- oxazole | 267.33 | 268.3 | 99.52 | ¹H NMR (400 MHz, DMSO-d6): δ 11.74 (s, 1H), 8.17 (s, 1H), 7.54 (s, 1H), 7.47 (d, J = 6.8 Hz, 1H), 7.10 (d, J = 7.2 Hz, 1H), 6.99-6.93 (m, 2H), 6.74 (s, 1H), 4.62 (d, J = 7.6 Hz, 1H), 3.25-3.21 (m, 1H), 2.99-2.93 (m, 1H), 2.21 (s, 6H). |
| E | 367 | 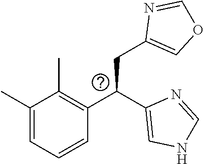 [SMILES: Cc1cccc([C@@H](Cc2cocn2)c2c[nH]cn2)c1C] | 4-[(R)-2-(4- imidazolyl)-2-(2,3- xylyl)ethyl]-1,3- oxazole | 267.33 | 268.3 | 99.92 | ¹H NMR (400 MHz, DMSO-d6): δ 11.76 (s, 1H), 8.17 (s, 1H), 7.53 (s, 1H), 7.48 (d, J = 6.8 Hz, 1H), 7.10 (d, J = 7.2 Hz, 1H), 6.99-6.91 (m, 2H), 6.75 (s 1H), 4.62 (d, J = 7.6 Hz, 1H), 3.25-3.21 (m, 1H), 2.98-2.93 (m, 1H), 2.21 (s, 6H). |
| E | 368 | 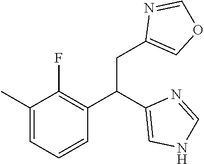 [SMILES: Cc1cccc(C(Cc2cocn2)c2c[nH]cn2)c1F] | 4-[2-(2-fluoro-3- tolyl)-2-(4- imidazolyl)ethyl]- 1,3-oxazole | 271.295 | 272.1 | 99.6 | 1H NMR (400 MHz, DMSO-d6) δ 11.86 (d, J = 71.5 Hz, 1H), 8.18 (d, J = 12.3 Hz, 1H), 7.68-7.47 (m, 2H), 7.22 (t, J = 7.0 Hz, 1H), 7.14-6.94 (m, 2H), 6.77 (d, J = 44.3 Hz, 1H), 4.64 (dt, J = 40.3, 7.9 Hz, 1H), 3.24 (d, J = 7.5 Hz, 1H), 3.04 (dd, J = 14.7, 8.2 Hz, 1H), 2.18 (d, J = 2.2 Hz, 3H). |
| E | 369 | 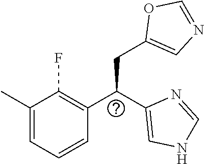 | 5-[(R)-2-(2-fluoro-3- tolyl)-2-(4- imidazolyl)ethyl]- 1,3-oxazole | 271.295 | 272.1 | 99.5 | 1H NMR (400 MHz, DMSO-d6) δ 11.83 (s, 1H), 8.13 (s, 1H), 7.53 (s, 1H), 7.22 (s, 1H), 7.08 (t, J = 7.3 Hz, 1H), 6.99 (t, J = 7.6 Hz, 1H), 6.86 (s, 1H), 6.65 (s, 1H), 4.57 (s, 1H), 3.50-3.41 (m, 1H), 3.29-3.20 (m, 1H), 2.19 (d, J = 2.0 Hz, 3H). |
| E | 370 | 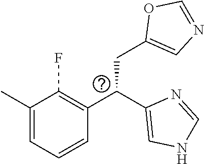 | 5-[(S)-2-(2-fluoro-3- tolyl)-2-(4- imidazolyl)ethyl]- 1,3-oxazole | 271.295 | 272.1 | 98.6 | 1H NMR (400 MHz, DMSO-d6) δ 11.83 (s, 1H), 8.13 (s, 1H), 7.53 (s, 1H), 7.22 (s, 1H), 7.08 (t, J = 7.3 Hz, 1H), 6.99 (t, J = 7.6 Hz, 1H), 6.86 (s, 1H), 6.65 (s, 1H), 4.57 (s, 1H), 3.50-3.41 (m, 1H), 3.29-3.20 (m, 1H), 2.19 (d, J = 2.0 Hz, 3H). |
| E | 371 | 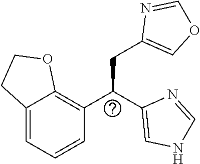 [SMILES: c1cc2c(c([C@@H](Cc3cocn3)c3c[nH]cn3)c1)OCC2] | 4-[(R)-2-(2,3- dihydro-1- benzofuran-7-yl)-2- (4-imidazolyl)ethyl] 1,3-oxazole | 281.315 | 282.1 | 99.0 | 1H NMR (400 MHz, DMSO-d6) δ 11.75 (s, 1H), 8.17 (s, 1H), 7.52 (s, 1H), 7.48 (s, 1H), 7.11-6.90 (m, 2H), 6.79-6.62 (m, 2H), 4.64-4.33 (m, 3H), 3.24-3.09 (m, 3H), 3.09-2.95 (m, 1H). |
| E | 372 | 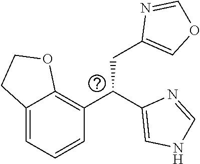 [SMILES: c1cc2c(c([C@H](Cc3cocn3)c3c[nH]cn3)c1)OCC2] | 4-[(S)-2-(2,3- dihydro-1- benzofuran-7-yl)-2- (4-imidazolyl)ethyl] 1,3-oxazole | 281.315 | 282.1 | 98.5 | 1H NMR (400 MHz, DMSO-d6) δ 11.75 (s, 1H), 8.17 (s, 1H), 7.52 (s, 1H), 7.48 (s, 1H), 7.11-6.90 (m, 2H), 6.79-6.62 (m, 2H), 4.64-4.33 (m, 3H), 3.24-3.09 (m, 3H), 3.09-2.95 (m, 1H). |
| E | 373 | 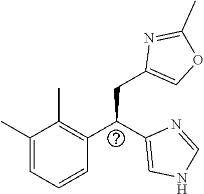 [SMILES: Cc1nc(C[C@@H](c2c[nH]cn2)c2cccc(C)c2C)co1] | 4-[(R)-2-(4- imidazolyl)-2-(2,3- xylyl)ethyl]-2- methyl-1,3-oxazole | 281.359 | 282.1 | 98.5 | ¹H NMR (400 MHz, DMSO-d₆) δ 11.72 (s, 1H), δ 7.47 (s, 1H), δ 7.37 (s, 1H), 7.17-6.85 (m, 3H), 6.75 (s, 1H), 4.59 (s, 1H), 3.17 (d, J = 14.9 Hz, 1H), 2.86 (dd, J = 14.7, 6.8 Hz, 1H), 2.31 (s, 3H), 2.22 (d, J = 5.5 Hz, 6H). |
| E | 374 | 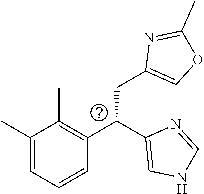 [SMILES: Cc1nc(C[C@H](c2c[nH]cn2)c2cccc(C)c2C)co1] | 4-[(S)-2-(4- imidazolyl)-2-(2,3- xylyl)ethyl]-2- methyl-1,3-oxazole | 281.359 | 282.15 | 97.6 | ¹H NMR (400 MHz, DMSO-d₆) δ 11.72 (s, 1H), δ 7.47 (s, 1H), δ 7.37 (s, 1H), 7.17-6.85 (m, 3H), 6.75 (s, 1H), 4.59 (s, 1H), 3.17 (d, J = 14.9 Hz, 1H), 2.86 (dd, J = 14.7, 6.8 Hz, 1H), 2.31 (s, 3H), 2.22 (d, J = 5.5 Hz, 6H). |
| E | 375 | 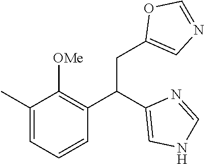 [SMILES: COc1c(C)cccc1C(Cc1cnco1)c1c[nH]cn1] | 5-[2-(4-imidazolyl)- 2-(2-methoxy-3- tolyl)ethyl]-1,3- oxazole | 283.331 | 284.05 | 95.8 | 1H NMR (400 MHz, DMSO-d6) δ 12.37 (s, 1H), 8.13 (d, J = 4.0 Hz, 2H), 7.65 (s, 1H), 7.24 (dd, J = 7.7, 1.9 Hz, 1H), 7.03 (dd, J = 7.6, 1.9 Hz, 1H), 6.97 (t, J = 7.5 Hz, 1H), 6.87 (s, 1H), 6.64 (s, 1H), 4.68 (t, J = 7.9 Hz, 1H), 3.63 (s, 3H),3.43 (dd, J = 15.3, 7.8 Hz, 1H), 3.25-3.13 (m, 1H), 2.21 (s, 3H). |
| E | 376 | 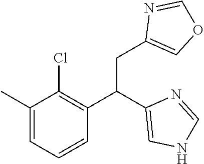 [SMILES: Cc1cccc(C(Cc2cocn2)c2c[nH]cn2)c1Cl] | 4-[2-(2-chloro-3- tolyl)-2-(4- imidazolyl)ethyl]- 1,3-oxazole | 287.75 | 288.1 | 97.2 | 1H NMR (400 MHz, DMSO-d6) δ 11.78 (s, 1H), 8.16 (s, 1H), 7.65-7.46 (m, 2H), 7.29 (s, 1H), 7.15 (d, J = 5.9 Hz, 2H), 6.80 (s, 1H), 4.93-4.80 (m, 1H), 3.29-3.20 (m, 1H), 3.08-2.97 (m, 1H), 2.31 (s, 3H). |
| E | 377 | 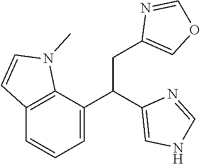 [SMILES: Cn1ccc2cccc(C(Cc3cocn3)c3c[nH]cn3)c21] | 4-[2-(4-imidazolyl)- 2-(1-methyl-7- indolyl)ethyl]-1,3- oxazole | 292.342 | 293.1 | 95.1 | 1H NMR (400 MHz, DMSO-d6) δ 11.79 (s, 1H), 8.23-8.13 (m, 1H), 7.72-7.49 (m, 2H), 7.41-7.08 (m, 3H), 6.91 (t, J = 7.5 Hz, 1H), 6.79 (d, J = 3.1 Hz, 1H), 6.42-6.31 (m, 1H), 5.48-5.29 (m, 1H), 4.14 (d, J = 4.0 Hz, 3H), 3.40-3.29 (m, 1H), 3.25-3.14 (m, 1H). |
| E | 378 | 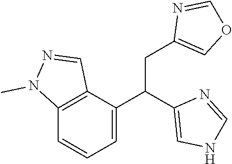 [SMILES: Cn1ncc2c(C(Cc3cocn3)c3c[nH]cn3)cccc21] | 4-[1-(4-imidazolyl)- 2-(1,3-oxazol-4- yl)ethyl]-1-methyl- 1H-indazole | 293.33 | 294.1 | 99.1 | ¹H NMR (400 MHz, DMSO-d₆) δ 11.82 (d, J = 57.5 Hz, 1H), 8.18 (d, J = 16.6 Hz, 1H), 8.11-8.02 (m, 1H), 7.70-7.46 (m, 2H), 7.39 (d, J = 8.4 Hz, 1H), 7.32-7.16 (m, 1H), 7.04 (d, J = 7.0 Hz, 1H), 6.90 (d, J = 25.8 Hz, 1H), 4.69 (t, J = 7.7 Hz, 1H), 3.99 (d, J = 6.2 Hz, 3H), 3.51-3.36 (m, 1H), 3.30-3.13 (m, 1H). |
| E | 379 | 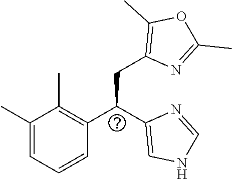 | 4-[(R)-2-(4- imidazolyl)-2-(2,3- xylyl)ethyl]-2,5- dimethyl-1,3- oxazole | 295.386 | 296.1 | 99.6 | ¹H NMR (400 MHz, DMSO-d6) δ 11.72 (s, 1H), 7.47 (s, 1H), 7.14 (s, 1H), 6.96 (d, J = 9.2 Hz, 2H), 6.75 (s, 1H), 4.55 (s, 1H), 3.13- 3.00 (m, 1H), 2.81-2.71 (m, 1H), 2.26 (s, 3H), 2.19 (d, J = 14.0 Hz, 6H), 1.90 (s, 3H). |
| E | 380 | 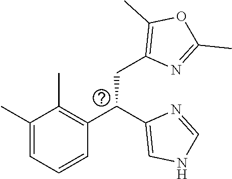 | 4-[(S)-2-(4- imidazolyl)-2-(2,3- xylyl)ethyl]-2,5- dimethyl-1,3- oxazole | 295.386 | 296.1 | 99.6 | ¹H NMR (400 MHz, DMSO-d6) δ 11.72 (s, 1H), 7.47 (s, 1H), 7.14 (s, 1H), 6.96 (d, J = 9.2 Hz, 2H), 6.75 (s, 1H), 4.55 (s, 1H), 3.13- 3.00 (m, 1H), 2.81-2.71 (m, 1H), 2.26 (s, 3H), 2.19 (d, J = 14.0 Hz, 6H), 1.90 (s, 3H). |
| E | 381 | 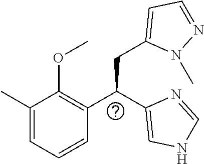 [SMILES: COc1c(C)cccc1[C@@H](Cc1ccnn1C)c1c[nH]cn1] | 5-[(R)-2-(4- imidazolyl)-2-(2- methoxy-3- tolyl)ethyl]-1- methylpyrazole | 296.374 | 297.15 | 98.9 | 1H NMR (400 MHz, DMSO-d6) δ 11.79 (s, 1H), 7.53 (d, J = 1.2 Hz, 1H), 7.29 (d, J = 7.6 Hz, 1H), 7.13 (d, J = 1.8 Hz, 1H), 7.04-6.92 (m, 2H), 6.80 (s, 1H), 5.78 (d, J = 1.8 Hz, 1H), 4.60 (t, J = 7.7 Hz, 1H), 3.69 (s, 3H), 3.59 (s, 3H), 3.40 (s, 1H), 3.16-3.08 (m, 1H), 2.21 (s, 3H). |
| E | 382 | 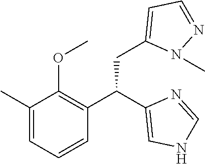 | 5-[(S)-2-(4- imidazolyl)-2-(2- methoxy-3- tolyl)ethyl]-1- methylpyrazole | 296.374 | 297.1 | 98.1 | 1H NMR (400 MHz, DMSO-d6) δ 11.79 (s, 1H), 7.53 (d, J = 1.2 Hz, 1H), 7.29 (d, J = 7.6 Hz, 1H), 7.13 (d, J = 1.8 Hz, 1H), 7.04-6.92 (m, 2H), 6.80 (s, 1H), 5.78 (d, J = 1.8 Hz, 1H), 4.60 (t, J = 7.7 Hz, 1H), 3.69 (s, 3H), 3.59 (s, 3H), 3.40 (s, 1H), 3.16-3.08 (m, 1H), 2.21 (s, 3H). |
| E | 383 | 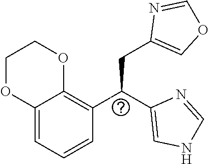 [SMILES: c1cc2c(c([C@@H](Cc3cocn3)c3c[nH]cn3)c1)OCCO2] | 4-[(R)-2-(2,3- dihydro-1,4- benzodioxin-5-yl)-2- (4-imidazolyl)ethyl] 1,3-oxazole | 297.314 | 298.1 | 99.6 | ¹H NMR (400 MHz, DMSO-d6) δ 11.75 (s, 1H), 8.17 (s, 1H), 7.49 (d, J = 5.1 Hz, 2H), 6.82-6.74 (m, 2H), 6.74-6.62 (m, 2H), 4.72- 4.60 (m, 1H), 4.30-4.14 (m, 4H), 3.25- 3.14 (m, 1H), 3.05-2.91 (m, 1H). |
| E | 384 | 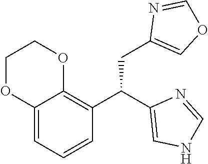 [SMILES: c1cc2c(c([C@H](Cc3cocn3)c3c[nH]cn3)c1)OCCO2] | 4-[(S)-2-(2,3- dihydro-1,4- benzodioxin-5-yl)-2- (4-imidazolyl)ethyl]- 1,3-oxazole | 297.314 | 298.05 | 99.4 | ¹H NMR (400 MHz, DMSO-d6) δ 11.75 (s, 1H), 8.17 (s, 1H), 7.49 (d, J = 5.1 Hz, 2H), 6.82-6.74 (m, 2H), 6.74-6.62 (m, 2H), 4.72- 4.60 (m, 1H), 4.30-4.14 (m, 4H), 3.25- 3.14 (m, 1H), 3.05-2.91 (m, 1H). |
| E | 385 | 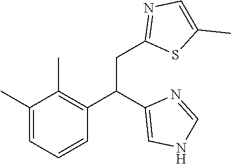 | 2-[2-(4-imidazolyl)- 2-(2,3-xylyl)ethyl]-5- methyl-1,3-thiazole | 297.427 | 298.05 | 98.8 | ¹H NMR (400 MHz, DMSO-d₆) δ 11.96- 11.61 (m, 1H), 7.56-7.43 (m, 1H), 7.33- 7.23 (m, 1H), 7.19 (dd, J = 7.3, 2.0 Hz, 1H), 7.09-6.90 (m, 2H), 6.84-6.64 (m, 1H), 4.82- 4.58 (m, 1H), 3.75-3.52 (m, 1H), 3.45- 3.33 (m, 1H), 2.36-2.26 (m, 3H), 2.26-2.10 (m, 6H). |
| E | 386 | 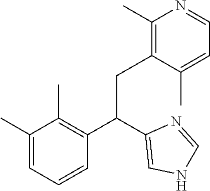 [SMILES: Cc1cccc(C(Cc2c(C)ccnc2C)c2c[nH]cn2)c1C] | 4-[2-(2,4-dimethyl- 3-pyridyl)-1-(2,3- xylyl)ethyl]imidazole | 305.425 | 306.15 | 98.5 | ¹H NMR (400 MHz, DMSO-d₆) δ 11.90- 11.56 (m, 1H), 8.15-7.97 (m, 1H), 7.55- 7.45 (m, 1H), 7.44-7.15 (m, 1H), 7.10-6.92 (m, 2H), 6.91-6.83 (m, 1H), 6.80 (s, 1H), 4.47-4.30 (m, 1H), 3.53-3.38 (m, 1H), 3.18- 3.05 (m, 1H), 2.27-2.03 (m, 6H), 1.95- 1.81 (m, 3H), 1.78-1.62 (m, 3H). |
| E | 387 | 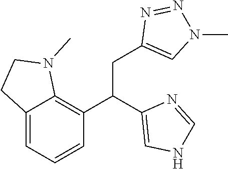 [SMILES: CN1CCc2cccc(C(Cc3cn(C)nn3)c3c[nH]cn3)c21] | 4-[2-(4-imidazolyl)- 2-(1-methyl-7- indolinyl)ethyl]-1- methyl-1H-1,2,3- triazole | 308.389 | 309.15 | 98.4 | 1H NMR (400 MHz, DMSO-d6) δ 11.81 (d, J = 59.4 Hz, 1H), 7.57-7.33 (m, 2H), 6.96 (dd, J = 29.4, 7.5 Hz, 1H), 6.89-6.66 (m, 2H), 6.60 (t, J = 7.5 Hz, 1H), 4.84-4.58 (m, 1H), 3.90 (d, J = 11.2 Hz, 3H), 3.24-3.21 (m, 1H), 3.20-3.00 (m, 3H), 2.95 (d, J = 17.1 Hz, 3H), 2.82 (dq, J = 24.2, 8.7, 8.2 Hz, 2H). |
| E | 388 | 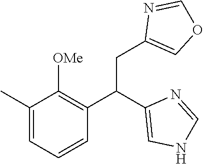 [SMILES: COc1c(C)cccc1C(Cc1cocn1)c1c[nH]cn1] | 4-[2-(4-imidazolyl)- 2-(2-methoxy-3- tolyl)ethyl]-1,3- oxazole-formic acid (1/1) | 329.356 | 284.1 | 98.5 | 1H NMR (400 MHz, Chloroform-d) δ 8.40 (s, 1H), 7.86 (s, 1H), 7.83 (s, 1H), 7.29 (d, J = 18.7 Hz, 1H), 7.12-7.03 (m, 2H), 7.02- 6.95 (m, 1H), 6.75 (s, 1H), 4.88-4.77 (m, 1H), 3.67 (s, 3H), 3.44-3.33 (m, 1H), 3.23- 3.12 (m, 1H), 2.29 (s, 3H). |
| E | 389 | 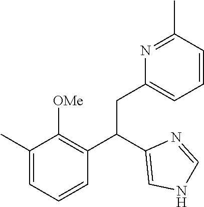 [SMILES: COc1c(C)cccc1C(Cc1cccc(C)n1)c1c[nH]cn1] | 4-[1-(2-methoxy-3- tolyl)-2-(6-methyl-2- pyridyl)ethyl]imida- zole-formic acid (1/1) | 353.422 | 308.15 | 98.6 | 1H NMR (400 MHz, DMSO-d6) δ 8.16 (s, 1H), 7.47 (s, 1H), 7.45-7.37 (m, 1H), 7.29 (d, J = 7.2 Hz, 1H), 7.01-6.90 (m, 3H), 6.84 (d, J = 7.6 Hz, 1H), 6.68 (s, 1H), 4.94-4.82 (m, 1H), 3.58 (s, 3H), 3.40 (s,1H), 3.22-3.16 (m, 1H), 2.39 (s, 3H), 2.18 (s, 3H). |

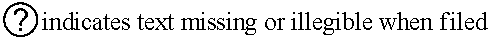

### Procedure F

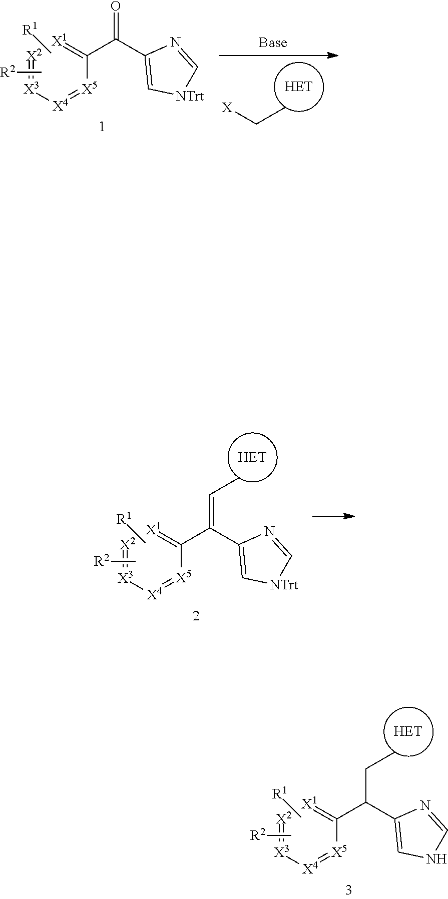

### Example of Procedure F: Synthesis of Compound 391

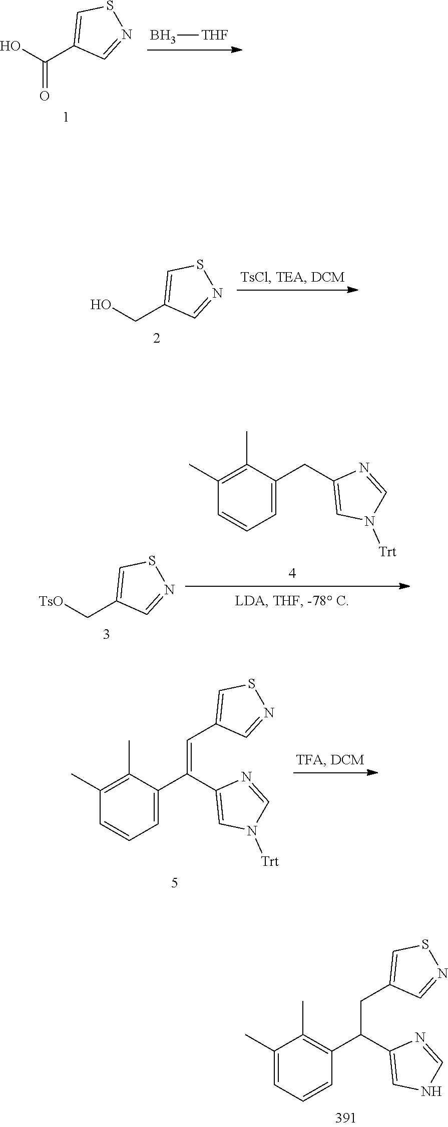

### Experimental Details

### 1. Synthesis of 2

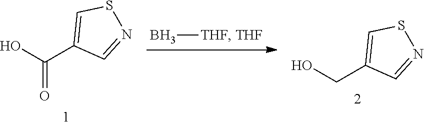

### 1,2-thiazol-4-ylmethanol

To a solution of 1,2-thiazole-4-carboxylic acid (900 mg, 6.98 mmol, 1.0 equiv) in THF (10 mL) was added and borane-tetrahydrofuran complex (14 mL, 13.96 mmol, 1 M in THF) dropwise. The solution was stirred for 3 h at room temperature under nitrogen atmosphere. The reaction was quenched with methanol at 0° C. The resulting mixture was concentrated under reduced pressure. The residue was purified by silica gel column chromatography, eluted with dichloromethane/methanol (10:1) to afford 1,2-thiazol-4-ylmethanol (400 mg, 49.88% yield) as a white solid.

### 2. Synthesis of 3

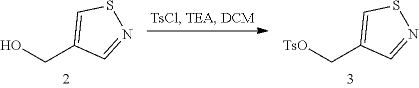 [SMILES: OCc1cnsc1 ; Cc1ccc(S(=O)(=O)OCc2cnsc2)cc1]

### 1,2-thiazol-4-ylmethyl 4-methylbenzenesulfonate

A solution of 1,2-thiazol-4-ylmethanol (400 mg, 3.48 mmol, 1.0 equiv), tosyl chloride (727 mg, 3.83 mmol, 1.10 equiv) and triethylamine (1.06 g, 10.44 mmol, 3.0 equiv) in dichloromethane (10 mL) was stirred for 3 h at room temperature under nitrogen atmosphere. The solution was quenched by the addition of water. The aqueous solution was extracted with dichloromethane (20 mL×3). The combined organic solution was dried over anhydrous sodium sulfate and concentrated under vacuum to give the residue which was purified by silica gel column chromatography, eluted with petroleum ether/ethyl acetate (5:1) to afford 1,2-thiazol-4-ylmethyl 4-methylbenzenesulfonate (210 mg, 22.44% yield) as a white solid.

### 3. Synthesis of 5

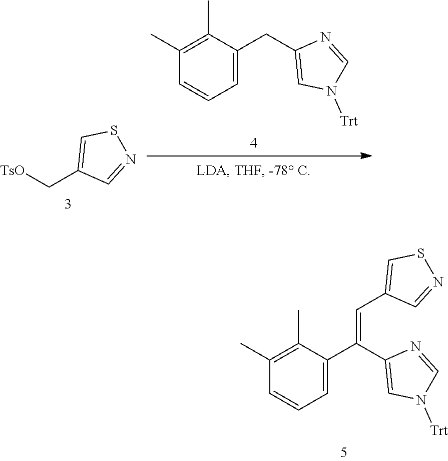

### 4-[2-(2,3-dimethylphenyl)-2-[1-(triphenylmethyl)imidazol-4-yl]ethyl]-1,2-thiazole

To a solution of 4-[(2,3-dimethylphenyl)methyl]-1-(triphenylmethyl)imidazole (202 mg, 0.47 mmol, 1.0 equiv) in THF (3 mL) was added lithium diisopropylamide (0.47 mL, 0.94 mmol, 2.0 equiv) dropwise at −78° C. The solution was stirred for 30 min at −78° C. under nitrogen atmosphere. To the above mixture was added 1,2-thiazol-4-ylmethyl 4-methylbenzenesulfonate (190 mg, 0.71 mmol, 1.5 equiv) in THF (2 mL) dropwise at 0° C. The resulting mixture was stirred for additional 2 h at room temperature. The reaction was quenched with water at 0° C. The resulting mixture was extracted with ethyl acetate (3×20 mL). The combined organic layers were washed with water (3×20 mL), dried over anhydrous sodium sulfate. After filtration, the filtrate was concentrated under reduced pressure. The residue was purified by silica gel column chromatography, eluted with petroleum ether/ethyl acetate (1:1-1:3) to afford 4-[2-(2,3-dimethylphenyl)-2-[1-(triphenylmethyl)imidazol-4-yl]ethyl]-1,2-thiazole (160 mg crude) as a white solid. LCMS (ESI) [M+H]⁺: 526.

### 4. Synthesis of PH-ALCP-A001-353

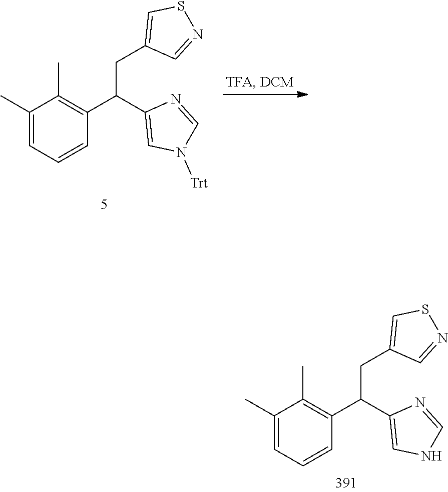

### 4-[2-(2,3-dimethylphenyl)-2-(1H-imidazol-4-yl)ethyl]-1,2-thiazole

To a solution of 4-[2-(2,3-dimethylphenyl)-2-[1-(triphenylmethyl)imidazol-4-yl]ethyl]-1,2-thiazole (160 mg, 0.30 mmol, 1.0 equiv) in dichloromethane (2 mL) was added trifluoroacetic acid (2 mL). The solution was stirred for 3 h at room temperature under nitrogen atmosphere. The resulting mixture was concentrated under vacuum. The crude product was purified by Prep-HPLC with the following conditions (Column: XBridge Prep Phenyl OBD Column 19*250 mm, 5 m; Mobile Phase A: 10 mmol/L NH₄HCO₃+0.05% NH₃H₂O, Mobile Phase B: ACN; Flow rate: 60 mL/min; Gradient: 5% B to 5% B in 1 min, 5% B to 27% B in 2 min, 27% to 40% B in 10 min; Wave Length: 254 nm/220 nm; RT1 (min): 9.5) to afford 4-[2-(2,3-dimethylphenyl)-2-(1H-imidazol-4-yl)ethyl]-1,2-thiazole (4.6 mg, 5.4% yield) as a light yellow oil. LCMS (ESI) [M+H]⁺:284.10. ¹H NMR (400 MHz, DMSO-d₆) δ 11.75 (s, 1H), 8.53-8.49 (m, 1H), 8.23-8.20 (m, 1H), 7.53-7.47 (m, 1H), 7.24-6.93 (m, 3H), 6.79-6.74 (m, 1H), 4.57-4.45 (m, 1H), 3.54-3.39 (m, 1H), 3.20-3.15 (m, 1H), 2.20-2.13 (m, 6H).

TABLE F

| | | | | Molecular | | | |
|---|---|---|---|---|---|---|---|
| | | | | weight | LCMS | | |
| Procedure | Name | Structure | IUPAC | (g/mol) | [M + H]+ | Purity (%) | 1H NMR |
| F | 390 | 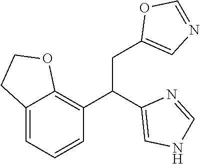 [SMILES: c1cc2c(c(C(Cc3cnco3)c3c[nH]cn3)c1)OCC2] | 5-[2-(2,3-dihydro-1- benzofuran-7-yl)-2- (4-imidazolyl)ethyl] 1,3-oxazole | 281.315 | 282.1 | 95.4 | ¹H NMR (400 MHz, DMSO-d₆) δ 12.39 (s, 1H), 8.13 (d, J = 5.5 Hz, 1H), 7.62 (s, 1H), 7.13-6.94 (m, 2H), 6.84 (s, 1H), 6.77-6.68 (m, 1H), 6.63 (s, 1H), 4.63- 4.37 (m, 3H), 3.41-3.38 (m, 1H), 3.25-3.22 (m, 1H), 3.18-3.09 (m, 2H). |
| F | 391 | 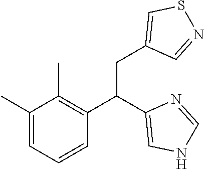 [SMILES: Cc1cccc(C(Cc2cnsc2)c2c[nH]cn2)c1C] | 4-[2-(4-imidazolyl)- 2-(2,3- xylyl)ethyl]isothiazole | 283.4 | 284.1 | 99.3 | 1H NMR (400 MHz, DMSO-d6) δ 11.75 (s, 1H), 8.53- 8.49 (m, 1H), 8.24-8.21 (m, 1H), 7.53-7.48 (m, 1H), 7.24 (d, J = 7.6 Hz, 3H), 6.79 (s, 1H), 4.47 (t, J = 7.8 Hz, 1H), 3.54-3.39 (m, 1H), 3.18 (dd, J = 14.3, 7.3 Hz, 1H), 2.18 (d, J = 17.3 Hz, 6 H). |
| F | 392 | 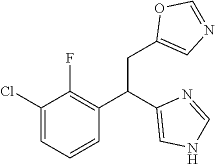 [SMILES: Fc1c(Cl)cccc1C(Cc1cnco1)c1c[nH]cn1] | 5-[2-(3-chloro-2- fluorophenyl)-2-(4- imidazolyl)ethyl]- 1,3-oxazole | 291.713 | 292 | 98.4 | 1H NMR (300 MHz, DMSO-d6) δ 8.15 (s, 1H), 7.56 (s, 1H), 7.48-7.35 (m, 2H), 7.20-7.08 (m, 1H), 6.96 (s, 1H), 6.69 (5, 1H), 4.66-4.50 (m, 1H), 3.56- 3.41 (m, 1H), 3.32 -3.13 (m, 1H). |
| F | 393 | 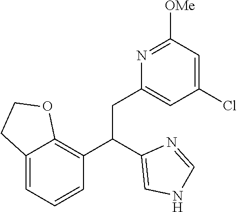 [SMILES: COc1cc(Cl)cc(CC(c2c[nH]cn2)c2cccc3c2OCC3)n1] | 4-[2-(4-chloro-6- methoxy-2-pyridyl)- 1-(2,3-dihydro-1- benzofuran-7- yl)ethyl]imidazole | 355.825 | 356.05 | 98.80% | ¹H NMR (400 MHz, DMSO-d₆) δ 11.95 (s, 1H), 7.51 (s, 1 H), 7.02 (s, 2H), 6.79 (d, J = 1.6 Hz, 1H), 6.76- 6.65 (m, 3H), 4.69 (t, J = 7.9 Hz, 1H), 4.49 (q, J = 8.6 Hz, 1H), 4.40 (q, J = 8.7 Hz, 1H), 3.80 (s, 3H), 3.37- 3.32 (m, 1H), 3.23 (m, 1H), 3.12 (t, J = 8.7 Hz, 2H). |
| F | 394 | 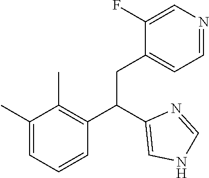 [SMILES: Cc1cccc(C(Cc2ccncc2F)c2c[nH]cn2)c1C] | 4-[2-(3-fluoro-4- pyridyl)-1-(2,3- xylyl)ethyl]imidazole | 295.361 | 296.1 | 99.50% | ¹H NMR (400 MHz, DMSO-d₆) δ 11.75 (s, 1H), 8.39 (d, J = 1.8 Hz, 1H), 8.18 (d, J = 4.9 Hz, 1H), 7.50 (s, 1H), 7.22 (d, J = 29.6 Hz, 2H), 6.98-6.78 (m, 2H), 6.78 (s, 1H), 4.61 (s, 1H), 3.19-3.10 (m, 1H), 2.18 (d, J = 11.2 Hz, 6H). |
| F | 395 | 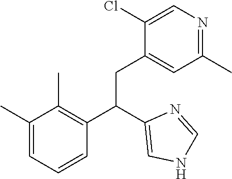 | 4-[2-(5-chloro-2- methyl-4-pyridyl)-1- (2,3- xylyl)ethyl]imidazole | 325.843 | 326.05 | 97.00% | ¹H NMR (400 MHz, DMSO-d₆) δ 12.00-11.50 (m, 1H), 8.35 (s, 1H), 7.50 (s, 1H), 7.27 (d, J = 7.7 Hz, 1H), 7.02 (t, J = 7.6 Hz, 1H), 6.96 (d, J = 7.4 Hz, 1H), 6.92 (s, 1H), 6.79 (s, 1H), 4.65 (t, J = 7.8 Hz, 1H), 3.47-3.42 (m, 1H), 3.14 (s, 1H), 2.27 (s, 3H), 2.18 (s, 3H), 2.11 (s, 3H). |
| F | 396 | 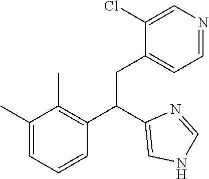 [SMILES: Cc1cccc(C(Cc2ccncc2Cl)c2c[nH]cn2)c1C] | 4-[2-(3-chloro-4- pyridyl)-1-(2,3- xylyl)ethyl]imidazole | 311.816 | 312 | 99.60% | ¹H NMR (400 MHz, DMSO-d₆) δ 8.51 (5, 1H), 8.24 (d, J = 4.8 Hz, 1H), 7.54 (s, 1H), 7.28 (d, J = 7.7 Hz, 1H), 7.12-6.99 (m, 2H), 6.96 (d, J = 7.3 Hz, 1H), 6.81 (s, 1H), 4.69 (s, 1 H), 3.25-3.23 (m, 1H), 3.22-3.20 (m, 1H), 2.18 (s, 3H), 2.12 (5, 3H). |
| F | 397 | 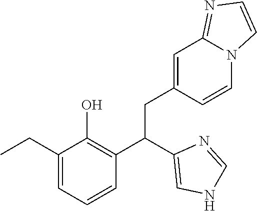 [SMILES: CCc1cccc(C(Cc2ccn3ccnc3c2)c2c[nH]cn2)c1O] | 2-[2-(1,3a-diaza-6- indenyl)-1-(4- imidazolyl)ethyl]-6- ethylphenol | 332.407 | 333.2 | 95.2 | ¹H NMR (400 MHz, DMSO-d₆) δ 8.29 (t, J = 17.0 Hz, 2H), 7.78 (d, J = 4.5 Hz, 2H), 7.44 (s, 1H), 7.16 (s, 1H), 7.05-6.84 (m, 3H), 6.62-6.45 (m, 2H), 4.34 (t, J = 8.1 Hz, 1H), 3.35-3.34 (m, 2H), 3.24-3.23 (m, 1H), 2.59 (q, J = 7.5 Hz, 2H), 1.13 (t, J = 7.5 Hz, 3H). |
| F | 398 | 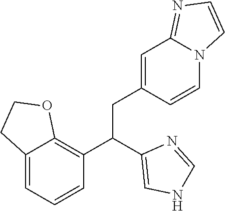 [SMILES: c1cc2c(c(C(Cc3ccn4ccnc4c3)c3c[nH]cn3)c1)OCC2] | 4-[2-(1,3a-diaza-6- indenyl)-1-(2,3- dihydro-1- benzofuran-7- yl)ethyl]imidazole | 330.391 | 331.1 | 96.80% | ¹H NMR (400 MHz, DMSO-d₆) δ 11.98-11.63 (m, 1H), 8.34 (d, J = 6.9 Hz, 1H), 7.78 (s, 1H), 7.60 7.36 (m, 2H), 7.28-6.90 (m, 3H), 6.86-6.58 (m, 3H), 4.64-4.28 (m, 3H), 3.41-3.34 (m, 1H), 3.25- 3.03 (m, 3H). |
| F | 399 | 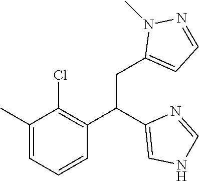 [SMILES: Cc1cccc(C(Cc2ccnn2C)c2c[nH]cn2)c1Cl] | 5-[2-(2-chloro-3- tolyl)-2-(4- imidazolyl)ethyl]-1- methylpyrazole | 300.793 | 301.05 | 99.50% | ¹H NMR (400 MHz, DMSO-d₆) δ 12.12-11.66 (m, 1H), 7.56 (s, 1H), 7.47-7.27 (m, 1H), 7.26-7.08 (m, 3H), 6.91-6.69 (m, 1H), 5.77 (d, J = 1.8 Hz, 1H), 4.93-4.68 (m, 1H), 3.70 (s, 3H), 3.48-3.39 (m, 1H), 3.17 (dd, J = 15.3, 7.2 Hz, 1H), 2.33 (s, 3 H). |
| F | 400 | 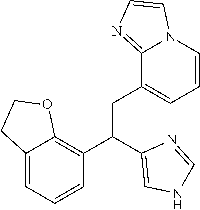 [SMILES: c1cc2c(c(C(Cc3cccn4ccnc34)c3c[nH]cn3)c1)OCC2] | 4-[2-(1,3a-diaza-7- indenyl)-1-(2,3- dihydro-1- benzofuran-7- yl)ethyl]imidazole | 330.391 | 331.1 | 98.00% | ¹H NMR (400 MHz, DMSO-d₆) δ 11.85-11.53 (m, 1H), 8.41-8.23 (m, 1H), 7.87 (s, 1H), 7.63-7.32 (m, 2H), 7.23-6.90 (m, 2H), 6.89-6.48 (m, 4H), 4.96-4.72 (m, 1H), 4.54-4.29 (m, 2H), 3.73- 3.59 (m, 1H), 3.52-3.38 (m, 1H), 3.10 (t, J = 8.8 Hz, 2H). |
| F | 401 | 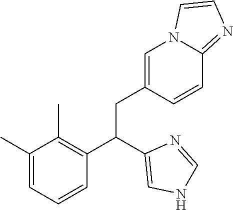 [SMILES: Cc1cccc(C(Cc2ccc3nccn3c2)c2c[nH]cn2)c1C] | 4-[2-(1,3a-diaza-5- indenyl)-1-(2,3- xylyl)ethyl]imidazole | 316.408 | 317.1 | 99.60% | ¹H NMR (400 MHz, DMSO-d₆) δ 11.89-11.65 (m, 1H), 8.28-8.17 (m, 1H), 7.83-7.74 (m, 1H), 7.54- 7.47 (m, 1H), 7.47-7.41 (m, 1H), 7.40-7.33 (m, 1H), 7.31-7.11 (m, 1H), 7.09-6.96 (m, 2H), 6.95- 6.89 (m, 1H), 6.82-6.71 (m, 1H), 4.65-4.48 (m, 1H), 3.43-3.34 (m, 1H), 3.11-3.00 (m, 1H), 2.25- 2.10 (m, 6H). |
| F | 402 | 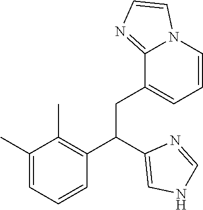 [SMILES: Cc1cccc(C(Cc2cccn3ccnc23)c2c[nH]cn2)c1C] | 4-[2-(1,3a-diaza-7- indenyl)-1-(2,3- xylyl)ethyl]imidazole | 316.408 | 317.1 | 99.40% | ¹H NMR (400 MHz, DMSO-d₆) δ 11.92-11.62 (m, 1H), 8.39-8.24 (m, 1H), 7.88 (s, 1H), 7.56 (5, 1H), 7.50-7.37 (m, 1H), 7.30-7.04 (m, 1H), 7.04- 6.72 (m, 4H), 6.72-6.60 (m, 1H), 5.30-4.96 (m, 1H), 3.67-3.48 (m, 1H), 3.38 (dd, J = 13.8, 7.1 Hz, 1H), 2.17 (s, 6H). |
| F | 403 | 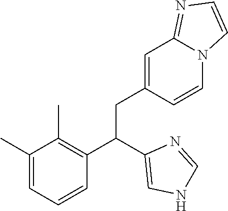 [SMILES: Cc1cccc(C(Cc2ccn3ccnc3c2)c2c[nH]cn2)c1C] | 4-[2-(1,3a-diaza-6- indenyl)-1-(2,3- xylyl)ethyl]imidazole | 316.408 | 317.15 | 99.80% | ¹H NMR (400 MHz, DMSO-d₆) δ 11.76 (s, 1H), 8.34 (d, J = 6.9 Hz, 1H), 7.78 (d, J = 1.0 Hz, 1H), 7.50 (d, J = 1.1 Hz, 1H), 7.44 (d, J = 1.2 Hz, 1H), 7.24 (d, J = 9.5 Hz, 2H), 7.01 (t, J = 7.6 Hz, 1H), 6.94 (d, J =7.4 Hz, 1H), 6.78 (s, 1H), 6.73 (dd, J = 7.0, 1.7 Hz, 1H), 4.61 (t, J = 7.8 Hz, 1H), 3.45-3.40 (m, 1H), 3.14 (dd, J = 13.9, 7.2 Hz, 1H), 2.29-2.17 (m, 6H). |

### Procedure G

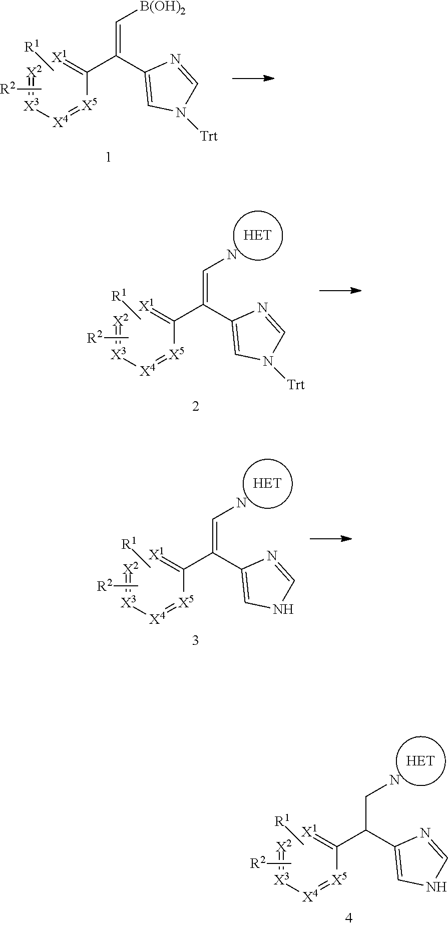

### Example of Procedure G: Synthesis of Compound 406

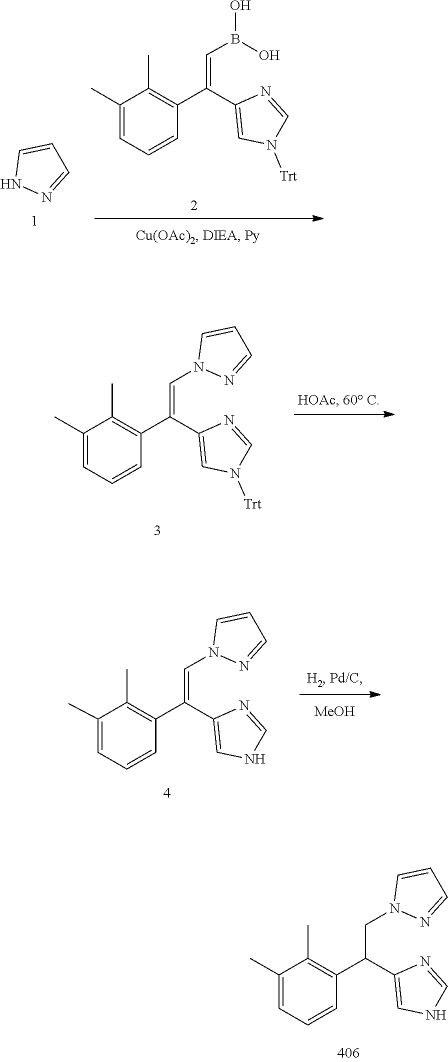

### Experimental Details

### 1. Synthesis of 406

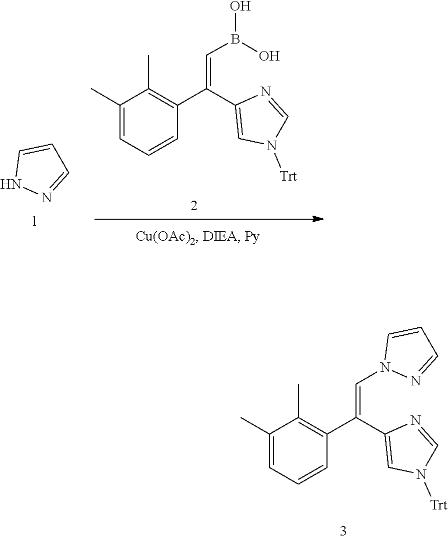

### 4-[(Z)-1-(2,3-dimethylphenyl)-2-(pyrazol-1-yl)ethenyl]-1-(triphenylmethyl)imidazole

A solution of pyrazole (200 mg, 2.94 mmol, 1.0 equiv) and (Z)-2-(2,3-dimethylphenyl)-2-[1-(triphenylmethyl)imidazol-4-yl]ethenylboronic acid (1.42 g, 2.94 mmol, 1.0 equiv), copper(II) acetate (1.60 g, 8.81 mmol, 3.0 equiv), N,N-diisopropylethylamine (1.10 g, 8.81 mmol, 3.0 equiv) in pyridine (10 mL) was stirred for overnight at 100° C. under air atmosphere. The mixture was filtered. The reaction was quenched by the addition of water. The resulting mixture was extracted with ethyl acetate (3×20 mL). The combined organic layers were washed with water (3×20 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% FA), 10% to 50% gradient in 20 min; detector, UV 254 nm to afford 4-[(Z)-1-(2,3-dimethylphenyl)-2-(pyrazol-1-yl)ethenyl]-1-(triphenylmethyl)imidazole (600 mg, 40.31% yield) as a yellow oil. LCMS (ESI) [M+H]⁺: 507.

### 2. Synthesis of 4

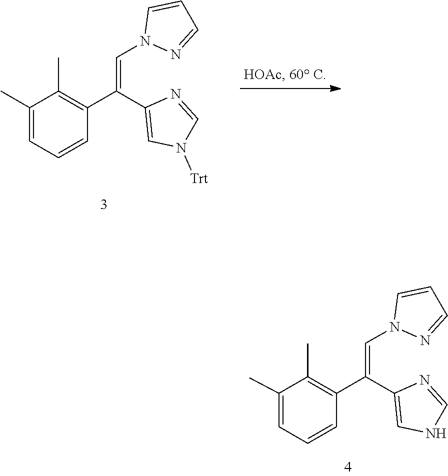

### (Z)-1-(2-(2,3-dimethylphenyl)-2-(1H-imidazol-4-yl)vinyl)-1H-pyrazole

A solution of 4-[(Z)-1-(2,3-dimethylphenyl)-2-(pyrazol-1-yl)ethenyl]-1-(triphenylmethyl)imidazole (600 mg, 1.08 mmol, 1.0 equiv) in acetic acid (5 mL) was stirred for 2 h at 60° C. under nitrogen atmosphere. The resulting mixture was concentrated under reduced pressure. The residue was purified by reversed-phase flash chromatography with the following conditions: column, C18 silica gel; mobile phase, acetonitrile in water (10 mmol/L NH₄HCO₃), 10% to 50% gradient in 20 min; detector, UV 220 nm to afford (Z)-1-(2-(2,3-dimethylphenyl)-2-(1H-imidazol-4-yl)vinyl)-1H-pyrazole (210 mg, 62.06% yield) as a yellow oil. LCMS (ESI) [M+H]⁺: 265.

### 3. Synthesis of 406

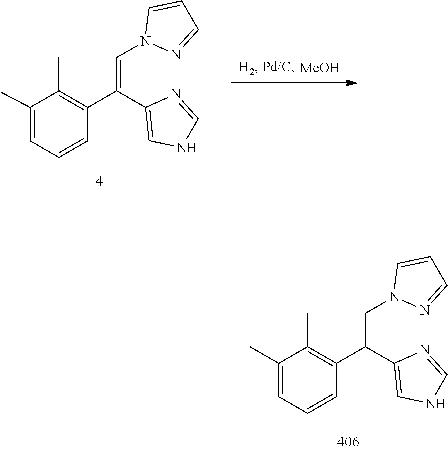 [SMILES: "MeOH" ; Cc1cccc(/C(=C/n2cccn2)c2c[nH]cn2)c1C ; Cc1cccc(C(Cn2cccn2)c2c[nH]cn2)c1C]

### 4-[1-(2,3-dimethylphenyl)-2-(pyrazol-1-yl)ethyl]-1H-imidazole

A solution of (Z)-1-(2-(2,3-dimethylphenyl)-2-(1H-imidazol-4-yl)vinyl)-1H-pyrazole (200 mg, 0.76 mmol, 1.0 equiv) and Pd/C (50 mg) in methanol (10 mL) was stirred for 6 h at room temperature under hydrogen atmosphere. The resulting mixture was filtered, the filter cake was washed with methanol (3×10 mL). The filtrate was concentrated under reduced pressure. The crude product was purified by Prep-HPLC with the following conditions (Column: YMC-Actus Triart C18 ExRS 30*150 mm, 5m; Mobile Phase A: Water (10 mmol/L NH₄HCO₃), Mobile Phase B: ACN; Flow rate: 60 mL/min; Gradient: 22% B to 46% B in 7 min; Wave Length: 254 nm/220 nm; RT1 (min): 6.42) to afford 4-[1-(2,3-dimethylphenyl)-2-(pyrazol-1-yl)ethyl]-1H-imidazole (81.6 mg, 38.99% yield) as a white solid. LCMS (ESI) [M+H]⁺: 267.15. ¹H NMR (400 MHz, DMSO-d6) δ 11.84 (d, J=29.1 Hz, 1H), 7.59-7.45 (m, 1H), 7.41-7.29 (m, 2H), 7.25-7.23 (m 1H), 7.05-6.99 (m, 1H), 6.99-6.93 (m, 1H), 6.83 (s, 1H), 6.05-6.03 (m, 1H), 4.89-4.87 (m, 1H), 4.78-4.66 (m, 1H), 4.48-4.46 (m, 1H), 2.19-2.15 (m, 6H).

TABLE G

| | | | | Mo- | | | |
|---|---|---|---|---|---|---|---|
| Pro- | | | | lecular | LCMS | Puri- | |
| ce- | | | | weight | [M + | ty | |
| dure | Name | Structure | IUPAC | (g/mol) | H]+ | (%) | 1H NMR |
| G | 406 | 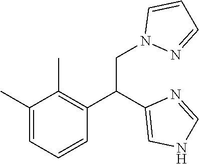 [SMILES: Cc1cccc(C(Cn2cccn2)c2c[nH]cn2)c1C] | 1-[2-(4-imi- dazolyl)-2-(2,3- xylyl)ethyl]pyra- zole | 266.348 | 267.15 | 96.4 | 1H NMR (400 MHz, DMSO-d6) δ 11.84 (d, J = 29.1 Hz, 1H), 7.59-7.45 (m, 1H), 7.41-7.29 (m, 2H), 7.25 (dd, J = 7.5, 1.8 Hz, 1H), 7.05-6.99 (m, 1H), 6.99-6.93 (m, 1H), 6.83 (s, 1H), 6.05 (dt, J = 21.2, 2.0 Hz, 1H), 4.89 (dt, J = 51.2, 7.6 Hz, 1H), 4.78-4.66 (m, 1H), 4.48 (dd, J = 13.3, 7.3 Hz, 1H), 2.19 (dd, J = 13.9, 9.2 Hz, 6H). |
| G | 408 | 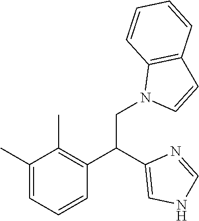 [SMILES: Cc1cccc(C(Cn2ccc3ccccc32)c2c[nH]cn2)c1C] | 4-[2-(1-indolyl)- 1-(2,3- xylyl)ethyl]imi- dazole | 315.42 | 316.2 | 96.9 | 1H NMR (400 MHz, DMSO-d6) δ 11.74 (s, 1H), 8.32 (s, 1H), 7.56 (s, 1H), 7.44 (dt, J = 7.9, 5.5 Hz, 3H), 7.13-7.01 (m, 3H), 7.01-6.89 (m, 2H), 6.82 (s, 1H), 6.24 (d, J = 3.2 Hz, 1H), 4.92-4.73 (m, 2H), 4.57 (dd, J = 12.7, 6.6 Hz, 1H), 2.14 (s, 3H), 1.99 (s, 3H). |

### Procedure H

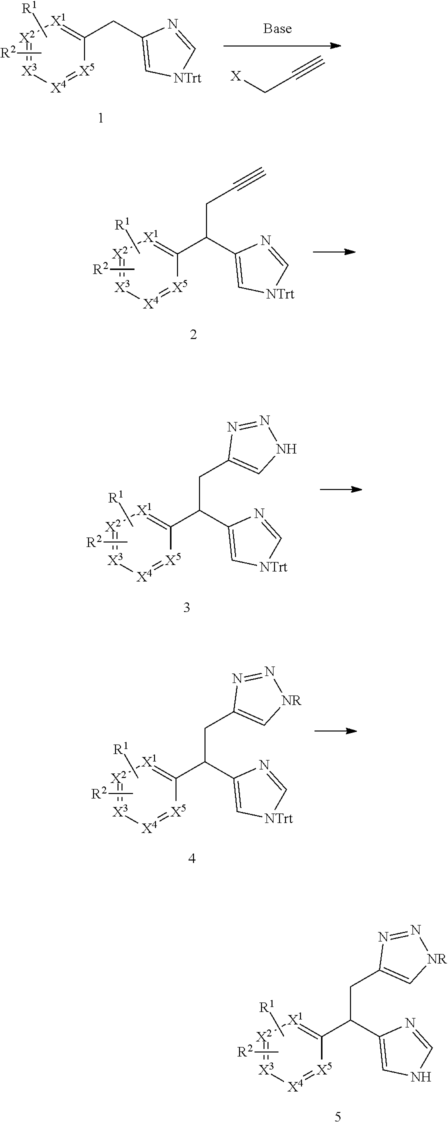

### Example of Procedure H: Synthesis of Compound 404

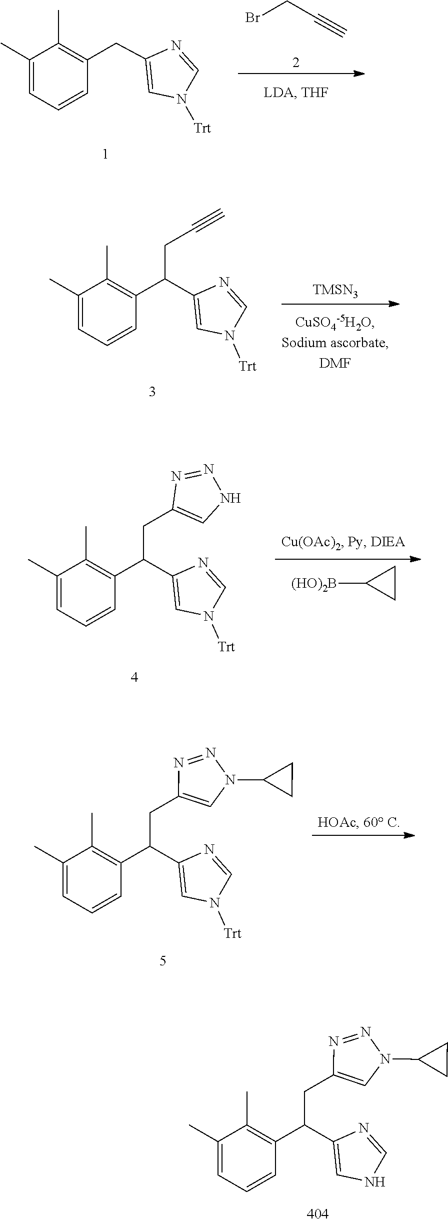

### Experimental Details

### 1. Synthesis of 3

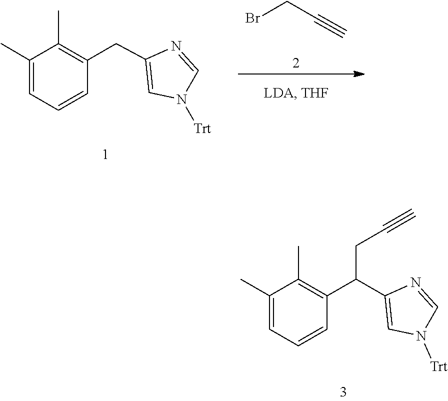

### 4-[1-(2,3-dimethylphenyl)but-3-yn-1-yl]-1-(triphenylmethyl)imidazole

A solution of 4-[(2,3-dimethylphenyl)methyl]-1-(triphenylmethyl)imidazole (2 g, 4.67 mmol, 1.0 equiv) in THF (20 mL) was treated with lithium diisopropylamide (4.67 mL, 9.33 mmol, 2.0 equiv) for 30 min at −78° C. under nitrogen atmosphere followed by the addition of propargyl bromide (1.11 g, 9.33 mmol, 2.0 equiv) dropwise at −78° C. The resulting mixture was stirred for additional 1 h at −78° C. The reaction was quenched by the addition of sat. ammonium chloride (aq.) at room temperature. The resulting mixture was extracted with ethyl acetate (3×50 mL). The combined organic layers were dried over anhydrous sodium sulfate. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 220 nm to afford 4-[1-(2,3-dimethylphenyl)but-3-yn-1-yl]-1-(triphenylmethyl)imidazole (1 g, 45.92% yield) as a white solid. LCMS (ESI) [M+H]⁺: 467.

### 2. Synthesis of 4

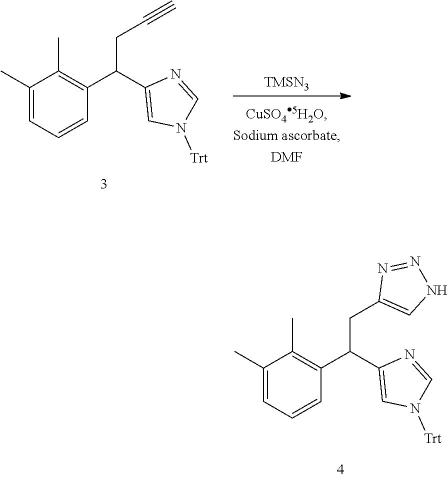

### 4-[2-(2,3-dimethylphenyl)-2-[1-(triphenylmethyl)imidazol-4-yl]ethyl]-1H-1,2,3-triazole

A mixture of 4-[1-(2-methylphenyl)but-3-yn-1-yl]-1-(triphenylmethyl)imidazole (990 mg, 2.1 mmol, 1.0 equiv) and azidotrimethylsilane (483 mg, 4.2 mmol, 2.0 equiv), copper(II) sulfate pentahydrate (80 mg, 0.32 mmol, 0.15 equiv) and sodium ascorbate (146 mg, 0.74 mmol, 0.35 equiv) in DMF (10 mL) was stirred for 1 h at 60° C. under nitrogen atmosphere. The reaction was quenched by the addition of water (2 mL) at room temperature. The aqueous solution was extracted with ethyl acetate (20 mL×3). The combined organic solution was dried over anhydrous sodium sulfate and concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 220 nm to afford 4-[2-(2,3-dimethylphenyl)-2-[1-(triphenylmethyl)imidazol-4-yl]ethyl]-1H-1,2,3-triazole (500 mg, 46.33% yield) as a yellow oil. LCMS (ESI) [M+H]⁺: 510.

### 3. Synthesis of 5

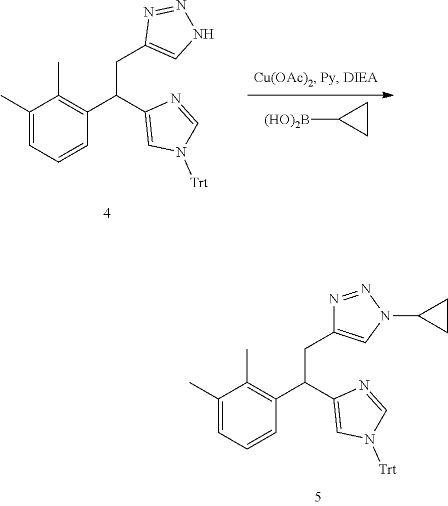

### 1-cyclopropyl-4-[2-(2,3-dimethylphenyl)-2-[1-(triphenylmethyl)imidazol-4-yl]ethyl]-1,2,3-triazole

A solution of 4-[2-(2,3-dimethylphenyl)-2-[1-(triphenylmethyl)imidazol-4-yl]ethyl]-1H-1,2,3-triazole (500 mg, 0.98 mmol, 1.0 equiv), cyclopropylboronic acid (843 mg, 9.81 mmol, 10.0 equiv), N,N-diisopropylethylamine (1268 mg, 9.81 mmol, 10.0 equiv) and copper(II) acetate (535 mg, 2.94 mmol, 3.0 equiv) in pyridine (5 mL) was stirred for overnight at 100° C. under air atmosphere. The reaction was quenched by the addition of water (20 mL) at room temperature. The aqueous solution was extracted with ethyl acetate (20 mL×3). The combined organic solution was dried over anhydrous sodium sulfate and concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 220 nm to afford 1-cyclopropyl-4-[2-(2,3-dimethylphenyl)-2-[1-(triphenylmethyl)imidazol-4-yl]ethyl]-1,2,3-triazole (200 mg, 37.08% yield) as a yellow oil. LCMS (ESI) [M+H]⁺: 550.

### 4. Synthesis of 404

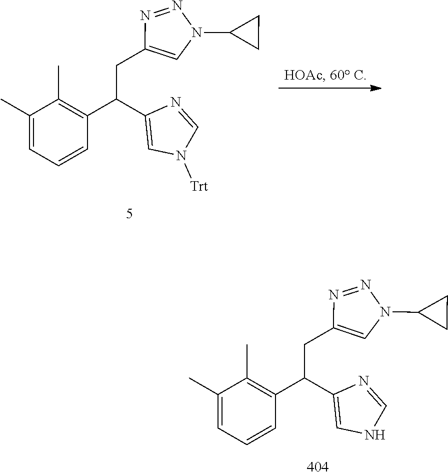

### 1-cyclopropyl-4-[2-(2,3-dimethylphenyl)-2-(1H-imidazol-4-yl)ethyl]-1,2,3-triazole

A solution of 1-cyclopropyl-4-[2-(2,3-dimethylphenyl)-2-[1-(triphenylmethyl)imidazol-4-yl]ethyl]-1,2,3-triazole (200 mg, 0.36 mmol, 1.0 equiv) in acetic acid (5 mL) was stirred for 1 h at 60° C. under air atmosphere. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 220 nm to afford 1-cyclopropyl-4-[2-(2,3-dimethylphenyl)-2-(1H-imidazol-4-yl)ethyl]-1,2,3-triazole (47.2 mg, 42.20% yield) as a yellow solid. LCMS (ESI) [M+H]⁺: 308.15. ¹H NMR (400 MHz, DMSO-d₆) δ 11.77 (br s, 1H), 7.52 (br s, 1H), 7.14 (d, J=13.1 Hz, 2H), 7.04-6.86 (m, 2H), 6.78 (br s, 1H), 4.57 (s, 1H), 3.98-3.93 (m, 1H), 3.27-3.24 (m, 1H), 3.10 (dd, J=14.7, 7.0 Hz, 1H), 2.20-2.17 (m, 6H), 1.11-0.93 (m, 4H).

TABLE H

| | | | | Mo- | | | |
|---|---|---|---|---|---|---|---|
| Pro- | | | | lecular | LCMS | Puri- | |
| ce- | | | | weight | [M + | ty | |
| dure | Name | Structure | IUPAC | (g/mol) | H]+ | (%) | 1H NMR |
| H | 404 | 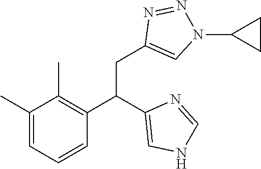 [SMILES: Cc1cccc(C(Cc2cn(C3CC3)nn2)c2c[nH]cn2)c1C] | 1-cyclopropyl- 4-[2-(4-imi- dazolyl)-2-(2,3- xylyl)ethyl]- 1H-1,2,3- triazole | 307.401 | 308.15 | 98.7 | ¹H NMR (400 MHz, DMSO-d₆) δ 11.77 (br s, 1H), 7.52 (br s, 1H), 7.14 (d, J = 13.1 Hz, 2H), 7.04-6.86 (m, 2H), 6.78 (br s, 1H), 4.57 (s, 1H), 3.98-3.93 (m, 1H), 3.27-3.24 (m, 1H), 3.10 (dd, J = 14.7, 7.0 Hz, 1H), 2.20-2.17 (m, 6H), 1.11-0.93 (m, 4H). |
| H | 407 | 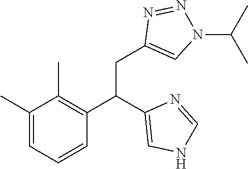 [SMILES: Cc1cccc(C(Cc2cn(C(C)C)nn2)c2c[nH]cn2)c1C] | 4-[2-(4-imi- dazolyl)- 2-(2,3- xylyl)ethyl]- 1-isopropyl- 1H- 1,2,3-triazole | 309.417 | 310.2 | 99.7 | ¹H NMR (400 MHz, DMSO- d₆) δ 11.72 (s, 1H), 7.59-7.40 (m, 2H), 7.19-6.91 (m, 3H), 6.79 (s, 1H), 4.72-4.60 (m, 1H), 4.54 (s, 1H), 3.41 (s, 1H), 3.07 (dd, J = 14.4, 7.1 Hz, 1H), 2.21 (d, J = 7.5 Hz, 3H), 2.16 (d, J = 5.4 Hz, 3H), 1.47-1.28 (m, 6H). |
| H | 409 | 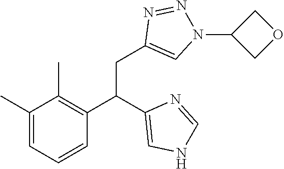 [SMILES: Cc1cccc(C(Cc2cn(C3COC3)nn2)c2c[nH]cn2)c1C] | 4-[2-(4-imi- dazolyl)- 2-(2,3- xylyl)ethyl]- 1-(3-oxetanyl)- 1H- 1,2,3-triazole | 323.4 | 324.2 | 99.3 | 1H NMR (400 MHz, DMSO- d6) δ 11.75 (s, 1H), 7.82 (d, J = 47.5 Hz, 1H), 7.52 (s, 1H), 7.19-7.17 (m, 1H), 7.00-6.81 (m, 3H), 5.71 (p, J = 6.7 Hz, 1H), 4.95 (t, J = 7.1 Hz, 2H), 4.77 (t, J = 6.4 Hz, 2H), 4.61 (d, J = 8.7 Hz, 1H), 3.45 (d, J = 14.0 Hz, 1H), 3.14 (dd, J = 14.8, 6.8 Hz, 1H), 2.31-2.11 (m, 6H). |

### Procedure I

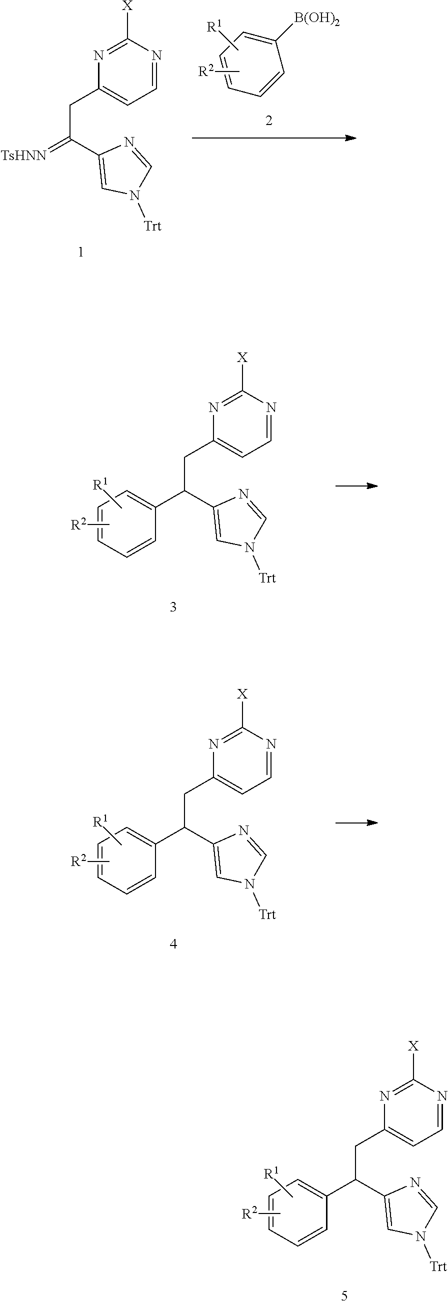

### Example of Procedure I: Synthesis of Compound 405

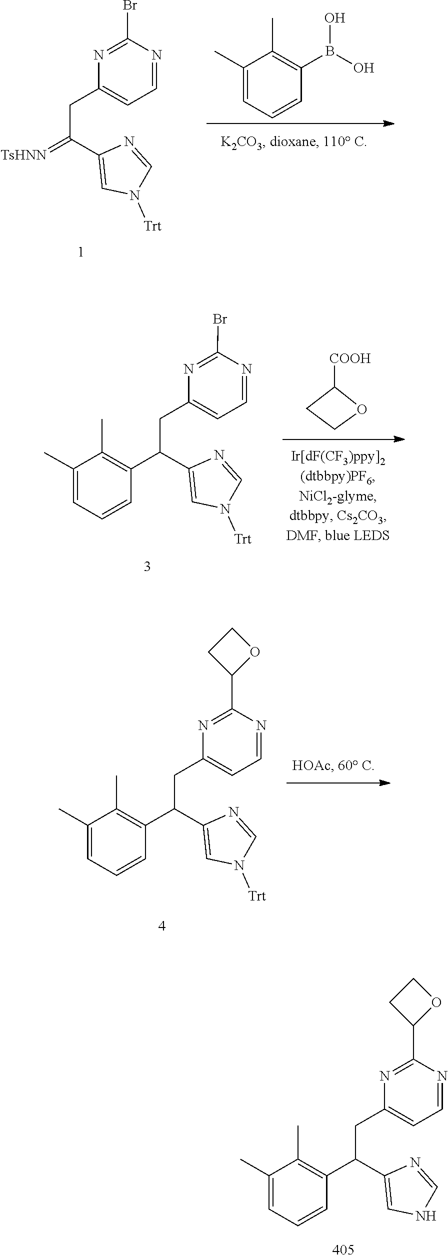

### Experimental Details

### 1. Synthesis of 3

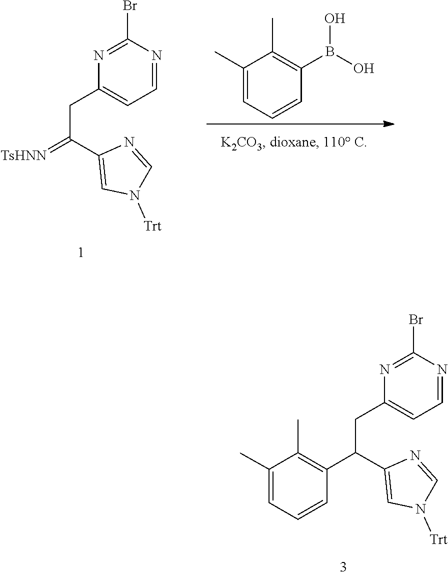

### 2-Bromo-4-[2-(2,3-dimethylphenyl)-2-[1-(triphenylmethyl)imidazol-4-yl]ethyl]pyrimidine

To a stirred mixture of N′-[(1E)-2-(2-bromopyrimidin-4-yl)-1-[1-(triphenylmethyl)imidazol-4-yl]ethylidene]-4-methylbenzenesulfonohydrazide (2.5 g, 3.69 mmol, 1.0 equiv) and 2,3-dimethylphenylboronic acid (1.1 g, 7.38 mmol, 2.0 equiv) in dioxane (60 mL) was added potassium carbonate (1.5 g, 11.07 mmol, 3.0 equiv) at room temperature under nitrogen atmosphere. The resulting mixture was stirred at 110° C. for 1 h under nitrogen atmosphere. The reaction was quenched by the addition of water at room temperature. The resulting mixture was extracted with ethyl acetate (3×50 mL). The combined organic layers were dried over anhydrous sodium sulfate. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 5% to 100% gradient in 30 min; detector, UV 254 nm. to afford 2-bromo-4-[2-(2,3-dimethylphenyl)-2-[1-(triphenylmethyl)imidazol-4-yl]ethyl]pyrimidine (500 mg, 22.60% yield) as a yellow solid. LCMS (ESI) [M+H]⁺: 599.

### 2. Synthesis of 4

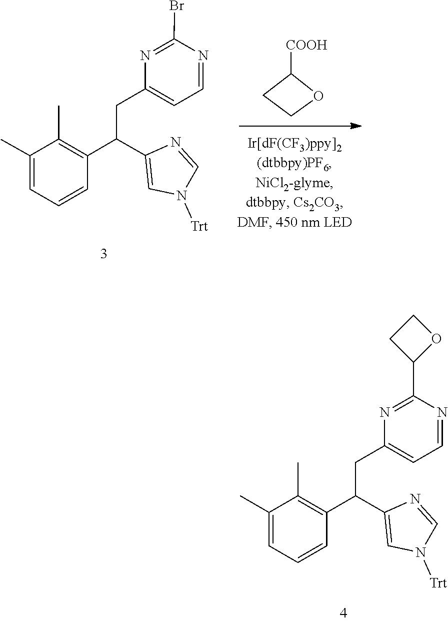

### 4-[2-(2,3-dimethylphenyl)-2-[1-(triphenylmethyl)imidazol-4-yl]ethyl]-2-(oxetan-2-yl)pyrimidine

Into a 10 mL vial were added 2-bromo-4-[2-(2,3-dimethylphenyl)-2-[1-(triphenylmethyl)imidazol-4-yl]ethyl]pyrimidine (300 mg, 0.50 mmol, 1.0 equiv), oxetane-2-carboxylic acid (77 mg, 0.75 mmol, 1.5 equiv), cesium carbonate (8 mg, 0.03 mmol, 1.5 equiv), Ir[dF(CF₃)ppy]₂(dtbpy))PF₆ (0.19 mg, 0.01 equiv), 4,4′-di-tert-butyl-2,2′-dipyridyl (7 mg, 0.03 mmol, 0.15 equiv) and DMF (5 mL) at room temperature under nitrogen atmosphere. The reaction mixture was sparged with nitrogen for 15 minutes before sealing with parafilm. The vial was stirred at 1500 rpm stir rate and irradiated under 450 nm LED modules at 100% light intensity with maxed fan speed of 1500 rpm stirring rate in a PennOC Integrated Photoreactor for 2 hours. The reaction was quenched with water at room temperature. The aqueous layer was extracted with ethyl acetate (3×10 mL). After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 100% gradient in 30 min; detector, UV 254 nm. This resulted in 4-[2-(2,3-dimethylphenyl)-2-[1-(triphenylmethyl)imidazol-4-yl]ethyl]-2-(oxetan-2-yl)pyrimidine (170 mg, 58.91% yield) as a yellow solid. LCMS (ESI) [M+H]⁺: 577.

### 3. Synthesis of 405

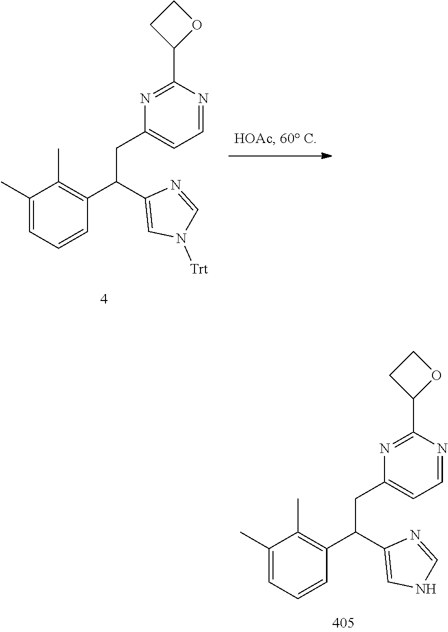

### 4-[2-(2,3-dimethylphenyl)-2-(1H-imidazol-4-yl)ethyl]-2-(oxetan-2-yl)pyrimidine

A mixture of 4-[2-(2,3-dimethylphenyl)-2-[1-(triphenylmethyl)imidazol-4-yl]ethyl]-2-(oxetan-2-yl)pyrimidine (120 mg, 0.21 mmol, 1.0 equiv) in acetic acid (10 mL) was stirred at 60° C. for 1h under nitrogen atmosphere. The resulting mixture was concentrated under reduced pressure. The crude product was purified by Prep-hPLC with the following conditions (Column: XSelect CSH Prep C18 OBD Column, 30*150 mm, 5m; Mobile Phase A: Water (0.1% FA), Mobile Phase B: ACN; Flow rate: 60 mL/min; Gradient: 4% B to 17% B in 9 min; Wave Length: 254 nm/220 nm; RT1 (min): 11.65) to afford 4-[2-(2,3-dimethylphenyl)-2-(1H-imidazol-4-yl)ethyl]-2-(oxetan-2-yl)pyrimidine (17.2 mg, 24.72% yield) as a yellow semi-solid. LCMS (ESI) [M+H]⁺: 335.10. ¹H NMR (400 MHz, DMSO-d₆) δ 11.99 (s, 1H), 8.59 (d, J=5.1 Hz, 1H), 8.19 (s, 1H), 7.52-7.46 (m, 1H), 7.16 (t, J=5.9 Hz, 2H), 6.95 (d, J=13.5, 7.3 Hz, 2H), 6.76-6.74 (m 1H), 5.66-5.59 (m, 1H), 4.90-4.88 (m, 1H), 4.71-4.59 (m, 2H), 3.57-3.48 (m, 1H), 3.28-3.26 (m, 1H), 3.00-2.88 (m, 1H), 2.87-2.75 (m, 1H), 2.24-2.18 (m, 6H).

TABLE I

| | | | | Mo- | | | |
|---|---|---|---|---|---|---|---|
| Pro- | | | | lecular | LCMS | | |
| ce- | | | | weight | [M + | Purity | |
| dure | Name | Structure | IUPAC | (g/mol) | H]+ | (%) | 1H NMR |
| 1 | 405 | 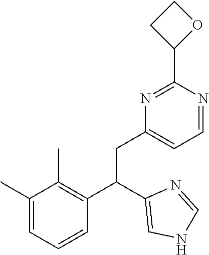 [SMILES: Cc1cccc(C(Cc2ccnc(C3CCO3)n2)c2c[nH]cn2)c1C] | 4-[2-(4-imidazolyl)- 2-(2,3-xylyl)eth- yl]-2-(2-oxe- tanyl)pyrimidine | 334.423 | 335.1 | 97.20% | ¹H NMR (400 MHz, DMSO- d₆) δ 12.00 (s, 1H), 8.59 (d, J = 5.1 Hz, 1H), 8.19 (s, 1H), 7.49 (t, J = 1.5 Hz, 1H), 7.16 (t, J = 5.9 Hz, 2H), 6.95 (dt, J = 13.4, 7.3 Hz, 2H), 6.76 (d, J = 2.7 Hz, 1H), 5.63- 5.60 (m, 1H), 4.90 (s, 1H), 4.65 (s, 2H), 3.59-3.50 (m, 1H), 3.30-3.24 (m, 1H), 3.03-2.88 (m, 1H), 2. 81 (m, 1H), 2.29-2.14 (m, 6H). |

### Biological Assays and Data

GTP [symbol] S Agonist Assays (ADRA2A, ADRA2B, and ADRA2C Receptors)

Membranes were prepared for GTP [symbol] S binding assays from CHO-K1 cells stably expressing human ADRA2A, ADRA2B, or ADRA2C receptor. Briefly, cells were grown in 5-layer cell culture flasks. Cells were washed with 50 mL of PBS and then dissociated with 0.05% trypsin. Dissociated cells were then collected by centrifugation at 650×g for 8 min at 4° C. Pellets were washed again in PBS by centrifugation and resuspended in 30 mL of 20 mM HEPES, 10 mM EDTA, pH 7.4. After sitting on ice for 30 min, membranes were collected by centrifugation at 50000×g for 15 min at 4° C., resuspended in 30 mL of 20 mM HEPES, 0.1 mM EDTA, pH 7.4 and collected again by centrifugation. Pellets were then washed twice by centrifugation in 30 mL of 20 mM HEPES, pH 7.4, and stored at −80° C. until the day of the assay. On the day of the assay, pellets were thawed on ice and resuspended in 1 mL of 20 mM HEPES, pH 7.4, 10 mM MgCl₂, 100 mM NaCl using dounce homogenization. Membrane protein concentration was determined by BCA protein assay (Promega, Madison, WI) according to Promega guidelines.

On the day of the assay, serial dilutions of test compounds were brought up in assay buffer (20 mM HEPES, 10 mM MgCl₂, 200 mM NaCl) at 10× final assay concentration. 50 μL/well of membrane homogenates (containing 5 μg of membrane protein for ADRA2A, ADRA2B, or 7.5 μg for ADRA2C) were prepared in assay buffer supplemented with GDP at 2× final assay concentration (5 μM GDP) and dispensed into 96 well polypropylene, U-bottom Greiner plates. Then, 10 μL of compounds were added to assay plates and incubated for 10 min at room temperature. 40 μL of 2.5 nM ³⁵S-GTP (Perkin Elmer, Waltham, MA) in assay buffer was added to assay mixtures. The binding reaction was incubated for 1 h at 37° C. Membranes in assay reactions were then transferred to GF/C filter plates (UniFilter, Perkin Elmer) and washed three times using a FilterMate Harvester (Perkin Elmer) with cold assay buffer containing no GDP. Filter plates were then dried at 40° C. overnight followed by the addition of 30 μL/well of Beta Plate Scintillation liquid. Plates were top and bottom sealed before being read on TopCount scintillation counter (Perkin Elmer, Waltham, MA).

### ADRA2A and ADRA2B Arrestin Agonist Assay Method

The arrestin assay uses frozen CHO-K1 cell lines stably expressing human ADRA2A and ADRA2B receptors. Cells are frozen following normal cell freezing protocols. Briefly, cells are washed with PBS and then dissociated with 0.25% trypsin. Dissociated cells are quenched in full media and then collected by centrifugation at 290×g for 5 minutes. Cells are resuspended in cell freeze media (10% DMSO, 90% Avantor Seradigm FB Essence Cat #10803-034) to a concentration of 2×10⁷ cells/mL 1 mL of cells are dispensed into cryovial tubes and frozen in a controlled rate cryo-freezer container at −80° C. Cells are stored at −80° C. (or in liquid N2 if stored >6 months) until the day of the assay.

On the day before the assay, frozen cells are thawed in a 37° C. water bath, brought up in 20 mL of full media, and collected via centrifugation at 290×g for 5 minutes. Cells are washed in PBS by centrifugation and resuspended in Opti-MEM (Gibco Cat #31985070) to a concentration of 1×10⁶ cells/mL 10 μl/well of cells (10,000 cells/well) are dispensed into 384 well corning plates, in triplicate. Cells are incubated overnight in a 37° C., 5% CO₂ cell incubator.

On the day of the assay, agonists are diluted in Opti-MEM to 3.5x of the final assay concentration. 4 μL of agonists are added to the cells and incubated for 2 hours at 37° C. in a 5% CO₂ cell incubator. 6 μL of arrestin detection reagents are dispensed onto the cells and the plates are incubated for 2 hours at room temperature. Arrestin detection reagent consists of 1-part Galacton-star substrate (Thermo Fisher Cat #T2739), 5-parts Emerald-II enhancer concentrate (Thermo Fisher Cat #T2382C) and in 25-parts buffer (100 mM Dibasic Potassium phosphate, 10 mM Monobasic Potassium Phosphate at pH 7.8) with 0.35% Triton X-100 (w/v). Plates are read on an Envision luminescence plate reader.

### Triaryl Agonists of Alpha-2 Adrenergic Receptor

| Code | EC50 | |
|---|---|---|
| **** | <10 | nM |
| *** | 11-50 | nM |
| ** | 51-500 | nM |
| * | >500 | nM |

| | GTPase - | Beta-Arrestin - |
|---|---|---|
| | Human Alpha2B | Human Alpha2B |
| Name | EC50 | EC50 |
| 1 | * | |
| 2 | **** | |
| 3 | **** | |
| 4 | ** | |
| 5 | **** | |
| 6 | ** | |
| 7 | *** | |
| 8 | * | |
| 9 | **** | |
| 10 | * | |
| 11 | **** | |
| 12 | * | |
| 13 | * | |
| 14 | ** | |
| 15 | **** | |
| 16 | **** | |
| 17 | ** | |
| 18 | * | |
| 19 | *** | |
| 20 | **** | |
| 21 | *** | |
| 22 | * | |
| 23 | **** | |
| 24 | * | |
| 25 | ** | |
| 26 | ** | |
| 27 | **** | |
| 28 | **** | |
| 29 | * | |
| 30 | * | |
| 31 | * | |
| 32 | * | |
| 33 | * | |
| 34 | * | |
| 35 | * | |
| 36 | *** | |
| 37 | *** | |
| 38 | * | |
| 39 | * | |
| 40 | *** | |
| 41 | **** | |
| 42 | ** | |
| 43 | * | |
| 44 | *** | |
| 45 | * | |
| 46 | * | |
| 47 | * | |
| 48 | *** | |
| 49 | ** | |
| 50 | * | |
| 51 | ** | |
| 52 | *** | |
| 53 | **** | |
| 54 | ** | |
| 55 | *** | |
| 56 | * | |
| 57 | * | |
| 58 | ** | |
| 59 | **** | |
| 60 | **** | |
| 61 | ** | |
| 62 | * | |
| 63 | **** | |
| 64 | * | |
| 65 | *** | |
| 66 | ** | |
| 67 | *** | |
| 68 | * | |
| 69 | **** | |
| 70 | **** | |
| 71 | * | |
| 72 | **** | |
| 73 | * | |
| 74 | * | |
| 75 | ** | |
| 76 | * | |
| 77 | **** | |
| 78 | * | |
| 79 | ** | |
| 80 | **** | |
| 81 | * | |
| 82 | ** | |
| 83 | **** | |
| 84 | **** | |
| 85 | *** | |
| 86 | **** | |
| 87 | * | |
| 88 | *** | |
| 89 | **** | |
| 90 | **** | |
| 91 | **** | |
| 92 | *** | |
| 93 | ** | |
| 94 | ** | |
| 95 | **** | |
| 96 | *** | |
| 97 | **** | |
| 98 | **** | |
| 99 | * | |
| 100 | *** | |
| 101 | ** | |
| 102 | *** | |
| 103 | * | |
| 104 | * | |
| 105 | * | |
| 106 | *** | |
| 107 | * | |
| 108 | ** | |
| 109 | **** | |
| 110 | **** | |
| 111 | ** | |
| 112 | ** | |
| 113 | ** | |
| 114 | **** | |
| 115 | ** | |
| 116 | *** | |
| 117 | ** | |
| 118 | *** | |
| 119 | **** | |
| 120 | * | |
| 121 | ** | |
| 122 | * | |
| 123 | **** | |
| 124 | *** | |
| 125 | *** | |
| 126 | **** | |
| 127 | **** | |
| 128 | ** | |
| 129 | **** | |
| 130 | **** | |
| 131 | *** | |
| 132 | **** | |
| 133 | *** | |
| 134 | **** | |
| 135 | * | |
| 136 | **** | |
| 137 | ** | |
| 138 | * | |
| 139 | | * |
| 140 | | * |
| 141 | | **** |
| 142 | | * |
| 143 | | **** |
| 144 | | * |
| 145 | | *** |
| 146 | | **** |
| 147 | | * |
| 148 | ** | |
| 149 | *** | |
| 150 | | **** |
| 151 | | ** |
| 152 | ** | |
| 153 | | **** |
| 154 | | *** |
| 155 | | **** |
| 156 | | **** |
| 157 | | **** |
| 158 | | **** |
| 159 | ** | |
| 160 | * | |
| 161 | | * |
| 162 | | * |
| 163 | | ** |
| 164 | **** | |
| 165 | | ** |
| 166 | * | |
| 167 | | **** |
| 168 | | *** |
| 169 | | **** |
| 170 | ** | |
| 171 | ** | |
| 172 | * | |
| 173 | * | |
| 174 | ** | |
| 175 | | * |
| 176 | | **** |
| 177 | ** | |
| 178 | *** | |
| 179 | | **** |
| 180 | | * |
| 181 | | *** |
| 182 | | *** |
| 183 | | * |
| 184 | ** | |
| 185 | **** | |
| 186 | **** | |
| 187 | | ** |
| 188 | * | |
| 189 | | * |
| 190 | | *** |
| 191 | | *** |
| 192 | | **** |
| 193 | **** | |
| 194 | *** | |
| 195 | | *** |
| 196 | ** | |
| 197 | | ** |
| 198 | *** | |
| 199 | * | |
| 200 | **** | |
| 201 | * | |
| 202 | | **** |
| 203 | * | |
| 204 | * | |
| 205 | | **** |
| 206 | | **** |
| 207 | | **** |
| 208 | | **** |
| 209 | | **** |
| 210 | | **** |
| 211 | | * |
| 212 | | **** |
| 213 | *** | |
| 214 | * | |
| 215 | | |
| 216 | | |
| 217 | * | |
| 218 | * | |
| 219 | * | |
| 220 | * | |
| 221 | * | |
| 222 | * | |
| 223 | * | |
| 224 | * | |
| 225 | * | |
| 226 | * | |
| 227 | * | |
| 228 | * | |
| 229 | * | |
| 230 | * | |
| 231 | * | |
| 232 | | * |
| 233 | | **** |
| 234 | | * |
| 235 | | **** |
| 236 | | ** |
| 237 | | * |
| 238 | | *** |
| 239 | | * |
| 240 | | **** |
| 241 | *** | |
| 242 | | * |
| 243 | | ** |
| 244 | | **** |
| 245 | | **** |
| 246 | | ** |
| 247 | | ** |
| 248 | | **** |
| 249 | | **** |
| 250 | | * |
| 251 | | ** |
| 252 | | *** |
| 253 | | * |
| 254 | | *** |
| 255 | | **** |
| 256 | | ** |
| 257 | | * |
| 258 | | *** |
| 259 | | *** |
| 260 | | ** |
| 261 | | **** |
| 262 | | *** |
| 263 | | ** |
| 264 | | ** |
| 265 | | *** |
| 266 | | * |
| 267 | | ** |
| 268 | | ** |
| 269 | | ** |
| 270 | | **** |
| 271 | **** | |
| 272 | | **** |
| 273 | | **** |
| 274 | | **** |
| 275 | | *** |
| 276 | | ** |
| 277 | *** | |
| 278 | | **** |
| 279 | *** | |
| 280 | | **** |
| 281 | | **** |
| 282 | | * |
| 283 | | **** |
| 284 | | *** |
| 285 | | *** |
| 286 | | **** |
| 287 | | **** |
| 288 | | **** |
| 289 | * | |
| 290 | **** | |
| 291 | | **** |
| 292 | | **** |
| 293 | | *** |
| 294 | | **** |
| 295 | | **** |
| 296 | | *** |
| 297 | | * |
| 298 | | **** |
| 299 | | * |
| 300 | | **** |
| 301 | | *** |
| 302 | | **** |
| 303 | | *** |
| 304 | | **** |
| 305 | | * |
| 306 | | **** |
| 307 | | **** |
| 308 | | *** |
| 309 | *** | |
| 310 | **** | |
| 311 | | **** |
| 312 | *** | |
| 313 | | **** |
| 314 | | **** |
| 315 | | **** |
| 316 | | **** |
| 317 | | **** |
| 318 | | **** |
| 319 | | **** |
| 320 | | * |
| 321 | | **** |
| 322 | | *** |
| 323 | | ** |
| 324 | * | |
| 325 | * | |
| 326 | ** | |
| 327 | *** | |
| 328 | ** | |
| 329 | **** | |
| 330 | **** | |
| 331 | **** | |
| 332 | **** | |
| 333 | **** | |
| 334 | *** | |
| 335 | **** | |
| 336 | **** | |
| 337 | | *** |
| 338 | * | |
| 339 | | ** |
| 340 | | ** |
| 341 | | **** |
| 342 | | **** |
| 343 | | **** |
| 344 | | **** |
| 345 | | **** |
| 346 | | * |
| 347 | | ** |
| 348 | | **** |
| 349 | | * |
| 350 | | ** |
| 351 | | ** |
| 352 | | * |
| 353 | | **** |
| 354 | | * |
| 355 | ** | |
| 356 | | *** |
| 357 | | * |
| 358 | | * |
| 359 | | *** |
| 360 | * | |
| 361 | * | |
| 362 | *** | |
| 363 | * | |
| 364 | | **** |
| 365 | * | |
| 366 | | **** |
| 367 | | * |
| 368 | | ** |
| 369 | | * |
| 370 | | *** |
| 371 | | ** |
| 372 | | ** |
| 373 | | *** |
| 374 | | * |
| 375 | | ** |
| 376 | | *** |
| 377 | | ** |
| 378 | | ** |
| 379 | | **** |
| 380 | | * |
| 381 | | * |
| 382 | | *** |
| 383 | | * |
| 384 | | *** |
| 385 | | **** |
| 386 | | * |
| 387 | | ** |
| 388 | | * |
| 389 | | ** |
| 390 | | *** |
| 391 | | **** |
| 392 | | *** |
| 393 | | *** |
| 394 | | **** |
| 395 | | **** |
| 396 | | **** |
| 397 | | * |
| 398 | | **** |
| 399 | | **** |
| 400 | | * |
| 401 | | **** |
| 402 | | *** |
| 403 | | *** |
| 404 | | **** |
| 405 | | **** |
| 406 | | *** |

| | | |
|---|---|---|
| | GTPase - | Beta-Arrestin - |
| | Human | Human Alpha2B |
| Name | Alpha2B EC50 | EC50 |

| | |
|---|---|
| 407 | *** |
| 408 | *** |
| 409 | **** |

If stereochemistry is not indicated, a name or structural depiction includes any stereoisomer or any mixture of stereoisomers.

Unless otherwise indicated, all numbers expressing quantities of ingredients, properties such as amounts, percentage, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Rounding any value to fewer significant digits is specifically contemplated herein. For example, the term “about 1.1” is intended to contemplate a value of “about 1.” Similarly, a value such as “about 100” is intended to contemplate a value of “about 1×10²,” indicating only a single significant digit, and that values should be rounded so that there is only a single significant digit.

The terms “a,” “an,” “the” and similar referents used in the context of describing the embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the embodiments and does not pose a limitation on the scope of any claim. No language in the specification should be construed as indicating any non-claimed element essential to the practice of the claims.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or to expedite prosecution. When any such inclusion or deletion occurs, the specification is deemed to contain the group as modified thus fulfilling the written description of all Markush groups if used in the appended claims.

Any use of the terms “comprising,” “comprises,” “having,” “includes,” “including,” or the like is intended to also contemplate use of “consisting essentially of,” “consists essentially of,” “consisting of,” or “consists of.”

Certain embodiments are described herein, including the best mode known to the inventors for carrying out the claimed embodiments. Of course, variations on these described embodiments will become apparent to those of ordinary skill in the art upon reading the foregoing description. The inventor expects skilled artisans to employ such variations as appropriate, and the inventors intend for the claimed embodiments to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A compound of formula I: 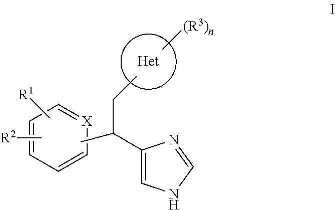
or a pharmaceutically acceptable salt thereof, wherein:
X is N or CR¹;
R¹ is H, F, Cl, Br, I, OH, or a moiety of a formula C₁₋₁₅H₀₋₃₅N₀₋₅O₀₋₆S₀₋₂F₀₋₁₆ which attaches to the phenyl ring or the pyridinyl ring with a carbon atom, a nitrogen atom, an oxygen atom, or a sulfur atom;
R² is H, F, Cl, Br, I, OH, or a moiety of a formula C₁₋₁₅H₀₋₃₅N₀₋₅O₀₋₆S₀₋₂F₀₋₁₆ which attaches to the phenyl ring or the pyridinyl ring with a carbon atom, a nitrogen atom, an oxygen atom, or a sulfur atom;
when R¹ and R² are on adjacent carbon atoms they, together with the phenyl ring to which they are attached, may form a bicyclic heteroaryl ring system that is optionally substituted with 1 or 2 substituents, wherein the substituents of the bicyclic heteroaryl ring system are independently F, Cl, Br, I, OH, NH₂, —OCH₃, —OCH₂CH₃, —CH₃, —C≡CH, —OCF₃, —OCHF₂, or —CF₃;
Each R³ is independently H, F, Cl, Br, I, OH, ═O, or a moiety of a formula C₁₋₁₅H₀₋₃₅N₀₋₅O₀₋₆S₀₋₂F₀₋₁₆; which attaches to the phenyl ring with a carbon atom, a nitrogen atom, an oxygen atom, or a sulfur atom;
n is 0, 1, 2, or 3; and
Het is heteroaryl with one or more ring heteroatoms independently selected from O, S and N.

2. The compound of claim 1, wherein R¹ and R², together with the phenyl ring to which they are attached, form a dihydroindolyl, a dihydrobenzodioxinyl, an indazolyl, an indolyl, a benzoxazolyl, a dihydrobenzodioxepinyl, a quinolinyl, or a dihydroisobenzofuranyl that is optionally substituted with 1 or 2 substituents, wherein the substituents of the dihydroindolyl, the dihydrobenzodioxinyl, the indazolyl, the indolyl, the benzoxazolyl, the dihydrobenzodioxepinyl, the quinolinyl, or the dihydroisobenzofuranyl are independently F, Cl, Br, I, OH, NH₂, —OCH₃, —OCH₂CH₃, —CH₃, —C≡CH, —OCF₃, —OCHF₂, or —CF₃

3. A compound of formula II: 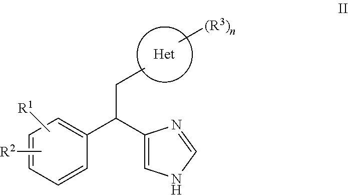
or a pharmaceutically acceptable salt thereof, wherein:
R¹ is H, F, Cl, Br, I, OH, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom;
R² is H, F, Cl, Br, I, OH, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom; when R¹ and R² are on adjacent carbon atoms they, together with the phenyl ring to which they are attached, may form a dihydrobenzofuranyl or a benzodioxolyl that is optionally substituted with 1 or 2 substituents, wherein the substituents of the dihydrobenzofuranyl or the benzodioxolyl are independently F, Cl, Br, I, OH, NH₂, —OCH₃, —OCH₂CH₃, —CH₃, —C≡CH, —OCF₃, —OCHF₂, or —CF₃;
Each R³ is independently H, F, Cl, Br, I, OH, ═O, or a moiety of a formula C₁₋₁₅H₀₋₃₅N₀₋₅O₀₋₆S₀₋₂F₀₋₁₆ which attaches to the phenyl ring with a carbon atom, a nitrogen atom, an oxygen atom, or a sulfur atom;
n is 0, 1, 2, or 3; and
Het is heteroaryl with one or more ring heteroatoms independently selected from O, S and N.
